(19) United States
(12) Patent Application Publication
(10) Pub. No.: US 2011/0160223 A1
(43) Pub. Date: Jun. 30, 2011

(54) NMDA Receptor Antagonists for the Treatment of Neuropsychiatric Disorders

(76) Inventors: Raymond J. Dingledine, Atlanta, GA (US); Stephen F. Traynelis, Decatur, GA (US)

(21) Appl. No.: 12/938,138

(22) Filed: Nov. 2, 2010

Related U.S. Application Data
(63) Continuation of application No. PCT/US09/43502, filed on May 11, 2009.
(60) Provisional application No. 61/127,098, filed on May 9, 2008.

(51) Int. Cl. A61K 31/495; A61K 31/18; A61K 31/496; A61K 31/423; A61K 31/138; A61K 31/451; A61K 31/4166; A61P 25/00

(52) U.S. Cl. 514/253.07; 514/255.03; 514/605; 514/253.01; 514/254.02; 514/375; 514/652; 514/327; 514/392

(57) ABSTRACT

Provided are pharmaceutical compositions and methods of treatment or prophylaxis of certain neuropsychiatric conditions, in particular mood disorders. The compounds are of the general Formula I-V as described herein.

### CROSS-REFERENCE TO RELATED APPLICATION

The application claims priority to U.S. Provisional Patent Application No. 61/127,098, filed May 9, 2008.

### FIELD OF THE INVENTION

The present invention provides certain NMDA receptor blockers, including pH-sensitive NMDA receptor blockers, in the treatment of neuropsychiatric disorders including depression, anxiety and other related diseases.

### BACKGROUND OF THE INVENTION

Glutamate and aspartate play dual roles in the central nervous system as essential amino acids and as the principal excitatory neurotransmitters (hereinafter referred to as excitatory amino acids or EAAs). There are at least four classes of EAA receptors: NMDA, AMPA (2-amino-3-(methyl-3-hydroxyisoxazol-4-yl)propanoic acid), kainate and metabotropic receptors. These EAA receptors mediate a wide range of signaling events that impact all physiological brain functions. For example, it has been reported that NMDA receptor antagonists produce an analgesic effect under certain conditions (Wong, et al. (1995) Acta Anaesthesiologica. Sinica 33, 227-232).

The NMDA subtype of glutamate-gated ion channels mediates excitatory synaptic transmission between neurons in the central nervous system (Dingledine et al. (1999), Pharmacological Reviews 51:7-61). NMDA receptors participate in a wide range of both physiological and pathological processes in the central nervous system. A high density of NMDA receptors has been found in the cortico-limbic regions of the brain which have been postulated to play a role in emotional functions, anxiety and depression (Tzschentke™ (2002) Amino Acids 23:147-152). Extensive studies have demonstrated antidepressant-like effects of various antagonists of the NMDA receptors. The antidepressant-like activity of competitive and non-competitive antagonists and inorganic inhibitors of NMDA receptor (zinc and magnesium) has been reported (see Decollogne, et al. (1997) Pharmacol Biochem Behav 58:261-268; Kroczka, et al. (2001) Brain Res Bull 55:297-300; Kroczka, et al. (2000) Pol J. Pharmacol. 52:403-406; Poleszak, et al. (2004) Pharmacol Biochem Behav 78:7-12; Poleszak, et al. (2007) Pharmacol Biochem Behav 88:158-164; Poleszak, et al. (2007) Pharmacol Rep 57:654-658; Przegaliñski, et al. (1997) Neuropharmacology 36:31-37; Przegaliñski, et al. (1998) Pol J Pharmacol 50: 349-354; Skolnick P Eur J Pharmacol 375:31-40; Skolnick, et al. (2001) Pharmacol Res 43:411-423; and Trullas, et al. (1990) Eur Pharmacol 185:1-10). Poleszak, et al. showed that the NMDA receptor binding of certain antagonists, specifically CGP 37849 and L-701,324, are directly related to their antidepressant-like effects (Poleszak, et al. (2007) Pharm. Reports 59:595-600).

NMDA receptors are composed of NR1, NR2 (A, B, C, and D), and NR3 (A and B) subunits, which determine the functional properties of native NMDA receptors. Expression of the NR1 subunit alone does not produce a functional receptor. Co-expression of one or more NR2 subunits is required to form functional channels. In addition to glutamate, the NMDA receptor requires the binding of a co-agonist, glycine, to allow the receptor to function. A glycine binding site is found on the NR1 and NR3 subunits, whereas the glutamate binding site is found on NR2 subunits. At resting membrane potentials, NMDA receptors are largely inactive due to a voltage-dependent block of the channel pore by magnesium ions. Depolarization releases this channel block and permits passage of calcium as well as other ions.

The NMDA receptor is modulated by a number of endogenous and exogenous compounds including, sodium, potassium and calcium ions that can not only pass through the NMDA receptor channel but also modulate the activity of receptors. Zinc blocks the channel through NR2A- and NR2B-containing receptors noncompetitive and voltage-independent manner. Polyamines can also either potentiate or inhibit glutamate-mediated responses.

Neuropsychiatric disorders including schizophrenia and bipolar disorder and mood disorders affect more than 60 million Americans each year. Four basic forms of mood disorders are major depression, cyclothymia (a mild form of bipolar disorder), SAD (seasonal affective disorder) and mania (euphoric, hyperactive, over inflated ego, unrealistic optimism.) About 20% of the U.S. population reports at least one depressive symptom in a given month, and 12% report two or more in a year. A survey conducted in 1992 found rates of major depression reaching 5% in the previous 30 days, 17% for a lifetime. Bipolar disorder is less common, occurring at a rate of 1% in the general population, but some believe the diagnosis is often overlooked because manic elation is too rarely reported as an illness.

Depression, formally called major depression, major depressive disorder or clinical depression, is a medical illness that involves the mind and body. Most health professionals today consider depression a chronic illness that requires long-term treatment, much like diabetes or high blood pressure. Although some people experience only one episode of depression, most have repeated episodes of depression symptoms throughout their life. Depression is also a common feature of mental illness, whatever its nature and origin. A person with a history of any serious psychiatric disorder has almost as high a chance of developing major depression as someone who has had major depression itself in the past. Most people with major depression also show some signs of anxiety, and 15-30% have panic attacks.

Depression is associated with physical illness as well. Some 25% of hospitalized medical patients have noticeable depressive symptoms and about 5% are suffering from major depression. Chronic medical conditions associated with depression include heart disease, cancer, vitamin deficiencies, diabetes, hepatitis, and malaria. Depression also is a common effect of neurological disorders, including Parkinson's and Alzheimer's diseases, multiple sclerosis, strokes, and brain tumors. Even moderate depressive symptoms are associated with a higher than average rate of arteriosclerosis, heart attacks, and high blood pressure. Depression can mimic medical illness and any illness feels worse to someone suffering from depression.

It's not known specifically what causes depression. As with many mental illnesses, it's thought that a variety of biochemical, genetic and environmental factors may cause depression. Despite the many advances that occurred from a better understanding of neuropharmacology, many psychiatric diseases remain untreated or inadequately treated with current pharmaceutical agents. In addition, many of the current agents interact with a number of cellular targets, potentially resulting in side effects that can greatly influence the overall outcome of therapy.

Numerous treatments for depression are available, including dozens of medications. Typical protocols include a selective serotonin reuptake inhibitor (SSRI). SSRIs include fluoxetine (Prozac, Sarafem), paroxetine (Paxil), sertraline (Zoloft), citalopram (Celexa) and escitalopram (Lexapro). Other common first choices for antidepressants include serotonin and norepinephrine reuptake inhibitors (SNRIs), norepinephrine and dopamine reuptake inhibitors (NDRIs), combined reuptake inhibitors and receptor blockers, and tetracyclic antidepressants. Tricyclic antidepressants (TCAs) are also effective, but because TCAs tend to have more numerous and more severe side effects, they are often less prescribed. Monoamine oxidase inhibitors (MAOIs) are often prescribed as a last resort, when other medications haven't worked.

Functional antagonists of the NMDA receptor complex exhibit antidepressant-like activity in the rodent test and models of depression. In 1990, Trullas and Skolnick demonstrated the antidepressant activity of AP-7, MK-801 and ACPC in the mouse forced swim test (FST) and tail suspension test (TST) (Trullas R, Skolnick P (1990) Eur J Pharmacol 185:1-10). Since then, a number of reports have confirmed and extended this finding. The NMDA antagonists are active in the FST in mice (Layer™, et al. (1995) Pharmacol Biochem Behav 52:621-627; Maj et al. (1992) Pol J Pharmacol 44:337-346) and rats (Moryl, et al. (1993) Pharmacol Toxicol 72:394-397; Przegaliñski, et al (1997) Neuropharmacology 36:31-37) and tail suspension test in mice (Layer, et al. (1995) Pharmacol Biochem Behav 52:621-627), and in learned helplessness (Meloni, et al. (1993) Pharmacol Biochem Behav 46:423-426), chronic unpredicted stress (Ossowska, et al. (1997) J Physiol Pharmacol 48:127-135), chronic mild stress (Papp, et al. Eur J Pharmacol 263:1-7), and bulbectomy models (Redmond, et al. (1997) Pharmacol Biochem Behav 58:355-359). NMDA antagonists also demonstrate efficacy in clinical studies. Ketamine appears effective in major depression (Berman, et al. (2000) Biol Psychiatry 47:351-354; Zarate, et al. (2006) Arch Gen Psychiatry 63:856-864), although the clinical efficacy of memantine is not as clear (Ferguson, et al. (2007) Clin Neuropharmacol 30:136-144; Zarate, et al. (2006) Am J Psychiatry 163:153-155). Furthermore, the palliative effect of non-specific NMDA antagonist (amantadine and zinc) supplementation to antidepressant therapy has been suggested. On the other hand, antidepressants induce adaptive changes in the NMDA receptor complex (Skolnick, et al. (1996) Pharmacopsychiatry 29:23-26; Skolnick, et al. (2001) Pharmacol Res 43:411-423). Alterations in this receptor complex were demonstrated in the animal paradigm used for antidepressant screening (FST), in models of depression (Nowak, et al. (1998) Pol J Pharmacol 50:365-369; Nowak, et al. (1995) J Neurochem 64:925-927) and suicide victims (Nowak, e t al. (1995) Brain Res 675:157-164). Thus, depression may be associated with enhanced NMDA signal transduction and the mechanism of antidepressant effect is related to reduction of this transmission.

U.S. Pat. No. 7,019,016 to Pfizer provides methods for treating certain disorders including depression which comprise administration of certain NR2B subunit selective NMDA antagonists. The disorders that can be treating by the invention include hearing loss, vision loss, neurodegeneration caused by epileptic seizures, neurotoxin poisoning, Restless Leg Syndrome, multi-system atrophy, non-vascular headache, and depression.

U.S. Pat. No. 5,710,168 claims the use of certain compounds having NR2B subunit selectivity for treating a disease or condition which is susceptible to treatment by blocking of NMDA receptor sites, including traumatic brain injury, spinal cord trauma, pain, psychotic conditions, drug addiction, migraine, hypoglycemia, anxiolytic conditions, urinary incontinence, and ischemic events arising from CNS surgery, open heart surgery or any procedure during which the function of the cardiovascular system is compromised.

U.S. Pat. No. 6,479,553 to AstraZeneca provides certain compounds, in particular memantine, budipine, amantidine, 5-aminocarbonyl-10,11-dihydro-5H-dibenzo[a,d]cyclohepten-5,10-imine, dextromethorphan and NPS 1506, and the compounds disclosed in EP 279 937 and EP 633 879, specifically (S)-1-phenyl-2-(2-pyridyl)ethanamine as potentially useful as antidepressant agents. In particular, the compounds were expected to be useful in the treatment of depression associated with neurodegenerative disorders such as Alzheimer's disease.

U.S. Pat. No. 6,432,985 to Hoffman La-Roche provides certain neuroprotective substituted piperidine compounds with activity as NMDA NR2B subtype selective antagonists.

PCT Publication No. WO 06/017409 to Merck & Co. provides certain 1,3-disubstituted heteroaryl compounds are N-methyl-D-aspartate receptor antagonists useful for treating neurological condition e.g. pain, Parkinson's disease, Alzheimer's disease, anxiety, epilepsy and stroke.

PCT Publication No. WO 02/072542 to Emory University describes a class of pH-dependent NMDA receptor antagonists that exhibit pH sensitivity tested in vitro using an oocyte assay and in an experimental model of epilepsy.

While NMDA-receptor antagonists might be useful to treat a number of very challenging disorders, to date, dose-limiting side effects have prevented clinical use of NMDA receptor antagonists for these conditions. Thus, despite the potential for glutamate antagonists to treat many serious diseases, the severity of the side effects have caused many to abandon hope that a well-tolerated NMDA receptor antagonist could be developed (Hoyte L. et al (2004) Curr. Mol. Med. 4(2): 131-136; Muir, K. W. and Lees, K. R. (1995) Stroke 26:503-513; Herrling, P. L., ed. (1997) “Excitatory amino acid clinical results with antagonists” Academic Press; Parsons et al. (1998) Drug News Perspective II: 523 569).

There remains a need for improved neuroprotective compounds and methods for the treatment and/or prophylaxis of neuropsychiatric disorders. In particular, there is a need for compounds that have enhanced efficacy in treatment of neuropsychiatric disorders. In addition, there remains a need for effective compounds that exhibit reduced side effects upon administration. In particular there is a need for improved treatments for depression and anxiety.

It is therefore an object of the present invention to provide new pharmaceutical compositions and methods for the treatment of neuropsychiatric disorders, and in particular for the treatment of depression and anxiety.

### SUMMARY OF THE INVENTION

Compounds of Formula I, II, III and IV are provided for the treatment or prophylaxis of neuropsychiatric disorders. In particular, compounds for us in the treatment or prophylaxis of depression or anxiety in a host at risk of or suffering from the disorder are provided. In certain instances, the disorders are specifically known to result from NMDA receptor activation. Certain NMDA receptor antagonists described herein have enhanced activity in brain tissue having lower-than-normal pH due to conditions associated with a mood disorder.

In one particular embodiment, methods of treatment or prophylaxis of neuropsychiatric disorders, in particular depression and anxiety are provided comprising administering a compound of Formula I or a pharmaceutically acceptable salt, ester, prodrug or derivative thereof, optionally in combination with a pharmaceutically acceptable carrier, to a host in need thereof:

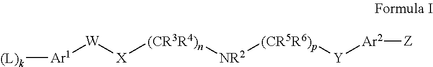

wherein the substituents are described herein. More typically, the compounds are of Formula A:

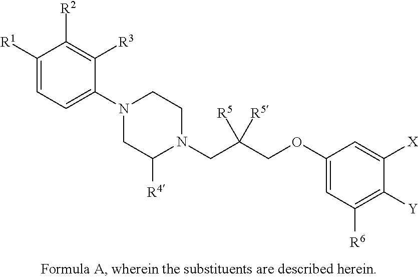

In a separate embodiment, methods of treatment or prophylaxis of neuropsychiatric disorders, in particular depression and anxiety are provided comprising administering a compound of Formula II or a pharmaceutically acceptable salt, ester, prodrug or derivative thereof, optionally in combination with a pharmaceutically acceptable carrier, to a host in need thereof:

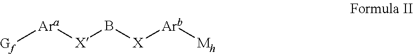

wherein the substituents are described herein. More typically, the compounds are of Formula B:

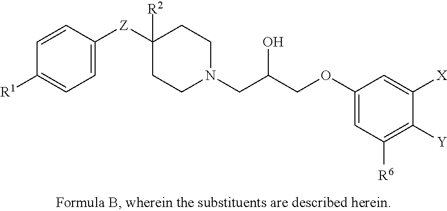

In certain embodiments, the compounds are used for the treatment of neuropsychiatric disorders, and in particular embodiments, neuropsychiatric mood disorders. These disorders include depression, bipolar disorders, seasonal affective disorders (SAD) and manias. In certain embodiments, the compounds are used for the treatment of depression in a host diagnosed with the disorder. In certain other embodiments, the compounds are used for treatment of a bipolar disorder in a host diagnosed with the disorder. The compounds can also be used to prevent or diminish future depressive or manic episodes. The compounds can be provided on a seasonal basis, especially in a host who has been diagnosed or is at risk of SAD or of depression.

In certain other embodiments, the compounds are useful in the treatment or prophylaxis of a neuropsychiatric disorder associated with a physiological insult. The disorder can include depression or bipolar disorder associated with an injury or with aging. The compounds may also be useful in treatment or prophylaxis of schizophrenia.

In certain embodiments, the compounds are administered to a host in need thereof. In certain other embodiments, the compounds are administered in combination or alternation with other compounds, in particular embodiments another compound useful in the treatment or prophylaxis of neuropsychiatric disorders.

### BRIEF DESCRIPTION OF THE FIGURES

FIG. 1 is graph of the immobility time (in seconds) of CD1 mice dosed with a test compound in a forced swim test. Structures of test compounds are shown in Table 26

FIG. 2 is graph of the immobility time (in seconds) of CD1 mice dosed with a test compound in a forced swim test.

FIG. 3 is a graph of the distance traveled by CD1 mice injected with a test compound in an open field activity test.

FIG. 4 is a graph of the motor performance of the CD1 mice on a rotorod after dosing with test compounds.

FIG. 5 is a graph of the cell toxicity of the test compounds as assessed by percent total LDH release.

FIG. 6 is a graph of the hERG binding IC₅₀ (μM) for selected compounds plotted against the patch clamp IC₅₀ (μM).

FIG. 7 is a graph of the QT interval (msec) correlated with the 10 g of the concentration of the test compound. Langendorff QT effects are shown for compounds NP10075, NP10239 and NP10076.

FIG. 8 is a graph of PCP discrimination test data for NP10031 and NP10097.

### DETAILED DESCRIPTION OF THE INVENTION

Certain compounds are provided as useful in the treatment or prophylaxis of neuropsychiatric disorders. Typically, these compounds act as NMDA antagonists. In particular, compounds of Formulas I, II, III and IV are provided for treatment of mood disorders including depression or anxiety. In certain instances, the disorders are specifically known to result from NMDA receptor activation. In certain embodiments, the compounds are allosteric NMDA inhibitors. In one embodiment, the IC₅₀ value of the compound is 0.01 to 10 μM, 0.01 to 9 μM, 0.01 to 8 μM, 0.01 to 7 μM, 0.01 to 6 μM, 0.01 to 5 μM, 0.01 to 4 μM, 0.01 to 3 μM, 0.01 to 2 μM, 0.01 to 1 μM, 0.05 to 7 μM, 0.05 to 6 μM, 0.05 to 5 μM, 0.05 to 4 μM, 0.05 to 3 μM, 0.05 to 2 μM, 0.05 to 1 μM, 0.05 to 0.5 μM, 0.1 to 7 μM, 0.1 to 6 μM, 0.1 to 5 μM, 0.1 to 4 μM, 0.1 to 3 μM, 0.1 to 2 μM, 0.1 to 1 μM, 0.1 to 0.5 μM, 0.1 to 0.4 μM, 0.1 to 0.3 μM, or 0.1 to 0.2 μM.

Certain NMDA receptor antagonists described herein have enhanced activity in tissue having lower-than-normal pH. Certain studies have indicated that pH may be altered in brains of individuals suffering from certain neuropsychiatric disorder (see e.g. Karolewicz, et al. (2004) J. Neurochem 91:1057-66. Xing, et al. (2002) Schizophr Res. 58:21-30.) A reduced brain pH can be harnessed as a switch to activate the neuroprotective agents described herein. In this way side effects are minimized in unaffected tissue since drug at these sites are less active.

In particular embodiments, the compound is pH sensitive. In specific embodiments, the compound exhibits a potency boost of at least 2, at least 3, at least 4, at least 5, at least 6, at least 7, at least 8, at least 9, at least 10, at least 15 or at least 20 when comparing the IC₅₀ at physiological pH versus the IC₅₀ diseased pH (i.e., (IC₅₀ at phys pH/IC₅₀ at diseased pH)).

In one embodiment, the compound has an IC₅₀ value of less than 10 μM at a pH of about 6 to about 9. In one embodiment, the compound has an IC₅₀ value of less than 10 μM at a pH of about 6.9. In another embodiment, the compound has an IC₅₀ value of less than 10 μM at a pH of about 7.6. In one embodiment, the compound has an IC₅₀ value of less than 10 μM at physiological pH. In one embodiment, the compound has an IC₅₀ value of less than 10 μM at ischemic pH.

In one embodiment, the IC₅₀ value of the compound is 0.01 to 10 μM, 0.01 to 9 μM, 0.01 to 8 μM, 0.01 to 7 μM, 0.01 to 6 μM, 0.01 to 5 μM, 0.01 to 4 μM, 0.01 to 3 μM, 0.01 to 2 μM, 0.01 to 1 μM, 0.05 to 7 μM, 0.05 to 6 μM, 0.05 to 5 μM, 0.05 to 4 μM, 0.05 to 3 μM, 0.05 to 2 μM, 0.05 to 1 μM, 0.05 to 0.5 μM, 0.1 to 7 μM, 0.1 to 6 μM, 0.1 to 5 μM, 0.1 to 4 μM, 0.1 to 3 μM, 0.1 to 2 μM, 0.1 to 1 μM, 0.1 to 0.5 μM, 0.1 to 0.4 μM, 0.1 to 0.3 μM, or 0.1 to 0.2 μM, and the ratio of the IC₅₀ values at pH 7.6 to pH 6.9 for the compound is greater than 1, 2, 3, 4, 5, 6, 7, 8, 9, 10, 15, 20, 25, 30, 40, 50, 60, 70, 80, 90, or 100.

In one embodiment, the IC₅₀ value of the compound is 0.01 to 10 μM, 0.01 to 9 μM, 0.01 to 8 μM, 0.01 to 7 μM, 0.01 to 6 μM, 0.01 to 5 μM, 0.01 to 4 μM, 0.01 to 3 μM, 0.01 to 2 μM, 0.01 to 1 μM, 0.05 to 7 μM, 0.05 to 6 μM, 0.05 to 5 μM, 0.05 to 4 μM, 0.05 to 3 μM, 0.05 to 2 μM, 0.05 to 1 μM, 0.05 to 0.5 μM, 0.1 to 7 μM, 0.1 to 6 μM, 0.1 to 5 μM, 0.1 to 4 μM, 0.1 to 3 μM, 0.1 to 2 μM, 0.1 to 1 μM, 0.1 to 0.5 μM, 0.1 to 0.4 μM, 0.1 to 0.3 μM, or 0.1 to 0.2 μM, and the ratio of the IC₅₀ values at pH 7.6 to pH 6.9 for the compound is between 1 and 100, 2 and 100, 3 and 100, 4 and 100, 5 and 100, 6 and 100, 7 and 100, 8 and 100, 9 and 100, 10 and 100, 15 and 100, 20 and 100, 25 and 100, 30 and 100, 40 and 100, 50 and 100, 60 and 100, 70 and 100, 80 and 100, or 90 and 100.

### DEFINITIONS

Whenever a term in the specification is identified as a range (i.e. C₁₋₄ alkyl), the range independently refers to each element of the range. As a non-limiting example, C₁₋₄ alkyl means, independently, C₁, C₂, C₃ or C₄ alkyl. Similarly, when one or more substituents are referred to as being “independently selected from” a group, this means that each substituent can be any element of that group, and any combination of these groups can be separated from the group. For example, if R¹ and R² can be independently selected from X, Y and Z, this separately includes the groups R¹ is X and R² is X; R¹ is X and R² is Y; R¹ is X and R² is Z; R¹ is Y and R² is X; R¹ is Y and R² is Y; R¹ is Y and R² is Z; R¹ is Z and R² is X; R¹ is Z and R² is Y; and R¹ is Z and R² is Z.

The term “alkyl” is used herein, unless otherwise specified, refers to a substituted or unsubstituted, saturated, straight, branched, or cyclic (also identified as cycloalkyl), primary, secondary, or tertiary hydrocarbon, including but not limited to those of C₁ to C₆. Illustrative examples of alkyl groups are methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless otherwise specified, the alkyl group can be unsubstituted or substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, thio, sulfonyl, ester, carboxylic acid, amide, phosphonyl, phosphinyl, thioether, oxime, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991. In certain embodiments, alkyl may be optionally substituted by one or more fluoro, chloro, bromo, iodo, hydroxy, heterocyclic, heteroaryl, carboxy, alkoxy, nitro, NH₂, N(alkyl)₂, NH(alkyl), alkoxycarbonyl, —N(H or alkyl)C(O)(H or alkyl), —N(H or alkyl)C(O)N(H or alkyl)₂, —N(H or alkyl)C(O)O(H or alkyl), —OC(O)N(H or alkyl)₂, —S(O)ₙ—(H or alkyl), —C(O)—N(H or alkyl)₂, cyano, alkenyl, cycloalkyl, acyl, hydroxyalkyl, heterocyclic, heteroaryl, aryl, aminoalkyl, oxo, carboxyalkyl, —C(O)—NH₂, —C(O)—N(H)O(H or alkyl), —S(O)₂—NH₂, —S(O)ₙ—N(H or alkyl)₂ and/or —S(O)₂—N(H or alkyl)₂.

The term “halo” or “halogen,” refers to chloro, bromo, iodo, or fluoro.

The term “heteroaryl” or “heteroaromatic,” refers to an aromatic that includes at least one sulfur, oxygen, nitrogen or phosphorus in the aromatic ring. The term “heterocyclic” refers to a non-aromatic cyclic group wherein there is at least one heteroatom, such as oxygen, sulfur, nitrogen, or phosphorus in the ring. Nonlimiting examples of heteroaryl and heterocyclic groups include furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, imidazole, 1,2,3-triazole, 1,2,4-triazole, oxazole, isoxazole, thiazole, isothiazole, pyrimidine or pyridazine, pteridinyl, aziridines, thiazole, isothiazole, oxadiazole, thiazine, pyridine, pyrazine, piperazine, piperidine, pyrrolidine, oxaziranes, phenazine, phenothiazine, morpholinyl, pyrazolyl, pyridazinyl, pyrazinyl, quinoxalinyl, xanthinyl, hypoxanthinyl, pteridinyl, 5-azacytidinyl, 5-azauracilyl, thiazolopyridinyl, imidazolopyridinyl, pyrrolopyrimidinyl, pyrazolopyrimidinyl, adenine, N⁶-alkylpurines, N⁶-benzylpurine, N⁶-halopurine, N⁶-vinylpurine, N⁶-acetylenic purine, N⁶-acyl purine, N⁶-hydroxyalkyl purine, N⁶-thioalkyl purine, thymine, cytosine, 6-azapyrimidine, 2-mercaptopyrmidine, uracil, N⁵-alkylpyrimidines, N⁵-benzylpyrimidines, N⁵-halopyrimidines, N⁵-vinylpyrimidine, N⁵-acetylenic pyrimidine, N⁵-acyl pyrimidine, N⁵-hydroxyalkyl purine, and N⁶-thioalkyl purine, and isoxazolyl. The heteroaromatic or heterocyclic group can be optionally substituted with one or more substituent selected from halogen, haloalkyl, alkyl, alkoxy, hydroxy, carboxyl derivatives, amido, amino, alkylamino, dialkylamino. The heteroaromatic can be partially or totally hydrogenated as desired. Nonlimiting examples include dihydropyridine and tetrahydrobenzimidazole. In some embodiment, the heteroaryl may be optionally substituted by one or more fluoro, chloro, bromo, iodo, hydroxy, heterocyclic, heteroaryl, carboxy, alkoxy, nitro, NH₂, N(alkyl)₂, NH(alkyl), alkoxycarbonyl, —N(H or alkyl)C(O)(H or alkyl), —N(H or alkyl)C(O)N(H or alkyl)₂, —N(H or alkyl)C(O)O(H or alkyl), —OC(O)N(H or alkyl)₂, —S(O)ₙ—(H or alkyl), —C(O)—N(H or alkyl)₂, cyano, alkenyl, cycloalkyl, acyl, hydroxyalkyl, heterocyclic, heteroaryl, aryl, aminoalkyl, oxo, carboxyalkyl, —C(O)—NH₂, —C(O)—N(H)O(H or alkyl), —S(O)₂—NH₂, —S(O)ₙ—N(H or alkyl)₂ and/or —S(O)₂—N(H or alkyl)₂. Functional oxygen and nitrogen groups on the heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-tolylsulfonyl.

The term “aryl,” unless otherwise specified, refers to a carbon based aromatic ring, including phenyl, biphenyl, or naphthyl. The aryl group can be optionally substituted with one or more moieties selected from the group consisting of hydroxyl, acyl, amino, halo, alkylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., “Protective Groups in Organic Synthesis,” John Wiley and Sons, Second Edition, 1991. In certain embodiments, the aryl group is optionally substituted by one or more fluoro, chloro, bromo, iodo, hydroxy, heterocyclic, heteroaryl, carboxy, alkoxy, nitro, NH₂, N(alkyl)₂, NH(alkyl), alkoxycarbonyl, —N(H or alkyl)C(O)(H or alkyl), —N(H or alkyl)C(O)N(H or alkyl)₂, —N(H or alkyl)C(O)O(H or alkyl), —OC(O)N(H or alkyl)₂, —S(O)ₙ—(H or alkyl), —C(O)—N(H or alkyl)₂, cyano, alkenyl, cycloalkyl, acyl, hydroxyalkyl, heterocyclic, heteroaryl, aryl, aminoalkyl, oxo, carboxyalkyl, —C(O)—NH₂, —C(O)—N(H)O(H or alkyl), —S(O)₂—NH₂, —S(O)ₙ—N(H or alkyl)₂ and/or —S(O)₂—N(H or alkyl)₂.

The term “aralkyl,” unless otherwise specified, refers to an aryl group as defined above linked to the molecule through an alkyl group as defined above.

The term “alkaryl,” unless otherwise specified, refers to an alkyl group as defend above linked to the molecule through an aryl group as defined above. Other groups, such as acyloxyalkyl, alkoxyalkyl, alkoxycarbonyl, alkoxycarbonylalkyl, alkylaminoalkyl, alkylthioalkyl, amidoalkyl, aminoalkyl, carboxyalkyl, dialkylaminoalkyl, haloalkyl, heteroaralkyl, heterocyclicalkyl, hydroxyalkyl, sulfonamidoalkyl, sulfonylalkyl and thioalkyl are named in a similar manner.

The term “alkoxy,” unless otherwise specified, refers to a moiety of the structure —O-alkyl, wherein alkyl is as defined above.

The term “acyl,” refers to a group of the formula C(O)R′ or “alkyl-oxy”, wherein R′ is an alkyl, aryl, alkaryl or aralkyl group, or substituted alkyl, aryl, aralkyl or alkaryl.

The term “alkenyl” The term “alkenyl” means a monovalent, unbranched or branched hydrocarbon chain having one or more double bonds therein. The double bond of an alkenyl group can be unconjugated or conjugated to another unsaturated group. Suitable alkenyl groups include, but are not limited to (C₂-C₈)alkenyl groups, such as vinyl, allyl, butenyl, pentenyl, hexenyl, butadienyl, pentadienyl, hexadienyl, 2-ethylhexenyl,2-propyl-2-butenyl,4-(2-methyl-3-butene)-pentenyl. An alkenyl group can be unsubstituted or substituted with one or two suitable substituents.

The term “carbonyl” refers to a functional group composed of a carbon atom double-bonded to an oxygen atom: —C═O, Similarly, C(O) or C(═O) refers to a carbonyl group.

The term “amino” refers to —NH₂, —NH(alkyl) or —N(alkyl)₂.

The term “thio” indicates the presence of a sulfur group. The prefix thio- denotes that there is at least one extra sulfur atom added to the chemical. The prefix ‘thio-’ can also be placed before the name of a ompoundto mean that an oxygen atom in the compound has been replaced by a sulfur atom. Although typically the term “thiol” is used to indicate the presence of —SH, in instances in which the sulfur atom would be have improper valance a radical if the hydrogen is improperly designated, the terms ‘thio’ and ‘thiol’ are used interchangeably, unless otherwise indicated.

The term “amido” indicates a group (H or alkyl)-C(O)—NH—.

The term “carboxy” designates the terminal group —C(O)OH.

The term “sulfonyl” indicates an organic radical of the general formula (H or alkyl)-S(═O)₂—(H or alkyl'), where there are two double bonds between the sulfur and oxygen.

The term “pharmaceutically acceptable salt” refers to salts or complexes that retain the desired biological activity of the compounds of the present invention and exhibit minimal undesired toxicological effects. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalcturonic acid; (b) base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylenediamine, D-glucosamine, tetraethylammonium, or ethylenediamine; or (c) combinations of (a) and (b); e.g., a zinc tannate salt or the like. Also included in this definition are pharmaceutically acceptable quaternary salts known by those skilled in the art, which specifically include the quaternary ammonium salt of the formula —NR⁺A⁻, wherein R is H or alkyl and A is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis.

It should be understood that the various possible stereoisomers of the groups mentioned above and herein are within the meaning of the individual terms and examples, unless otherwise specified. As an illustrative example, “1-methyl-butyl” exists in both (R) and the (S) form, thus, both (R)-1-methyl-butyl and (S)-1-methyl-butyl is covered by the term “1-methyl-butyl”, unless otherwise specified.

### Compounds

In one embodiment, methods of treatment or prophylaxis of neuropsychiatric disorders, in particular depression and anxiety, are provided comprising administering a compound of Formula I or a pharmaceutically acceptable salt, ester, prodrug or derivative thereof to a host in need thereof:

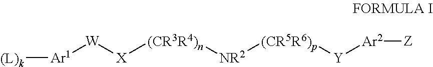

wherein:
each L is independently C₁-C₆ alkyl, C₁-C₆ alkoxy, C(═O)—(C₁-C₆)-alkyl, C₁-C₆ haloalkyl, alkaryl, hydroxy, —O-alkyl, —O-aryl, —SH, —S-alkyl, —S-aryl, fluoro, chloro, bromo, iodo, nitro, or cyano; or two L groups may be taken together with Ar¹ to form: a dioxolane ring or a cyclobutane ring;
k=0, 1, 2, 3, 4 or 5;
each Ar¹ and Ar² is independently aryl or heteroaryl;
W is a bond, C₁-C₄ alkyl, or C₂-C₄ alkenyl;
X is a bond, NR¹ or O;
each R¹ and R² is independently H, C₁-C₆ alkyl, C₂-C₆ alkenyl or C₆-C₁₂ aralkyl; or

R¹ and R² can be taken together to form a 5-8 membered ring;

each R³ and R⁴ is independently H, C₁-C₆ alkyl, C₁-C₆ alkoxy, C(═O)—(C₁-C₆)-alkyl, C₁-C₆ haloalkyl, hydroxy, fluoro, chloro, bromo, iodo, nitro, or cyano; or CR³R⁴ is C═O;
n and p are each independently 1, 2, 3 or 4;
each R⁵ and R⁶ is independently H, C₁-C₆ alkyl, C₁-C₆ alkoxy, C(═O)—(C₁-C₆)-alkyl, C₁-C₆ haloalkyl, hydroxy, fluoro, chloro, bromo, iodo, nitro, or cyano; or CR⁵R⁶ is C═O or C═CH₂;
or wherein —NR²—(CR⁵R⁶)ₚ— can be

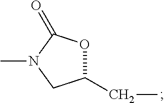

Y is a bond, O, S, SO, SO₂, CH₂, NH, N(C₁-C₆ alkyl), or NHC(═O);
Z is OH, NR⁶R⁷, NR⁸SO₂(C₁-C₆ alkyl), NR⁸C(O)NR⁶R⁷, NR⁸C(S)NR⁶R⁷, NR⁸C(O)O(C₁-C₆ alkyl), NR⁸-dihydrothiazole, or NR⁸-dihydroimidazole; wherein each R⁶, R⁷ and R⁸ is independently H, C₁-C₆ alkyl or C₆-C₁₂ aralkyl; or

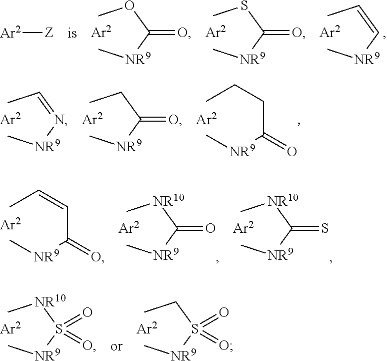

wherein R⁹ and R¹⁰ are each independently H, C₁-C₆ alkyl, aralkyl.

In one embodiment, when Y is NHC(═O), Z is not OH or NR⁸SO₂(C₁-C₆ alkyl). In one subembodiment, when R¹ and R² are taken together to form a 5-8 membered ring so that —NR¹—(CR³R⁴)ₙ—NR²— is

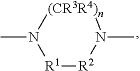

Y—Ar² is not NH-heteroaryl. In another subembodiment, when R¹ and R² are taken together to form a 5-8 membered ring so that —NR¹—(CR³R⁴)ₙ—NR²— is

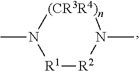

### Y is not NHC(═O).

In one embodiment, X is NR¹. In another embodiment, X is O. In another embodiment, X is a bond. In a particular subembodiment, X is a bond, n is 1, R³ and R⁴ are both H, and W is C₂ alkenyl.

In particular subembodiment, Ar¹ is phenyl, pyridyl, pyrimidinyl, thiophenyl, imidazolyl, furanyl, indolyl, benzothiophenyl, benzofuranyl, or benzoimidazolyl.

In another particular subembodiment, L is C₁-C₄ alkyl, C₁-C₄ alkoxy, C(═O)—(C₁-C₄)-alkyl, C₁-C₆ haloalkyl, hydroxy, fluoro, chloro, bromo, iodo, nitro, or cyano. In a further subembodiment, L is methyl, trifluoromethyl, methoxy, nitro, fluoro, chloro or hydroxy. In one further subembodiment, there are one, two or three L groups substituting Ar¹. In one subembodiment, Ar¹ is substituted with one fluoro group. In one subembodiment, Ar¹ is substituted with two fluoro groups. In one subembodiment, Ar¹ is substituted with one fluoro group and one chloro group. In one subembodiment, Ar¹ is substituted with one chloro group. In one subembodiment, Ar¹ is substituted with two chloro groups. In one subembodiment, Ar¹ is substituted with one methyl group. In one subembodiment, Ar¹ is substituted with one trifluoromethyl group.

In one subembodiment, Ar¹ is phenyl. In one subembodiment, Ar¹ is phenyl and is substituted with an L group at the 2, 3, or 4 position. In another subembodiment, Ar¹ is phenyl and is substituted with L groups at the 2 and 4 positions. In another subembodiment, Ar¹ is phenyl and is substituted with L groups at the 3 and 4 positions.

In one subembodiment, Ar¹ is pyridyl. In another subembodment, Ar¹ is 2-pyridyl, 3-pyridyl, or 4-pyridyl.

In one embodiment, Ar¹ is a bicyclic group wherein the W group is attached to the heterocyclic ring.

In one embodiment, W is a bond. In another embodiment, W is CH₂. In another embodiment, W is C₂-C₄ alkenyl.

In one embodiment, each R¹ and R² is independently H or C₁-C₄ alkyl, for example, methyl, ethyl, n-propyl, isopropyl, n-butyl, sec-butyl or tert-butyl. In one embodiment, R¹ and R² are both H. In one embodiment, R¹ and R² are both C₁-C₄ alkyl, for example n-butyl. In another embodiment, R¹ and R² can be taken together to form a 5-8 membered ring so that —NR¹—(CR³R⁴)ₙ—NR²— is

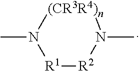

In one embodiment, n is 2. In one embodiment, n is 3. In another embodiment, R¹ and R² are each CH₂. In a subembodiment, CR³R⁴ is CH₂ and n is 2. In a subembodiment, CR³R⁴ in CH₂ and n is 3. In a subembodiment, CR³R⁴ is C═O and n is 1.

In one embodiment,

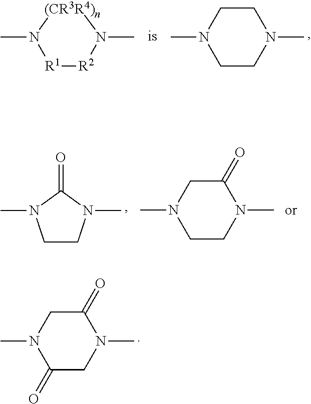

In one embodiment,

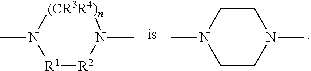

In another embodiment,

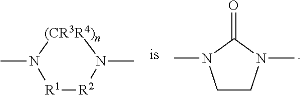

In one embodiment, each R⁵ and R⁶ is independently H, C₁-C₄ alkyl, C₁-C₄ alkoxy, C(═O)—(C₁-C₄)-alkyl, C₁-C₄ haloalkyl, hydroxy, fluoro, chloro, bromo, iodo, nitro, or cyano. In one embodiment, CR⁵R⁶ is C═O or C═CH₂. In one embodiment, p is 2, 3, or 4. In another embodiment, p is 3. In one embodiment, R⁵ and R⁶ are H. In another embodiment, one of R⁵ and R⁶ is hydroxy. In another embodiment, CR⁵R⁶ is C═CH₂. In another embodiment, CR⁵R⁶ is C═O. In one embodiment, (CR⁵R⁶)ₚ is selected from the group consisting of

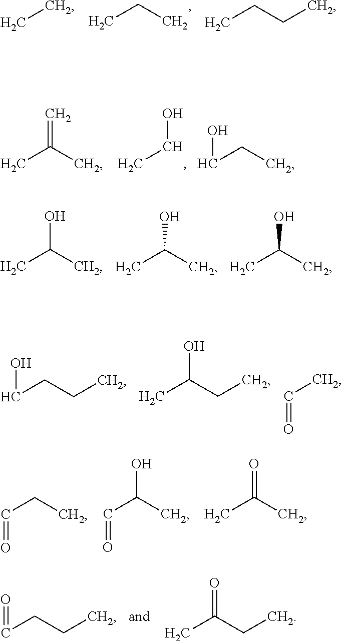

Compounds of Formula I can include compounds wherein when p is greater than 1, each (CR⁵R⁶) can be independently selected, for example, in one embodiment p is 2 and one (CR⁵R⁶) is C═O and the other (CR⁵R⁶) is CH₂. In one embodiment, R⁵ is not fluoro. In another embodiment, R⁶ is not fluoro.

In one embodiment, —NR²—(CR⁵R⁶)ₚ— is

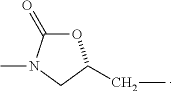

In a particular subembodiment, the compound is

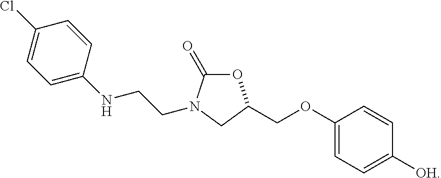

In another particular subembodiment, the compound is

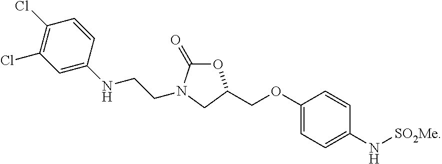

In one embodiment, Y is a bond, O or CH₂. In one embodiment, Y is O. In another embodiment, Y is CH₂. In one embodiment, Y is not NH. In another embodiment, Y is not NHC(═O).

In one embodiment, Ar² is aryl. In one embodiment, Ar² is aryl, but not phenyl or heteroaryl. In one embodiment Ar² is phenyl. In one subembodiment, Ar² is phenyl and is substituted with a Z group at the 4 position. In one embodiment, Ar² is not heteroaryl. In one embodiment, Ar² is aryl, but not phenyl or heteroaryl.

In one embodiment, Z is OH, NR⁶R⁷, NR⁸SO₂(C₁-C₆ alkyl), NR⁸C(O)NR⁶R⁷, NR⁸C(S)NR⁶R⁷, NR⁸C(O)O(C₁-C₆ alkyl), NR⁸-dihydrothiazole, or NR⁸-dihydroimidazole.

In one embodiment, Ar²—Z is

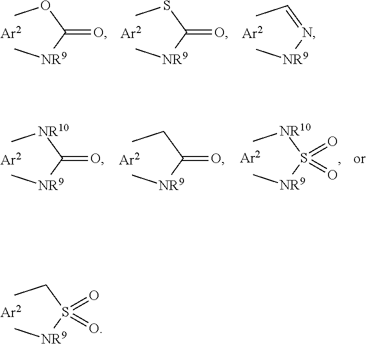

In one subembodiment,

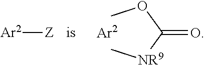

In one subembodiment, Ar²—Z is

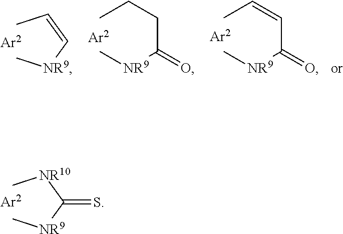

In one subembodiment, R⁹ and R¹⁰ are each H.

In one embodiment, Z is NR⁸C(O)NR⁶R⁷, for example NHC(O)NH₂ or NHC(O)N(CH₃)₂.

In another embodiment, Z and Ar² are taken together and selected from the group consisting of:

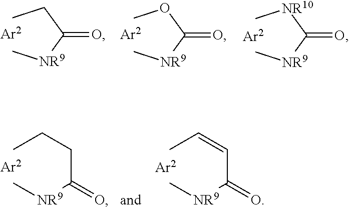

In one embodiment, the compound is a compound of Formula I, or a pharmaceutically acceptable salt, ester, prodrug or derivative thereof, wherein:

L is C₁-C₆ alkyl, C₁-C₆ alkoxy, C(═O)—(C₁-C₆)-alkyl, C₁-C₆ haloalkyl, alkaryl, hydroxy, —O-alkyl, —O-aryl, —SH, —S-alkyl, —S-aryl, fluoro, chloro, bromo, iodo, nitro, or cyano; or two L groups may be taken together with Ar¹ to form a dioxolane ring or a cyclobutane ring;
k=0, 1, 2, 3, 4 or 5;
Ar¹ is phenyl, pyridyl, pyrimidinyl, thiophenyl, imidazolyl, furanyl, indolyl, benzothiophenyl, benzofuranyl, benzoimidazolyl;
Ar² is phenyl;
W is a bond, C₁-C₄ alkyl, or C₂-C₄ alkenyl;
each R¹ and R² is independently H, C₁-C₄ alkyl; or

R¹ and R² can be taken together to form a 5-8 membered ring;

each R³ and R⁴ is independently H, C₁-C₆ alkyl, C₁-C₆ alkoxy, C(═O)—(C₁-C₆)-alkyl, C₁-C₆ haloalkyl, hydroxy, fluoro, chloro, bromo, iodo, nitro, or cyano; or CR³R⁴ is C═O;
n=1, 2, 3 or 4;
each R⁵ and R⁶ is independently H, C₁-C₆ alkyl, C₁-C₆ alkoxy, C(═O)—(C₁-C₆)-alkyl, C₁-C₆ haloalkyl, hydroxy, fluoro, chloro, bromo, iodo, nitro, or cyano; or CR⁵R⁶ is C═O, C═CH₂;
Y is a bond, O, S, SO, SO₂, CH₂, NH, N(C₁-C₆ alkyl), NHC(═O);
Z is OH, NH₂, NHSO₂(C₁-C₄ alkyl), NHC(O)NR⁶R⁷, NR⁸C(S)NR⁶R⁷, NHC(O)O(C₁-C₄ alkyl), NH-dihydrothiazole, or NH-dihydroimidazole; wherein each R⁶ and R⁷ is independently H, C₁-C₆ alkyl; or

### Ar²—Z is

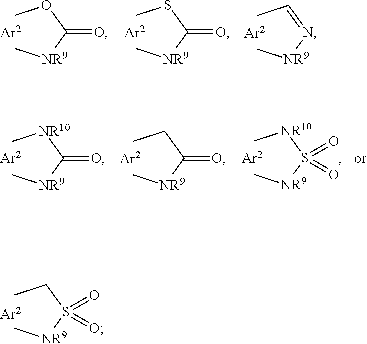

wherein R⁹ and R¹⁰ are each independently H or C₁-C₄ alkyl.

In one embodiment, the compound is a compound of Formula I, or a pharmaceutically acceptable salt, ester, prodrug or derivative thereof, wherein:

L is C₁-C₆ alkyl, C₁-C₆ alkoxy, C(═O)—(C₁-C₆)-alkyl, C₁-C₆ haloalkyl, alkaryl, hydroxy, —O-alkyl, —O-aryl, —SH, —S-alkyl, —S-aryl, fluoro, chloro, bromo, iodo, nitro, or cyano; or two L groups may be taken together with Ar¹ to form a dioxolane ring or a cyclobutane ring;
k=0, 1, 2, 3, 4 or 5;
Ar¹ is phenyl, pyridyl, pyrimidinyl, thiophenyl, imidazolyl, furanyl, indolyl, benzothiophenyl, benzofuranyl, benzoimidazolyl;
Ar² is phenyl;
W is a bond, C₁-C₄ alkyl, or C₂-C₄ alkenyl;
each R¹ and R² is independently H, C₁-C₄ alkyl; or

R¹ and R² can be taken together to form a 5-8 membered ring;

each R³ and R⁴ is independently H, C₁-C₆ alkyl, C₁-C₆ alkoxy, C(═O)—(C₁-C₆)-alkyl, C₁-C₆ haloalkyl, hydroxy, fluoro, chloro, bromo, iodo, nitro, or cyano; or CR³R⁴ is C═O;
n=1, 2, 3 or 4;
each R⁵ and R⁶ is independently H, C₁-C₆ alkyl, C₁-C₆ alkoxy, C(═O)—(C₁-C₆)-alkyl, C₁-C₆ haloalkyl, hydroxy, fluoro, chloro, bromo, iodo, nitro, or cyano; or CR⁵R⁶ is C═O, C═CH₂;
Y is a bond, O, S, SO, SO₂, CH₂, NH, N(C₁-C₆ alkyl), NHC(═O);
Z is OH, NH₂, NHSO₂(C₁-C₄ alkyl), NHC(O)NR⁶R⁷, NR⁸C(S)NR⁶R⁷, NHC(O)O(C₁-C₄ alkyl), NH-dihydrothiazole, or NH-dihydroimidazole; wherein each R⁶ and R⁷ is independently H, C₁-C₆ alkyl; or

### Ar²—Z is

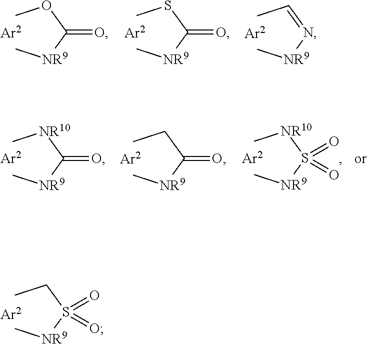

wherein R⁹ and R¹⁰ are each independently H or C₁-C₄ alkyl.

In one embodiment, the compound is a compound of Formula I, or a pharmaceutically acceptable salt, ester, prodrug or derivative thereof, wherein:

L is C₁-C₄ alkyl, C₁-C₄ alkoxy, C(═O)—(C₁-C₄)-alkyl, C₁-C₄ haloalkyl, alkaryl, hydroxy, —O-alkyl, —O-aryl, —SH, —S-alkyl, —S-aryl, fluoro, chloro, bromo, iodo, or nitro; or two L groups may be taken together to form a dioxolane ring with Ar¹;
k=0, 1, 2, 3, 4 or 5;
Ar¹ is phenyl or pyridyl;
Ar² is phenyl;
W is a bond or C₁-C₄ alkyl;

### X is NR¹;

each R¹ and R² is independently H or C₁-C₄ alkyl; or

R¹ and R² can be taken together to form a 5-8 membered ring;

each R³ and R⁴ is independently H or C₁-C₄ alkyl; or CR³R⁴ is C═O;
n=2 or 3;
each R⁵ and R⁶ is independently H, C₁-C₄ alkyl or OH; or CR⁴R⁵ is C═O or C═CH₂;

### Y is O or CH₂;

Z is OH, NH₂, NHSO₂(C₁-C₄ alkyl), NHC(O)NR⁶R⁷, NR⁸C(S)NR⁶R⁷, NHC(O)O(C₁-C₄ alkyl), NH-dihydrothiazole, or NH-dihydroimidazole; wherein each R⁶ and R⁷ is independently H or C₁-C₄ alkyl; or Ar²—Z is

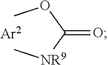

R⁹ is H or C₁-C₄ alkyl.

In one embodiment, the compound is a compound of Formula I, or a pharmaceutically acceptable salt, ester, prodrug or derivative thereof, wherein:

L is C₁-C₄ alkyl, C₁-C₄ alkoxy, C(═O)—(C₁-C₄)-alkyl, C₁-C₄ haloalkyl, alkaryl, hydroxy, —O-alkyl, —O-aryl, —SH, —S-alkyl, —S-aryl, fluoro, chloro, bromo, iodo, or nitro; or
two L groups may be taken together to form a dioxolane ring with Ar¹;
k=0, 1, 2, 3, 4 or 5;
Ar¹ is phenyl or pyridyl;
Ar² is phenyl;
W is a bond or C₁-C₄ alkyl;

### X is O;

R² is H or C₁-C₄ alkyl;
each R³ and R⁴ is independently H or C₁-C₄ alkyl; or CR³R⁴ is C═O;
n=2 or 3;
each R⁵ and R⁶ is independently H, C₁-C₄ alkyl or OH; or CR⁴R⁵ is C═O or C═CH₂;

### Y is O or CH₂;

Z is OH, NH₂, NHSO₂(C₁-C₄ alkyl), NHC(O)NR⁶R⁷, NHC(O)O(C₁-C₄ alkyl), NH-dihydrothiazole, or NH-dihydroimidazole; wherein each R⁶ and R⁷ is independently H or C₁-C₄ alkyl; or Ar²—Z is

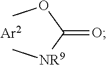

R⁹ is H or C₁-C₄ alkyl.

In one embodiment, the compound is a compound of Formula I, or a pharmaceutically acceptable salt, ester, prodrug or derivative thereof, wherein:

L is C₁-C₄ alkyl, C₁-C₄ alkoxy, C(═O)—(C₁-C₄)-alkyl, C₁-C₄ haloalkyl, hydroxy, fluoro, chloro, bromo, iodo, or nitro; or
two L groups may be taken together to form a dioxolane ring with Ar¹;
k=0, 1, 2, 3, 4 or 5;
Ar¹ is phenyl or pyridyl;
Ar² is phenyl;
W is C₂-C₄ alkenyl;
X is a bond;
R² is H or C₁-C₄ alkyl;
each R³ and R⁴ is independently H or C₁-C₄ alkyl; or CR³R⁴ is C═O;
n=1, 2 or 3;
each R⁵ and R⁶ is independently H, C₁-C₄ alkyl or OH; or CR⁴R⁵ is C═O or C═CH₂;

### Y is O or CH₂;

Z is OH, NH₂, NHSO₂(C₁-C₄ alkyl), NHC(O)NR⁶R⁷, NR⁸C(S)NR⁶R⁷, NHC(O)O(C₁-C₄ alkyl), NH-dihydrothiazole, or NH-dihydroimidazole; wherein each R⁶ and R⁷ is independently H or C₁-C₄ alkyl; or Ar²—Z is

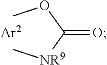

R⁹ is H or C₁-C₄ alkyl.

In one embodiment, the compound is selected from the compounds in Table 1.

TABLE 1

| Compound | NAME |
|---|---|
| 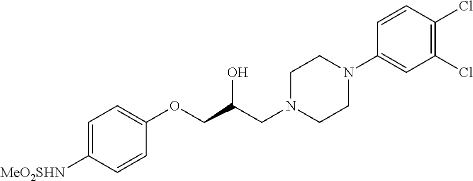 | N-(4-{3-[4-(3,4-Dichloro-phenyl)-piperazin-1-yl]-2- (S)-hydroxy-propoxy}-phenyl)-methanesulfonamide |
| 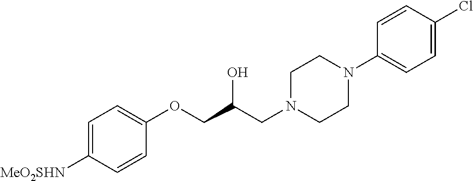 | N-(4-{3-[4-(4-Chloro-phenyl)-piperazin-1-yl]-2-(S)- hydroxy-propoxy}-phenyl)-methanesulfonamide |
| 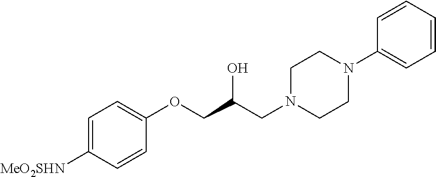 | N-(4-{3-[4-phenyl-piperazin-1-yl]-2-(S)-hydroxy- propoxy}-phenyl)-methanesulfonamide |
| 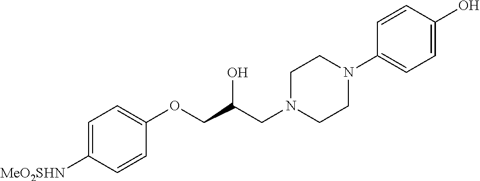 | N-(4-{3-[4-(4-Hydroxy-phenyl)-piperazin-1-yl]-2- (S)-hydroxy-propoxy}-phenyl)- methanesulfonamide |
| 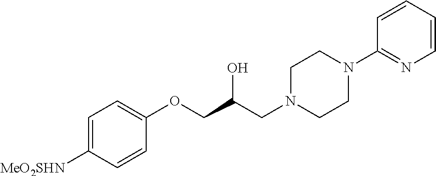 | N-(4-{3-[4-(2-Pyridyl)-piperazin-1-yl]-2-(S)- hydroxy-propoxy}-phenyl)-methanesulfonamide |
| 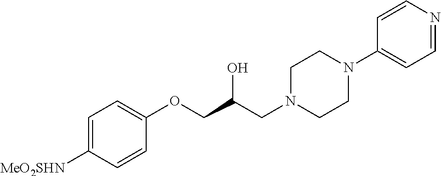 | N-(4-{3-[4-(4-Pyridyl)-piperazin-1-yl]-2-(S)- hydroxy-propoxy}-phenyl)-methanesulfonamide |
| 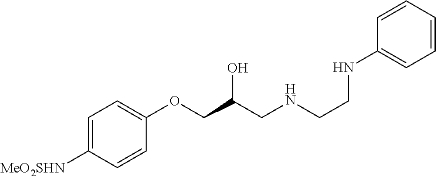 | N-{4-[2-(S)-Hydroxy-3-(2-phenylamino- ethylamino)-propoxy]-phenyl}-methanesulfonamide |
| 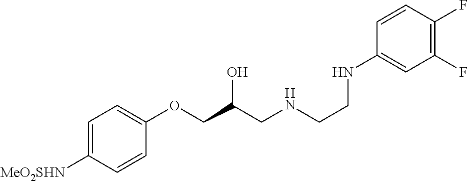 | N-{4-[2-(S)-Hydroxy-3-(2-(3,4-difluoro- phenyl)amino-ethylamino)-propoxy]-phenyl}- methanesulfonamide |
| 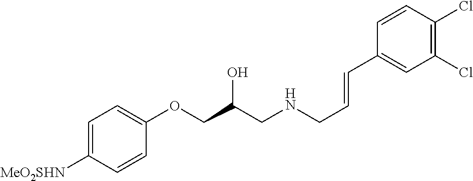 | N-(4-{3-[3-(3,4-Dichloro-phenyl)-allylamino]-2- (S)-hydroxy-propoxy}-phenyl)-methanesulfonamide |
| 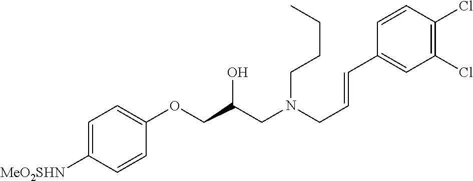 | N-[4-(3-{Butyl-[3-(3,4-dichloro-phenyl)-allyl]- amino}-2-(S)-hydroxy-propoxy)-phenyl]- methanesulfonamide |
| 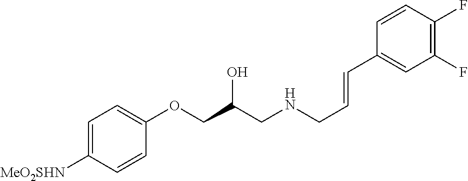 | N-(4-{3-[3-(3,4-Difluoro-phenyl)-allylamino]-2-(S)- hydroxy-propoxy}-phenyl)-methanesulfonamide |

In one embodiment, the compound is selected from the compounds in Table 2.

TABLE 2

| Compound | NAME |
|---|---|
| 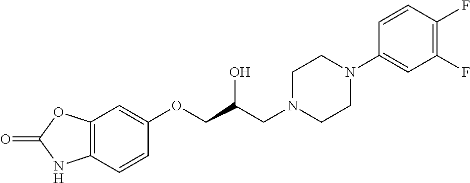 | 6-{3-[4-(3,4-Difluoro-phenyl)-piperazin-1-yl]-2-(S)- hydroxy-propoxy}-3H-benzooxazol-2-one |
| 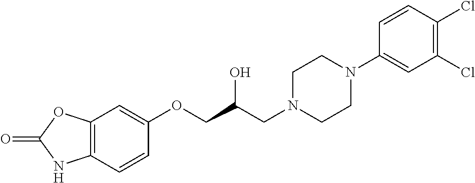 | 6-{3-[4-(3,4-Dichloro-phenyl)-piperazin-1-yl]-2- (S)-hydroxy-propoxy}-3H-benzooxazol-2-one |
| 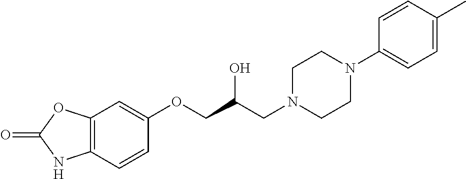 | 6-{3-[4-(4-Methyl-phenyl)-piperazin-1-yl]-2-(S)- hydroxy-propoxy}-3H-benzooxazol-2-one |
| 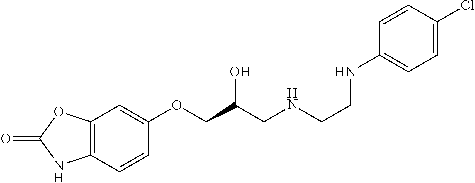 | 6-{3-[2-(4-Chloro-phenylamino)-ethylamino]-2-(S)- hydroxy-propoxy}-3H-benzooxazol-2-one |

In one embodiment, the compound is selected from the compounds in Table 3.

TABLE 3

| Compound | NAME |
|---|---|
| 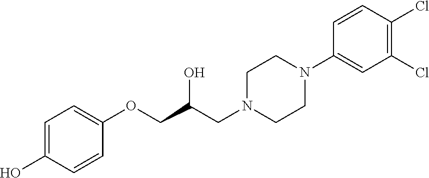 | 4-{3-[4-(3,4-Dichloro-phenyl)-piperazin-1-yl]-2- (S)-hydroxy-propoxy}-phenol |
| 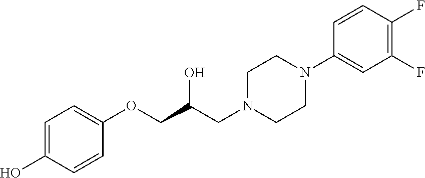 | 4-{3-[4-(3,4-Difluoro-phenyl)-piperazin-1-yl]-2- (S)-hydroxy-propoxy}-phenol |
| 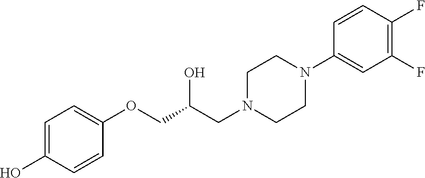 | 4-{3-[4-(3,4-Difluoro-phenyl)-piperazin-1-yl]-2- (R)-hydroxy-propoxy}-phenol |
| 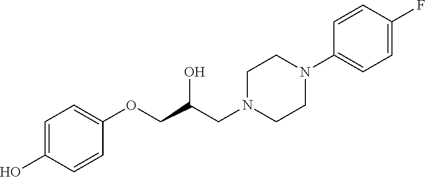 | 4-{3-[4-(4-Fluoro-phenyl)-piperazin-1-yl]-2-(S)- hydroxy-propoxy}-phenol |
| 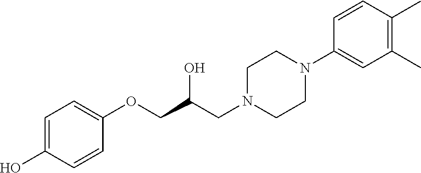 | 4-{3-[4-(3,4-Dimethyl-phenyl)-piperazin-1-yl]-2- (S)-hydroxy-propoxy}-phenol |
| 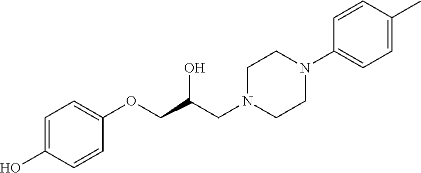 | 4-{3-[4-(4-Methyl-phenyl)-piperazin-1-yl]-2-(S)- hydroxy-propoxy}-phenol |
| 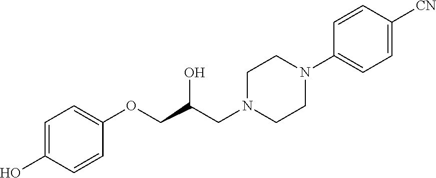 | 4-{3-[4-(4-Cyano-phenyl)-piperazin-1-yl]-2-(S)- hydroxy-propoxy}-phenol |
| 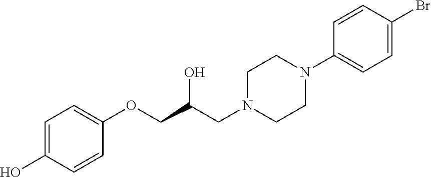 | 4-{3-[4-(4-Bromo-phenyl)-piperazin-1-yl]-2-(S)- hydroxy-propoxy}-phenol |
| 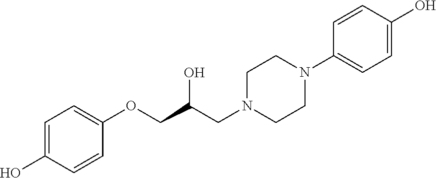 | 4-{3-[4-(4-Hydroxy-phenyl)-piperazin-1-yl]-2-(S)- hydroxy-propoxy}-phenol |
| 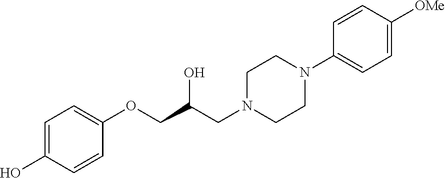 | 4-{3-[4-(4-Methoxy-phenyl)-piperazin-1-yl]-2-(S)- hydroxy-propoxy}-phenol |
| 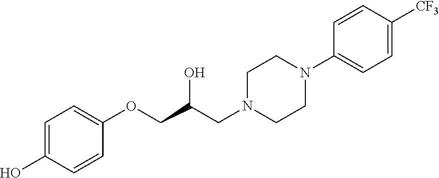 | 4-{3-[4-(4-Trifluoromethyl-phenyl)-piperazin-1-yl]- 2-(S)-hydroxy-propoxy}-phenol |
| 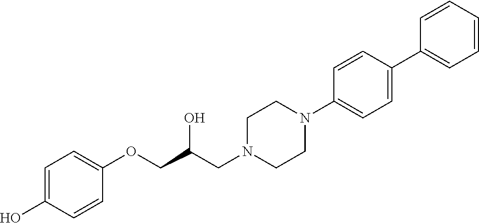 | 4-{3-[4-(4-Biphenyl)-piperazin-1-yl]-2-(S)- hydroxy-propoxy}-phenol |
| 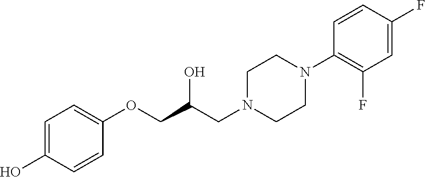 | 4-{3-[4-(2,4-Difluoro-phenyl)-piperazin-1-yl]-2-(S)- hydroxy-propoxy}-phenol |
| 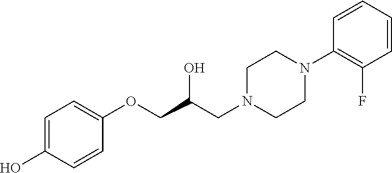 | 4-{3-[4-(2-Fluoro-phenyl)-piperazin-1-yl]-2-(S)- hydroxy-propoxy}-phenol |
| 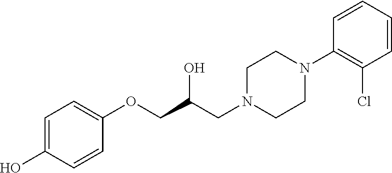 | 4-{3-[4-(2-Chloro-phenyl)-piperazin-1-yl]-2-(S)- hydroxy-propoxy}-phenol |
| 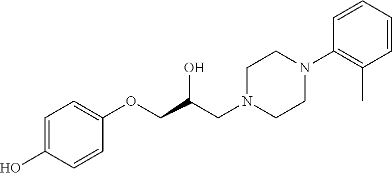 | 4-{3-[4-(2-Chloro-phenyl)-piperazin-1-yl]-2-(S)- hydroxy-propoxy}-phenol |
| 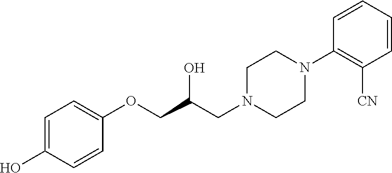 | 4-{3-[4-(2-Cyano-phenyl)-piperazin-1-yl]-2-(S)- hydroxy-propoxy}-phenol |
| 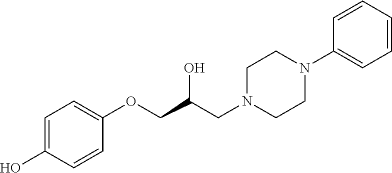 | 4-{3-[4-Phenyl-piperazin-1-yl]-2-(S)-hydroxy- propoxy}-phenol |
| 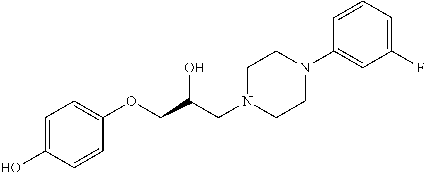 | 4-{3-[4-(3-Fluoro-phenyl)-piperazin-1-yl]-2-(S)- hydroxy-propoxy}-phenol |
| 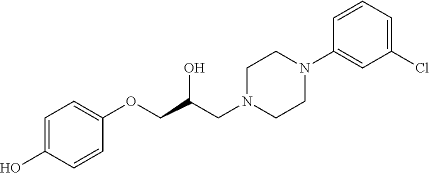 | 4-{3-[4-(3-Chloro-phenyl)-piperazin-1-yl]-2-(S)- hydroxy-propoxy}-phenol |
| 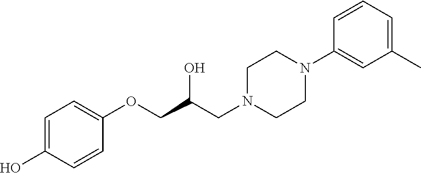 | 4-{3-[4-(3-Methyl-phenyl)-piperazin-1-yl]-2-(S)- hydroxy-propoxy}-phenol |
| 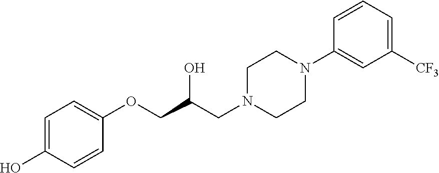 | 4-{3-[4-(3-Trifluoromethyl-phenyl)-piperazin-1-yl]- 2-(S)-hydroxy-propoxy}-phenol |
| 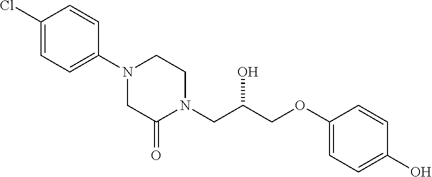 | |

In one embodiment, the compound is selected from the compounds in Table 4.

TABLE 4

| Compound | NAME |
|---|---|
| 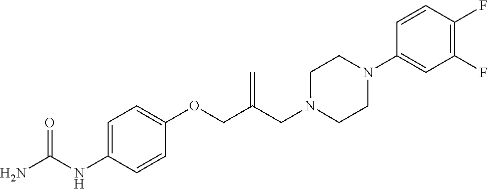 | (4-{2-[4-(3,4-Difluoro-phenyl)-piperazin-1- ylmethyl]-allyloxy}-phenyl)-urea |
| 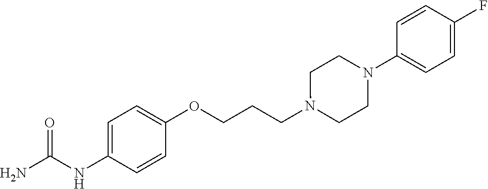 | (4-{3-[4-(4-Fluoro-phenyl)-piperazin-1-yl]- propoxy}-phenyl)-urea |
| 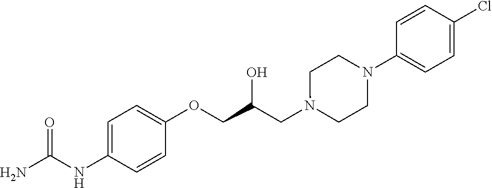 | (4-{3-[4-(4-Chloro-phenyl)-piperazin-1-yl]-2- hydroxy-propoxy}-phenyl)-urea |
| 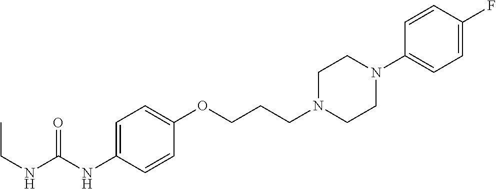 | 1-Ethyl-3-(4-{3-[4-(4-fluoro-phenyl)-piperazin-1- yl]-propoxy}-phenyl)-urea |
| 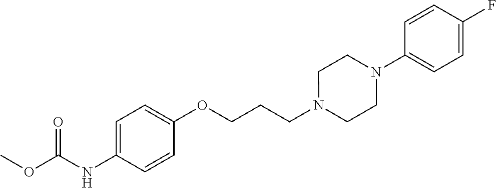 | (4-{3-[4-(4-Fluoro-phenyl)-piperazin-1-yl]- propoxy}-phenyl)-carbamic acid methyl ester |
| 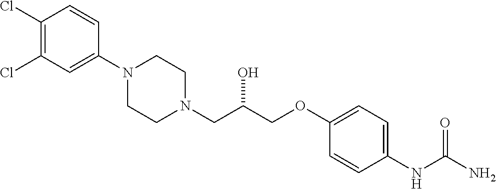 | (S)-1-(4-(3-(4-(3,4-dichlorophenyl)piperazin-1-yl)- 2-hydroxypropoxy)phenyl)urea |
| 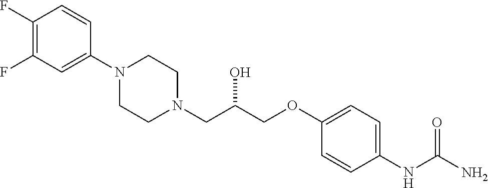 | (S)-1-(4-(3-(4-(3,4-difluorophenyl)piperazin-1-yl)- 2-hydroxypropoxy)phenyl)urea |
| 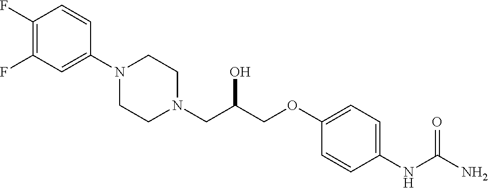 | (R)-1-(4-(3-(4-(3,4-difluorophenyl)piperazin-1-yl)- 2-hydroxypropoxy)phenyl)urea |
| 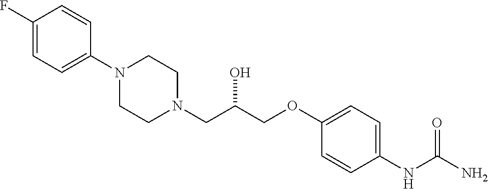 | (S)-1-(4-(3-(4-(4-fluorophenyl)piperazin-1-yl)-2- hydroxypropoxy)phenyl)urea |
| 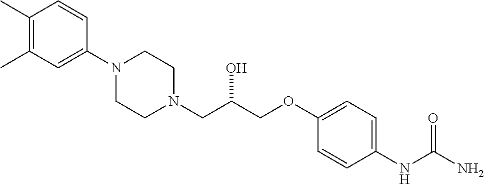 | (S)-1-(4-(3-(4-(3,4-dimethylphenyl)piperazin-1-yl)- 2-hydroxypropoxy)phenyl)urea |
| 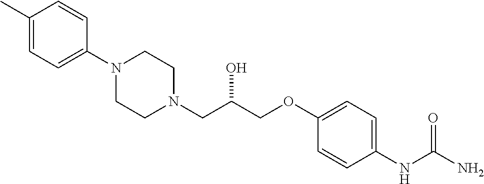 | (S)-1-(4-(2-hydroxy-3-(4-p-tolylpiperazin-1- yl)propoxy)phenyl)urea |
| 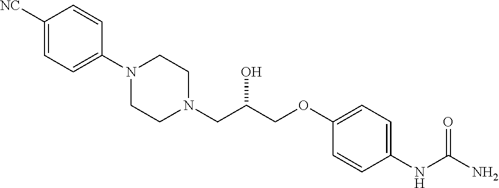 | (S)-1-(4-(3-(4-(4-cyanophenyl)piperazin-1-yl)-2- hydroxypropoxy)phenyl)urea |
| 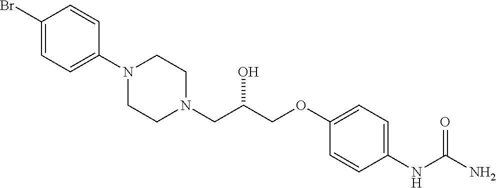 | (S)-1-(4-(3-(4-(4-bromophenyl)piperazin-1-yl)-2- (S)-hydroxypropoxy)phenyl)urea |
| 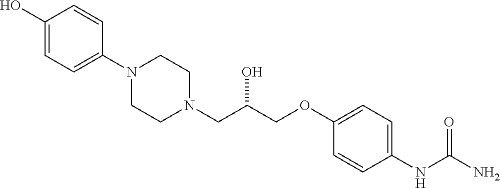 | (S)-1-(4-(2-hydroxy-3-(4-(4- hydroxyphenyl)piperazin-1-yl)propoxy)phenyl)urea |
| 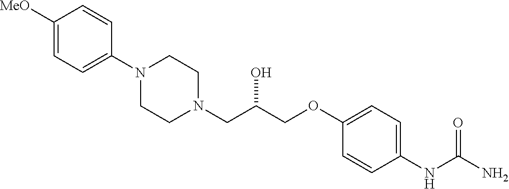 | (S)-1-(4-(2-hydroxy-3-(4-(4- methoxyphenyl)piperazin-1-yl)propoxy)phenyl)urea |
| 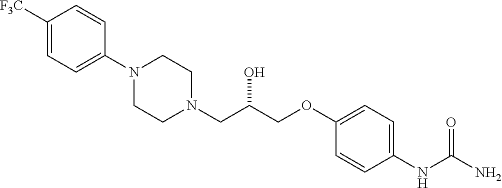 | (S)-1-(4-(2-hydroxy-3-(4-(4- (trifluoromethyl)phenyl)piperazin-1- yl)propoxy)phenyl)urea |
| 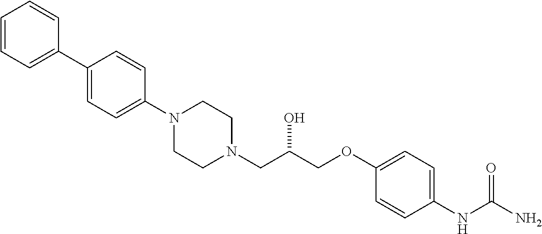 | (S)-1-(4-(3-(4-(biphenyl-4-yl)piperazin-1-yl)-2- hydroxypropoxy)phenyl)urea |
| 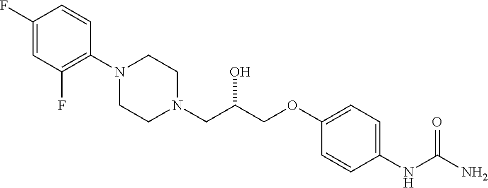 | (S)-1-(4-(3-(4-(2,4-difluorophenyl)piperazin-1-yl)- 2-hydroxypropoxy)phenyl)urea |
| 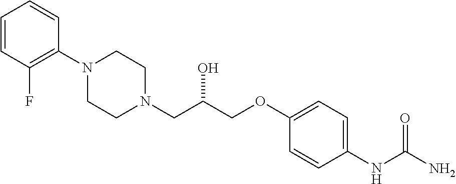 | (S)-1-(4-(3-(4-(2-fluorophenyl)piperazin-1-yl)-2- hydroxypropoxy)phenyl)urea |
| 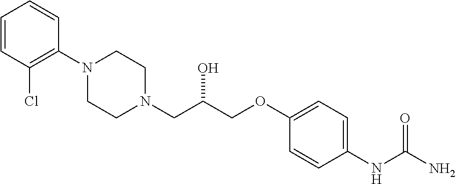 | (S)-1-(4-(3-(4-(2-chlorophenyl)piperazin-1-yl)-2- hydroxypropoxy)phenyl)urea |
| 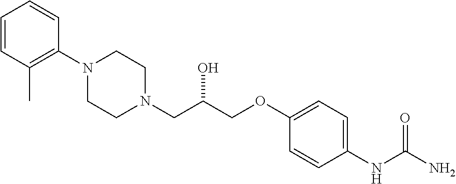 | (S)-1-(4-(2-hydroxy-3-(4-o-tolylpiperazin-1- yl)propoxy)phenyl)urea |
| 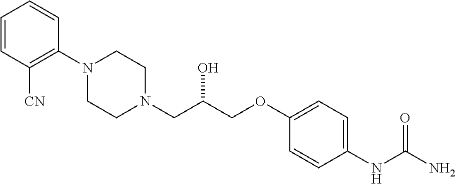 | (S)-1-(4-(3-(4-(2-cyanophenyl)piperazin-1-yl)-2- hydroxypropoxy)phenyl)urea |
| 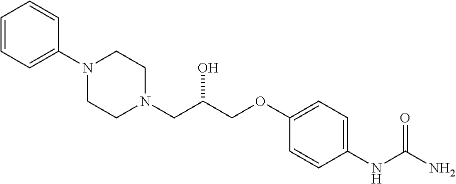 | (S)-1-(4-(2-hydroxy-3-(4-phenylpiperazin-1- yl)propoxy)phenyl)urea |
| 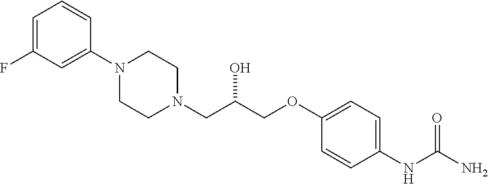 | (S)-1-(4-(3-(4-(3-fluorophenyl)piperazin-1-yl)-2- hydroxypropoxy)phenyl)urea |
| 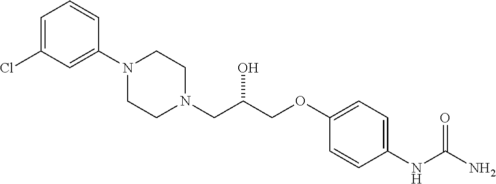 | (S)-1-(4-(3-(4-(3-chlorophenyl)piperazin-1-yl)-2- (S)-hydroxypropoxy)phenyl)urea |
| 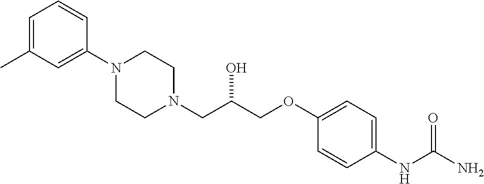 | (S)-1-(4-(2-hydroxy-3-(4-m-tolypiperazin-1- yl)propoxy)phenyl)urea |
| 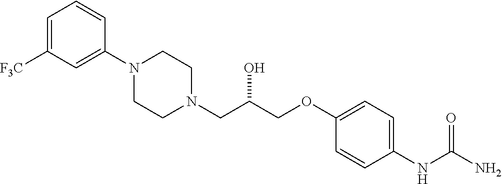 | (S)-1-(4-(2-hydroxy-3-(4-(3- (trifluoromethyl)phenyl)piperazin-1- yl)propoxy)phenyl)urea |
| 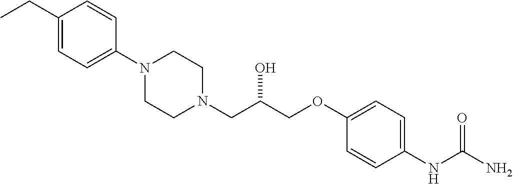 | (S)-1-(4-(3-(4-(4-ethylphenyl)piperazin-1-yl)-2- hydroxypropoxy)phenyl)urea |
| 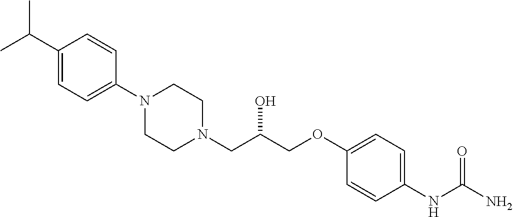 | (S)-1-(4-(3-(4-(4-isopropylphenyl)piperazin-1-yl)-2- hydroxypropoxy)phenyl)urea |
| 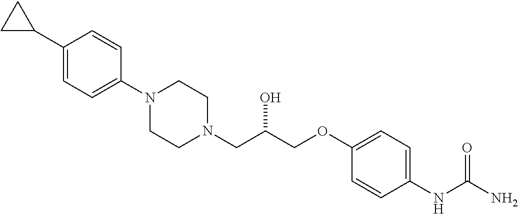 | (S)-1-(4-(3-(4-(4-cyclopropylphenyl)piperazin-1- yl)-2-hydroxypropoxy)phenyl)urea |
| 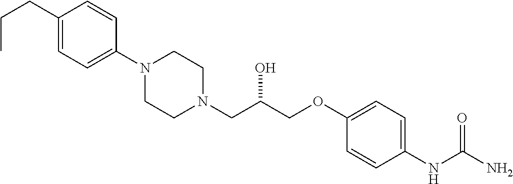 | (S)-1-(4-(3-(4-(4-propylphenyl)piperazin-1-yl)-2- hydroxypropoxy)phenyl)urea |
| 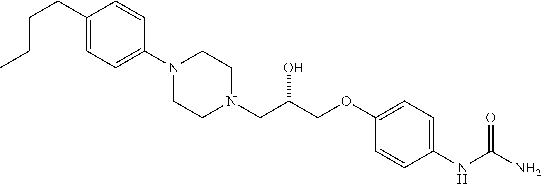 | (S)-1-(4-(3-(4-(4-butylphenyl)piperazin-1-yl)-2- hydroxypropoxy)phenyl)urea |
| 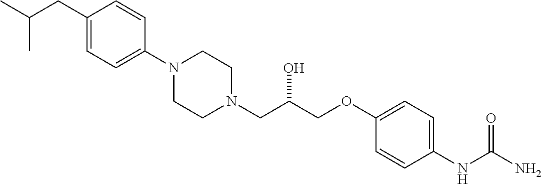 | (S)-1-(4-(3-(4-(4-isobutylphenyl)piperazin-1-yl)-2- hydroxypropoxy)phenyl)urea |
| 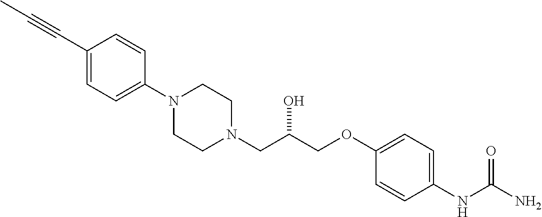 | (S)-1-(4-(2-hydroxy-3-(4-(4-(prop-1- ynyl)phenyl)piperazin-1-yl)propoxy)phenyl)urea |
| 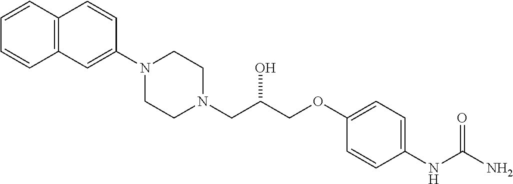 | (S)-1-(4-(3-(4-(2-naphthyl)piperazin-1-yl)-2- hydroxypropoxy)phenyl)urea |
| 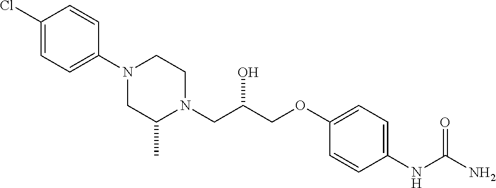 | 1-(4-((S)-3-((R)-4-(4-chlorophenyl)-2- methylpiperazin-1-yl)-2- hydroxypropoxy)phenyl)urea |
| 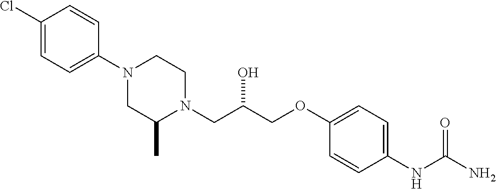 | 1-(4-((S)-3-((S)-4-(4-chlorophenyl)-2- methylpiperazin-1-yl)-2- hydroxypropoxy)phenyl)urea |
| 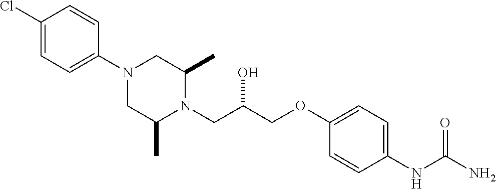 | 1-(4-((S)-3-4-(4-chlorophenyl)-cis-2,6- dimethylpiperazin-1-yl)-2- hydroxypropoxy)phenyl)urea |
| 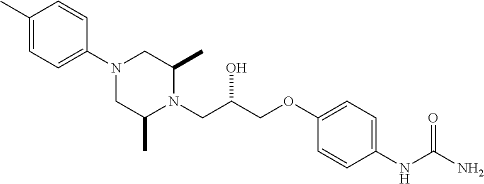 | 1-(4-((S)-3-(cis-2,6-dimethyl-4-p-tolylpiperazin-1- yl)-2-hydroxypropoxy)phenyl)urea |
| 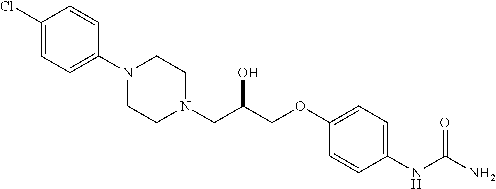 | (R)-1-(4-(3-(4-(4-chlorophenyl)piperazin-1-yl)-2- hydroxypropoxy)phenyl)urea |
| 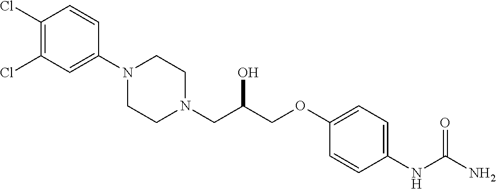 | (R)-1-(4-(3-(4-(3,4-dichlorophenyl)piperazin-1-yl)- 2-hydroxypropoxy)phenyl)urea |
| 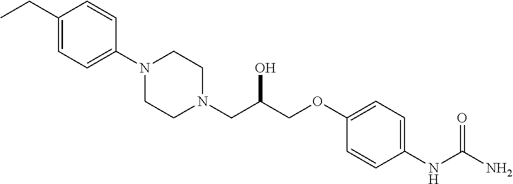 | (R)-1-(4-(3-(4-(4-ethylphenyl)piperazin-1-yl)-2- hydroxypropoxy)phenyl)urea |
| 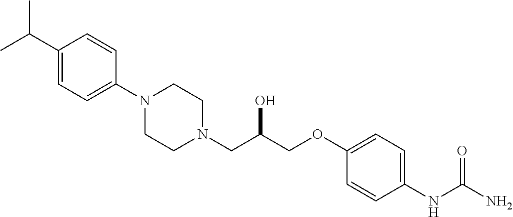 | (R)-1-(4-(3-(4-(4-isopropylphenyl)piperazin-1-yl)- 2-hydroxypropoxy)phenyl)urea |
| 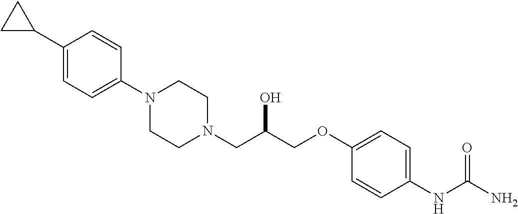 | (R)-1-(4-(3-(4-(4-cyclopropylphenyl)piperazin-1- yl)-2-hydroxypropoxy)phenyl)urea |
| 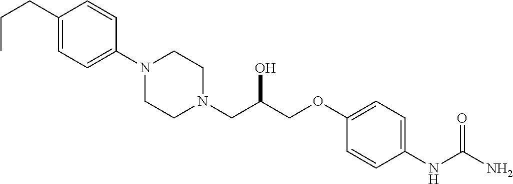 | (R)-1-(4-(3-(4-(4-propylphenyl)piperazin-1-yl)-2- hydroxypropoxy)phenyl)urea |
| 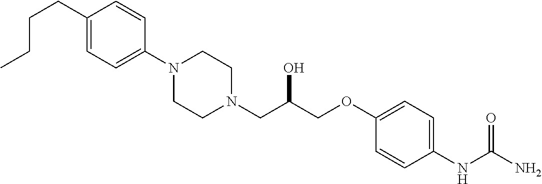 | (R)-1-(4-(3-(4-(4-butylphenyl)piperazin-1-yl)-2- hydroxypropoxy)phenyl)urea |
| 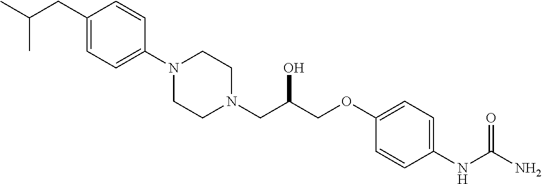 | (R)-1-(4-(3-(4-(4-isobutylphenyl)piperazin-1-yl)-2- hydroxypropoxy)phenyl)urea |
| 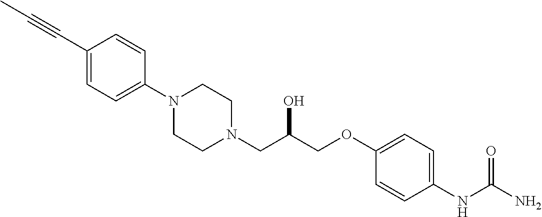 | (R)-1-(4-(2-hydroxy-3-(4-(4-(prop-1- ynyl)phenyl)piperazin-1-yl)propoxy)phenyl)urea |
| 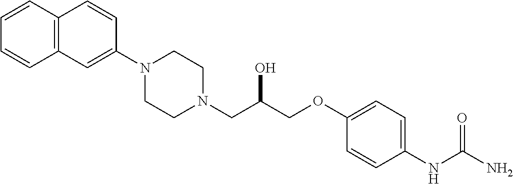 | (R)-1-(4-(3-(4-(2-naphthyl)piperazin-1-yl)-2- hydroxypropoxy)phcnyl)urea |
| 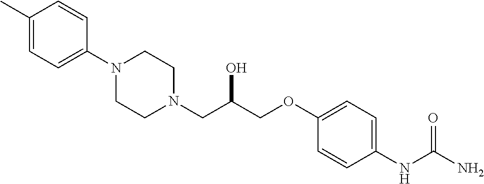 | (R)-1-(4-(3-(4-(4-methylphenyl)piperazin-1-yl)-2- hydroxypropoxy)phenyl)urea |
| 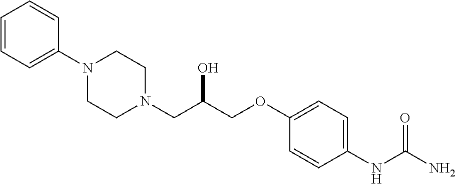 | (R)-1-(4-(3-(4-phenyl-piperazin-1-yl)-2- hydroxypropoxy)phenyl)urea |
| 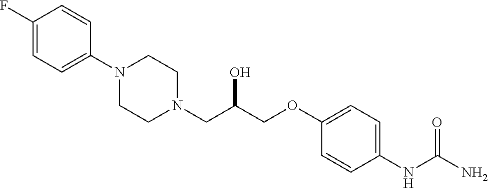 | (R)-1-(4-(3-(4-(4-fluorophenyl)piperazin-1-yl)-2- hydroxypropoxy)phenyl)urea |

In one embodiment, the compound is selected from the compounds in Table 5.

TABLE 5

| Compound | Name |
|---|---|
| 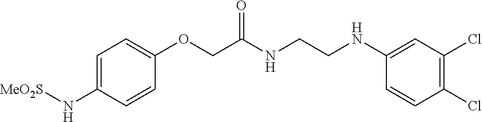 | N-[2-(3,4-Dichloro-phenylamino)-ethyl]-2-(4- methanesulfonylamino-phenoxy)-acetamide |
| 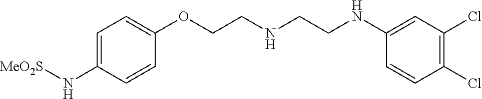 | N-(4-{2-[2-(3,4-Dichloro-phenylamino)- ethylamino]-ethoxy}-phenyl)- methanesulfonamide |
| 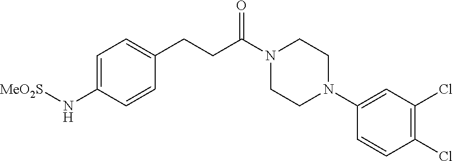 | N-(4-{3-[4-(3,4-Dichloro-phenyl)-piperazin-1- yl]-3-oxo-propyl}-phenyl)-methanesulfonamide |
| 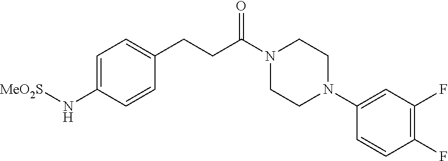 | N-(4-{3-[4-(3,4-DiFluoro-phenyl)-piperazin-1- yl]-3-oxo-propyl}-phenyl)-methanesulfonamide |
| 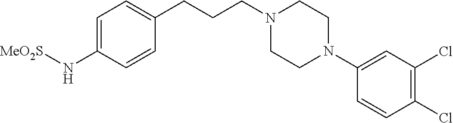 | N-(4-{3-[4-(3,4-Dichloro-phenyl)-piperazin-1- yl]-propyl}-phenyl)-methanesulfonamide |
| 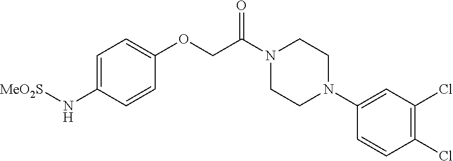 | N-(4-{2-[4-(3,4-Dichloro-phenyl)-piperazin-1- yl]-2-oxo-ethoxy}-phenyl)-methanesulfonamide |
| 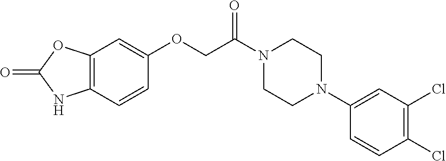 | 6-{2-[4-(3,4-Dichloro-phenyl)-piperazin-1- yl]-2-oxo-ethoxy}-3H-benzooxazol-2-one |
| 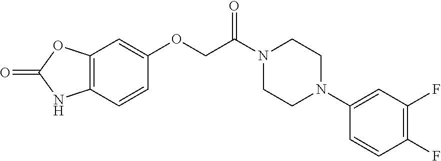 | 6-{2-[4-(3,4-DiFluoro-phenyl)-piperazin-1- yl]-2-oxo-ethoxy}-3H-benzooxazol-2-one |
| 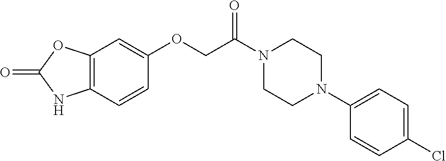 | 6-{2-[4-(4-Chloro-phenyl)-piperazin-1- yl]-2-oxo-ethoxy}-3H-benzooxazol-2-one |
| 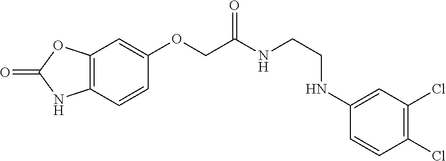 | N-[2-(3,4-Dichloro-phenylamino)-ethyl]-2-(2- oxo-2,3-dihydro-benzooxazol-6-yloxy)- acetamide |
| 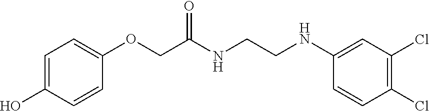 | N-[2-(3,4-Dichloro-phenylamino)-ethyl]-2-(4- hydroxy-phenoxy)-acetamide |
| 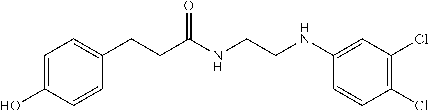 | N-[2-(3,4-Dichloro-phenylamino)-ethyl]-3-(4- hydroxy-phenyl)-propionamide |
| 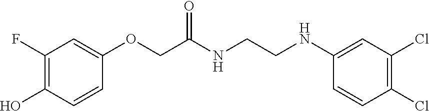 | N-[2-(3,4-Dichloro-phenylamino)-ethyl]-2-(3- fluoro-4-hydroxy-phenoxy)-acetamide |
| 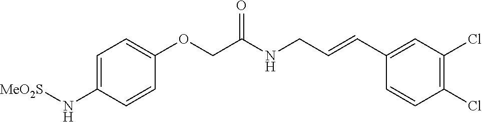 | N-[3-(3,4-Dichloro-phenyl)-allyl]-2-(4- methanesulfonylamino-phenoxy)-acetamide |
| 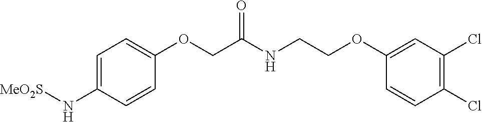 | N-[2-(3,4-Dichloro-phenoxy)-ethyl]-2-(4- methanesulfonylamino-phenoxy)-acetamide |
| 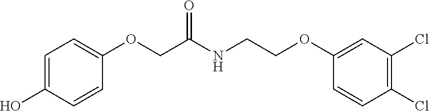 | N-[2-(3,4-Dichloro-phenoxy)-ethyl]-2-(4- hydroxy-phenoxy)-acetamide |
| 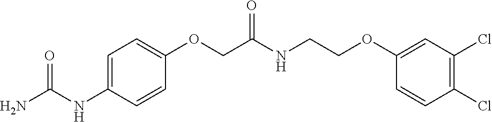 | N-[2-(3,4-Dichloro-phenoxy)-ethyl]-2-(4- ureido-phenoxy)-acetamide |
| 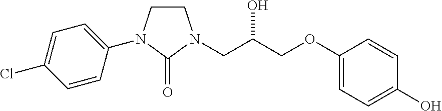 | (S)-1-(4-chlorophenyl)-3-(2-hydroxy-3-(4- hydroxyphenoxy)propyl)imidazolidin-2-one |
| 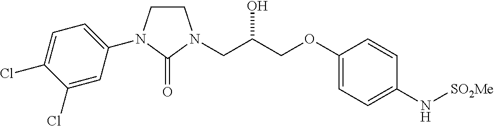 | (S)-N-(4-(3-(3-(3,4-dichlorophenyl)-2- oxoimidazolidin-1-yl)-2- hydroxypropoxy)phenyl)methanesulfonamide |
| 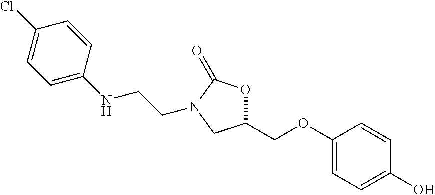 | (S)-3-(2-(4-chlorophenylamino)ethyl)-5-((4- hydroxyphenoxy)methyl)oxazolidin-2-one |
| 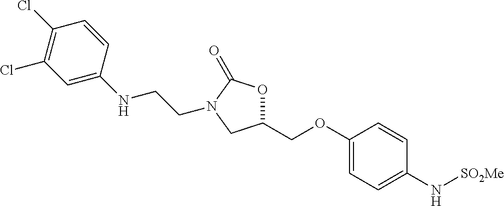 | (S)-N-(4-((3-(2-(3,4- dichlorophenylamino)ethyl)-2-oxooxazolidin-5- yl)methoxy)phenyl)methanesulfonamide |

In one embodiment, the compound is selected from Table 6.

TABLE 6

| Compound | NAME |
|---|---|
| 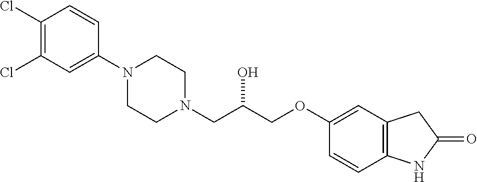 | (S)-5-(3-(4-(3,4-dichlorophenyl)piperazin-1-yl)-2- hydroxypropoxy)indolin-2-one |
| 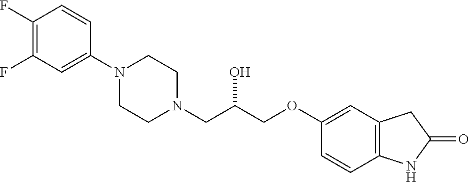 | (S)-5-(3-(4-(3,4-difluorophenyl)piperazin-1-yl)-2- hydroxypropoxy)indolin-2-one |
| 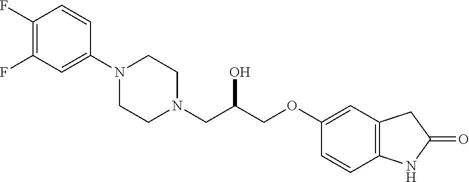 | (R)-5-(3-(4-(3,4-difluorophenyl)piperazin-1-yl)-2- hydroxypropoxy)indolin-2-one |
| 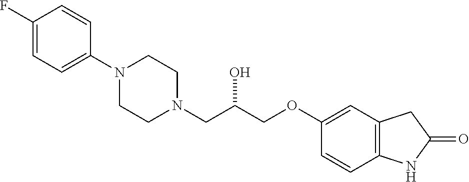 | (S)-5-(3-(4-(4-fluorophenyl)piperazin-1-yl)-2- hydroxypropoxy)indolin-2-one |
| 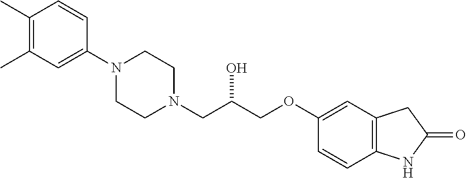 | (S)-5-(3-(4-(3,4-dimethylphenyl)piperazin-1-yl)-2- hydroxypropoxy)indolin-2-one |
| 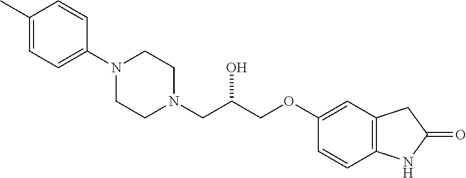 | (S)-5-(2-hydroxy-3-(4-p-tolylpiperazin-1- yl)propoxy)indolin-2-one |
| 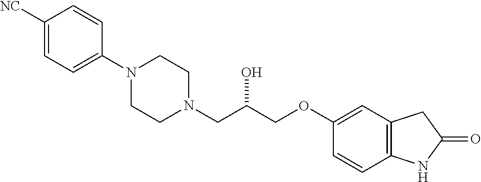 | (S)-4-(4-(2-hydroxy-3-(2-oxoindolin-5- yloxy)propyl)piperazin-1-yl)benzonitrile |
| 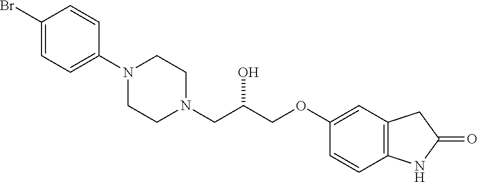 | (S)-5-(3-(4-(4-bromophenyl)piperazin-1-yl)-2- hydroxypropoxy)indolin-2-one |
| 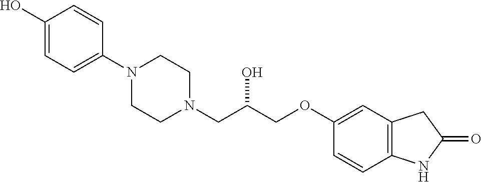 | (S)-5-(2-hydroxy-3-(4-(4-hydroxyphenyl)piperazin-1- yl)propoxy)indolin-2-one |
| 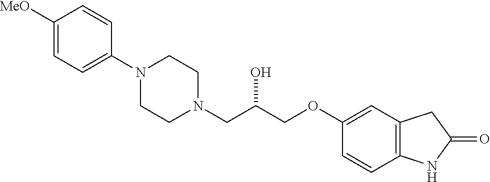 | (S)-5-(2-hydroxy-3-(4-(4-methoxyphenyl)piperazin-1- yl)propoxy)indolin-2-one |
| 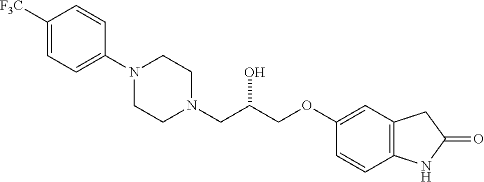 | (S)-5-(2-hydroxy-3-(4-(4- (trifluoromethyl)phenyl)piperazin-1- yl)propoxy)indolin-2-one |
| 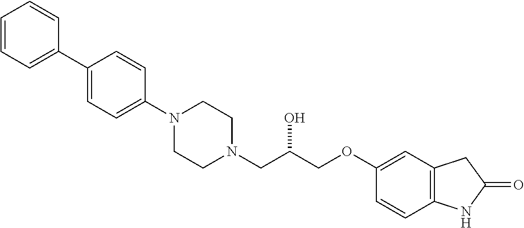 | (S)-5-(3-(4-(biphenyl-4-yl)piperazin-1-yl)-2- hydroxypropoxy)indolin-2-one |
| 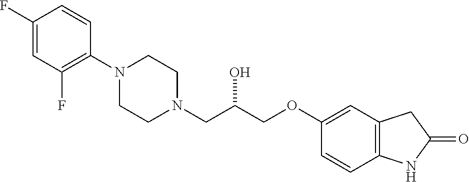 | (S)-5-(3-(4-(2,4-difluorophenyl)piperazin-1-yl)-2- hydroxypropoxy)indolin-2-one |
| 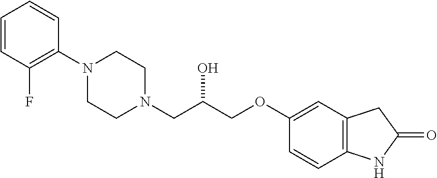 | (S)-5-(3-(4-(2-fluorophenyl)piperazin-1-yl)-2- hydroxypropoxy)indolin-2-one |
| 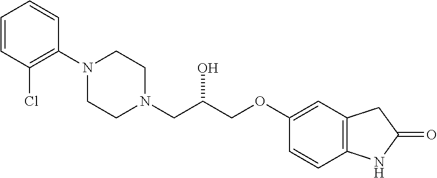 | (S)-5-(3-(4-(2-chlorophenyl)piperazin-1-yl)-2- hydroxypropoxy)indolin-2-one |
| 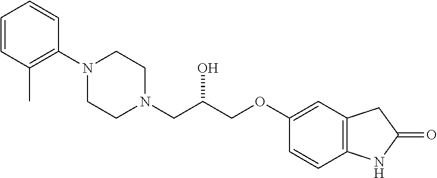 | (S)-5-(2-hydroxy-3-(4-o-tolylpiperazin-1- yl)propoxy)indolin-2-one |
| 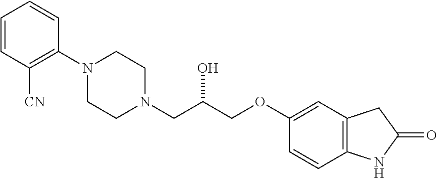 | (S)-2-(4-(2-hydroxy-3-(2-oxoindolin-5- yloxy)propyl)piperazin-1-yl)benzonitrile |
| 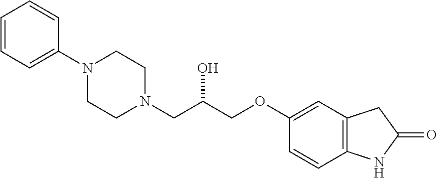 | (S)-5-(2-hydroxy-3-(4-phenylpiperazin-1- yl)propoxy)indolin-2-one |
| 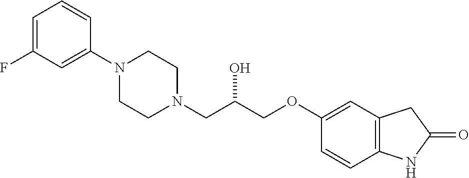 | (S)-5-(3-(4-(3-fluorophenyl)piperazin-1-yl)-2- hydroxypropoxy)indolin-2-one |
| 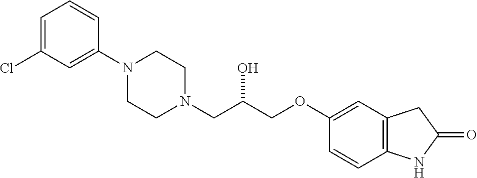 | (S)-5-(3-(4-(3-chlorophenyl)piperazin-1-yl)-2- hydroxypropoxy)indolin-2-one |
| 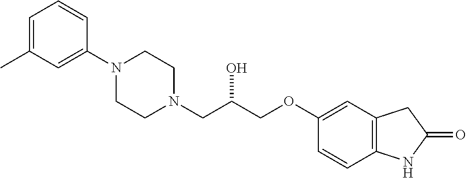 | (S)-5-(2-hydroxy-3-(4-m-tolylpiperazin-1- yl)propoxy)indolin-2-one |
| 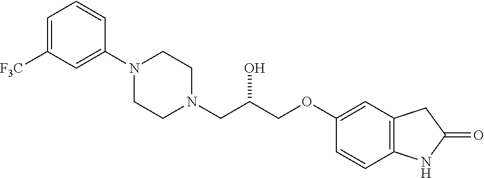 | (S)-5-(2-hydroxy-3-(4-(3- (trifluoromethyl)phenyl)piperazin-1- yl)propoxy)indolin-2-one |
| 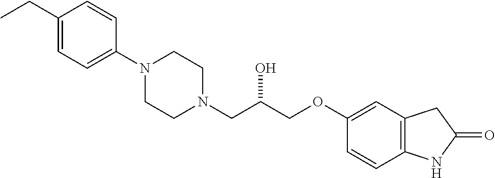 | (S)-5-(3-(4-(4-ethylphenyl)piperazin-1-yl)-2- hydroxypropoxy)indolin-2-one |
| 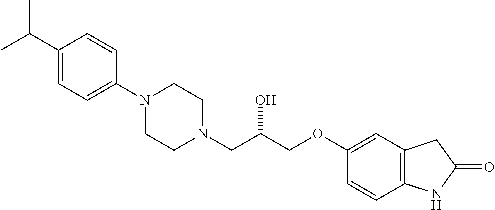 | (S)-5-(2-hydroxy-3-(4-(4-isopropylphenyl)piperazin- 1-yl)propoxy)indolin-2-one |
| 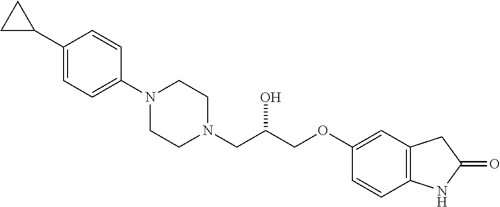 | (S)-5-(3-(4-(4-cyclopropylphenyl)piperazin- 1-yl)-2-hydroxypropoxy)indolin-2-one |
| 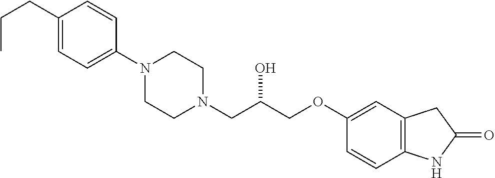 | (S)-5-(2-hydroxy-3-(4-(4-propylphenyl)piperazin- 1-yl)propoxy)indolin-2-one |
| 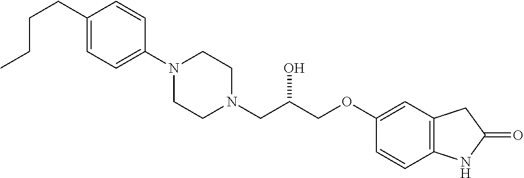 | (S)-5-(3-(4-(4-butylphenyl)piperazin- 1-yl)-2-hydroxypropoxy)indolin-2-one |
| 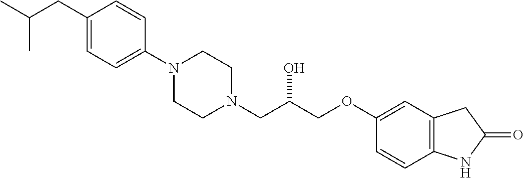 | (S)-5-(2-hydroxy-(3-(4-(4-isobutylphenyl)piperazin- 1-yl)propoxy)indolin-2-one |
| 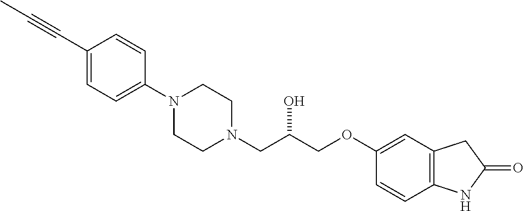 | (S)-5-(2-hydroxy-3-(4-(4-(prop-1- ynyl)phenyl)piperazin-1-yl)propoxy)indolin-2-one |
| 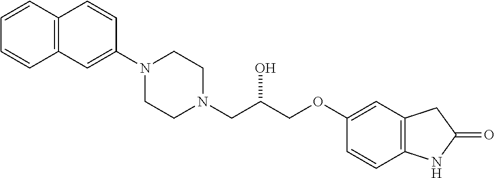 | (S)-5-(2-hydroxy-3-(4-(naphthalen-2-yl)piperazin-1- yl)propoxy)indolin-2-one |
| 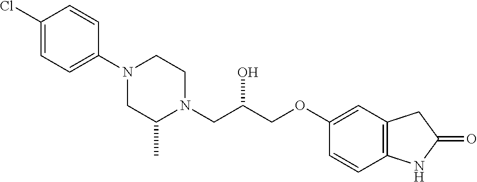 | 5-((S)-3-((R)-4-(4-chlorophenyl)-2-methylpiperazin-1- yl)-2-hydroxypropoxy)indolin-2-one |
| 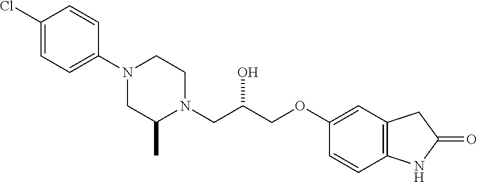 | 5-((S)-3-((S)-4-(4-chlorophenyl)-2-methylpiperazin-1- yl)-2-hydroxypropoxy)indolin-2-one |
| 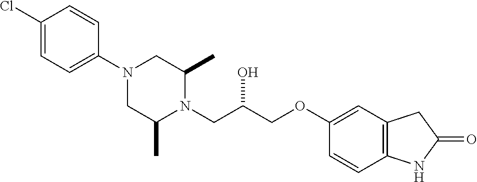 | 5-((S)-3-((2S,6R)-4-(4-chlorophenyl)-2,6- dimethylpiperazin-1-yl)-2-hydroxypropoxy)indolin-2- one |
| 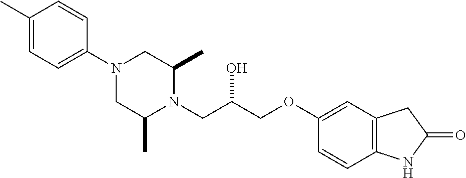 | 5-((S)-3-((2S,6R)-2,6-dimethyl-4-p-tolylpiperazin-1- yl)-2-hydroxypropoxy)indolin-2-one |
| 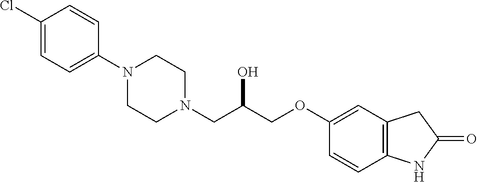 | (R)-5-(3-(4-(4-chlorophenyl)piperazin-1-yl)-2- hydroxypropoxy)indolin-2-one |
| 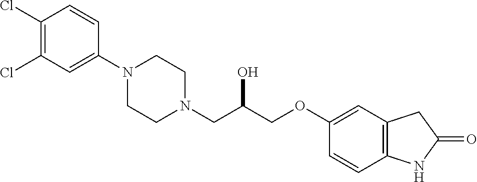 | (R)-5-(3-(4-(3,4-dichlorophenyl)piperazin-1-yl)-2- hydroxypropoxy)indolin-2-one |
| 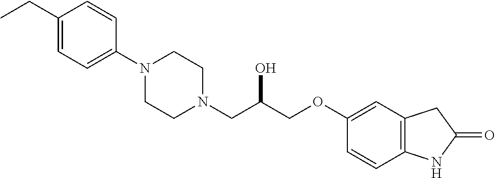 | (R)-5-(3-(4-(4-ethylphenyl)piperazin-1-yl)-2- hydroxypropoxy)indolin-2-one |
| 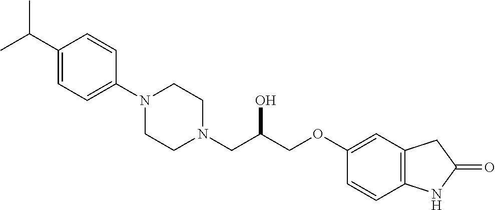 | (R)-5-(2-hydroxy-3-(4-(4-isopropylphenyl)piperazin- 1-yl)propoxy)indolin-2-one |
| 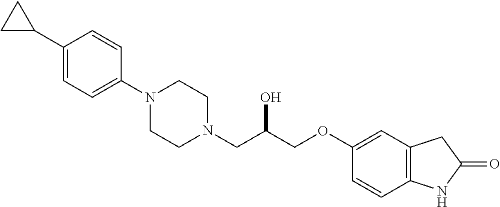 | (R)-5-(3-(4-(4-cyclopropylphenyl)piperazin-1-yl)-2- hydroxypropoxy)indolin-2-one |
| 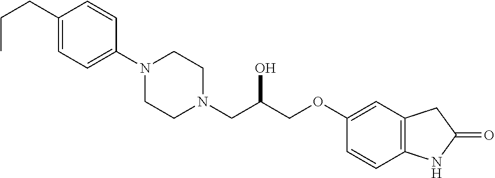 | (R)-5-(2-hydroxy-3-(4-(4-propylphenyl)piperazin-1- yl)propoxy)indolin-2-one |
| 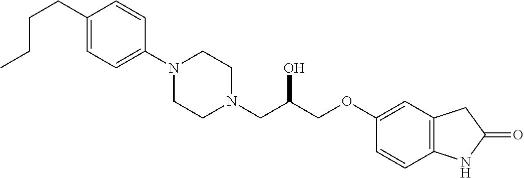 | (R)-5-(3-(4-(4-butylphenyl)piperazin-1-yl)-2- hydroxypropoxy)indolin-2-one |
| 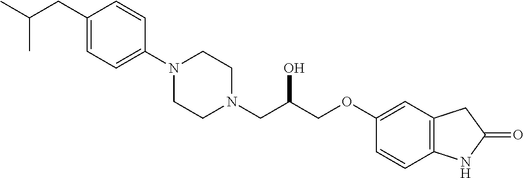 | (R)-5-(2-hydroxy-3-(4-(4-isobutylphenyl)piperazin-1- yl)propoxy)indolin-2-one |
| 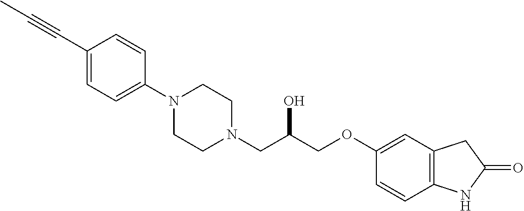 | (R)-5-(2-hydroxy-3-(4-(4-(prop-1- ynyl)phenyl)piperazin-1-yl)propoxy)indolin-2-one |
| 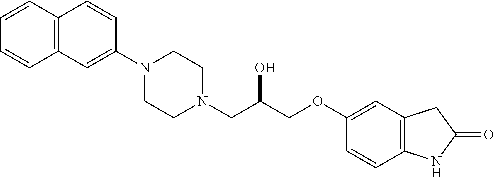 | (R)-5-(2-hydroxy-3-(4-(naphthalen-2-yl)piperazin-1- yl)propoxy)indolin-2-one |
| 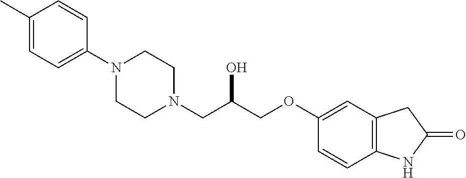 | (R)-5-(2-hydroxy-(3-(4-p-tolylpiperazin-1- yl)propoxy)indolin-2-one |
| 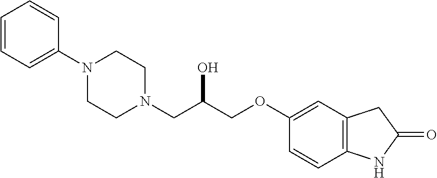 | (R)-5-(2-hydroxy-3-(4-phenylpiperazin-1- yl)propoxy)indolin-2-one |
| 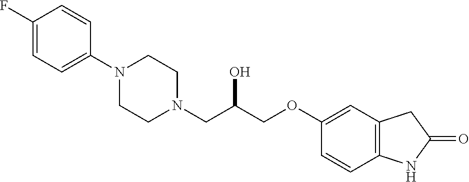 | (R)-5-(3-(4-(4-fluorophenyl)piperazin-1-yl)-2- hydroxypropoxy)indolin-2-one |

In one embodiment, the compound is selected from Table 7.

TABLE 7

| Compound | NAME |
|---|---|
| 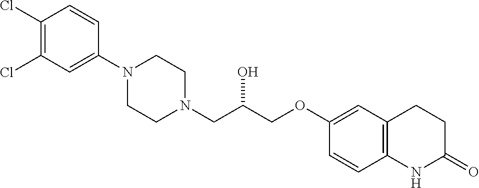 | (S)-6-(3-(4-(3,4-dichlorophenyl)piperazin-1-yl)-2- hydroxypropoxy)-3,4-dihydroquinolin-2(1H)-one |
| 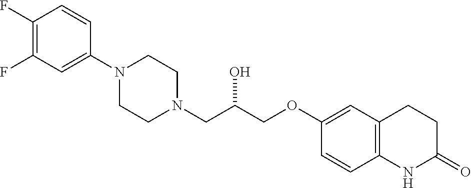 | (S)-6-(3-(4-(3,4-dichlorophenyl)piperazin-1-yl)-2- hydroxypropoxy)-3,4-dihydroquinolin-2(1H)-one |
| 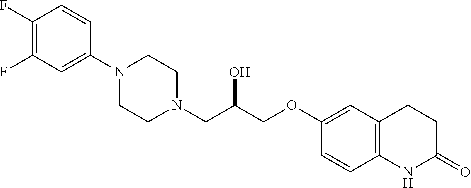 | (R)-6-(3-(4-(3,4-difluorophenyl)piperazin-1-yl)-2- hydroxypropoxy)-3,4-dihydroquinolin-2(1H)-one |
| 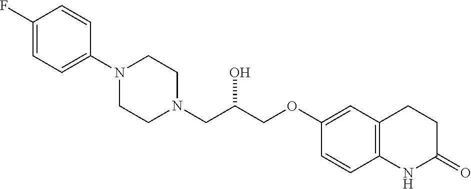 | (S)-6-(3-(4-(4-fluorophenyl)piperazin-1-yl)-2- hydroxypropoxy)-3,4-dihydroquinolin-2(1H)-one |
| 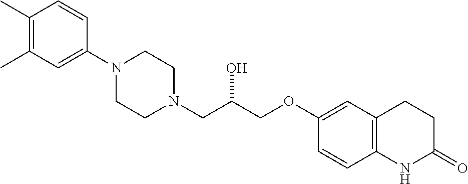 | (S)-6-(3-(4-(3,4-dimethylphenyl)piperazin-1-yl)-2- hydroxypropoxy)-3,4-dihydroquinolin-2(1H)-one |
| 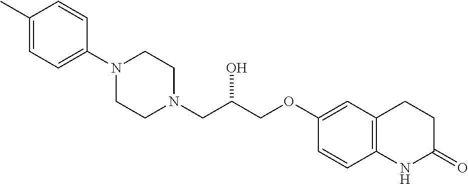 | (S)-6-(2-hydroxy-3-(4-p-tolylpiperazin-1-yl)propoxy)- 3,4-dihydroquinolin-2(1H)-one |
| 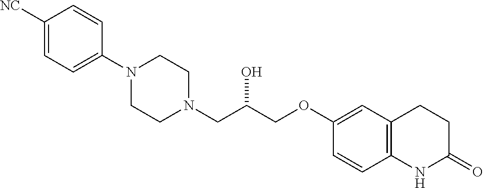 | (S)-4-(4-(2-hydroxy-3-(2-oxo-1,2,3,4- tetrahydroquinolin-6-yloxy)propyl)piperazin-1- yl)benzonitrile |
| 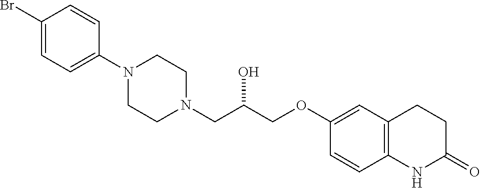 | (S)-6-(3-(4-(4-bromophenyl)piperazin-1-yl)-2- hydroxypropoxy)-3,4-dihydroquinolin-2(1H)-one |
| 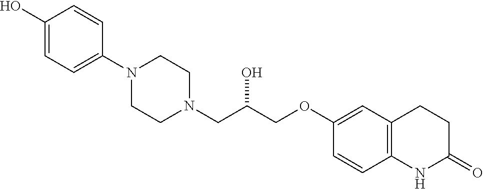 | (S)-6-(2-hydroxy-3-(4-(4-hydroxyphenyl)piperazin-1- yl)propoxy)-3,4-dihydroquinolin-2(1H)-one |
| 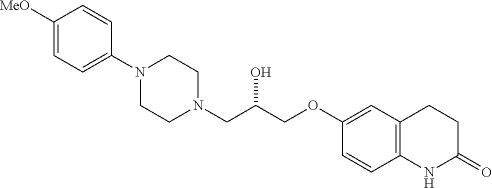 | (S)-6-(2-hydroxy-3-(4-(4-methoxyphenyl)piperazin-1- yl)propoxy)-3,4-dihydroquinolin-2(1H)-one |
| 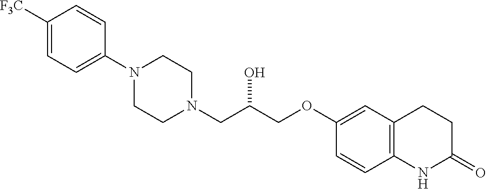 | (S)-6-(2-hydroxy-3-(4-(4- (trifluoromethyl)phenyl)piperazin-1-yl)propoxy)-3,4- dihydroquinolin-2(1H)-one |
| 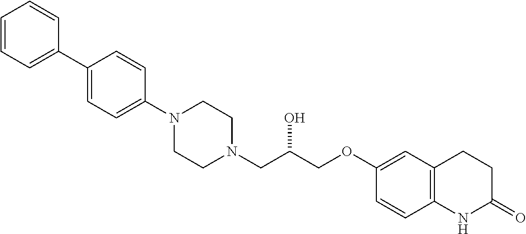 | (S)-6-(3-(4-(biphenyl-4-yl)piperazin-1-yl)-2- hydroxypropoxy)-3,4-dihydroquinolin-2(1H)-one |
| 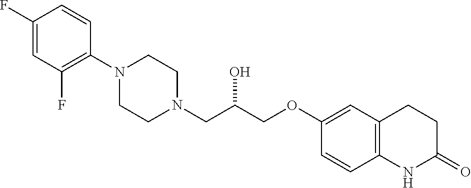 | (S)-6-(3-(4-(2,4-difluorophenyl)piperazin-1-yl)-2- hydroxypropoxy)-3,4-dihydroquinolin-2(1H)-one |
| 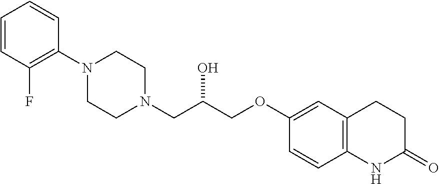 | (S)-6-(3-(4-(2-fluorophenyl)piperazin-1-yl)-2- hydroxypropoxy)-3,4-dihydroquinolin-2(1H)-one |
| 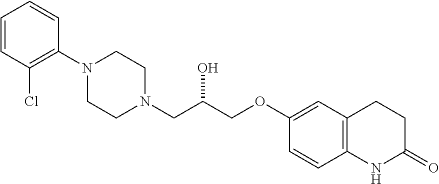 | (S)-6-(3-(4-(2-chlorophenyl)piperazin-1-yl)-2- hydroxypropoxy)-3,4-dihydroquinolin-2(1H)-one |
| 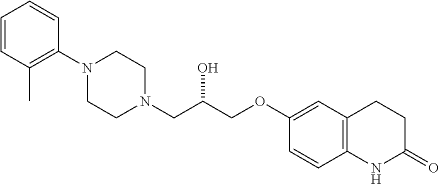 | (S)-6-(2-hydroxy-3-(4-o-tolylpiperazin-1-yl)propoxy)- 3,4-dihydroquinolin-2(1H)-one |
| 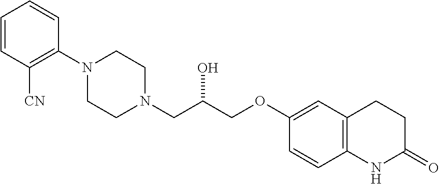 | (S)-2-(4-(2-hydroxy-3-(2-oxo-1,2,3,4- tetrahydroquinolin-6-yloxy)propyl)piperazin-1- yl)benzonitrile |
| 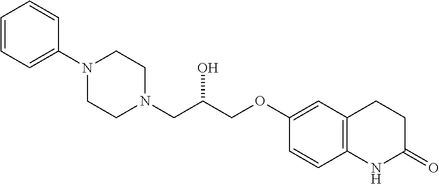 | (S)-6-(2-hydroxy-3-(4-phenylpiperazin-1-yl)propoxy)- 3,4-dihydroquinolin-2(1H)-one |
| 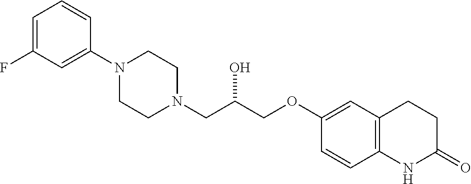 | (S)-6-(3-(4-(3-fluoropheny1)piperazin-1-yl)-2- hydroxypropoxy)-3,4-dihydroquinolin-2(1H)-one |
| 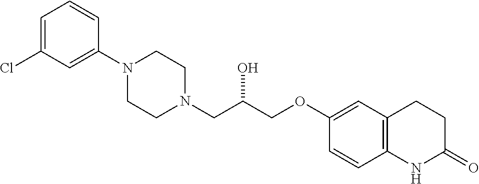 | (S)-6-(3-(4-(3-chlorophenyl)piperazin-1-yl)-2- hydroxypropoxy)-3,4-dihydroquinolin-2(1H)-one |
| 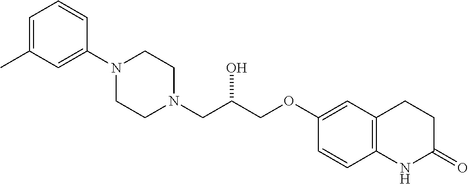 | (S)-6-(2-hydroxy-3-(4-m-tolypiperazin-1-yl)propoxy)- 3,4-dihydroquinolin-2(1H)-one |
| 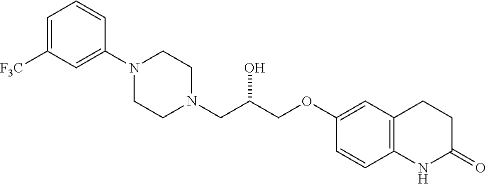 | (S)-6-(2-hydroxy-3-(4-(3- (trifluoromethyl)phenyl)piperazin-1-yl)propoxy)-3,4- dihydroquinolin-2(1H)-one |
| 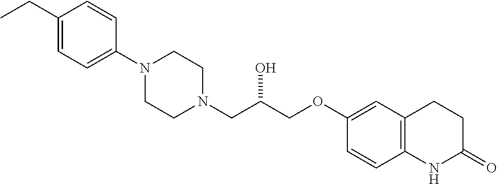 | (S)-6-(3-(4-(4-ethylphenyl)piperazin-1-yl)-2- hydroxypropoxy)-3,4-dihydroquinolin-2(1H)-one |
| 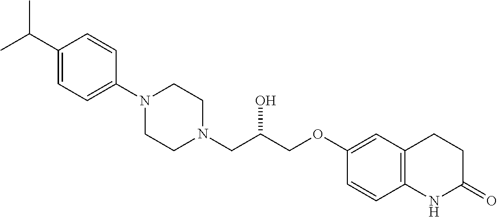 | (S)-6-(2-hydroxy-3-(4-(4-isopropylphenyl)piperazin- 1-yl)propoxy)-3,4-dihydroquinolin-2(1H)-one |
| 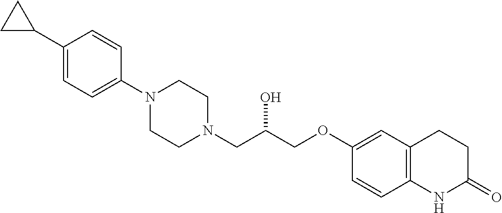 | (S)-6-(3-(4-(4-cyclopropylphenyl)piperazin-1-yl)-2- hydroxypropoxy)-3,4-dihydroquinolin-2(1H)-one |
| 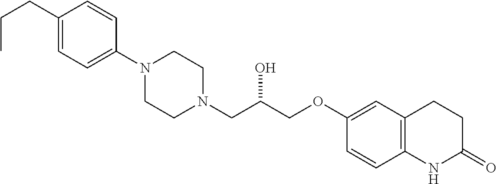 | (S)-6-(2-hydroxy-3-(4-(4-propylphenyl)piperazin-1- yl)propoxy)-3,4-dihydroquinolin-2(1H)-one |
| 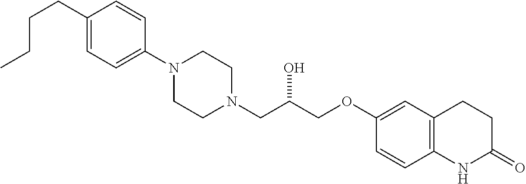 | (S)-6-(3-(4-(4-butylphenyl)piperazin-1-yl)-2- hydroxypropoxy)-3,4-dihydroquinolin-2(1H)-one |
| 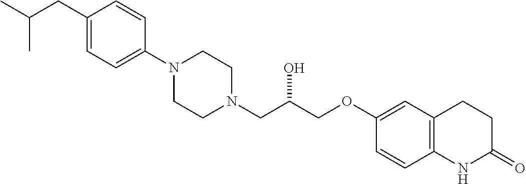 | (S)-6-(2-hydroxy-3-(4-(4-isobutylphenyl)piperazin-1- yl)propoxy)-3,4-dihydroquinolin-2(1H)-one |
| 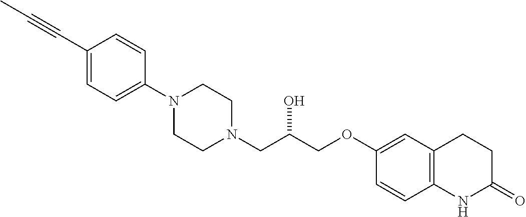 | (S)-6-(2-hydroxy-3-(4-(4-(prop-1- ynyl)phenyl)piperazin-1-yl)propoxy)-3,4- dihydroquinolin-2(1H)-one |
| 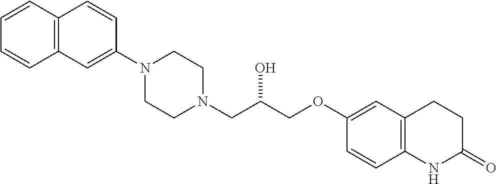 | (S)-6-(2-hydroxy-3-(4-(naphthalen-2-yl)-piperazin-1- yl)propoxy)-3,4-dihydroquinolin-2(1H)-one |
| 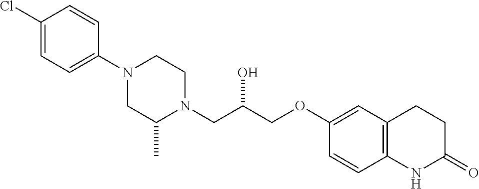 | 6-((S)-3-((R)-4-(4-chlorophenyl)-2-methylpiperazin-1- yl)-2-hydroxypropoxy)-3,4-dihydroquinolin-2(1H)- one |
| 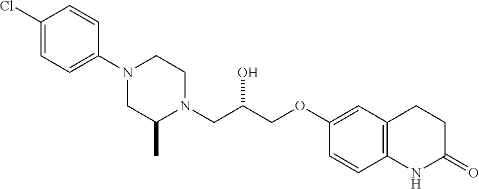 | 6-((S)-3-((S)-4-(4-chlorophenyl)-2-methylpiperazin-1- yl)-2-hydroxypropoxy)-3,4-dihydroquinolin-2(1H)- one |
| 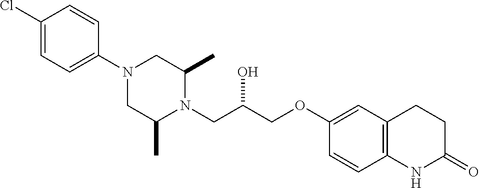 | 6-((S)-3-((2S,6R)-4-(4-chlorophenyl)-2,6- dimethylpiperazin-1-yl)-2-hydroxypropoxy)-3,4- dihydroquinolin-2(1H)-one |
| 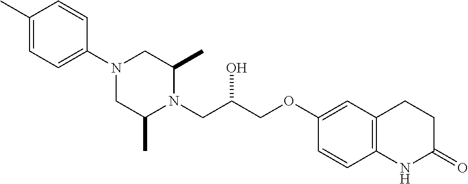 | 6-((S)-3-((2S,6R)-2,6-dimethyl-4-p-tolylpiperazin-1- yl)-2-hydroxypropoxy)-3,4-dihydroquinolin-2(1H)- one |
| 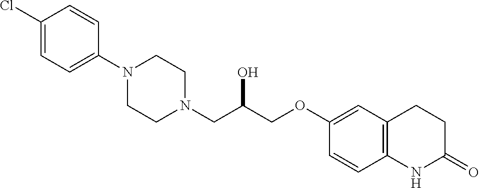 | (R)-6-(3-(4-(4-chlorophenyl)piperazin-1-yl)-2- hydroxypropoxy)-3,4-dihydroquinolin-2(1H)-one |
| 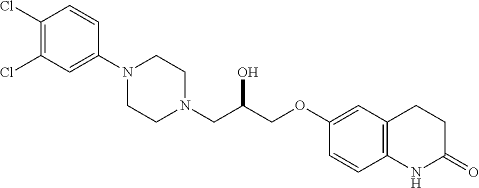 | (R)-6-(3-(4-(3,4-dichlorophenyl)piperazin-1-yl)-2- hydroxypropoxy)-3,4-dihydroquinolin-2(1H)-one |
| 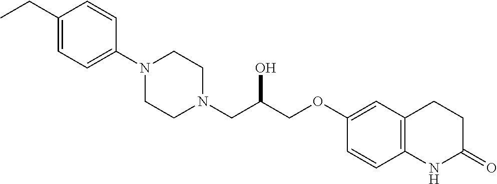 | (R)-6-(3-(4-(4-ethylphenyl)piperazin-1-yl)-2- hydroxypropoxy)-3,4-dihydroquinolin-2(1H)-one |
| 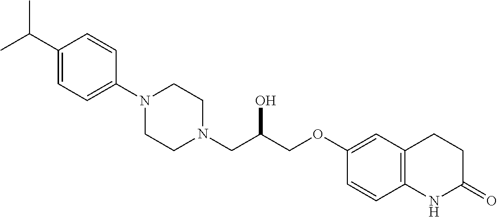 | (R)-6-(2-hydroxy-3-(4-(4-isopropylphenyl)piperazin- 1-yl)propoxy)-3,4-dihydroquinolin-2(1H)-one |
| 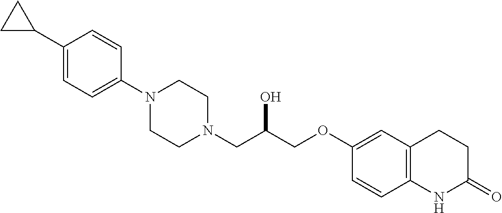 | (R)-6-(3-(4-(4-cyclopropylphenyl)piperazin-1-yl)-2- hydroxypropoxy)-3,4-dihydroquinolin-2(1H)-one |
| 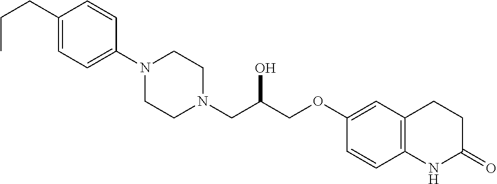 | (R)-6-(2-hydroxy-3-(4-(4-propylphenyl)piperazin-1- yl)propoxy)-3,4-dihydroquinolin-2(1H)-one |
| 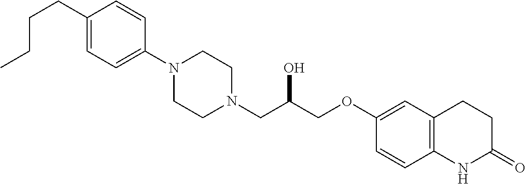 | (R)-6-(3-(4-(4-butylphenyl)piperazin-1-yl)-2- hydroxypropoxy)-3,4-dihydroquinolin-2(1H)-one |
| 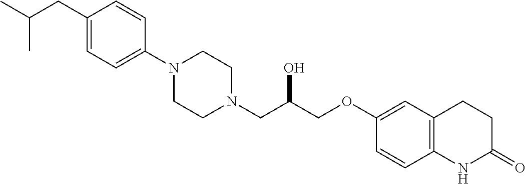 | (R)-6-(2-hydroxy-3-(4-(4-isobutylphenyl)piperazin-1- yl)propoxy)-3,4-dihydroquinolin-2(1H)-one |
| 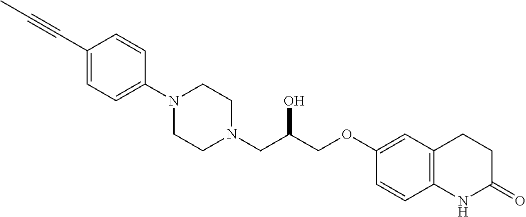 | (R)-6-(2-hydroxy-3-(4-(4-(prop-1- ynyl)phenyl)piperazin-1-yl)propoxy)-3,4- dihydroquinolin-2(1H)-one |
| 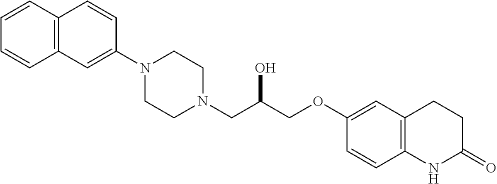 | (R)-6-(2-hydroxy-3-(4-(naphthalen-2-yl)piperazin-1- yl)propoxy)-3,4-dihydroquinolin-2(1H)-one |
| 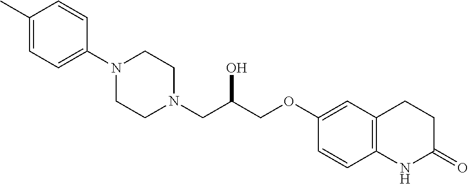 | (R)-6-(2-hydroxy-3-(4-p-tolylpiperazin-1-yl)propoxy)- 3,4-dihydroquinolin-2(1H)-one |
| 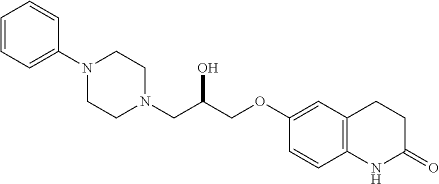 | (R)-6-(2-hydroxy-3-(4-phenylpiperazin-1- yl)propoxy)-3,4-dihydroquinolin-2(1H)-one |
| 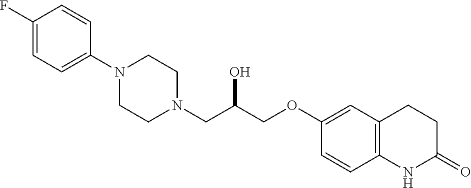 | (R)-6-(3-(4-(4-fluorophenyl)piperazin-1-yl)-2- hydroxypropoxy)-3,4-dihydroquinolin-2(1H)-one |

In another embodiment, the compound is selected from Table 8.

TABLE 8

| Compound | NAME |
|---|---|
| 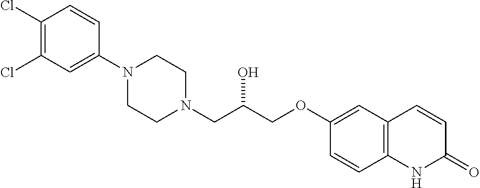 | (S)-6-(3-(4-(3,4-dichlorophenyl)piperazin-1-yl)-2- hydroxypropoxy)quinolin-2(1H)-one |
| 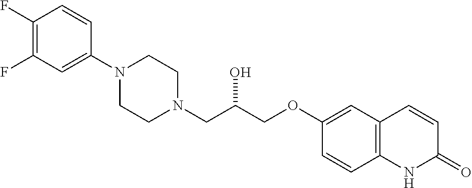 | (S)-6-(3-(4-(3,4-difluorophenyl)piperazin-1-yl)-2- hydroxypropoxy)quinolin-2(1H)-one |
| 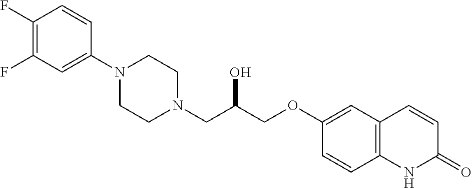 | (R)-6-(3-(4-(3,4-difluorophenyl)piperazin-1-yl)-2- hydroxypropoxy)quinolin-2(1H)-one |
| 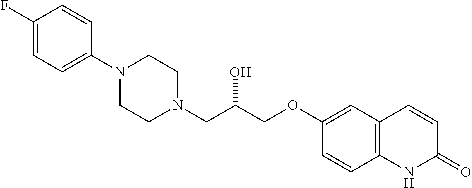 | (S)-6-(3-(4-(4-fluorophenyl)piperazin-1-yl)-2- hydroxypropoxy)quinolin-2(1H)-one |
| 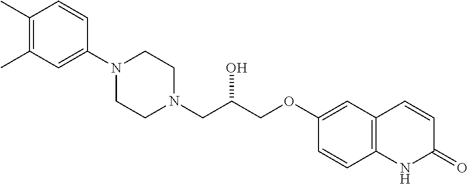 | (S)-6-(3-(4-(3,4-dimethylphenyl)piperazin-1-yl)-2- hydroxypropoxy)quinolin-2(1H)-one |
| 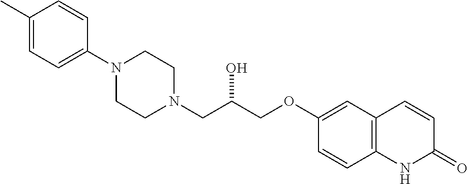 | (S)-6-(2-hydroxy-3-(4-p-tolylpiperazin-1- yl)propoxy)quinolin-2(1H)-one |
| 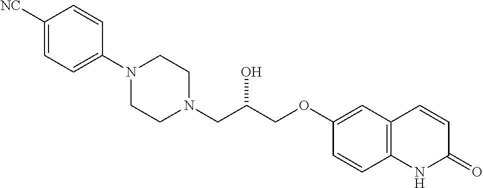 | (S)-4-(4-(2-hydroxy-3-(2-oxo-1,2-dihydroquinolin-6- yloxy)propyl)piperazin-1-yl)benzonitrile |
| 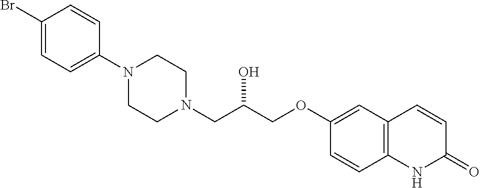 | (S)-6-(3-(4-(4-bromophenyl)piperazin-1-yl)-2- hydroxypropoxy)quinolin-2(1H)-one |
| 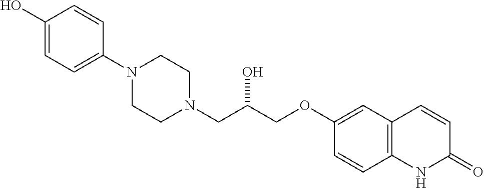 | (S)-6-(2-hydroxy-3-(4-(4-hydroxyphenyl)piperazin-1- yl)propoxy)quinolin-2(1H)-one |
| 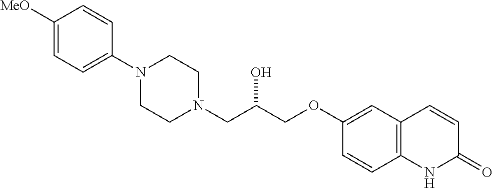 | (S)-6-(2-hydroxy-3-(4-(4-methoxyphenyl)piperazin-1- yl)propoxy)quinolin-2(1H)-one |
| 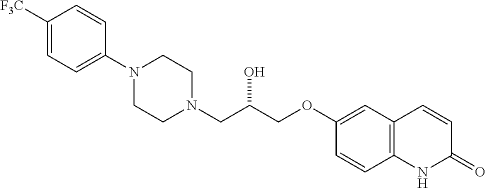 | (S)-6-(2-hydroxy-3-(4-(4- (trifluoromethyl)phenyl)piperazin-1- yl)propoxy)quinolin-2(1H)-one |
| 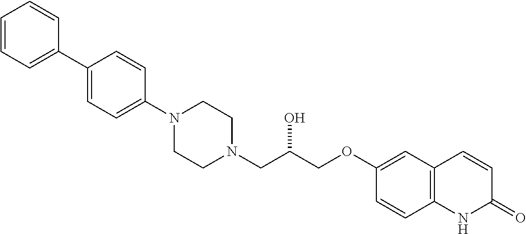 | (S)-6-(3-(4-(biphenyl-4-yl)piperazin-1-yl)-2- hydroxypropoxy)quinolin-2(1H)-one |
| 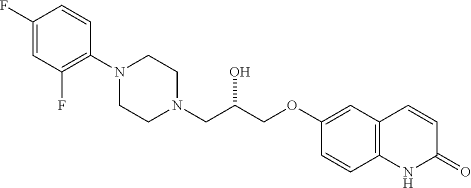 | (S)-6-(3-(4-(2,4-difluorophenyl)piperazin-1-yl)-2- hydroxypropoxy)quinolin-2(1H)-one |
| 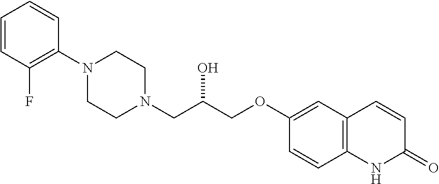 | (S)-6-(3-(4-(2-fluorophenyl)piperazin-1-yl)-2- hydroxypropoxy)quinolin-2(1H)-one |
| 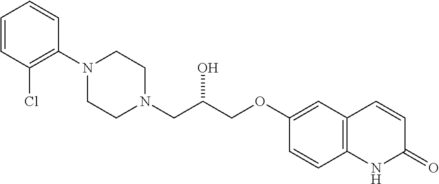 | (S)-6-(3-(4-(2-chlorophenyl)piperazin-1-yl)-2- hydroxypropoxy)quinolin-2(1H)-one |
| 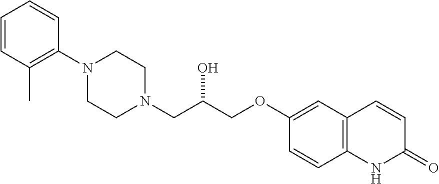 | (S)-6-(2-hydroxy-3-(4-o-tolylpiperazin-1- yl)propoxy)quinolin-2(1H)-one |
| 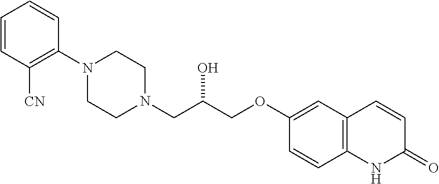 | (S)-2-(4-(2-hydroxy-3-(2-oxo-1,2-dihydroquinolin-6- yloxy)propyl)piperazin-1-yl)benzonitrile |
| 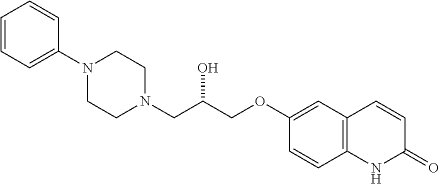 | (S)-6-(2-hydroxy-3-(4-phenylpiperazin-1- yl)propoxy)quinolin-2(1H)-one |
| 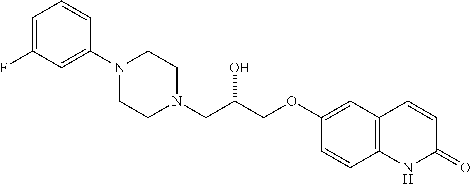 | (S)-6-(3-(4-(3-fluorophenyl)piperazin-1-yl)-2- hydroxypropoxy)quinolin-2(1H)-one |
| 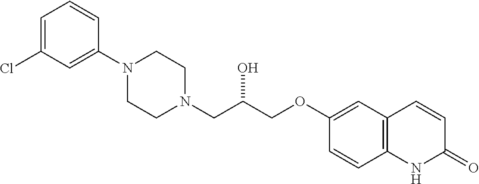 | (S)-6-(3-(4-(3-chlorophenyl)piperazin-1-yl)-2- hydroxypropoxy)quinolin-2(1H)-one |
| 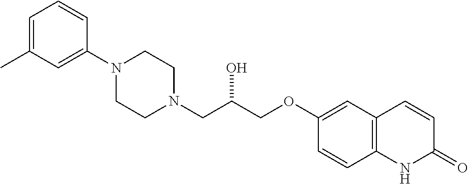 | (S)-6-(2-hydroxy-3-(4-m-tolypiperazin-1- yl)propoxy)quinolin-2(1H)-one |
| 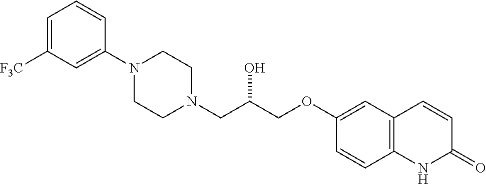 | (S)-6-(2-hydroxy-3-(4-(3- (trifluoromethyl)phenyl)piperazin-1- yl)propoxy)quinolin-2(1H)-one |
| 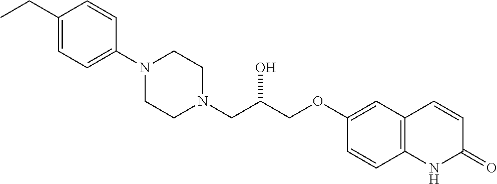 | (S)-6-(3-(4-(4-ethylphenyl)piperazin-1-yl)-2- hydroxypropoxy)quinolin-2(1H)-one |
| 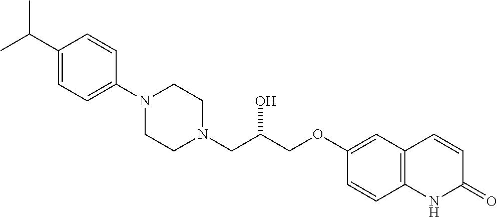 | (S)-6-(2-hydroxy-3-(4-(4-isopropylphenyl)piperazin- 1-yl)propoxy)quinolin-2(1H)-one |
| 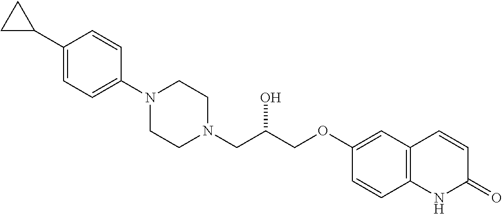 | (S)-6-(3-(4-(4-cyclopropylphenyl)piperazin-1-yl)-2- hydroxypropoxy)quinolin-2(1H)-one |
| 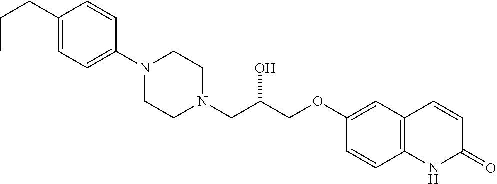 | (S)-6-(2-hydroxy-3-(4-(4-propylphenyl)piperazin-1- yl)propoxy)quinolin-2(1H)-one |
| 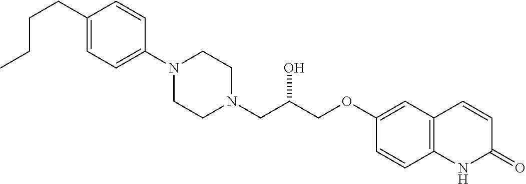 | (S)-6-(3-(4-(4-butylphenyl)piperazin-1-yl)-2- hydroxypropoxy)quinolin-2(1H)-one |
| 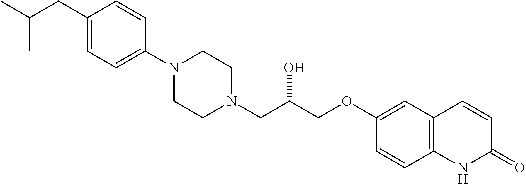 | (S)-6-(2-hydroxy-3-(4-(4-isobutylphenyl)piperazin-1- yl)propoxy)quinolin-2(1H)-one |
| 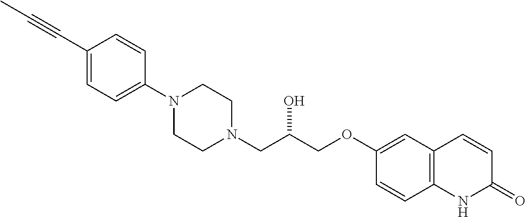 | (S)-6-(2-hydroxy-3-(4-(4-(prop-1- ynyl)phenyl)piperazin-1-yl)propoxy)quinolin-2(1H)- one |
| 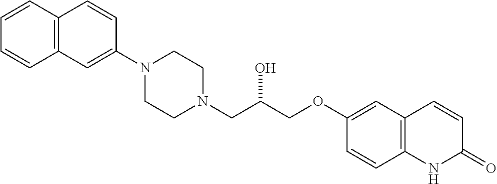 | (S)-6-(2-hydroxy-3-(4-(naphthalen-2-yl)piperazin-1- yl)propoxy)quinolin-2(1H)-one |
| 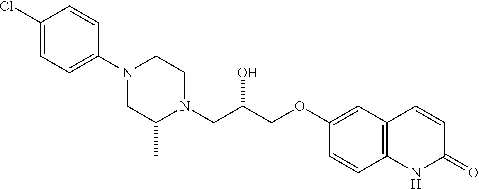 | 6-((S)-3-((R)-4-(4-chlorophenyl)-2-methylpiperazin-1- yl)-2-hydroxypropoxy)quinolin-2(1H)-one |
| 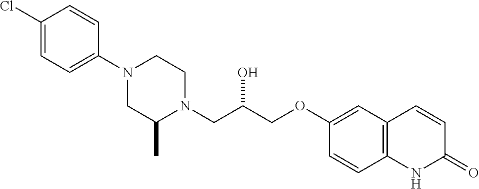 | 6-((S)-3-((S)-4-(4-chlorophenyl)-2-methylpiperazin-1- yl)-2-hydroxypropoxy)quinolin-2(1H)-one |
| 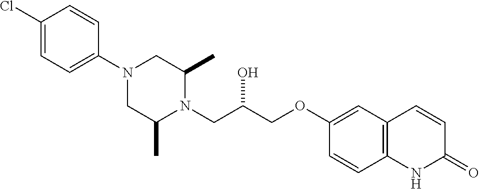 | 6-((S)-3-((2S,6R)-4-(4-chlorophenyl)-2,6- dimethylpiperazin-1-yl)-2-hydroxypropoxy)quinolin- 2(1H)-one |
| 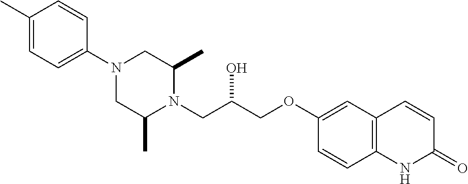 | 6-((S)-3-((2S,6R)-2,6-dimethyl-4-p-tolylpiperazin-1- yl)-2-hydroxypropoxy)quinolin-2(1H)-one |
| 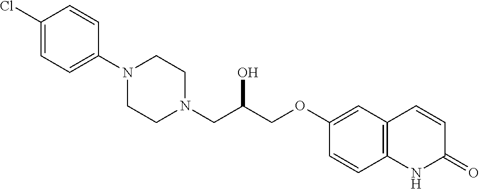 | (R)-6-(3-(4-(4-chlorophenyl)piperazin-1-yl)-2- hydroxypropoxy)quinolin-2(1H)-one |
| 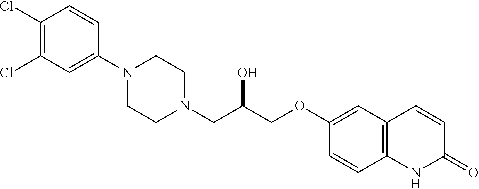 | (R)-6-(3-(4-(3,4-dichlorophenyl)piperazin-1-yl)-2- hydroxypropoxy)quinolin-2(1H)-one |
| 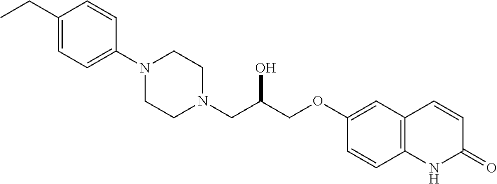 | (R)-6-(3-(4-(4-ethylphenyl)piperazin-1-yl)-2- hydroxypropoxy)quinolin-2(1H)-one |
| 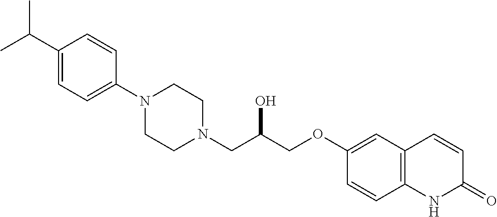 | (R)-6-(2-hydroxy-3-(4-(4-isopropylphenyl)piperazin- 1-yl)propoxy)quinolin-2(1H)-one |
| 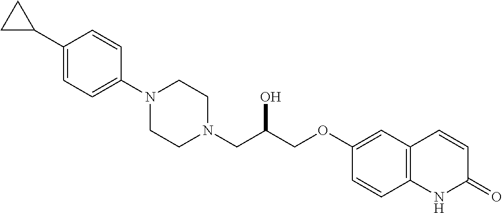 | (R)-6-(3-(4-(4-cyclopropylphenyl)piperazin-1-yl)-2- hydroxypropoxy)quinolin-2(1H)-one |
| 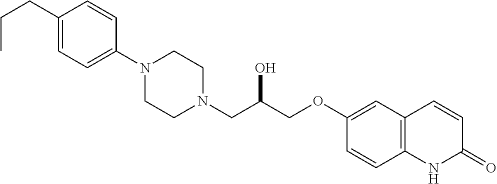 | (R)-6-(2-hydroxy-3-(4-(4-propylphenyl)piperazin-1- yl)propoxy)quinolin-2(1H)-one |
| 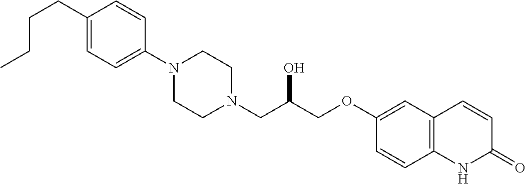 | (R)-6-(3-(4-(4-butylphenyl)piperazin-1-yl)-2- hydroxypropoxy)quinolin-2(1H)-one |
| 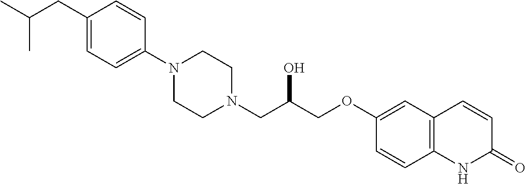 | (R)-6-(2-hydroxy-3-(4-(4-isobutylphenyl)piperazin-1- yl)propoxy)quinolin-2(1H)-one |
| 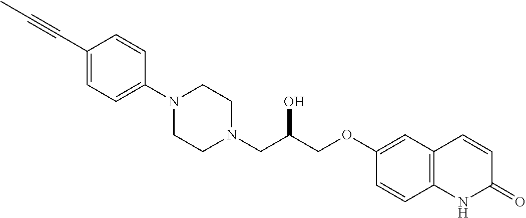 | (R)-6-(2-hydroxy-3-(4-(4-(prop-1- ynyl)phenyl)piperazin-1-yl)propoxy)quinolin-2(1H)- one |
| 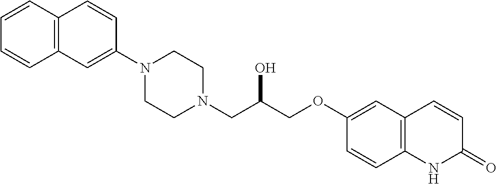 | (R)-6-(2-hydroxy-3-(4-(naphthalen-2-yl)piperazin-1- yl)propoxy)quinolin-2(1H)-one |
| 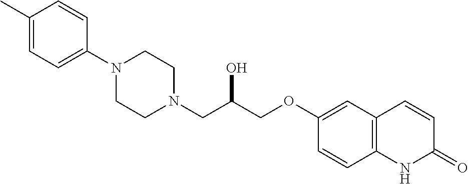 | (R)-6-(2-hydroxy-3-(4-p-tolylpiperazin-1-yl) propoxy)quinolin-2(1H)-one |
| 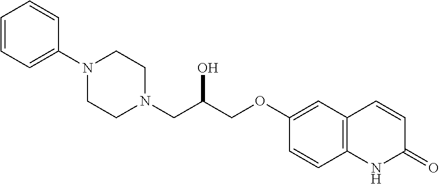 | (R)-6-(2-hydroxy-3-(4-phenylpiperazin-1- yl)propoxy)quinolin-2(1H)-one |
| 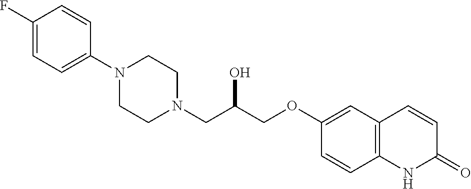 | (R)-6-(3-(4-(fluorophenyl)piperazin-1-yl)-2- hydroxypropoxy)quinolin-2(1H)-one |
| 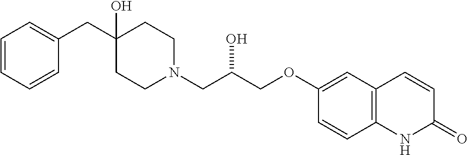 | (S)-6-(3-(4-benzyl-4-hydroxypiperidin-1-yl)-2- hydroxypropoxy)quinolin-2(1H)-one |
| 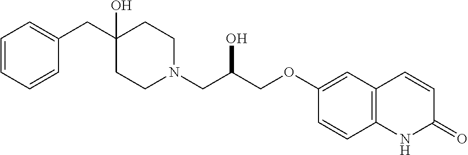 | (R)-6-(3-(4-benzyl-4-hydroxypiperidin-1-yl)-2- hydroxypropoxy)quinolin-2(1H)-one |
| 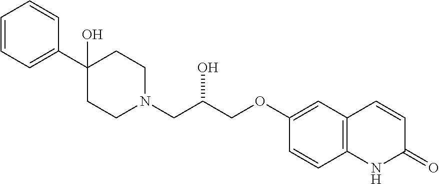 | (S)-6-(2-hydroxy-3-(4-hydroxy-4-phenylpiperidin-1- yl)propoxy)quinolin-2(1H)-one |

In another embodiment, the compound is selected from Table 9.

TABLE 9

| Compound | Name |
|---|---|
| 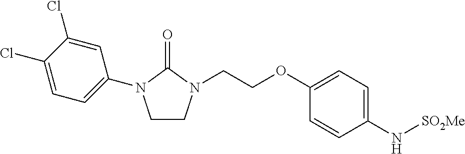 | N-(4-(2-(3-(3,4-dichlorophenyl)-2-oxoimidazolidin- 1-yl)ethoxy)phenyl)methanesulfonamide |
| 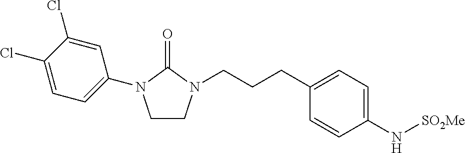 | N-(4-(3-(3-(3,4-dichlorophenyl)-2-oxoimidazolidin- 1-yl)propyl)phenyl)methanesulfonamide |
| 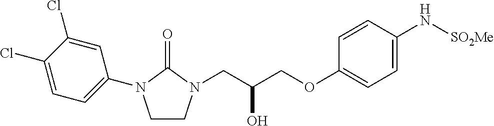 | (S)-N-(4-(3-(3-(3,4-dichlorophenyl)-2- oxoimidazolidin-1-yl)-2- hydroxypropoxy)phenyl)methanesulfonamide |
| 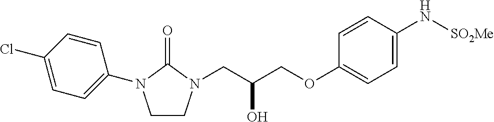 | (S)-N-(4-(3-(3-(4-chlorophenyl)-2-oxoimidazolidin- 1-yl)-2- hydroxypropoxy)phenyl)methanesulfonamide |
| 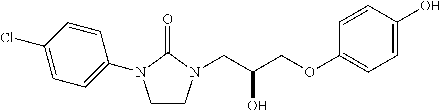 | (S)-1-(4-chlorophenyl)-3-(2-hydroxy-3-(4- hydroxyphenoxy)propyl)imidazolidin-2-one |
| 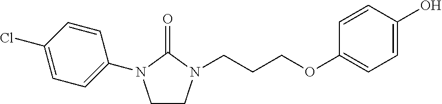 | 1-(4-chlorophenyl)-3-(3-(4- hydroxyphenoxy)propyl)imidazolidin-2-one |
| 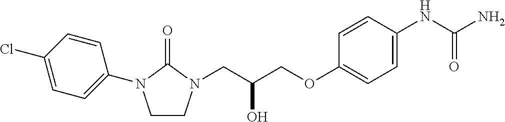 | (S)-1-(4-(3-(3-(4-chlorophenyl)-2-oxoimidazolidin- 1-yl)-2-hydroxypropoxy)phenyl)urea |

In another embodiment, the compound is selected from Table 10.

TABLE 10

| Compound | Name |
|---|---|
| 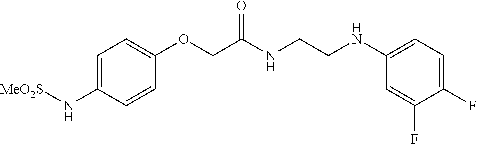 | N-(2-(3,4-difluorophenylamino)ethyl)-2-(4- (methylsulfonamido)phenoxy)acetamide |
| 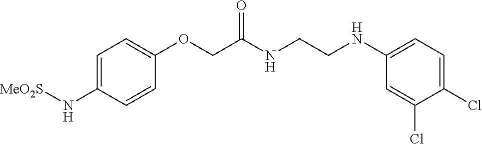 | N-(2-(3,4-dichlorophenylamino)ethyl)-2-(4- (methylsulfonamido)phenoxy)acetamide |
| 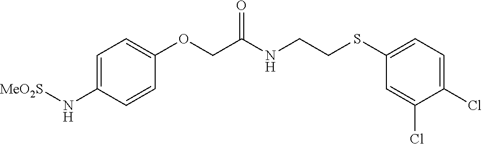 | N-(2-(3,4-dichlorophenylthio)ethyl)-2-(4- (methylsulfonamido)phenoxy)acetamide |
| 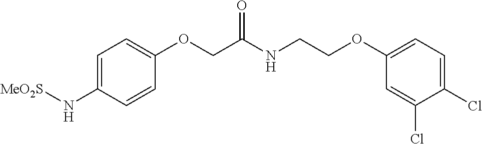 | N-(2-(3,4-dichlorophenoxy)ethyl)-2-(4- (methylsulfonamido)phenoxy)acetamide |
| 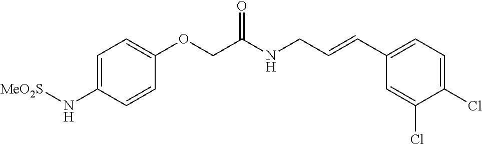 | (E)-N-(3-(3,4-dichlorophenyl)allyl)-2-(4- (methylsulfonamido)phenoxy)acetamide |
| 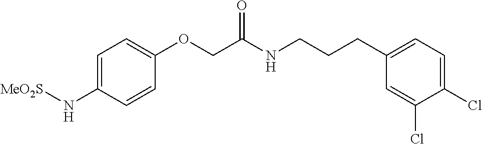 | N-(3-(3,4-dichlorophenyl)propyl)-2-(4- (methylsulfonamido)phenoxy)acetamide |
| 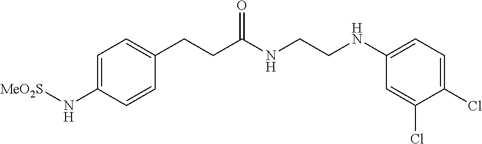 | N-(2-(3,4-dichlorophenylamino)ethyl)-3-(4- (methylsulfonamido)phenyl)propanamide |
| 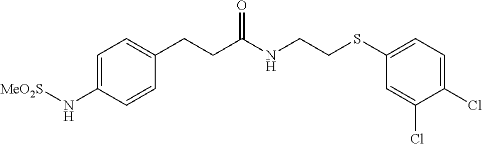 | N-(2-(3,4-dichlorophenylthio)ethyl)-3-(4- (methylsulfonamido)phenyl)propanamide |
| 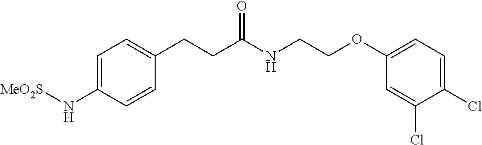 | N-(2-(3,4-dichlorophenoxy)ethyl)-3-(4- (methylsulfonamido)phenyl)propanamide |
| 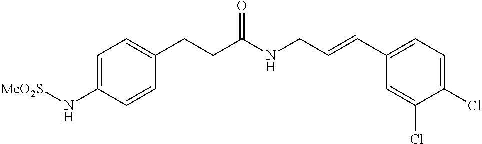 | (E)-N-(3-(3,4-dichlorophenyl)allyl)-3-(4- (methylsulfonamido)phenyl)propanamide |
| 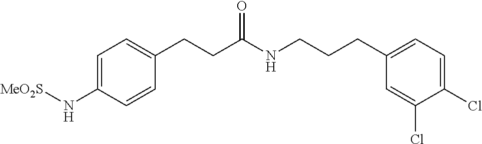 | N-(3-(3,4-dichlorophenyl)propyl)-3-(4- (methylsulfonamido)phenyl)propanamide |
| 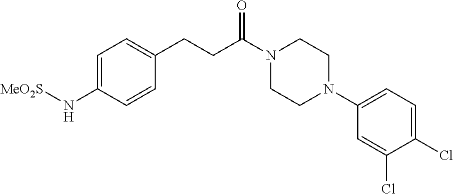 | N-(4-(3-(4-(3,4-dichlorophenyl)piperazin-1-yl)-3- oxopropyl)phenyl)methanesulfonamide |
| 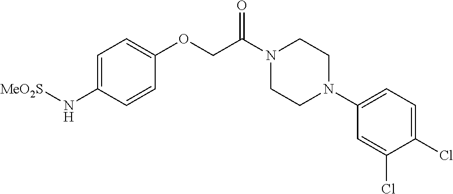 | N-(4-(2-(4-(3,4-dichlorophenyl)piperazin-1-yl)-2- oxoethoxy)phenyl)methanesulfonamide |
| 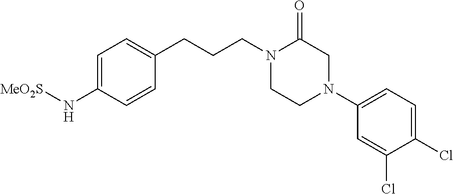 | N-(4-(3-(4-(3,4-dichlorophenyl)-2-oxopiperazin-1- yl)propyl)phenyl)methanesulfonamide |
| 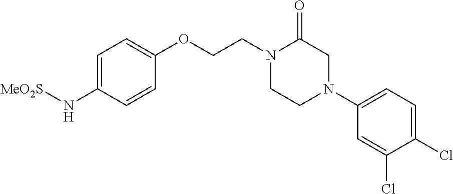 | N-(4-(2-(4-(3,4-dichlorophenyl)-2-oxopiperazin-1- yl)ethoxy)phenyl)methanesulfonamide |
| 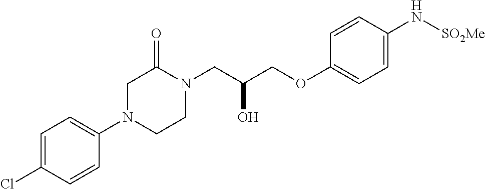 | (S)-N-(4-(3-(4-(4-chlorophcnyl)-2-oxopiperazin-1- yl)-2-hydroxypropoxy)phenyl)methanesulfonamide |
| 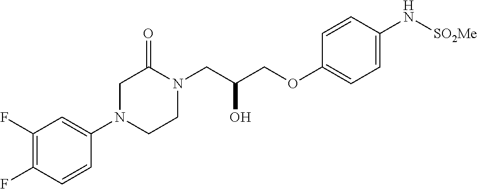 | (S)-N-(4-(3-(4-(3,4-difluorophcnyl)-2-oxopiperazin- 1-yl)-2- hydroxypropoxy)phenyl)methanesulfonamide |
| 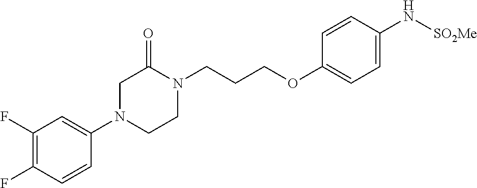 | N-(4-(3-(4-(3,4-difluorophenyl)-2-oxopiperazin-1- yl)propoxy)phenyl)methanesulfonamide |
| 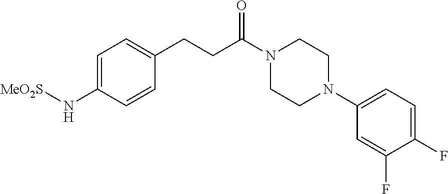 | N-(4-(3-(4-(3,4-difluorophenyl)piperazin-1-yl)-3- oxopropyl)phenyl)methanesulfonamide |
| 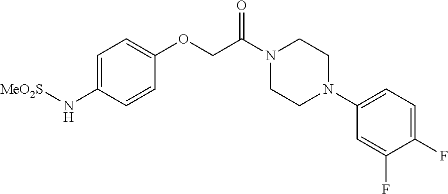 | N-(4-(2-(4-(3,4-difluorophenyl)piperazin-1-yl)-2- oxoethoxy)phenyl)methanesulfonamide |
| 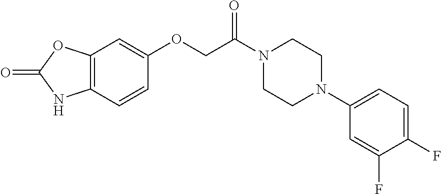 | 6-(2-(4-(3,4-difluorophenyl)piperazin-1-yl)-2- oxoethoxy)benzo[d]oxazol-2(3H)-one |
| 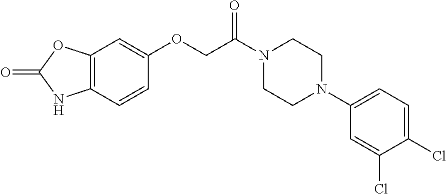 | 6-(2-(4-(3,4-dichlorophenyl)piperazin-1-yl)-2- oxoethoxy)benzo[d]oxazol-2(3H)-one |
| 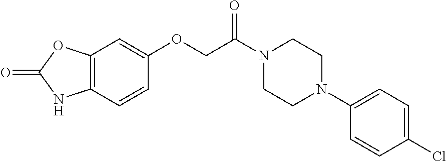 | 6-(2-(4-(4-Chlorophenyl)piperazin-1-yl)-2- oxoethoxy)benzo[d]oxazol-2(3H)-one |
| 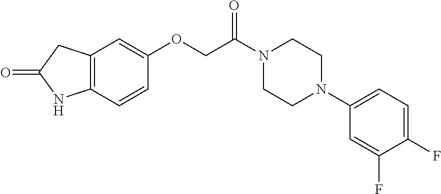 | 5-(2-(4-(3,4-difluorophenyl)piperazin-1-yl)-2- oxoethoxy)indolin-2-one |
| 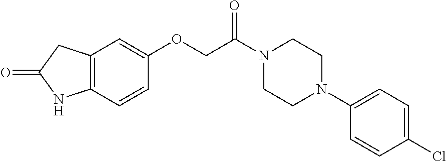 | 5-(2-(4-(4-chlorophenyl)piperazin-1-yl)-2- oxoethoxy)indolin-2-one |
| 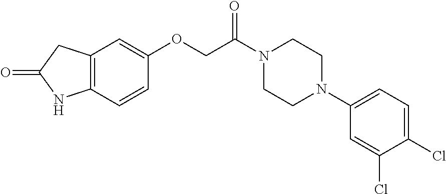 | 5-(2-(4-(3,4-dichlorophenyl)piperazin-1-yl)-2- oxoethoxy)indolin-2-one |
| 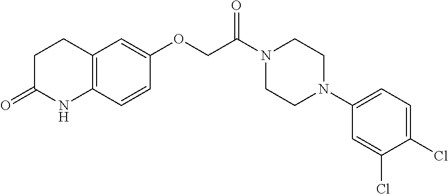 | 6-(2-(4-(3,4-dichlorophenyl)piperazin-1-yl)-2- oxoethoxy)-3,4-dihydroquinolin-2(1H)-one |
| 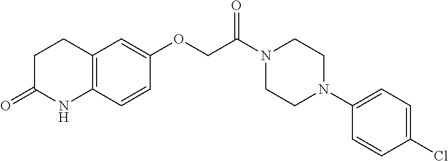 | 6-(2-(4-(4-chlorophenyl)piperazin-1-yl)-2- oxoethoxy)-3,4-dihydroquinolin-2(1H)-one |
| 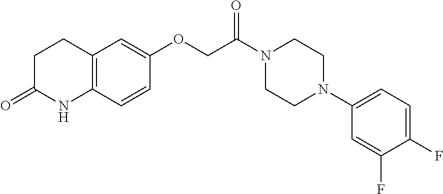 | 6-(2-(4-(3,4-difluorophenyl)piperazin-1-yl)-2- oxoethoxy)-3,4-dihydroquinolin-2(1H)-one |
| 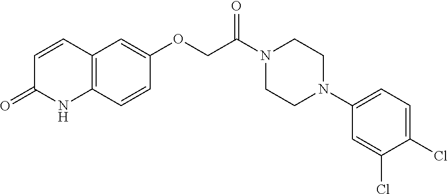 | 6-(2-(4-(3,4-dichlorophenyl)piperazin-1-yl)-2- oxoethoxy)quinolin-2(1H)-one |
| 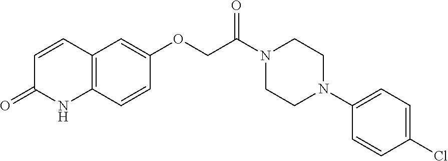 | 6-(2-(4-(4-chlorophenyl)piperazin-1-yl)-2- oxoethoxy)quinolin-2(1H)-one |
| 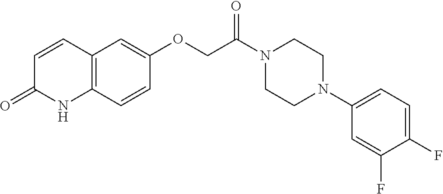 | 6-(2-(4-(3,4-difluorophenyl)piperazin-1-yl)-2- oxoethoxy)quinolin-2(1H)-one |
| 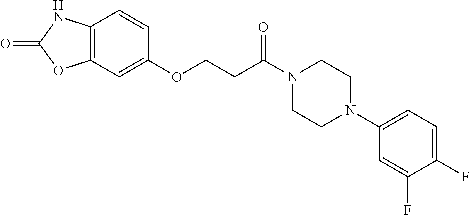 | 6-(3-(4-(3,4-difluorophenyl)piperazin-1-yl)-3- oxopropoxy)benzo[d]oxazol-2(3H)-one |
| 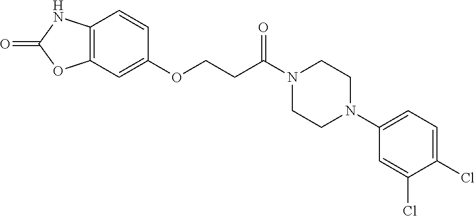 | 6-(3-(4-(3,4-dichlorophenyl)piperazin-1-yl)-3- oxopropoxy)benzo[d]oxazol-2(3H)-one |
| 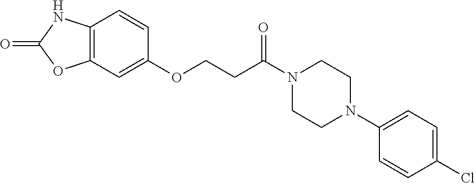 | 6-(3-(4-(4-chlorophenyl)piperazin-1-yl)-3- oxopropoxy)benzo[d]oxazol-2(3H)-one |
| 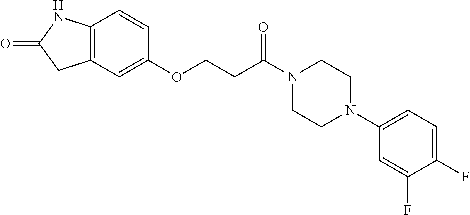 | 5-(3-(4-(3,4-difluorophenyl)piperazin-1-yl)-3- oxopropoxy)indolin-2-one |
| 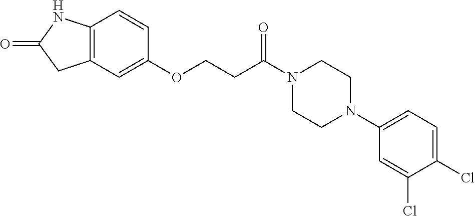 | 5-(3-(4-(3,4-dichlorophenyl)piperazin-1-yl)-3- oxopropoxy)indolin-2-one |
| 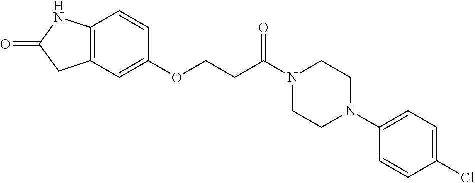 | 5-(3-(4-(4-chlorophenyl)piperazin-1-yl)-3- oxopropoxy)indolin-2-one |
| 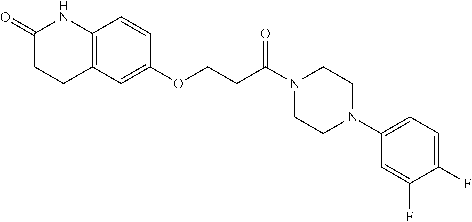 | 6-(3-(4-(3,4-difluorophenyl)piperazin-1-yl)-3- oxopropoxy)-3,4-dihydroquinolin-2(1H)-one |
| 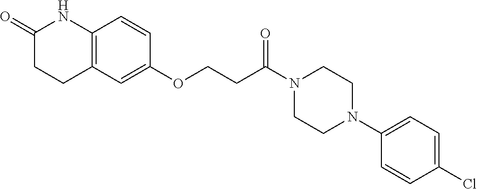 | 6-(3-(4-(4-chlorophenyl)piperazin-1-yl)-3- oxopropoxy)-3,4-dihydroquinolin-2(1H)-one |
| 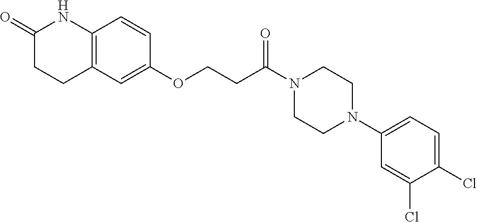 | 6-(3-(4-(3,4-dichlorophenyl)piperazin-1-yl)-3- oxopropoxy)-3,4-dihydroquinolin-2(1H)-one |
| 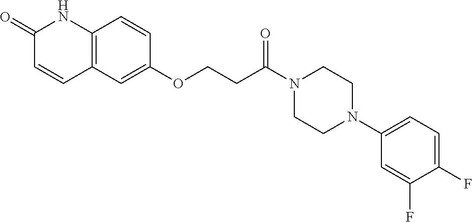 | 6-(3-(4-(3,4-difluorophenyl)piperazin-1-yl)-3- oxopropoxy)quinolin-2(1H)-one |
| 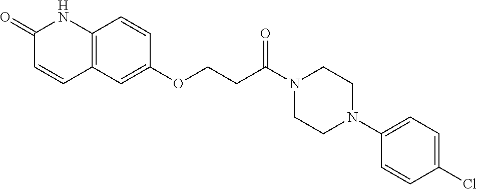 | 6-(3-(4-(4-chlorophenyl)piperazin-1-yl)-3- oxopropoxy)quinolin-2(1H)-one |
| 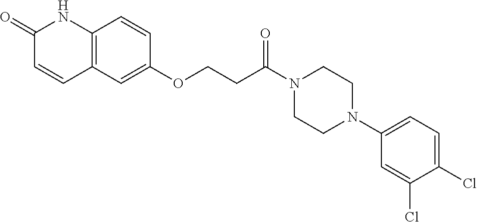 | 6-(3-(4-(3,4-dichlorophenyl)piperazin-1-yl)-3- oxopropoxy)quinolin-2(1H)-one |
| 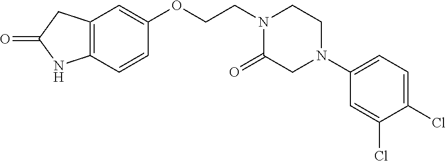 | 5-(2-(4-(3,4-dichlorophenyl)-2-oxopiperazin-1- yl)ethoxy)indolin-2-one |
| 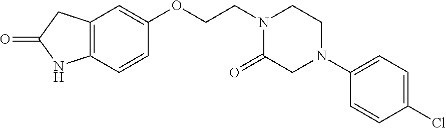 | 5-(2-(4-(4-chlorophenyl)-2-oxopiperazin-1- yl)ethoxy)indolin-2-one |
| 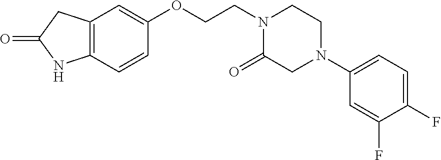 | 5-(2-(4-(3,4-difluorophenyl)-2-oxopiperazin-1- yl)ethoxy)indolin-2-one |
| 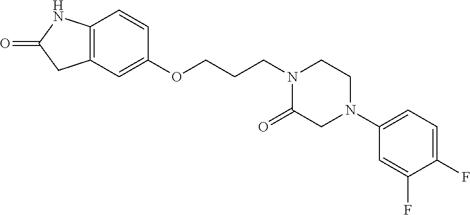 | 5-(3-(4-(3,4-difluorophenyl)-2-oxopiperazin-1- yl)propoxy)indolin-2-one |
| 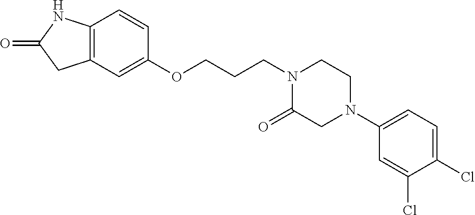 | 5-(3-(4-(3,4-dichlorophenyl)-2-oxopiperazin-1- yl)propoxy)indolin-2-one |
| 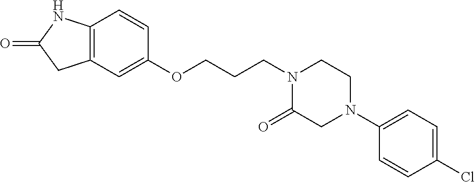 | 5-(3-(4-(4-chlorophenyl)-2-oxopiperazin-1- yl)propoxy)indolin-2-one |
| 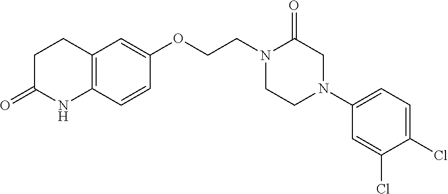 | 6-(2-(4-(3,4-dichlorophenyl)-2-oxopiperazin-1- yl)ethoxy)-3,4-dihydroquinolin-2(1H)-one |
| 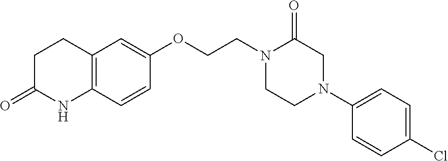 | 6-(2-(4-(4-chlorophenyl)-2-oxopiperazin-1- yl)ethoxy)-3,4-dihydroquinolin-2(1H)-one |
| 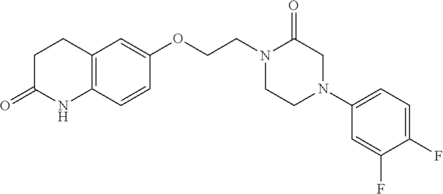 | 6-(2-(4-(3,4-difluorophenyl)-2-oxopiperazin-1- yl)ethoxy)-3,4-dihydroquinolin-2(1H)-one |
| 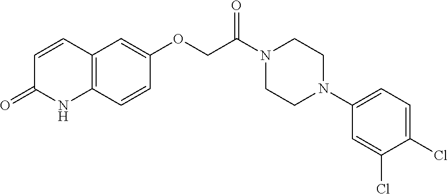 | 6-(2-(4-(3,4-dichlorophenyl)piperazin-1-yl)-2- oxoethoxy)quinolin-2(1H)-one |
| 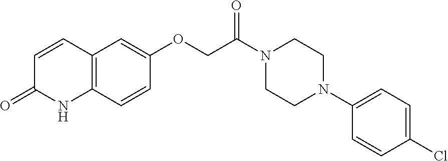 | 6-(2-(4-(4-chlorophenyl)piperazin-1-yl)-2- oxoethoxy)quinolin-2(1H)-one |
| 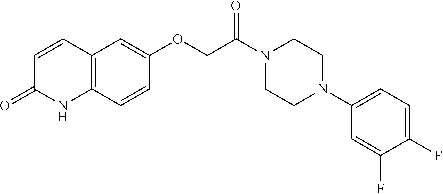 | 6-(2-(4-(3,4-difluorophenyl)piperazin-1-yl)-2- oxoethoxy)quinolin-2(1H)-one |
| 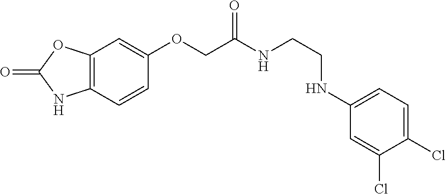 | N-(2-(3,4-dichlorophenylamino)ethyl)-2-(2-oxo- 2,3-dihydrobenzo[d]oxazol-6-yloxy)acetamide |
| 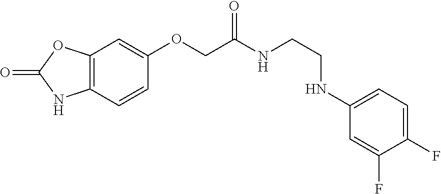 | N-(2-(3,4-difluorophenylamino)ethyl)-2-(2-oxo-2,3- dihydrobenzo[d]oxazol-6-yloxy)acetamide |
| 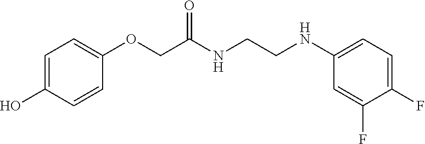 | N-(2-(3,4-difluorophenylamino)ethyl-2-(4- hydroxyphenoxy)acetamide |
| 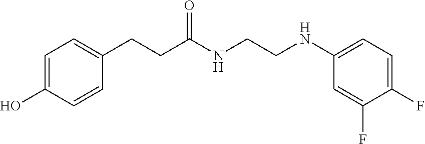 | N-(2-(3,4-difluorophenylamino)ethyl)-3-(4- hydroxyphenyl)propanamide |
| 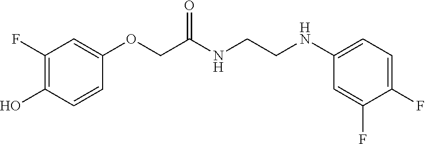 | N-(2-(3,4-difluorophenylamino)ethyl)-2-(3-fluoro- 4-hydroxyphenoxy)acetamide |

In one embodiment, the compound is not

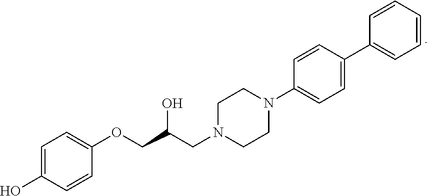

In another embodiment, the compound is not

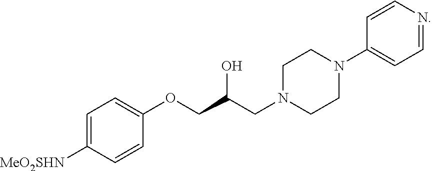

In one embodiment, the compound has an IC₅₀ value of 600 nM or less. In one embodiment, the compound has an IC₅₀ value of 600 nM or less at pH 6.9 or an ischemic pH. In one embodiment, the compound is selected from Table 11.

TABLE 11

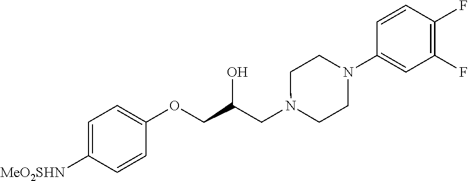

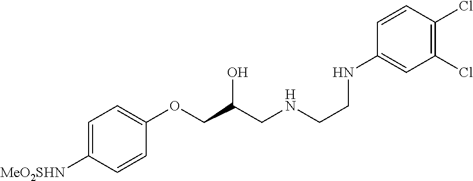

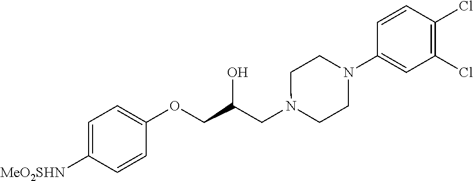

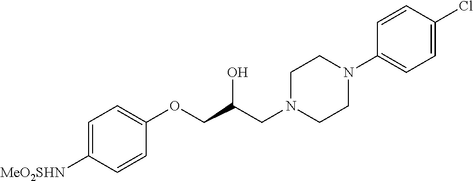

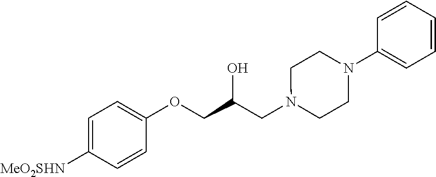

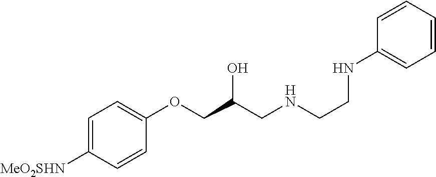

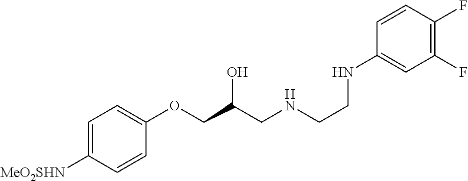

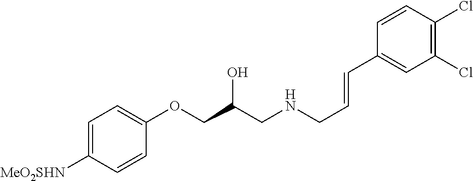

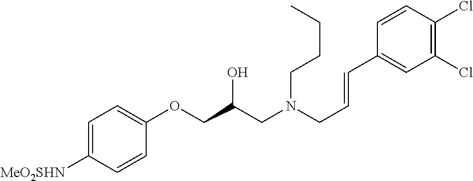

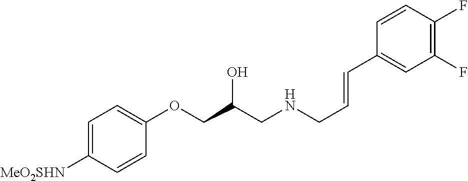

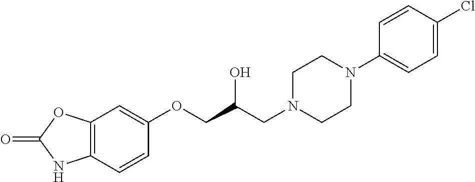

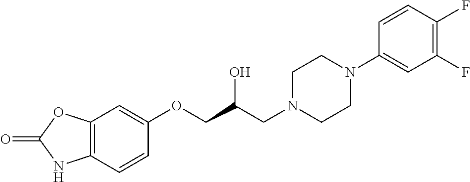

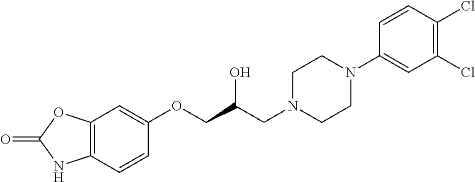

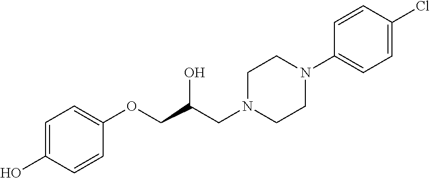

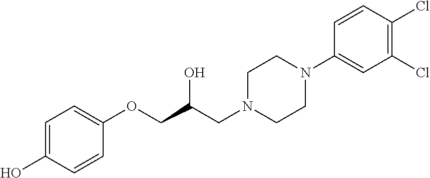

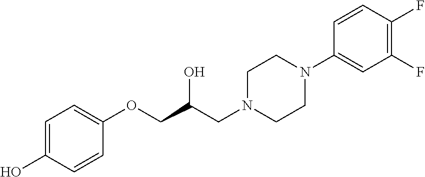

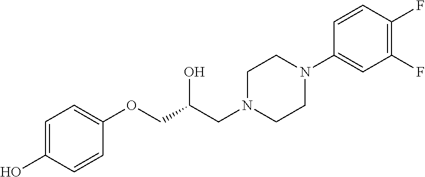

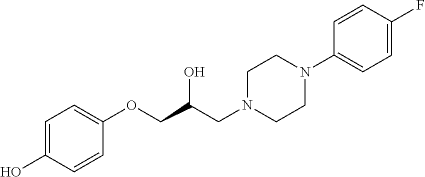

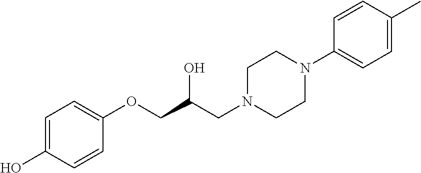

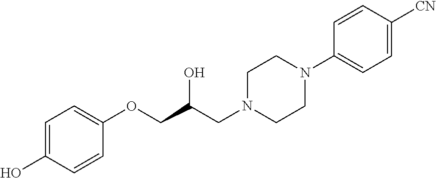

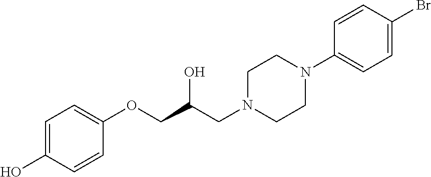

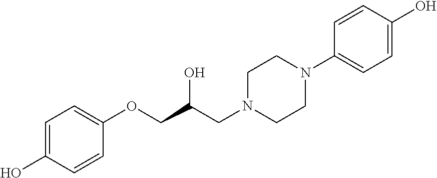

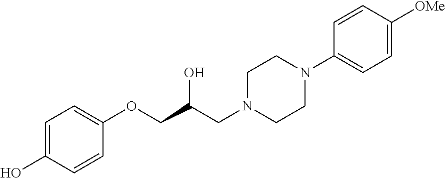

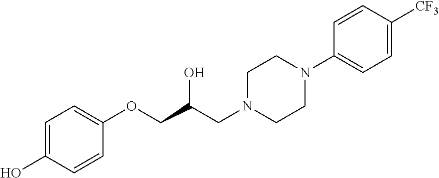

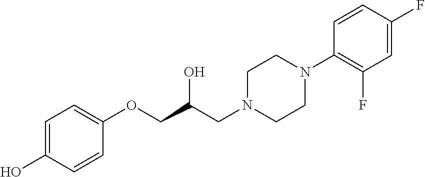

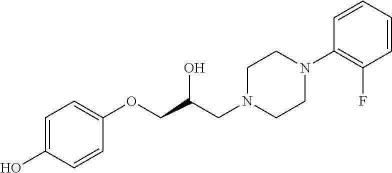

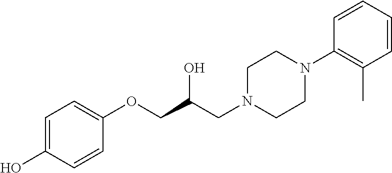

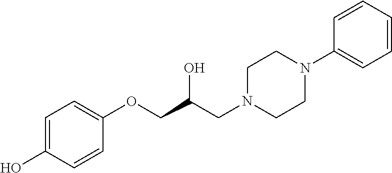

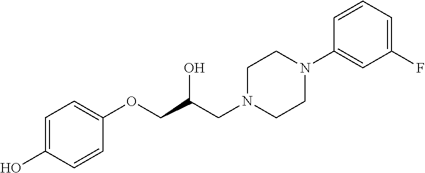

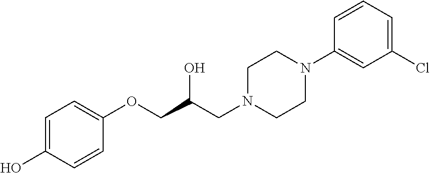

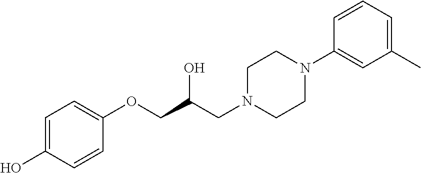

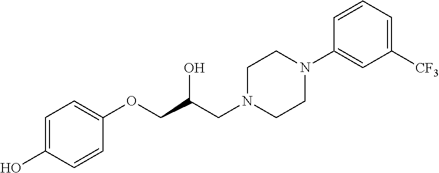

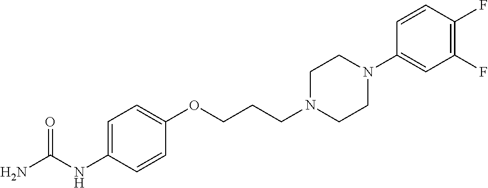

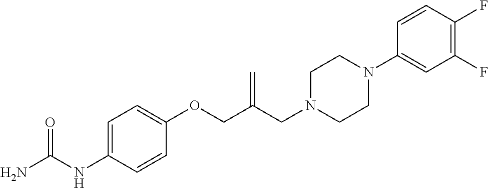

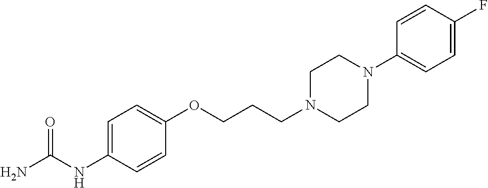

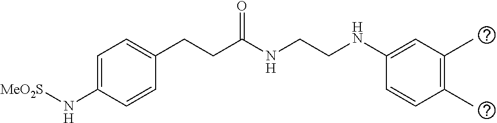

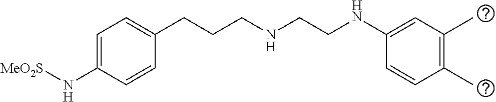

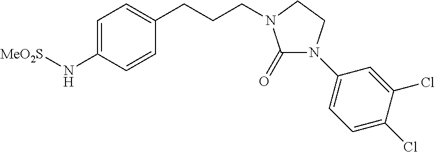

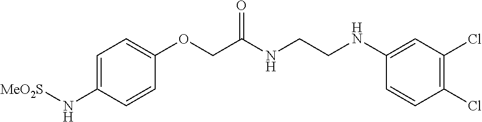

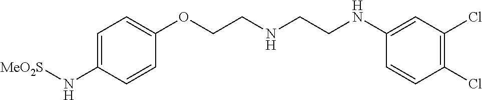

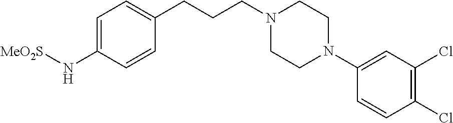

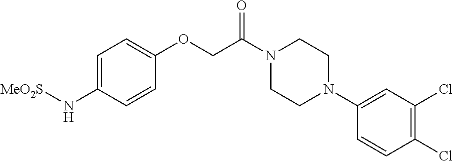

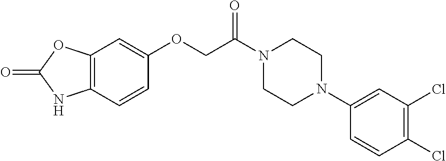

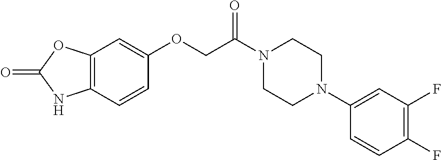

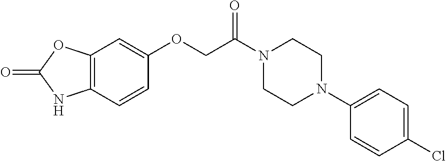

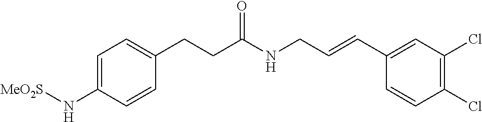

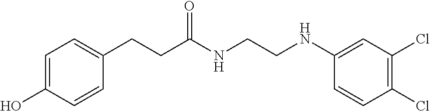

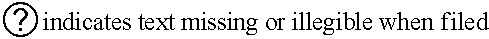

In one embodiment, the compound has an IC₅₀ value of 600 nM or less at pH 7.6 or a physiological pH. In one embodiment, the compound is selected from Table 12.

TABLE 12

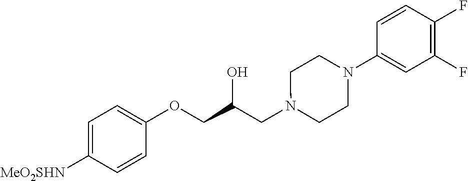

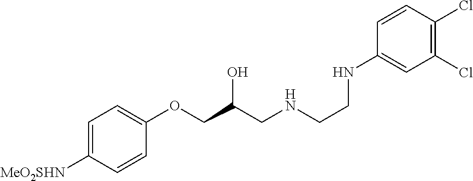

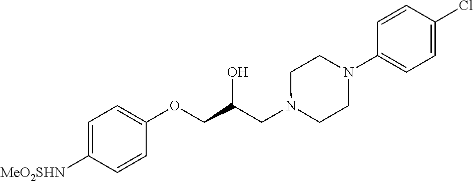

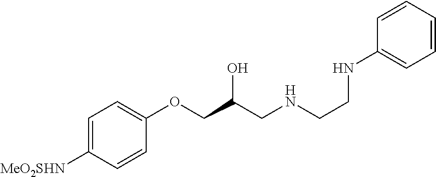

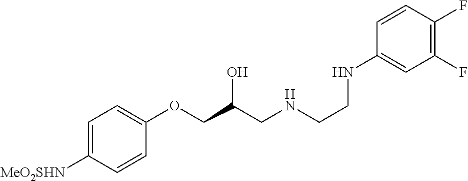

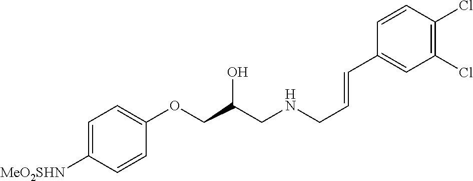

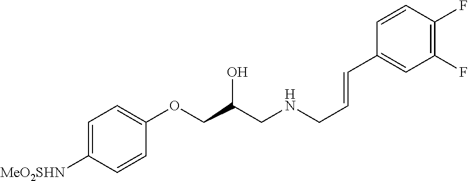

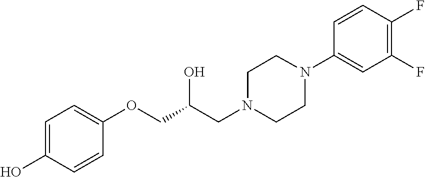

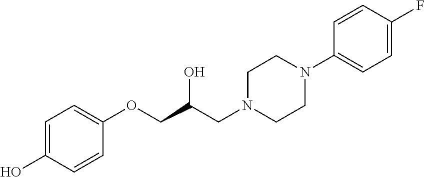

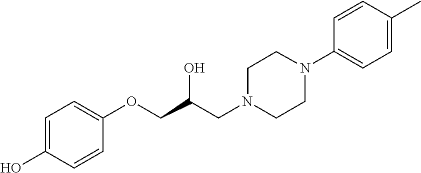

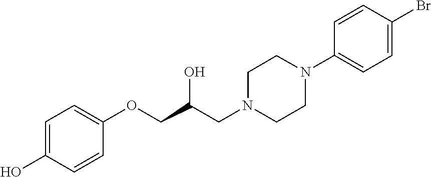

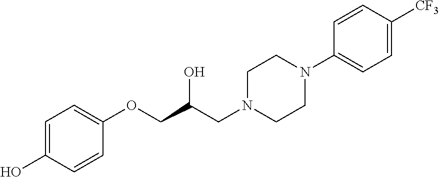

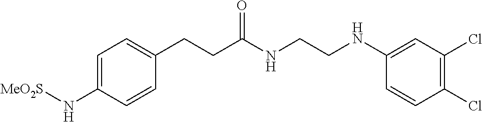

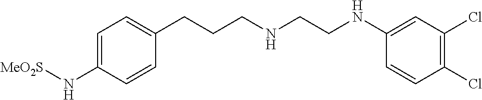

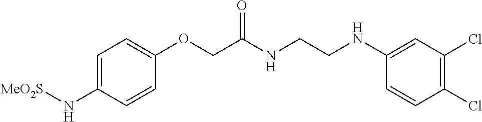

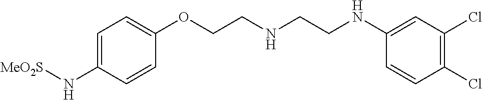

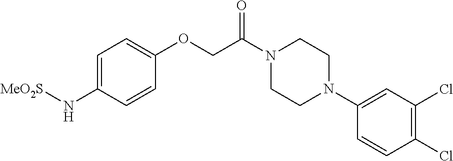

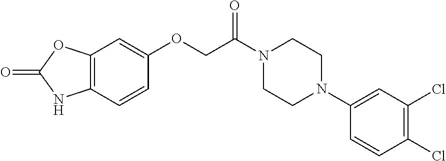

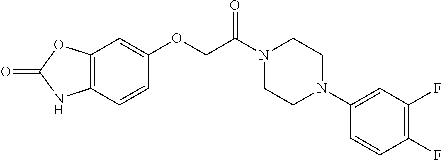

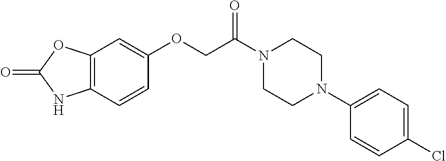

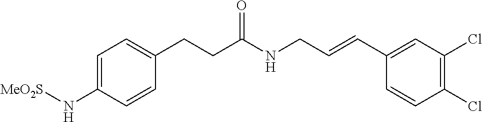

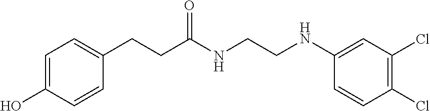

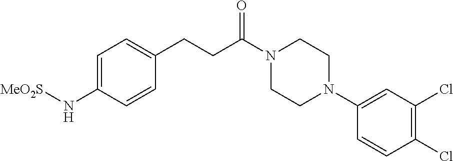

In one embodiment, the compound has a pH boost of 5 or more. In one embodiment, the compound is selected from Table 13.

TABLE 13

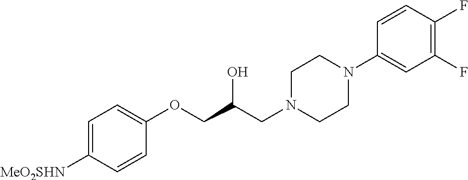

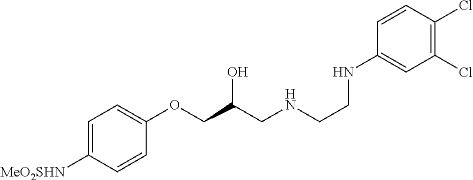

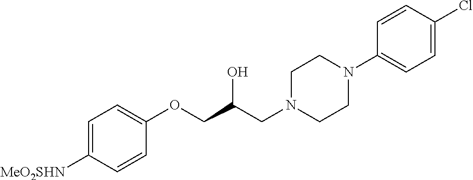

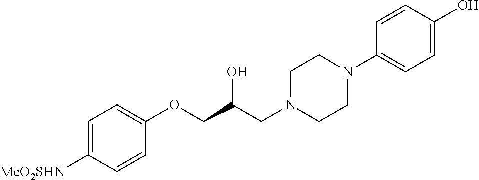

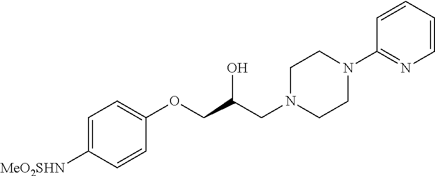

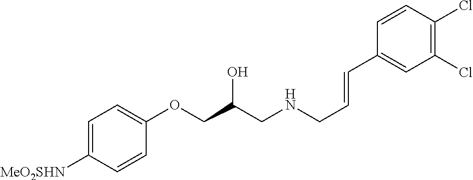

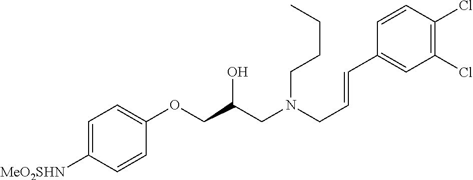

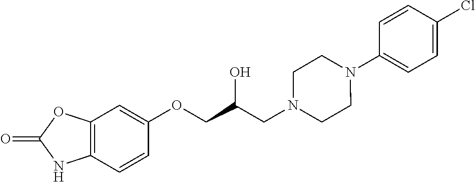

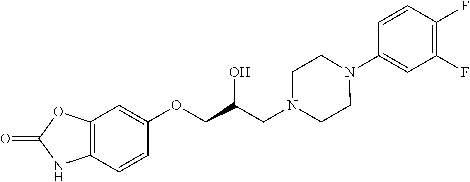

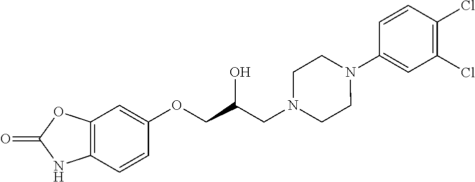

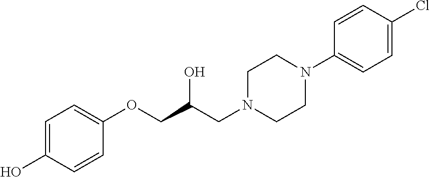

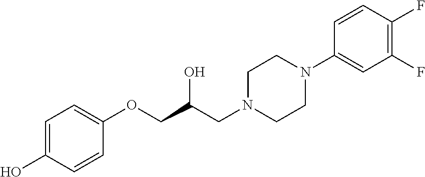

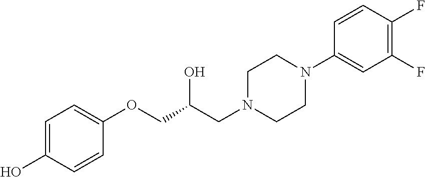

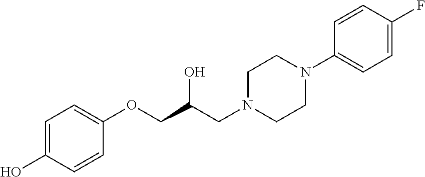

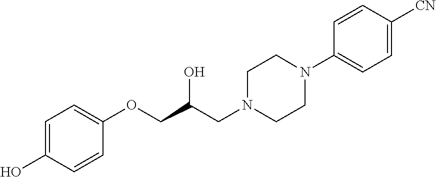

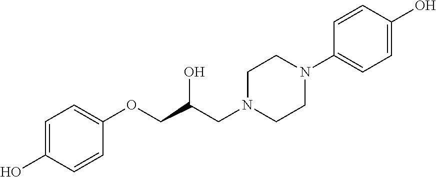

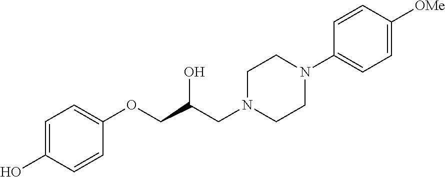

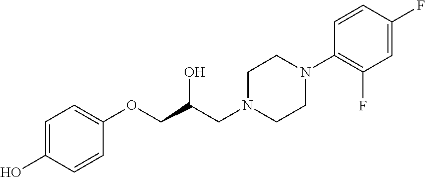

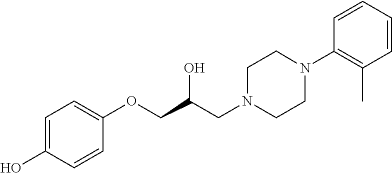

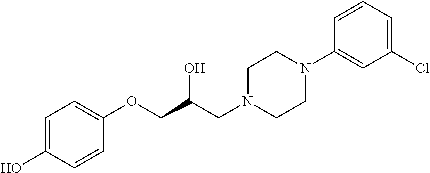

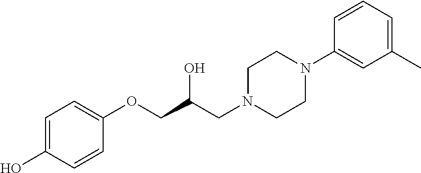

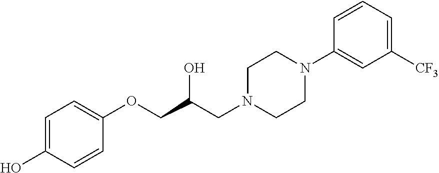

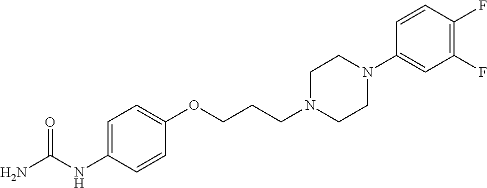

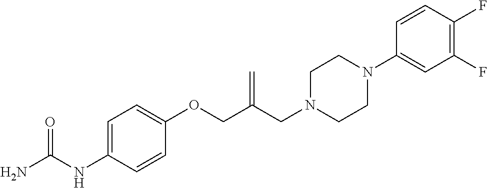

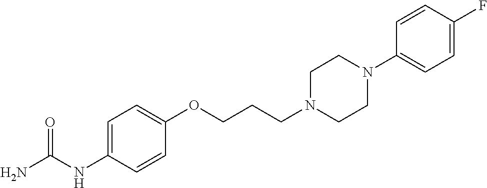

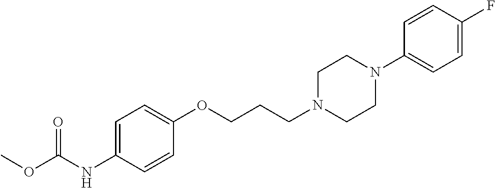

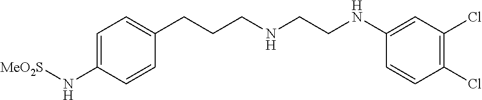

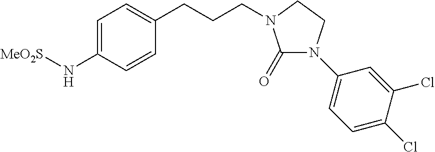

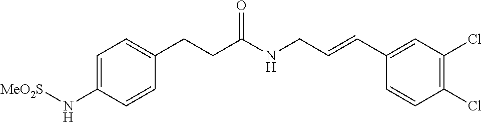

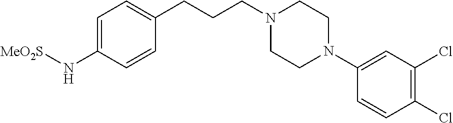

In one embodiment, the compound is selected from the group consisting of:

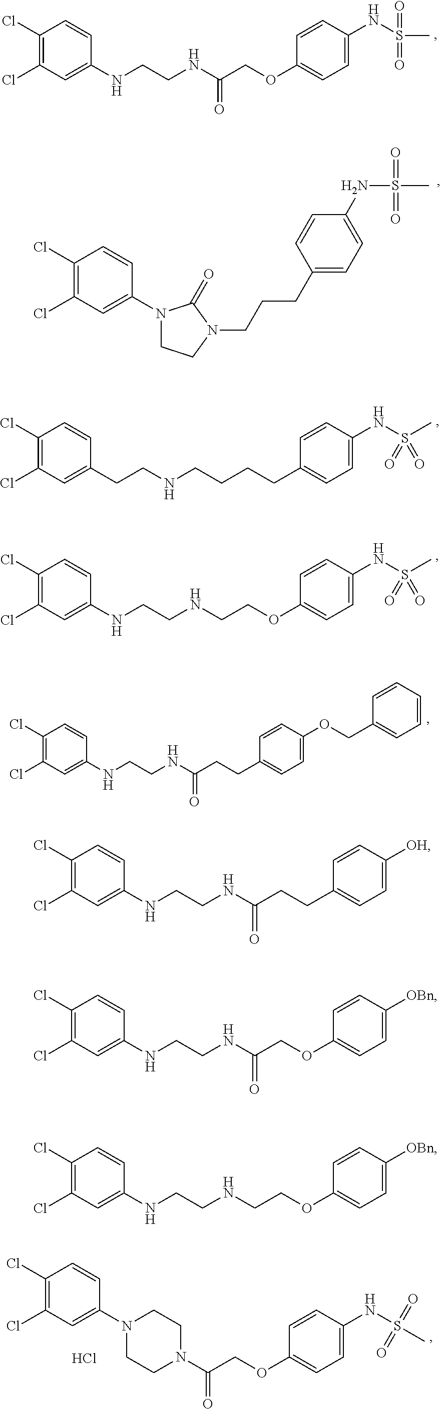

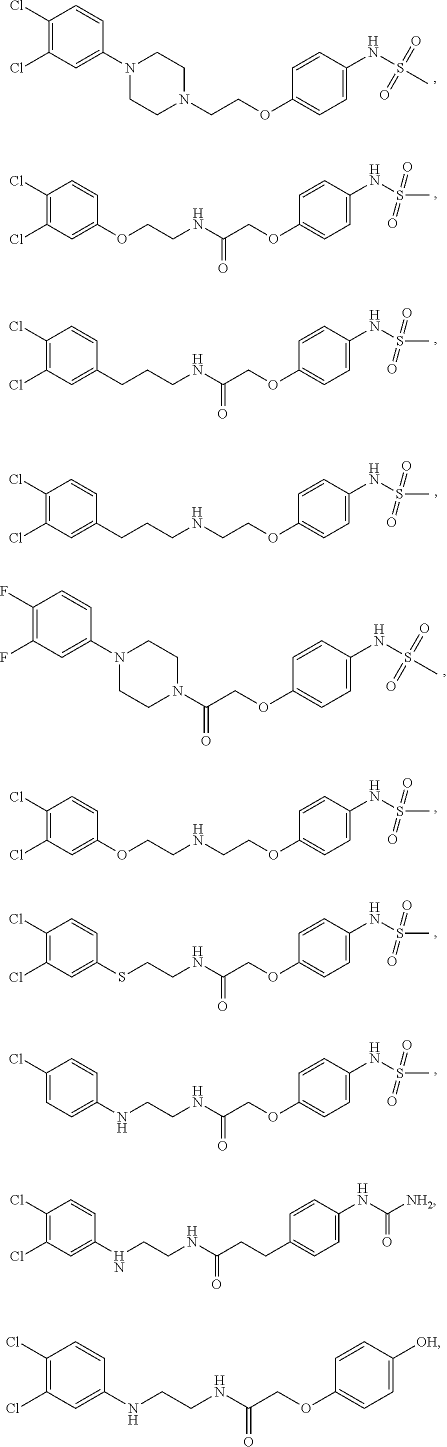

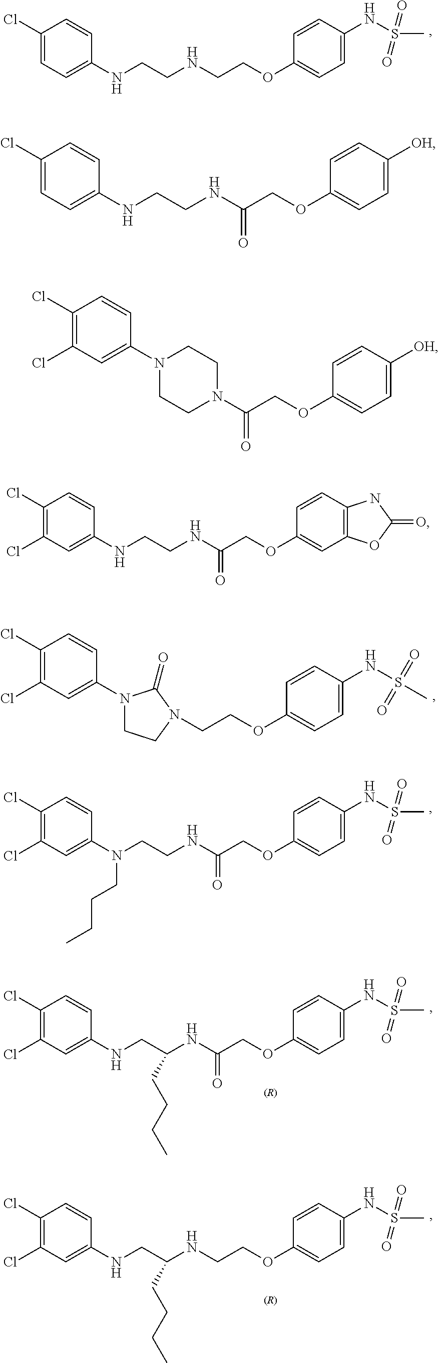

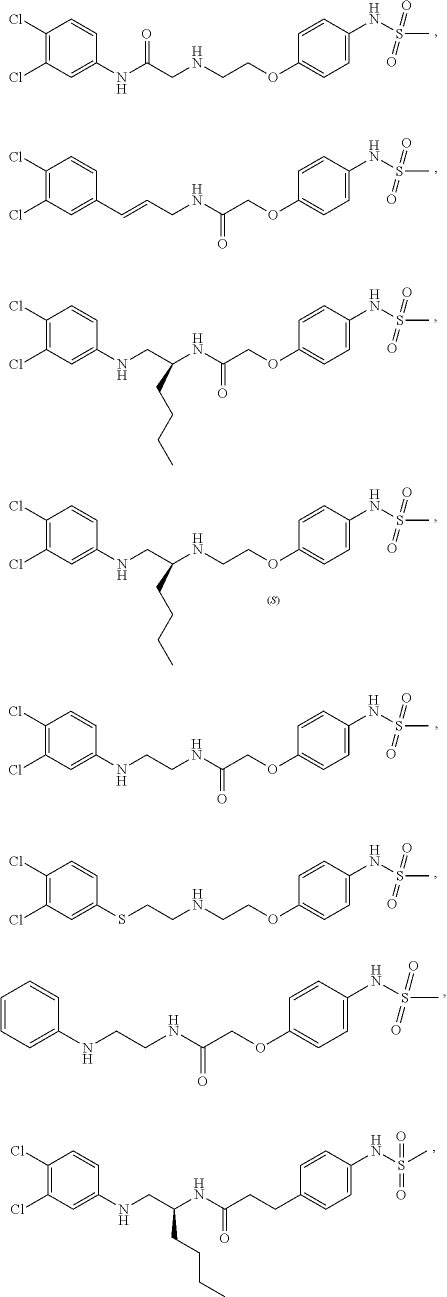

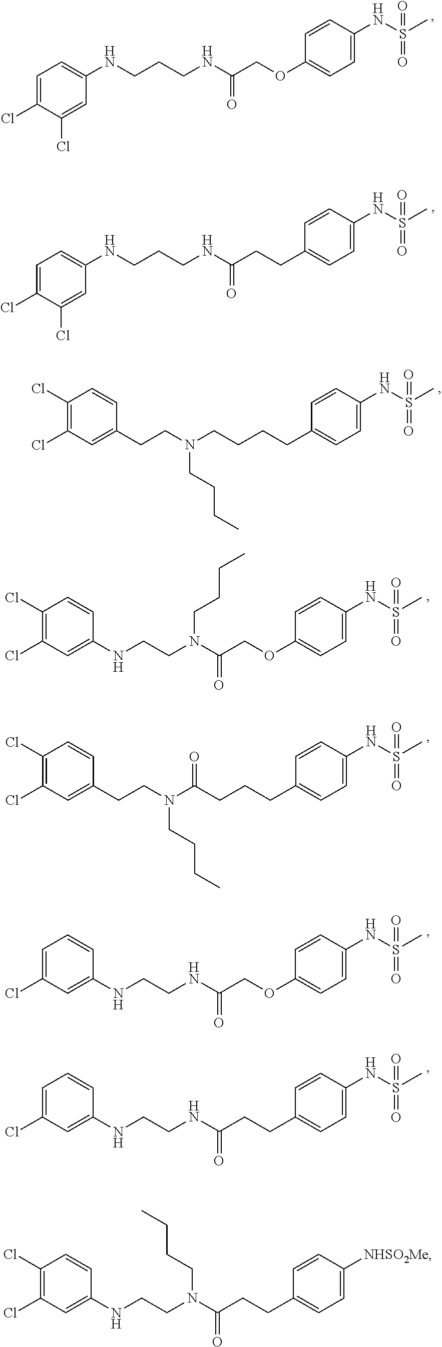

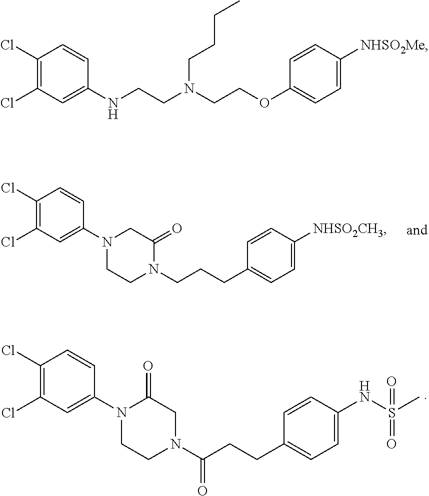

In one embodiment, the compound is

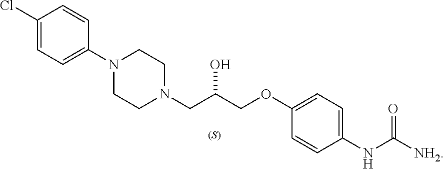

In one embodiment, the compound is selected from the group consisting of:

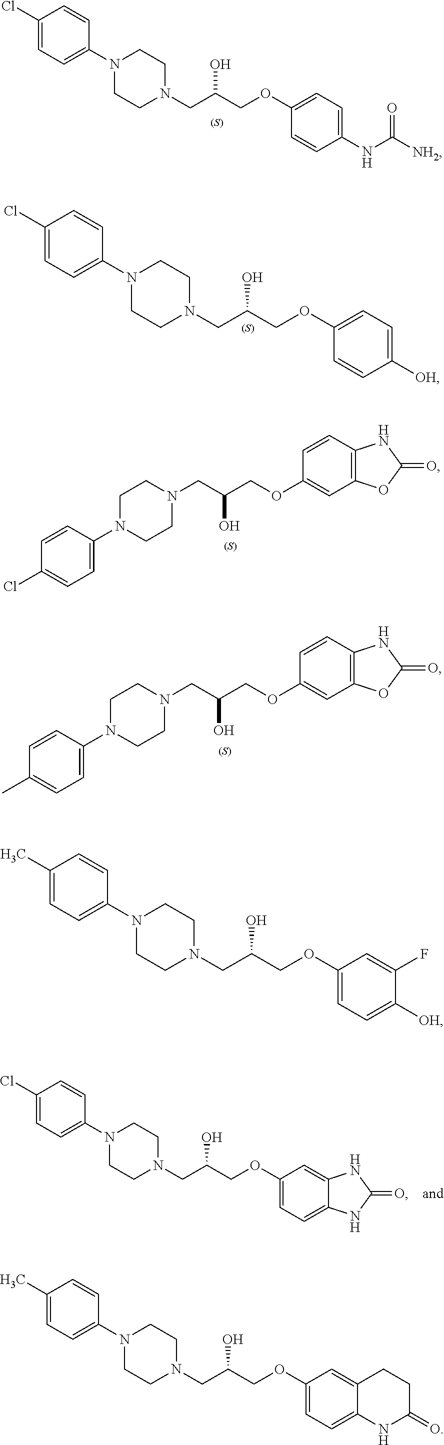

In another embodiment, the compound is selected from the group consisting of:

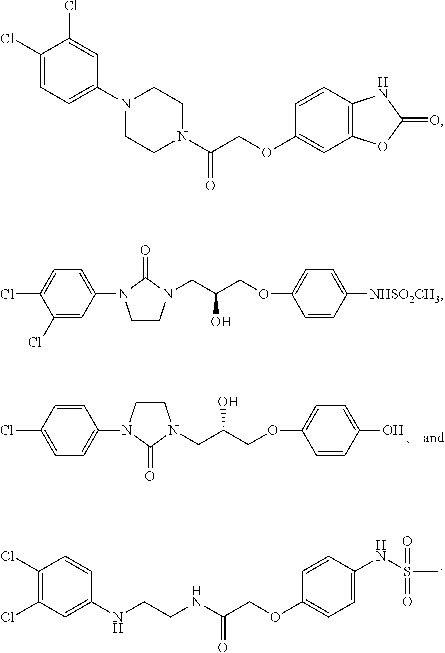

In one embodiment, the compound has an IC₅₀ of 600 nM or less and a pH boost of 5 or more. In a particular embodiment, the compound is

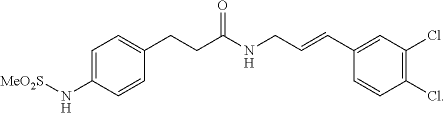

In another embodiment, the compound is

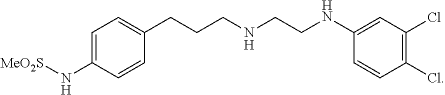

In another embodiment, the compound is,

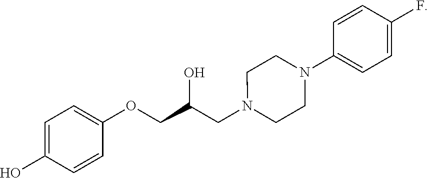

In another embodiment, the compound is,

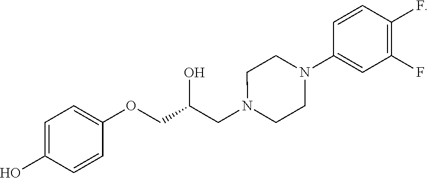

In a particular embodiment, the compound is

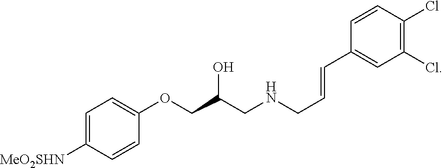

In another particular embodiment, the compound is

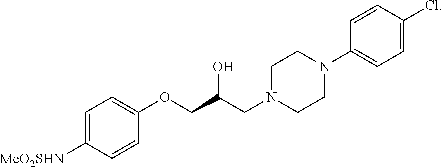

In another embodiment, the compound is,

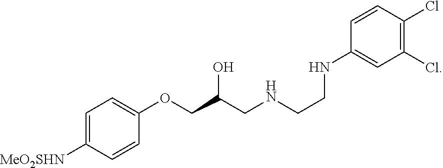

In another embodiment, the compound is,

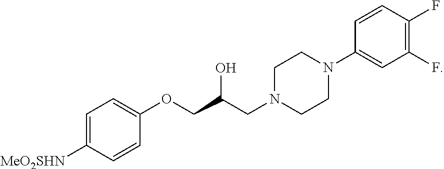

In one embodiment, the compound is selected from the group consisting of:

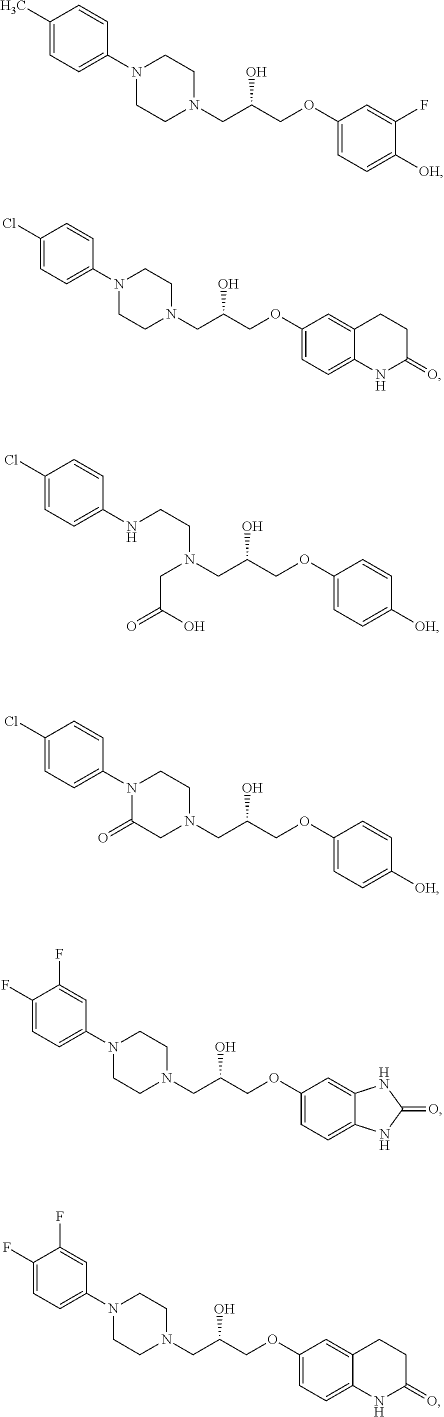

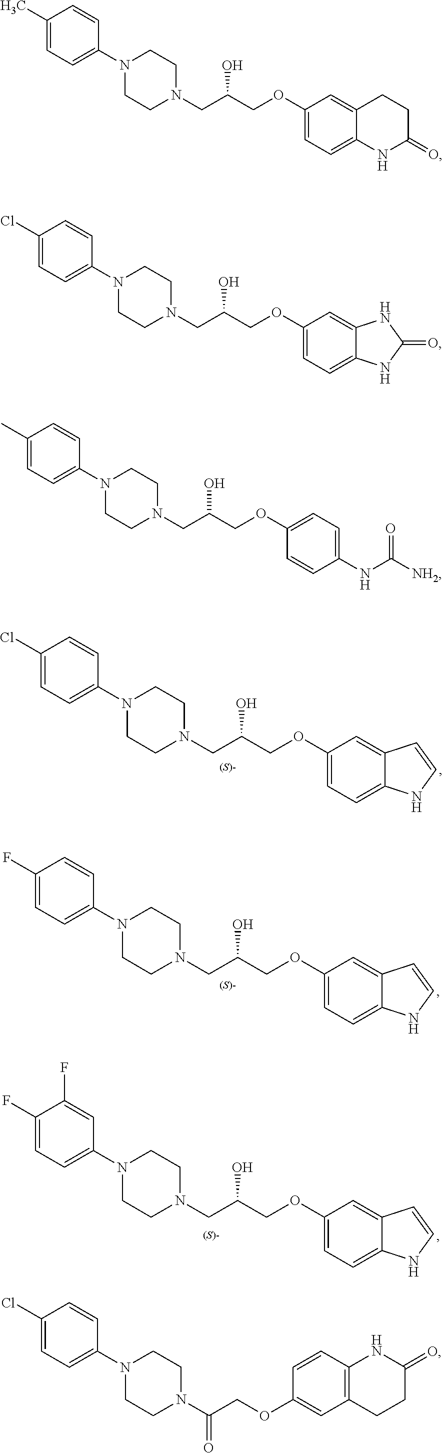

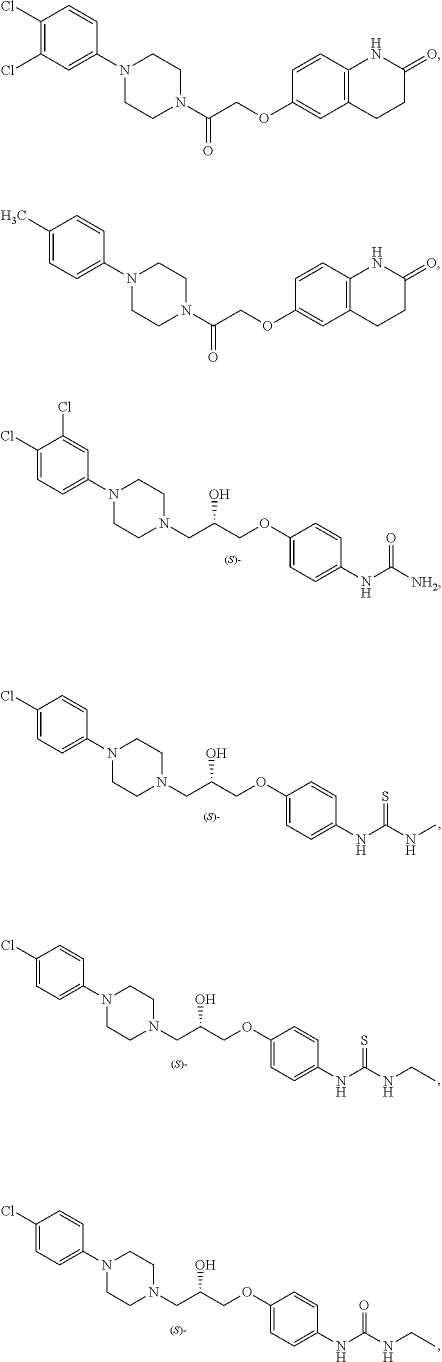

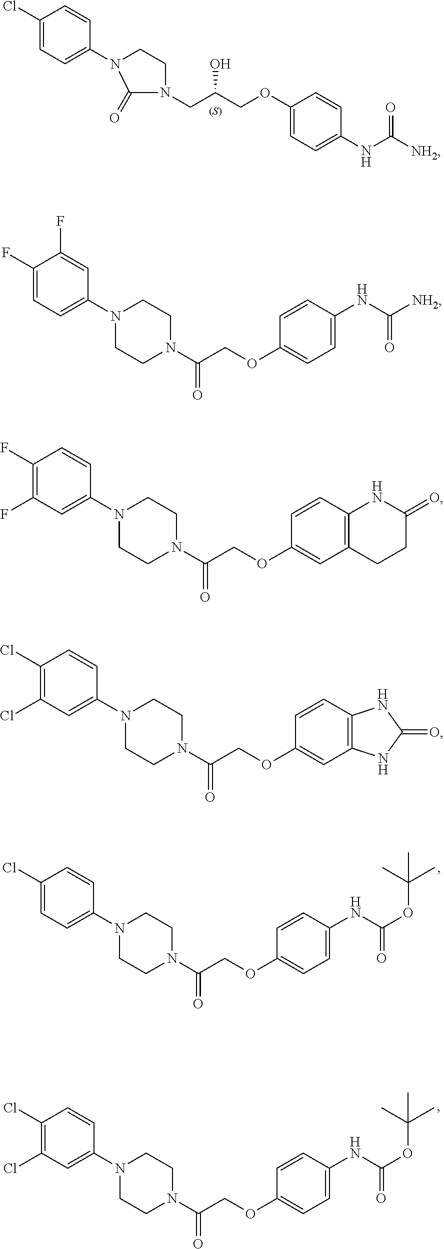

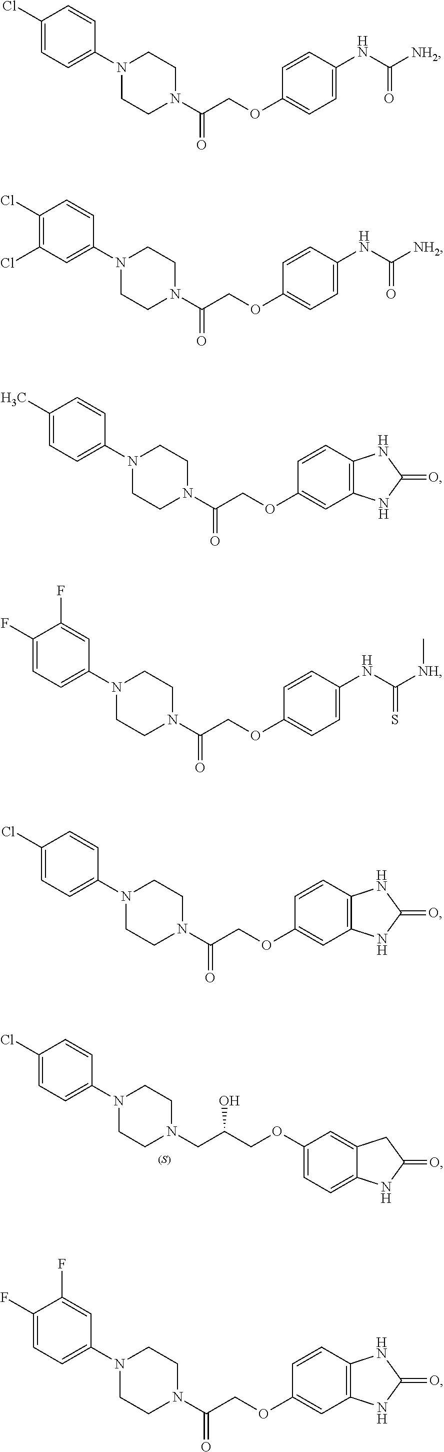

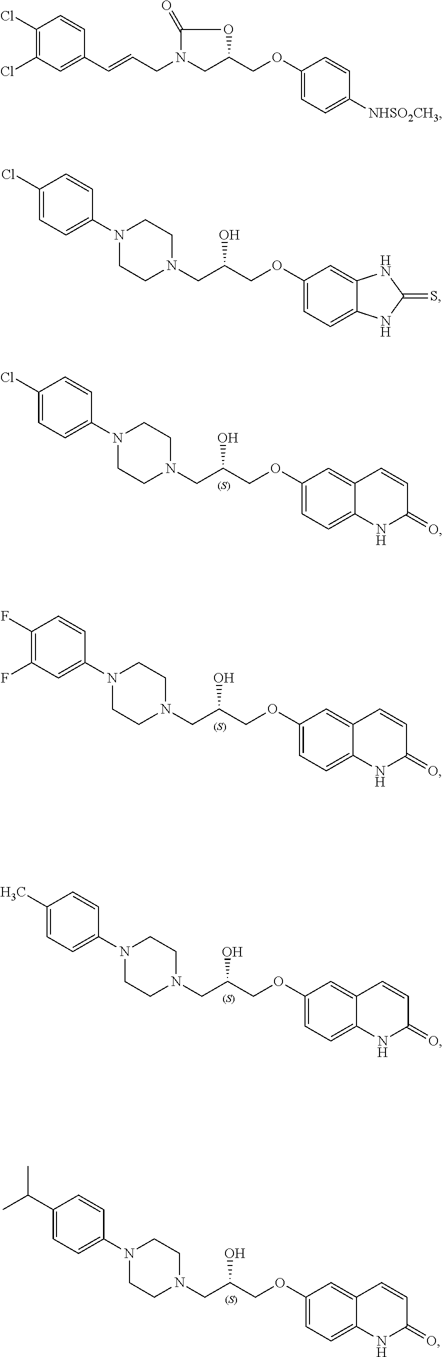

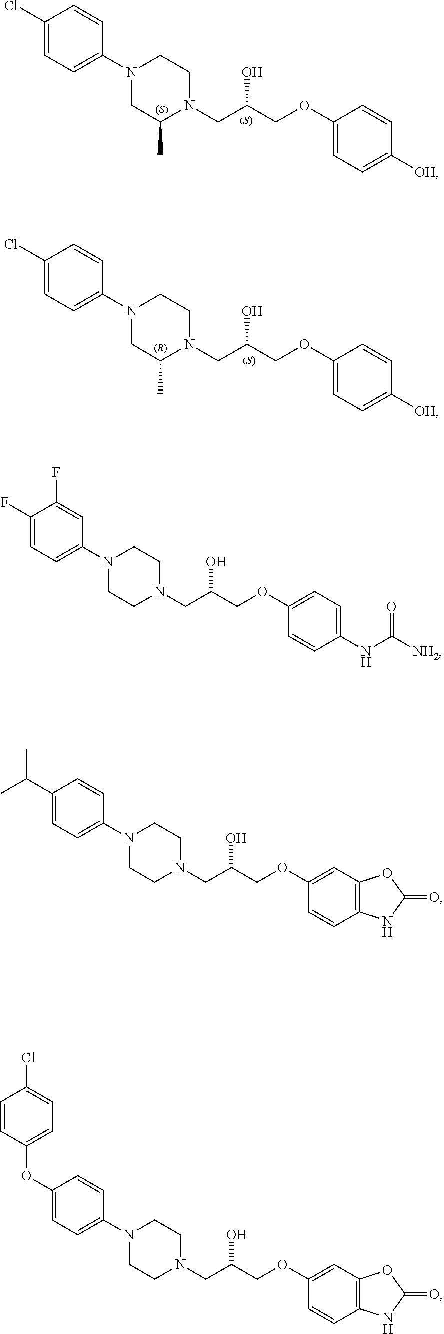

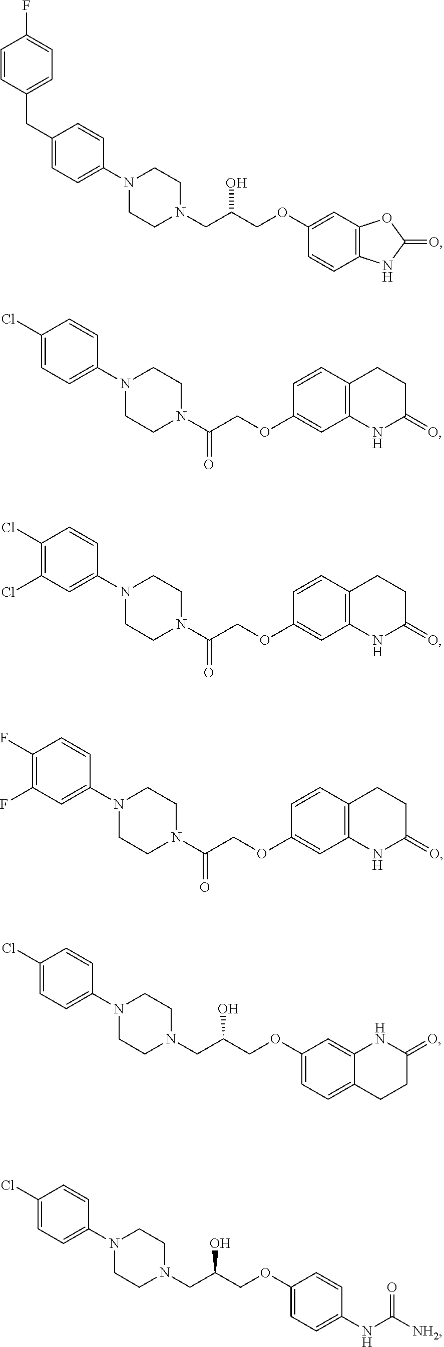

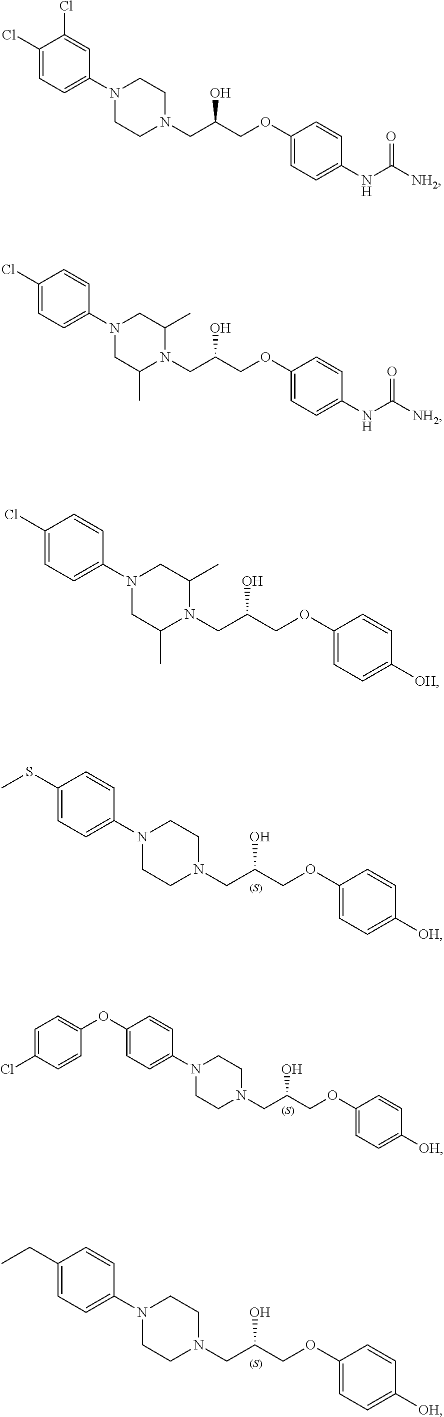

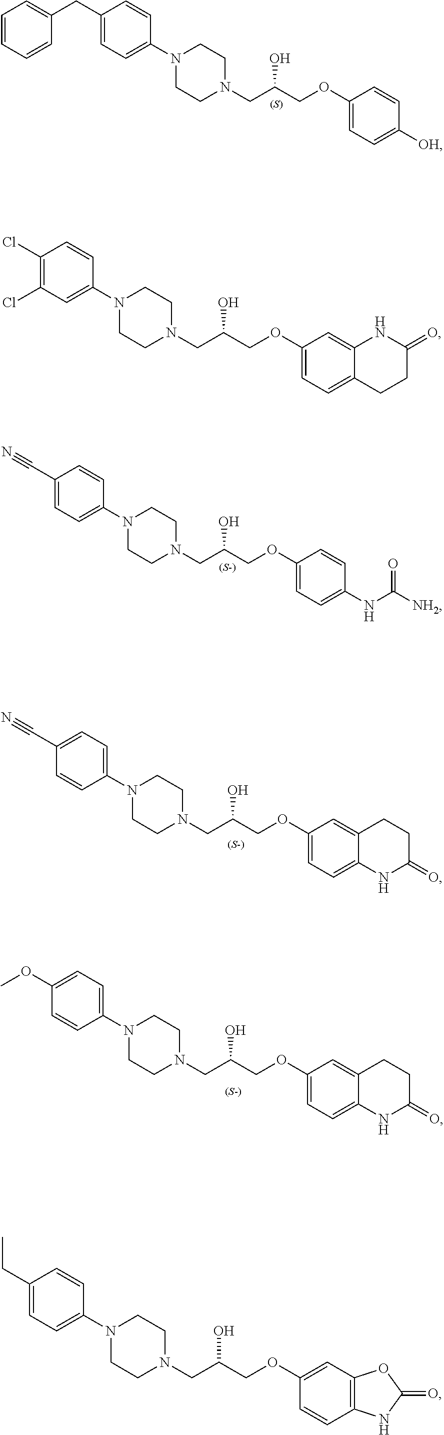

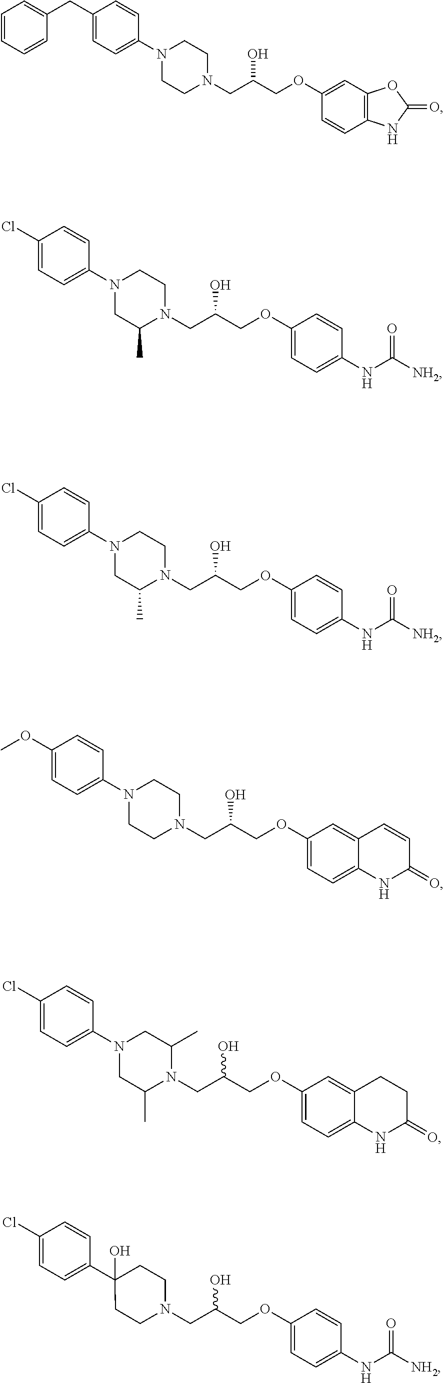

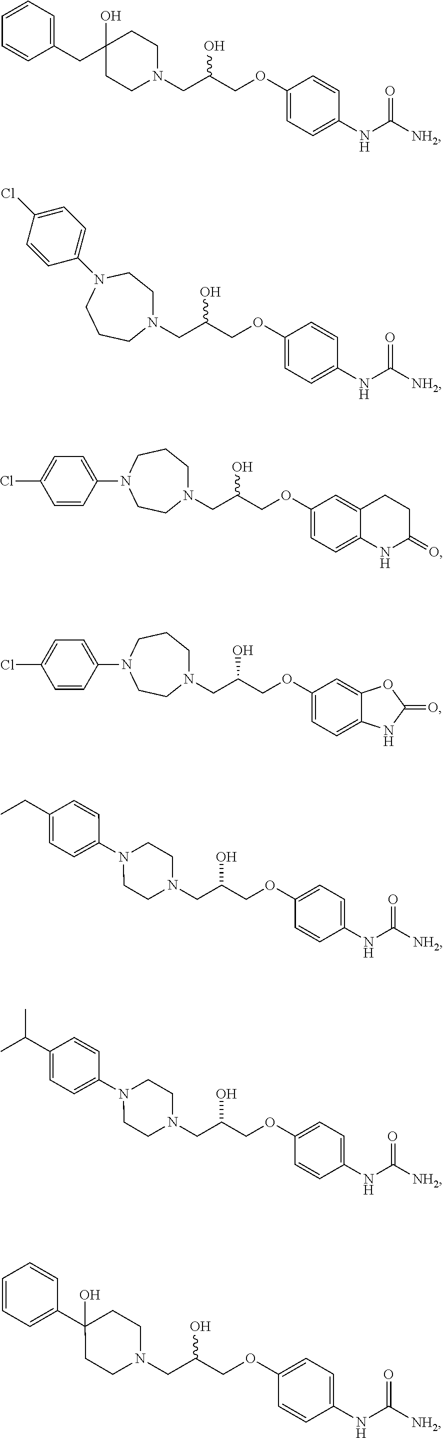

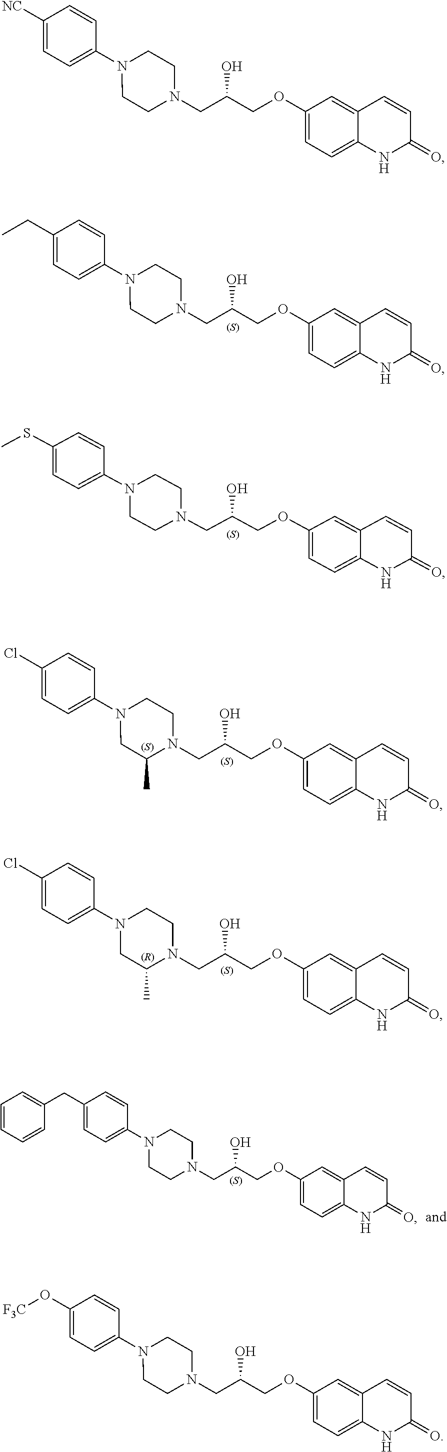

### Formula II

In one embodiment, methods of treatment or prophylaxis of neuropsychiatric disorders, in particular depression and anxiety are provided comprising administering a compound of Formula II or a pharmaceutically acceptable salt, ester, prodrug or derivative thereof to a host in need thereof:

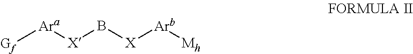

wherein:
each G is independently F, Cl, Br, I, C₁-C₄ alkyl, C₁-C₄ alkoxy, C₆-C₁₂ aralkyl, —O-aryl, —S-aryl, —NH-aryl;
f=0, 1, 2, 3, 4 or 5;
Ar^{a} and Ar^{b} are each independently aryl or heteroaryl;
B is selected from the group consisting of:

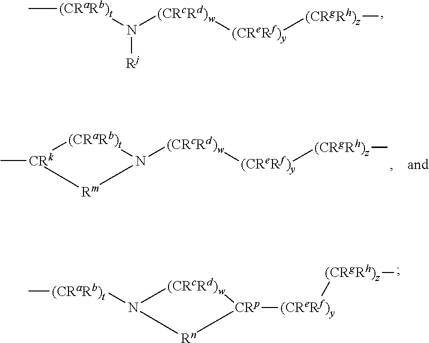

wherein R^{a}, R^{b}, R^{c}, R^{d}, R^{e}, R^{f}, R^{g}, R^{h}, R^{k} and R^{p} are each independently selected from H, C₁-C₆ alkyl, C₁-C₆ alkoxy, OH or halo;
R^{j} is H, C₁-C₆ alkyl, OH or P(O)(OC₁-C₄ alkyl)₂;
R^{m} is C₁-C₄ alkyl or C₂-C₄ alkenyl;
Rⁿ is C₁-C₄ alkyl, C₂-C₄ alkenyl, C₆-C₁₂ aralkyl, —CH₂O—, —CH(C₁-C₆ alkyl)O—, —CH(C₂-C₁₂ aralkyl)O—;
t, w, y and z each=0, 1, 2, or 3;
X and X′ are independently selected from a bond, O, S, SO, SO₂, CH₂, NH, N(C₁-C₆ alkyl), and NHC(═O);
M is OH, F, Cl, Br, I, NH₂, NR^{q}R^{r}, NO₂, O(C₁-C₆ alkyl), OCF₃, CN, C(O)OH, C(O)O(C₁-C₆ alkyl), C₆-C₁₂ aralkyl, NR^{s}C(O)CR^{t}₃, NR⁸SO₂(C₁-C₆ alkyl), or NR^{u}C(O)NR^{v}₂, wherein each R^{q}, R^{r}, R^{s}, R^{u} and R^{v} is each independently H or C₁-C₆ alkyl; and each R^{t} is independently H, C₁-C₆ alkyl or halo; or two M groups may be taken together with Ar^{b} to form:

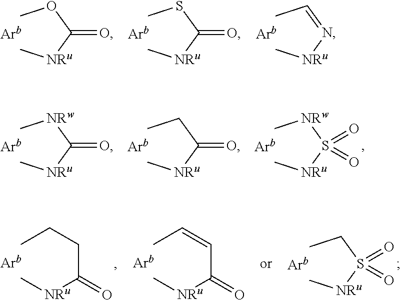

and wherein R^{u}
and R^{w} are independently H, C₁-C₆ alkyl or C₆-C₁₂ aralkyl; and
h=1, 2, 3, 4 or 5.

In some embodiments, when B contains a piperidin-4-ol or a pyrrolidin-2-ol moiety, and Ar^{a} and Ar^{b} are each phenyl, M is not OH at the para position on Ar^{b}.

In some embodiments of Formula II, each G is independently F, Cl, Br, I, C₁-C₄ alkyl, C₁-C₄ alkoxy, C₆-C₁₂ aralkyl, —O-aryl, —S-aryl, —NH-aryl;

f=0, 1, 2, 3, 4 or 5;

Ar^{a} and Ar^{b} are each independently aryl or heteroaryl;

B is

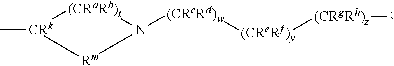

wherein R^{a-h}, R^{k} and R^{p} are each independently selected from H, C₁-C₆ alkyl, C₁-C₆ alkoxy, OH or halo;

R^{j} is H, C₁-C₆ alkyl, OH or P(O)(OC₁-C₄ alkyl)₂;

R^{m} is C₁-C₄ alkyl or C₂-C₄ alkenyl; P Rⁿ is C₁-C₄ alkyl, C₂-C₄ alkenyl, C₆-C₁₂ aralkyl, —CH₂O—, —CH(C₁-C₆ alkyl)O—, —CH(C₂-C₁₂ aralkyl)O—;

t, w, y and z each=0, 1, 2, or 3;

X and X′ are independently selected from a bond, O, S, SO, SO₂, CH₂, NH, N(C₁-C₆ alkyl), and NHC(═O);

M is OH, F, Cl, Br, I, NH₂, NR^{q}R^{r}, NO₂, O(C₁-C₆ alkyl), OCF₃, CN, C(O)OH, C(O)O(C₁-C₆ alkyl), C₆-C₁₂ aralkyl, NR^{s}C(O)CR^{t}₃, NR⁸SO₂(C₁-C₆ alkyl), or NR^{u}C(O)NR^{v}₂, wherein each R^{q}, R^{r}, R^{s}, R^{u} and R^{v} is each independently H or C₁-C₆ alkyl; and each R^{t} is independently H, C₁-C₆ alkyl or halo; or two M groups may be taken together with Ar^{b} to form:

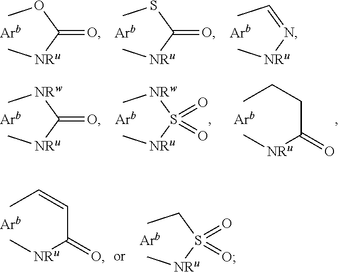

and wherein R^{u} and R^{w} are independently H, C₁-C₆ alkyl or C₆-C₁₂ aralkyl; and

h=1, 2, 3, 4 or 5.

In one embodiment, G is F or Cl. In another embodiment, f is 1 or 2.

In one embodiment, Ar^{a} is phenyl. In another embodiment, Ar^{b} is phenyl. In another embodiment, Ar^{a} and Ar^{b} are each phenyl. In one embodiment, Ar^{a} is phenyl and is substituted with two G groups. In a subembodiment, both G groups are Cl. In another subembodiment, both G groups are F. In another subembodiment, one G group is Cl and the other G group is F. In one embodiment, G is selected from the group consisting of C₆-C₁₂ aralkyl, —O-aryl, —S-aryl, and —NH-aryl.

In one embodiment, B is

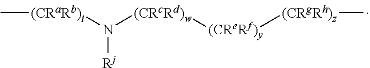

In a subembodiment, R^{a}, R^{b}, R^{c}, R^{d}, R^{e}, R^{g} and R^{h} are H; R^{j} is H, C₁-C₆ alkyl, OH or P(O)(OC₁-C₄ alkyl)₂; R^{f} is H, halo or OH; t is 0, 1, 2, or 3; and w, y and z are each 1.

In one embodiment, B is

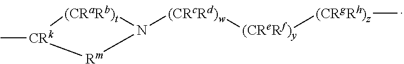

In a subembodiment, R^{a}, R^{b}, R^{c}, R^{d}, R^{e}, R^{g} and R^{h} are H; R^{f} and R^{k} are independently H, halo or OH; R^{m} is C₁-C₄ alkyl; t is 1, 2, or 3; and w, y and z are each 1. In specific subembodiments, B is

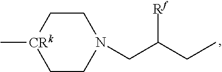

and R^{f} and R^{k} are independently H or OH. In certain subembodiments, Ar^{b} is substituted with one, two or three M groups, wherein the M group is independently selected from OH, F, Cl, Br, I, or NR^{u}C(O)NR^{v}₂, wherein each R^{u} and R^{v} is each independently H or C₁-C₆ alkyl or two M groups may be taken together with Ar^{b} to form

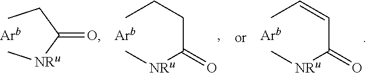

In certain subembodiments, X′ is selected from a bond, O, S, CH₂, NH. In particular subembodiments, f is 1 and G is at a para position of Ar^{a}.

In one embodiment, B is

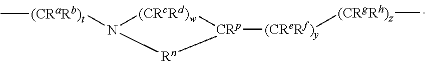

In a subembodiment, R^{a}, R^{b}, R^{c}, R^{d}, R^{e}, R^{g} and R^{h} are H; R^{f} is H, halo or OH; R^{p} is H, halo or OH; Rⁿ is —CH₂O—; t is 0, 1, 2, or 3; and w, y and z are each 1.

In one embodiment, the sum of w, y and z does not exceed 6. In one embodiment, the sum of w, y and z is 2, 3, 4, 5 or 6.

In one embodiment, X is a bond, O, S or CH₂. In another embodiment, X is O. In another embodiment, X is CH₂.

In one embodiment, X′ is a bond, NH, S or CH₂. In another embodiment, X′ is a bond. In another embodiment, X′ is S. In another embodiment, X′ is NH. In another embodiment, X′ is CH₂.

In one embodiment, M is OH. In another embodiment, M is F or Cl. In another embodiment, M is O(C₁-C₆ alkyl), for example OCH₃, OCH₂CH₃, O(CH₂)₂CH₃, OCH(CH₃)₂ or OC(CH₃)₃. In another embodiment, M is NH₂. In another embodiment, M is NR^{q}R^{r}. In another embodiment, M is NO₂. In another embodiment, M is OCF₃. In one embodiment, M is CN. In one embodiment, M is C(O)OH. In one embodiment, M is C(O)O(C₁-C₆ alkyl), for example C(O)OCH₃, C(O)OCH₂CH₃, C(O)O(CH₂)₂CH₃, C(O)OCH(CH₃)₂ or C(O)OC(CH₃)₃. In one embodiment, M is C₆-C₁₂ aralkyl, for example CH₂-phenyl. In one embodiment, M is NR^{s}C(O)CR^{t}₃. In a subembodiment, R^{s} is H. In a subembodiment, R^{t} is H or Cl. In one embodiment, M is NR^{u}(O)NR^{v}₂, for example, NHC(O)NH₂. In a subembodiment, R^{u} is H and R^{v} is H or alkyl.

In one embodiment, two M groups may be taken together with Ar^{b} to form:

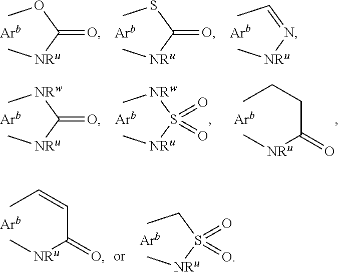

In a subembodiment, two M groups may be taken together with Ar^{b} to form:

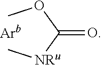

In one embodiment, R^{u} and R^{w} are both H. In one embodiment, h is 1 or 2.

In one embodiment, the compound is a compound of Formula II, or a pharmaceutically acceptable salt, ester, prodrug or derivative thereof, wherein:

each G is independently F, Cl, Br or I;
f is 0, 1, 2, 3, 4, or 5;
Ar^{a} and Ar^{b} are each independently selected from the group consisting of phenyl, pyridyl, pyrimidinyl, thiophenyl, imidazolyl, furanyl, indolyl, benzothiophenyl, benzofuranyl, benzoimidazolyl;
B is selected from the group consisting of:

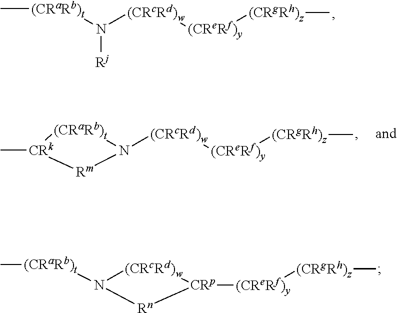

wherein R^{a}, R^{b}, R^{c}, R^{d}, R^{e}, R^{f}, R^{g}, R^{h}, R^{k} and R^{p} are each independently selected from H, C₁-C₆ alkyl, OH, or halo;
R^{j} is H, C₁-C₆ alkyl, C₂-C₁₂ aralkyl, or OH;
R^{m} is C₁-C₄ alkyl or C₂-C₄ alkenyl;
Rⁿ is C₁-C₄ alkyl, C₂-C₄ alkenyl, C₆-C₁₂ aralkyl, —CH₂O—, —CH(C₁-C₆ alkyl)O—, —CH(C₂-C₁₂ aralkyl)O—;
t, w, y and z each=0, 1, 2, or 3;
X is a bond, CH₂ or O;
X′ is a bond, CH₂, S or NH;
M is OH, F, Cl, Br, I, NH₂, NR^{q}R^{r}, NO₂, O(C₁-C₆ alkyl), OCF₃, CN, C(O)OH, C(O)O(C₁-C₆ alkyl), C₆-C₁₂ aralkyl, NR^{s}C(O)CR^{t}₃, or NR^{u}C(O)NR^{v}₂, wherein each R^{q}, R^{r}, R^{s}, R^{u} and R^{v} is each independently H or C₁-C₆ alkyl; and each R^{t} is independently H, C₁-C₆ alkyl or halo; or two M groups may be taken together with Ar^{b} to form:

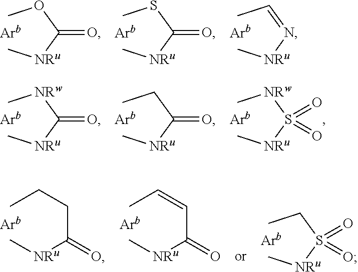

and wherein R^{u} and R^{w} are independently H or C₁-C₄ alkyl; and
h=1, 2 or 3.

In one embodiment, the compound is a compound of Formula II, or a pharmaceutically acceptable salt, ester, prodrug or derivative thereof, wherein:

each G is independently F, Cl, Br or I;
f=0, 1, 2, 3, 4 or 5;
Ar^{a} and Ar^{b} are each phenyl;
B is selected from the group consisting of:

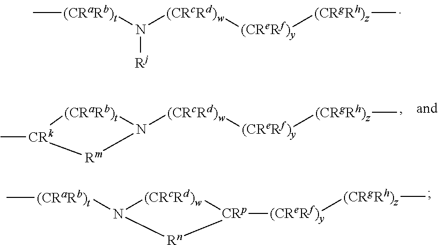

wherein R^{a}, R^{b}, R^{c}, R^{d}, R^{e}, R^{f}, R^{g}, R^{h}, R^{k} and R^{p} are each independently selected from H, C₁-C₆ alkyl, OH, or halo;
R^{j} is H, C₁-C₆ alkyl, or OH;
R^{m} is C₁-C₄ alkyl or C₂-C₄ alkenyl;
Rⁿ is C₁-C₄ alkyl, C₂-C₄ alkenyl, C₆-C₁₂ aralkyl, —CH₂O—, —CH(C₁-C₆ alkyl)O—, —CH(C₂-C₁₂ aralkyl)O—;
t, w, y and z each=0, 1, 2, or 3;
X is a bond, CH₂ or O;
X′ is a bond, CH₂, S or NH;
M is OH, F, Cl, Br, I, NH₂, NR^{q}R^{r}, NO₂, O(C₁-C₆ alkyl), OCF₃, CN, C(O)OH, C(O)O(C₁-C₆ alkyl), C₆-C₁₂ aralkyl, NR^{s}C(O)CR^{t}₃; wherein each R^{q}, R^{r}, and R^{s} is each independently H or C₁-C₆ alkyl; and each R^{t} is independently H, C₁-C₆ alkyl or halo; or two M groups may be taken together with Ar^{b} to form:

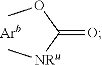

and wherein R^{u} is H or C₁-C₄ alkyl; and
h=1, 2 or 3.

In one embodiment, M is NR^{u}C(O)NR^{v}₂, for example NHC(O)NH₂ or NHC(O)N(CH₃)₂.

In another embodiment, Ar^{b}-M is selected from the group consisting of:

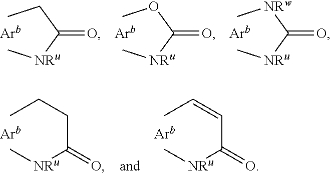

In one embodiment, the compound is

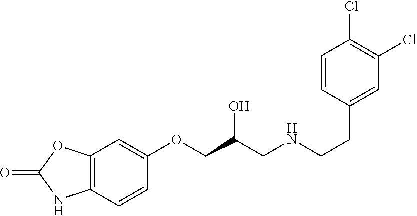

### or 6-{3-[2-(3,4-Dichloro-phenyl)-ethylamino]-2-(S)-hydroxy-propoxy}-3H-benzooxazol-2-one.

In one embodiment, the compound is

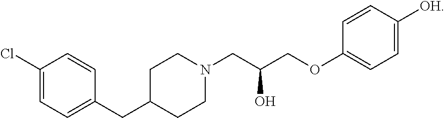

In one embodiment, the compound is selected from the compounds in Table 14.

TABLE 14

| Compound | NAME |
|---|---|
| 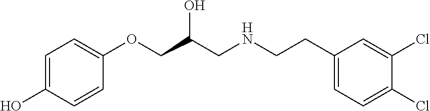 | 4-{3-[2-(3,4-Dichloro-phenyl)-ethylamino]-2-(S)- hydroxy-propoxy}-phenol |
| 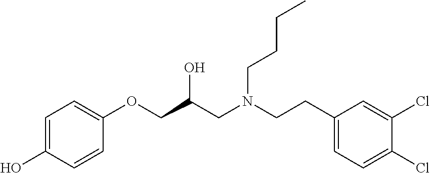 | 4-(3-{Butyl-[2-(3,4-dichloro-phenyl-ethyl]- amino}-2-(S)-hydroxy-propoxy)-phenol |
| 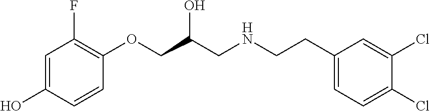 | 4-{3-[2-(3,4-Dichloro-phenyl)-ethylamino]-2-(S)- hydroxy-propoxy}-3-fluoro-phenol |
| 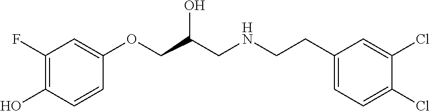 | 4-{3-[2-(3,4-Dichloro-phenyl)-ethylamino]-2-(S)- hydroxy-propoxy}-2-fluoro-phenol |
| 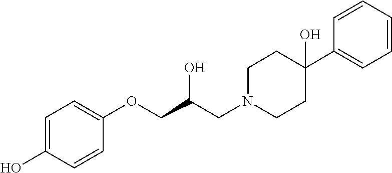 | 1-[2-(S)-Hydroxy-3-(4-hydroxy-phenoxy)-propyl]- 4-phenyl-piperidin-4-ol |
| 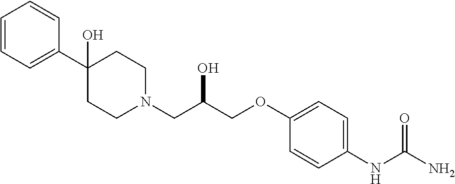 | (R)-1-(4-(2-hydroxy-3-(4-hydroxy-4- phenylpiperidin-1-yl)propoxy)phenyl)urea |
| 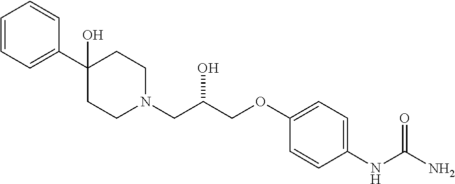 | (S)-1-(4-(2-hydroxy-3-(4-hydroxy-4- phenylpiperidin-1-yl)propoxy)phenyl)urea |
| 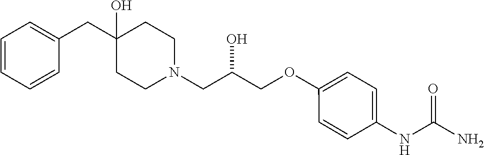 | (S)-1-(4-(3-(4-benzyl-4-hydroxypiperidin-1-yl)-2- hydroxypropoxy)phenyl)urea |
| 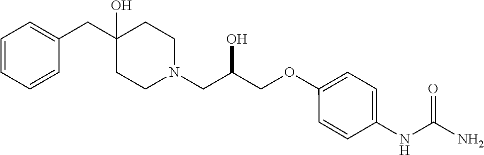 | (R)-1-(4-(3-(4-benzyl-4-hydroxypiperidin-1-yl)-2- hydroxypropoxy)phenyl)urea |
| 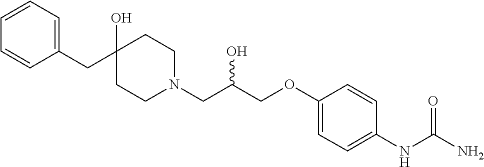 | 1-(4-(3-(4-benzyl-4-hydroxypiperidin-1-yl)-2- hydroxypropoxy)phenyl)urea |
| 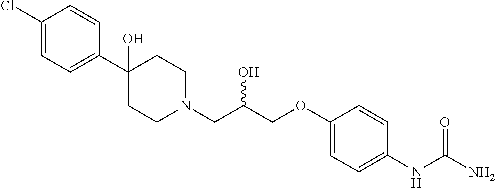 | 1-(4-(3-(4-(4-chlorobenzyl)-4-hydroxypiperidin-1- yl)-2-hydroxypropoxy)phenyl)urea |
| 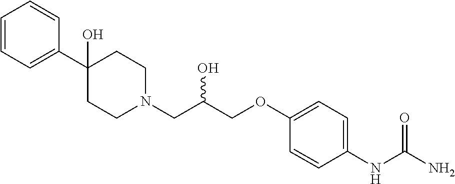 | 1-(4-(2-hydroxy-3-(4-hydroxy-4-phenylpiperidin-1- yl)propoxy)phenyl)urea |
| 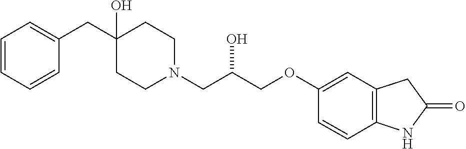 | (S)-5-(3-(4-benzyl-4-hydroxypiperidin-1-yl)-2- hydroxypropoxy)indolin-2-one |
| 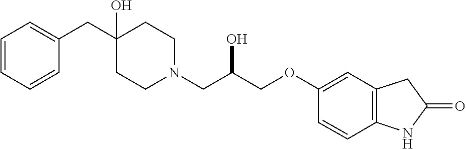 | (R)-5-(3-(4-benzyl-4-hydroxypiperidin-1-yl)-2- hydroxypropoxy)indolin-2-one |
| 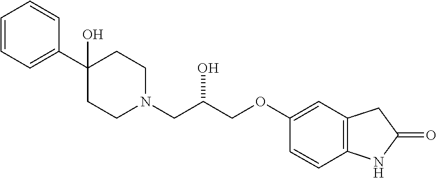 | (S)-5-(2-hydroxy-3-(4-hydroxy-4-phenylpiperidin- 1-yl)propoxy)indolin-2-one |
| 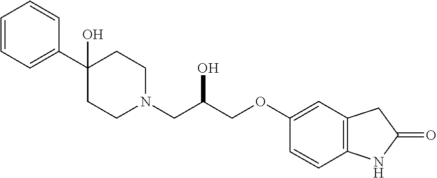 | (R)-5-(2-hydroxy-3-(4-hydroxy-4-phenylpiperidin- 1-yl)propoxy)indolin-2-one |
| 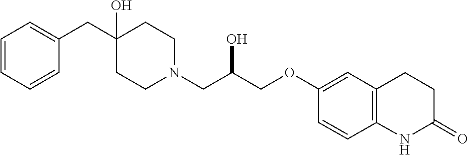 | (R)-6-(3-(4-benzyl-4-hydroxypiperidin-1-yl)-2- hydroxypropoxy)-3,4-dihydroquinolin-2(1H)-one |
| 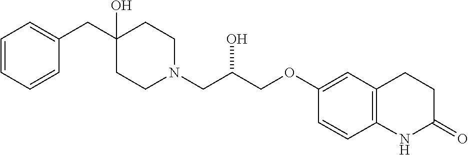 | (S)-6-(3-(4-benzyl-4-hydroxypiperidin-1-yl)-2- hydroxypropoxy)-3,4-dihydroquinolin-2(1H)-one |
| 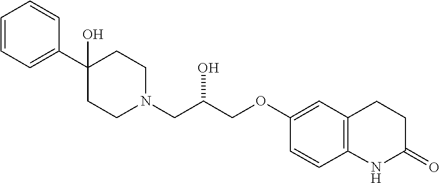 | (S)-6-(2-hydroxy-3-(4-hydroxy-4-phenylpiperidin- 1-yl)propoxy)-3,4-dihydroquinolin-2(1H)-one |
| 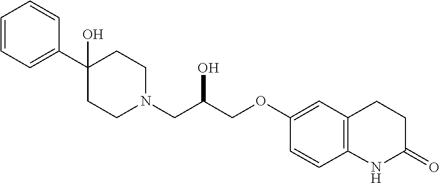 | (R)-6-(2-hydroxy-3-(4-hydroxy-4-phenylpiperidin- 1-yl)propoxy)-3,4-dihydroquinolin-2(1H)-one |
| 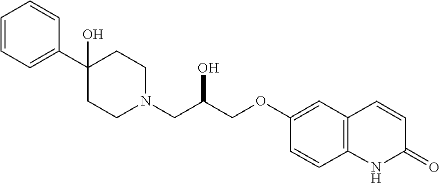 | (R)-6-(2-hydroxy-3-(4-hydroxy-4-phenylpiperidin- 1-yl)propoxy)quinolin-2(1H)-one |

In one embodiment, the compound has an IC₅₀ value of 600 nM or less. In one embodiment, the compound has an IC₅₀ value of 600 nM or less at pH 6.9 or an ischemic pH. In one embodiment, the compound is selected from Table 15.

TABLE 15

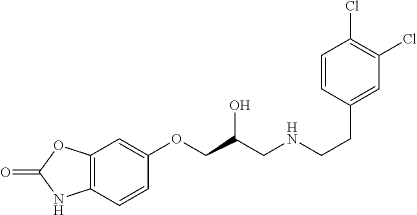

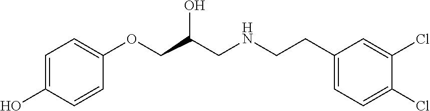

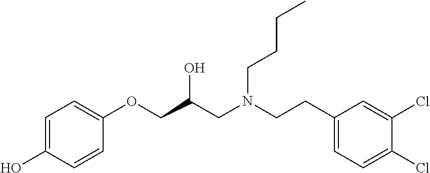

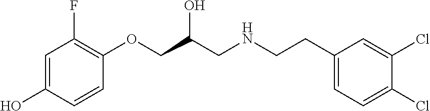

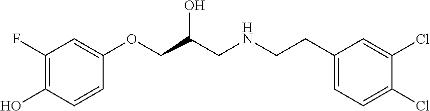

In one embodiment, the compound has a pH boost of 5 or more. In one embodiment, the compound is

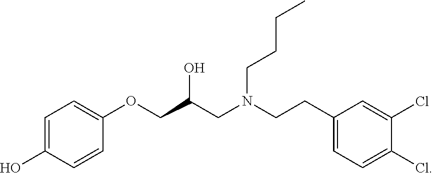

In one embodiment, the compound is selected from the group consisting of:

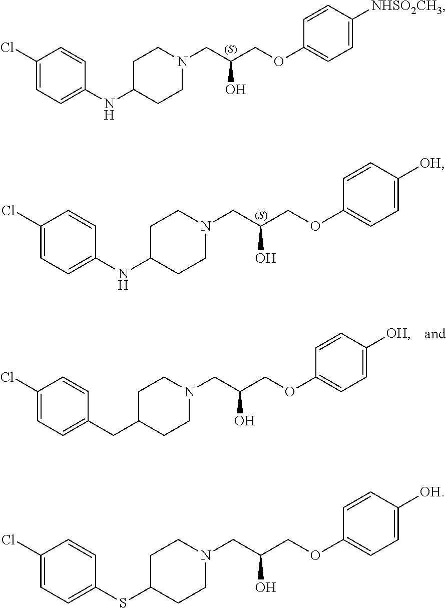

In one embodiment, the compound is

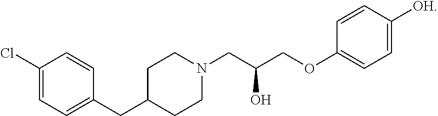

In another embodiment, the compound is selected from the group consisting of:

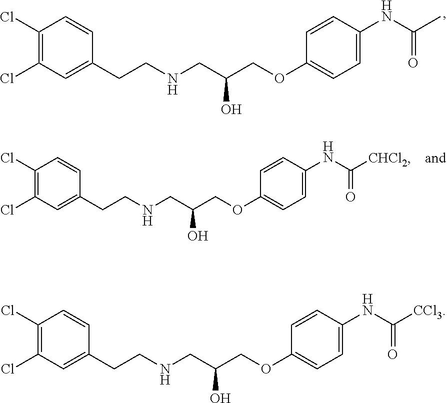

In another embodiment the compound is

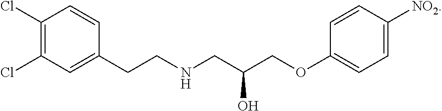

In one embodiment, one or more of R^{c}, R^{d}, R^{e}, R^{f}, R^{g}, and R^{h} is an OH group which creates a stereogenic center. In a particular subembodiment, one of R^{c}, R^{d}, R^{e}, R^{f}, R^{g}, and R^{h} is an OH group which creates a stereogenic center. In another subembodiment, the OH group at one of R^{c}, R^{d}, R^{e}, R^{f}, R^{g}, and R^{h} is in the R configuration. In another subembodiment, the OH group at one of R^{c}, R^{d}, R^{e}, R^{f}, R^{g}, and R^{h} is in the S configuration.

In certain embodiments, the binding to both hERG and alpha-1 adrenergic receptors can be modulated by changing the G substituent or G substituents. In particular, for compounds wherein Ar^{a} is phenyl, the binding to both hERG and alpha-1 adrenergic receptors can be modulated by changing the substitution at the 3 and/or 4 positions. In one embodiment, the Ar^{a} phenyl is substituted at the 3 and/or 4 position with, for example, fluorine or chlorine. In certain embodiments, substitution at the 3 and/or 4 positions of the Ara phenyl can increase potency.

In certain embodiments, both hERG and alpha-1 adrenergic binding can be reduced by substituting N at the R^{j} position with C₂-C₁₂ aralkyl. In a particular subembodiment, R^{j} is benzyl.

In certain embodiments, alpha-1 adrenergic binding is reduced when R^{j} is C₁-C₆ alkyl.

When Ar^{b} is phenyl, para substitution of the M substituent is particularly preferred. Additional M substitutents on the Ar^{b} phenyl are preferred at one or more ortho positions. Additional substitution on the Ar^{b} phenyl at one or more meta positions can reduce potency.

In certain embodiments, the Ar^{a} phenyl is not substituted by two fluoro groups. In one embodiment, the Ar^{a} phenyl is not substituted by two methyl groups. In one embodiment, the Ar^{a} phenyl is not substituted by one halo group. In one embodiment, the Ar^{a} phenyl is not substituted by one fluoro or alkyl group at the C-2 position. In one embodiment, the Ar^{a} phenyl is not substituted by a OH or NO₂ group.

In one embodiment, when Ar^{a} and Ar^{b} are both phenyl, at least one of for h is not 0. In one embodiment, when Ar^{a} and Ar^{b} are both phenyl, f is not 0. In one embodiment, when Ar^{a} and Ar^{b} are both phenyl, h is not 0. In one embodiment, when Ar^{a} and Ar^{b} are both phenyl, X is not CH₂. In one embodiment, when Ar^{a} and Ar^{b} are both phenyl, X′ is not CH₂. In another embodiment, M is not OH. In one embodiment, the compound is not

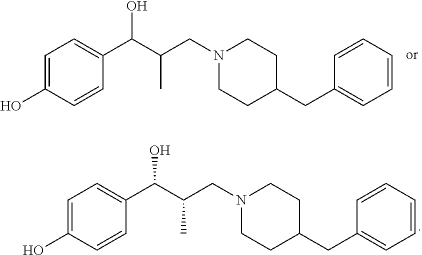

In one embodiment, M is not aralkoxy. In one embodiment, the compound is not

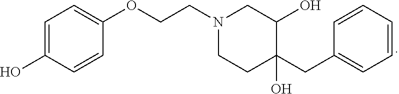

In one embodiment, B does not contain a piperidinyl moiety. In another embodiment, when B contains a piperidinyl moiety, and Ar^{a} and Ar^{b} are both phenyl, M is not OH. In one embodiment, when B contains a piperidinyl moiety, M is NR^{u}C(O)NR^{v}₂, for example, NHC(O)NH₂. In a subembodiment, R^{u} is H and R^{v} is H or alkyl. In one embodiment, when B contains a piperidinyl moiety, X is not CH₂. In one embodiment, when B contains a piperidinyl moiety, X′ is not CH₂. In one embodiment, R^{k} is not OH. In one embodiment, R^{p} is not OH.

In one embodiment, when B contains a hydroxy-substituted-piperidinyl moiety, X is not CH₂. In one embodiment, when B contains a hydroxy-substituted-piperidinyl moiety, X′ is not CH₂. In one embodiment, B does not contain a hydroxy-substituted-piperidinyl moiety.

In one embodiment, X is not SO₂. In another embodiment, X′ is not SO₂. In one embodiment, when B contains a piperidinyl moiety, X is not SO₂. In one embodiment, when B contains a piperidinyl moiety, X′ is not SO₂.

In one embodiment, X is not S. In another embodiment, X′ is not S. In one embodiment, when B contains a piperidinyl moiety, X is not S. In one embodiment, when B contains a piperidinyl moiety, X′ is not S.

In another embodiment, M is not OCH₃ or OCF₃. In another embodiment, M is not NO₂. In one embodiment, when B contains a nitrogen-containing heterocycle, Ar^{b}—X is not heteroaryl-NH. In another embodiment, when B contains a nitrogen-containing heterocycle, Ar^{a}—X′ is not heteroaryl-NH.

In one embodiment, when B contains a nitrogen-containing heterocycle, X is not NH(C═O). In another embodiment, when B contains a nitrogen-containing heterocycle, X′ is not NH(C═O).

### Formula III

In one embodiment, methods of treatment or prophylaxis of neuropsychiatric disorders, in particular depression and anxiety are provided comprising administering a compound of Formula III or a pharmaceutically acceptable salt, ester, prodrug or derivative thereof to a host in need thereof:

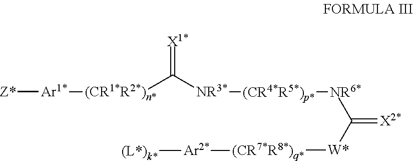

wherein:
Z* is OH, NR¹⁰*R¹¹*, NR¹²*SO₂R¹¹*, NR¹²*C(O)NR¹⁰*R¹¹*, NR¹²*C(O)OR¹⁰*, NR¹²*-dihydrothiazole, or NR¹²*-dihydroimidazole; wherein each R¹⁰*, R¹¹* and R¹²* is independently H, C₁-C₆ alkyl or C₆-C₁₂ aralkyl; or Ar¹*—Z* is

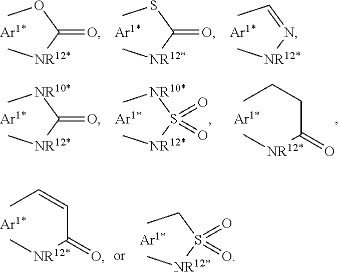

Ar¹* and Ar²* are each independently aryl or heteroaryl;
R¹*, R²*, R⁴*, R⁵*, R⁷*, R⁸* are independently H, OH or C₁-C₄ alkyl;
n*=1, 2, 3 or 4;
p*=0, 1, 2 or 3;
q*=0.1 or 2;
R³* and R⁶* are each independently H or C₁-C₄ alkyl;
X¹* and X²* are each independently O, S, N(C₁-C₄ alkyl) or C(H or C₁-C₄ alkyl)₂;
W* is NR⁹* or CR¹³*R¹⁴*; wherein R⁹*, R¹³* and R¹⁴* are each independently is H or C₁-C₄ alkyl;
each L* is independently C₁-C₆ alkyl, C₁-C₆ alkoxy, C(═O)—(C₁-C₆)-alkyl, C₁-C₆ haloalkyl, hydroxy, fluoro, chloro, bromo, iodo, nitro, or cyano; or two L groups may be taken together with Ar²* to form a dioxolane ring or a cyclobutane ring;
k*=0, 1, 2, 3, 4 or 5;

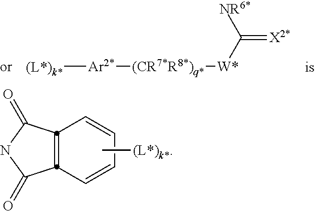

In one embodiment, Z* is OH, NR¹²*SO₂R¹¹*; wherein R¹²* is H or C₁-C₄ alkyl, and R¹¹* is C₁-C₄ alkyl or C₇-C₁₀ aralkyl. In one embodiment, Z* is OH. In another embodiment, Z* is NR¹²*SO₂R¹¹*, for example, NHSO₂CH₃.

In one embodiment, Z* is NR¹²*C(O)NR¹⁰*R¹¹* or Ar¹*—Z* is

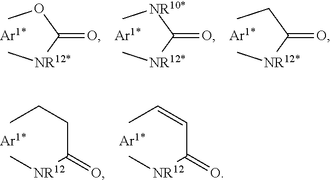

In one embodiment, Ar¹* and Ar²* are each phenyl.

In one embodiment, R¹*, R²*, R⁴*, R⁵*, R⁷*, R⁸* are H.

In a particular embodiment, n* is 2.

In one embodiment, p* is 0, 1 or 2. In another embodiment, p* is 0. In another embodiment, p* is 1. In another embodiment, p* is 2.

In one embodiment, q* is 0. In another embodiment, q* is 1. In another embodiment, q* is 2.

In one embodiment, R³* and R⁶* are both H. In one embodiment, R⁶* is C₁-C₄ alkyl, for example methyl, ethyl, propyl, isopropyl, n-butyl, sec-butyl, or tert-butyl.

In one embodiment, X* is S. In one embodiment, X* is O.

In one embodiment, W* is NR⁷*, for example NH. In another embodiment, W* is CR¹³*R¹⁴*, for example CH₂.

In one embodiment, each L* is independently selected from C₁-C₄ alkyl, F, Cl, Br, I, or C₁-C₄ haloalkyl, for example, Cl, CH₃ or CF₃. In one embodiment, k* is 1. In another embodiment, k* is 2.

In one embodiment, the compound is a compound of Formula III, or a pharmaceutically acceptable salt, ester, prodrug or derivative thereof, wherein:

### Z* is OH, NHSO₂CH₃;

Ar¹* is phenyl;
R¹*, R²*, R⁴*, R⁵* are independently H or C₁-C₄ alkyl;
n*=2;
p*=0, 1 or 2;
q*=0, 1 or 2;
R³* and R⁶* are each independently H or C₁-C₄ alkyl;

### X* is O or S;

W* is NR⁷* or CR¹³*R¹⁴*; wherein R⁷*, R¹³* and R¹⁴* are each independently is H or C₁-C₄ alkyl;
Ar²* is phenyl;
each L* is independently selected from C₁-C₄ alkyl, F, Cl, Br, I, C₁-C₄ haloalkyl;
k*=0, 1, 2, 3, 4 or 5;

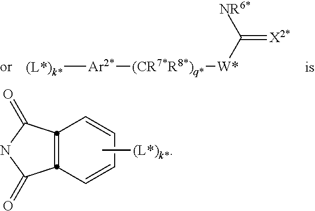

In one embodiment, the compound is selected from the group consisting of:

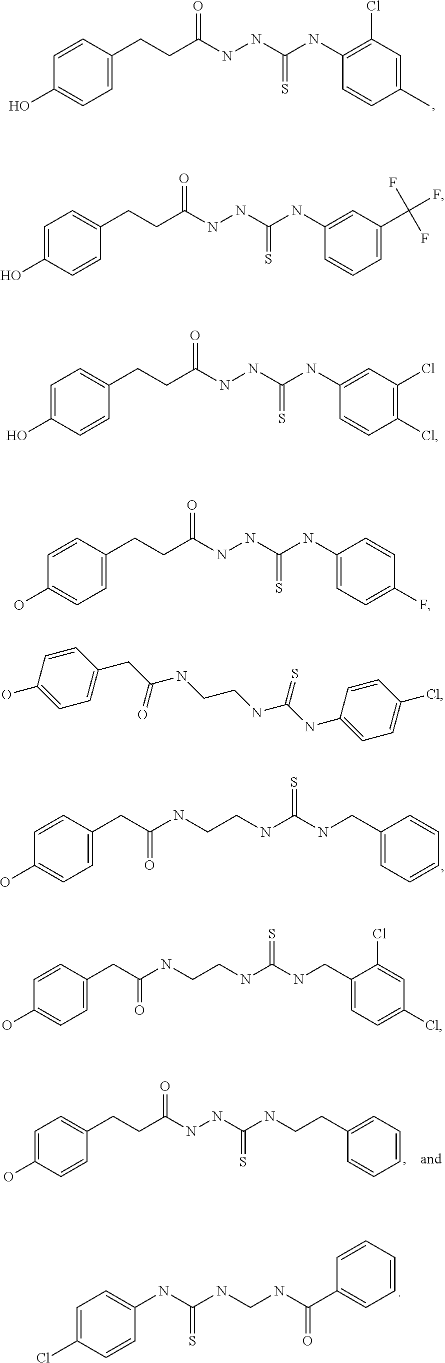

In another embodiment, the compound is selected from the group consisting of:

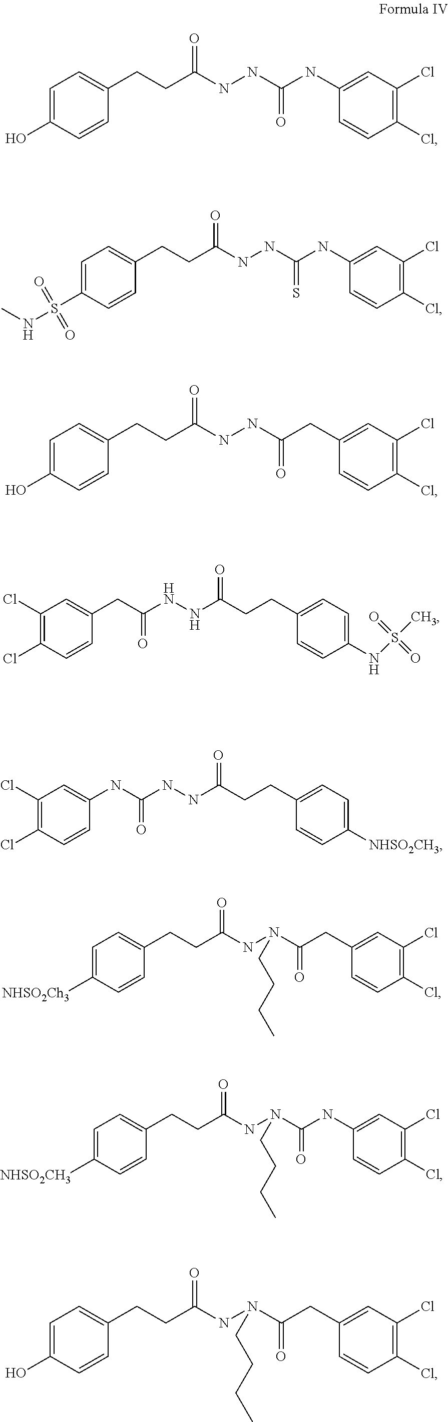

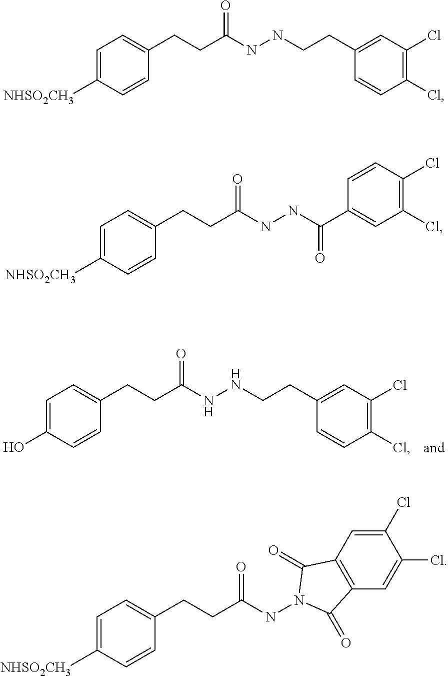

In one embodiment, methods of treatment or prophylaxis of neuropsychiatric disorders, in particular depression and anxiety are provided comprising administering a compound of Formula IV or a pharmaceutically acceptable salt, ester, prodrug or derivative thereof to a host in need thereof:

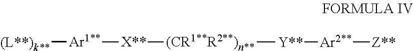

wherein:
each L** is independently C₁-C₆ alkyl, C₁-C₆ alkoxy, C(═O)—(C₁-C₆)-alkyl, C₁-C₆ haloalkyl, hydroxy, fluoro, chloro, bromo, iodo, nitro, or cyano; or two L** groups may be taken together with Ar¹** to form: a dioxolane ring or a cyclobutane ring;
k**=0, 1, 2, 3, 4 or 5;
each Ar¹** and Ar²** is independently aryl or heteroaryl;
X** is S, O or NR³; wherein R³ is H, C₁-C₆ alkyl, or C₆-C₁₂ aralkyl;
each R¹** and R²** is independently H, C₁-C₆ alkyl, C₁-C₆ alkoxy, C₆-C₁₂ aralkyl, C(═O)—(C₁-C₆)-alkyl, C₁-C₆ haloalkyl, hydroxy, fluoro, chloro, bromo, iodo, nitro, or cyano; or CR¹R² can be C═O or C═CH₂;
n**=1, 2, 3 or 4;
Y** is a bond, O, S, SO, SO₂, CH₂, NH, N(C₁-C₆ alkyl), or NHC(═O);
Z** is OH, NR⁶**R⁷**, NR⁸**SO₂(C₁-C₆ alkyl), NR⁸**C(O)NR⁶**R⁷**, NR⁸**C(O)O(C₁-C₆ alkyl), NR⁸**-dihydrothiazole, or NR⁸**-dihydroimidazole; wherein each R⁶**, R⁷** and R⁸** is independently H, C₁-C₆ alkyl or C₆-C₁₂ aralkyl; or

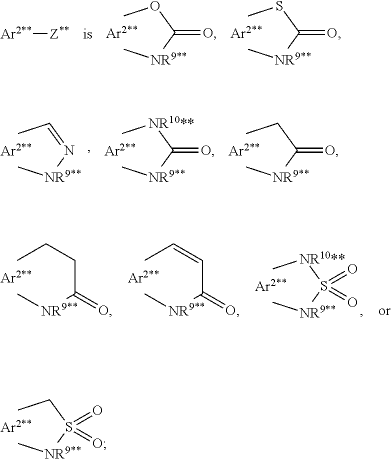

wherein R⁹** and R¹⁰** are each independently H, C₁-C₆ alkyl, aralkyl.

In particular subembodiment, Ar¹** is phenyl, pyridyl, pyrimidinyl, thiophenyl, imidazolyl, furanyl, indolyl, benzothiophenyl, benzofuranyl, or benzoimidazolyl. In one embodiment, Ar²** is phenyl. In another embodiment, Ar¹** is benzoimidazolyl. In a particular subembodiment, Ar²** is phenyl and Ar¹** is a heteroaryl, for example benzoimidazolyl. In one embodiment, Ar¹** is a bicyclic group wherein the X** group is attached to the heterocyclic ring.

In one embodiment, X** is S. In one embodiment, X** is O. In one embodiment, X** is NR³**, for example NH.

In another particular subembodiment, L** is C₁-C₄ alkyl, C₁-C₄ alkoxy, C(═O)—(C₁-C₄)-alkyl, C₁-C₆ haloalkyl, hydroxy, fluoro, chloro, bromo, iodo, nitro, or cyano. In a further subembodiment, L** is methyl, trifluoromethyl, methoxy, nitro, fluoro, chloro or hydroxy. In one further subembodiment, there are one, two or three L** groups substituting Ar¹**. In one subembodiment, Ar¹** is substituted with one fluoro group. In one subembodiment, Ar¹** is substituted with two fluoro groups. In one subembodiment, Ar¹** is substituted with one fluoro group and one chloro group. In one subembodiment, Ar¹** is substituted with one chloro group. In one subembodiment, Ar¹** is substituted with two chloro groups. In one subembodiment, Ar¹** is substituted with one methyl group. In one subembodiment, Ar¹** is substituted with one trifluoromethyl group.

In one embodiment, each R¹** and R²** is independently H or C₁-C₄ alkyl, for example, methyl, ethyl, n-propyl, isopropyl, n-butyl, sec-butyl or tert-butyl. In one embodiment, R¹** and R²** are both H. In one embodiment, one R¹** or R²** is hydroxy. In one embodiment, n** is 2, 3, or 4. In one embodiment, n** is 3.

In one embodiment, one CR¹**R²** is C═O or C═CH₂. In one embodiment, (CR¹**R²**)_{n**} is selected from the group consisting of

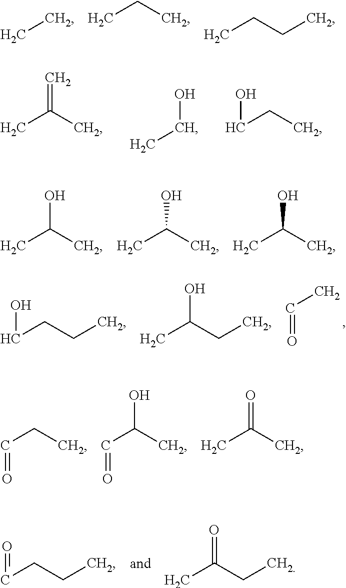

In an particular embodiment, (CR¹**R²**)_{n**} is

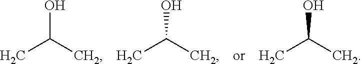

In one embodiment, Y** is a bond, O or CH₂. In one embodiment, Y** is O. In one subembodiment, Ar²** is phenyl and is substituted with a Z** group at the 4 position.

In one embodiment, Z** is OH, NR⁶**R⁷**, NR⁸**SO₂(C₁-C₆ alkyl), NR⁸**C(O)NR⁶**R⁷**, NR⁸**C(O)O(C₁-C₆ alkyl), NR⁸**-dihydrothiazole, or NR⁸**-dihydroimidazole. In one subembodiment, Ar²** is phenyl and is substituted with a Z** group at the 4 position. In one embodiment, Ar²**—Z** is

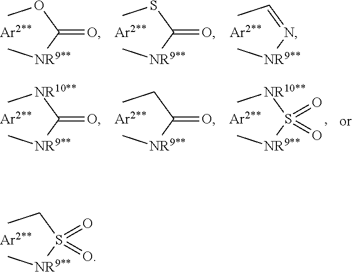

In one embodiment, Ar²**—Z** is

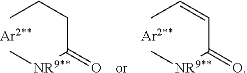

In one subembodiment, Ar²**—Z** is

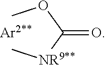

In one subembodiment, R⁹** and R¹⁰** are each H.

In one embodiment, the compound is a compound of Formula IV, or a pharmaceutically acceptable salt, ester, prodrug or derivative thereof, wherein:

L** is C₁-C₄ alkyl, C₁-C₄ alkoxy, C(═O)—(C₁-C₄)-alkyl, C₁-C₄ haloalkyl, hydroxy, fluoro, chloro, bromo, iodo, or nitro;
k**=0, 1, 2, 3, 4 or 5;
Ar¹** is selected from the group consisting of phenyl, pyridyl, pyrimidinyl, thiophenyl, imidazolyl, furanyl, indolyl, benzothiophenyl, benzofuranyl, or benzoimidazolyl;
Ar²** is phenyl;

### X** is S;

each R¹** and R²** is independently H, hydroxy or C₁-C₄ alkyl; or CR¹**R²** is C═O;
n**=2, 3 or 4;

### Y** is O;

Z** is OH, NH₂, NHSO₂(C₁-C₄ alkyl), NHC(O)NR⁶**R⁷**, NHC(O)O(C₁-C₄ alkyl), NH-dihydrothiazole, or NH-dihydroimidazole; wherein each R⁶** and R⁷** is independently H or C₁-C₄ alkyl; or Ar²**—Z** is

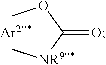

R⁹** is H or C₁-C₄ alkyl.

In one embodiment, the compound is selected from the group consisting of:

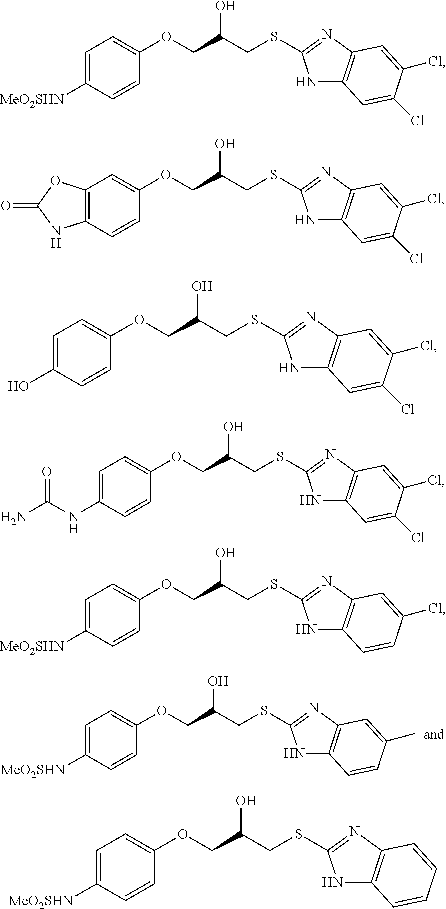

In one embodiment, the compound is

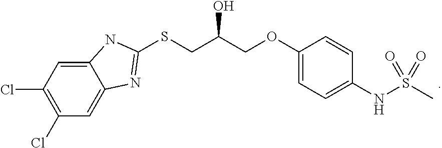

In one embodiment, the compound is selected from the group consisting of:

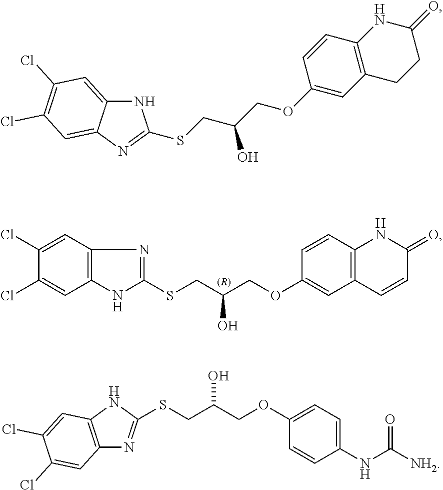

In one embodiment, the compound is

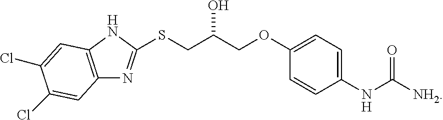

In another embodiment, the compound is selected from the group consisting of:

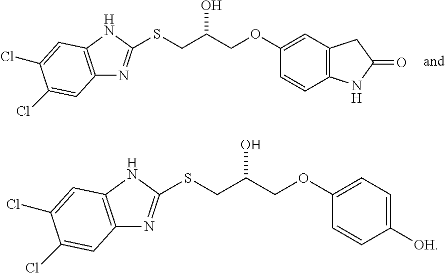

In another embodiment, the compound is selected from Table 16.

TABLE 16

| Compound | Name |
|---|---|
| 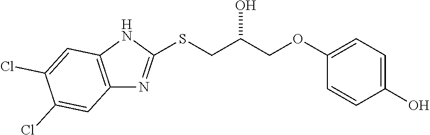 | (R)-4-(3-(5,6-dichloro-1H-benzo[d]imidazol-2-ylthio)- 2-hydroxypropoxy)phenol |
| 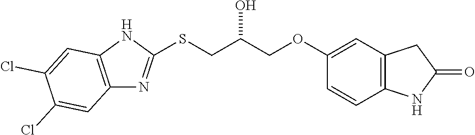 | (R)-5-(3-(5,6-dichloro-1H-benzo[d]imidazol-2-ylthio)- 2-hydroxypropoxy)indolin-2-one |
| 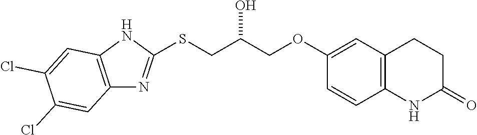 | (R)-6-(3-(5,6-dichloro-1H-benzo[d]imidazol-2-ylthio)- 2-hydroxypropoxy)-3,4-dihydroquinolin-2(1H)-one |
| 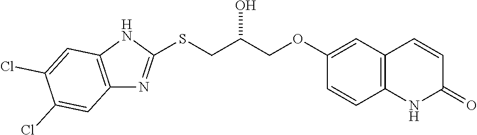 | (R)-6-(3-(5,6-dichloro-1H-benzo[d]imidazol-2-ylthio)- 2-hydroxypropoxy)quinolin-2(1H)-one |
| 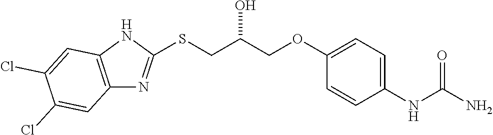 | (R)-1-(4-(3-(5,6-dichloro-1H-benzo[d]imidazol-2-ylthio)- 2-hydroxypropoxy)phenyl)urea |
| 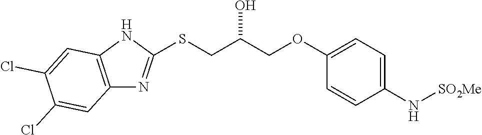 | (R)-N-(4-(3-(5,6-dichloro-1H-benzo[d]imidazol-2-ylthio)- 2-hydroxypropoxy)phenyl)methanesulfonamide |
| 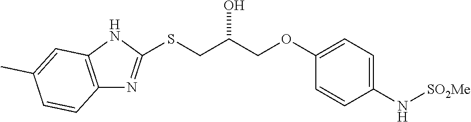 | (R)-N-(4-(2-hydroxy-3-(6-methyl-1H-benzo[d]imidazol- 2-ylthio)propoxy)phenyl)methanesulfonamide |

### Formula V

In one embodiment, methods of treatment or prophylaxis of neuropsychiatric disorders, in particular depression and anxiety are provided comprising administering a compound of Formula V or a pharmaceutically acceptable salt, ester, prodrug or derivative thereof to a host in need thereof:

Ar′—W′—B′—W″—Y′—Ar″—Z′

### Formula V

wherein B′ is selected from the group consisting of:

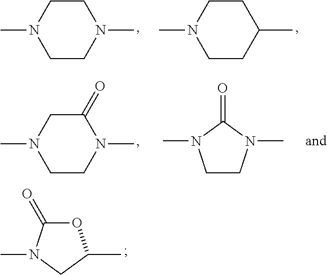

W′ is a bond or C₁-C₄ alkyl;
W″ is C₁-C₄ alkyl, C₁-C₄ hydroxyalkyl, C₁-C₄ haloalkyl or C(═O)—C₁-C₄ alkyl;
Y′ is selected from a bond, O, S, CH₂ and N;
Ar′ is an substituted or unsubstituted aromatic or nonaromatic cycloalkyl which optionally may include 0-3 heteroatoms;
Ar″ is an aromatic or nonaromatic cycloalkyl which optionally may include 0-3 heteroatoms;
Z′ is NRC(O)NR₂ wherein each R is independently selected from H, C₁-C₆ alkyl or C₆-C₁₂ aralkyl; or
Ar″—Z′ are taken together and selected from the group consisting

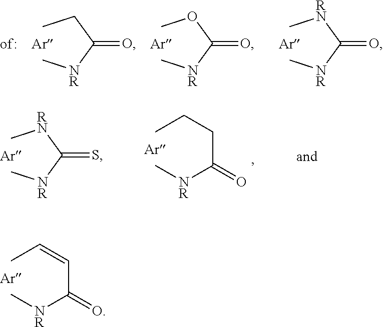

In one embodiment, Ar′ is substituted by (L′)_{k′} wherein each L′ is independently C₁-C₆ alkyl, C₁-C₆ alkoxy, C(═O)—(C₁-C₆)-alkyl, C₁-C₆ haloalkyl, alkaryl, hydroxy, —O-alkyl, —O-aryl, —SH, —S-alkyl, —S-aryl, fluoro, chloro, bromo, iodo, nitro, or cyano; or two L′ groups may be taken together with Ar′ to form a dioxolane ring or a cyclobutane ring; and k′=1, 2, 3, 4 or 5.

In one embodiment B′ is

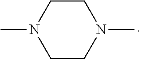

In another embodiment, B′ is

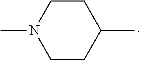

In another embodiment, B′ is

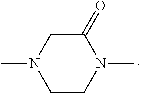

In another embodiment, B′ is

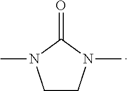

In another embodiment, B′ is

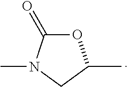

In one embodiment, W′ is a bond. In another embodiment, W′ is C₁-C₄ alkyl, for example methylene, ethylene, or propylene. In a particular subembodiment, W′ is CH₂.

In one embodiment W″ is C₁-C₄ alkyl, for example methylene, ethylene, propylene, methylpropylene, or butylene. In another embodiment, W″ is C₁-C₄ hydroxyalkyl, for example hydroxymethylene, hydroxyethylene, or hydroxypropylene. In a particular subembodiment, W″ is —CH₂, CH(OH)—CH₂—. In another embodiment, W″ is C₁-C₄ haloalkyl, for example fluoroethylene, fluoropropylene, chloroethylene, or chloropropylene. In another embodiment, W″ is C(═O)—C₁-C₄ alkyl, for example —C(═O)—CH₂— or —C(═O)—CH₂—CH₂—.

In one embodiment, Ar′ is an aromatic cycloalkyl, for example phenyl. In another embodiment, Ar′ is an nonaromatic cycloalkyl, for example cyclopentyl or cyclohexyl. In another embodiment, Ar′ is an aromatic cycloalkyl including 1-3 heteroatoms, for example pyrrole, furan, thiophene, pyridine, pyrimidine, pyrazine, pyridazine. Heteroatoms include but are not limited to N, S and O. In another embodiment, Ar′ is a nonaromatic cycloalkyl including 1-3 heteroatoms, for example pyrrolidine, pyrroline, dihydrofuran, tetrahydrofuran, dihydrothiophene, tetrahydrothiophene, piperidine, tetrahydropyran, pyran, thiane, thiiine, piperazine, oxazine, dithiane, or dioxane. In another embodiment, Ar′ is an aromatic or nonaromatic cycloalkyl including 1 heteroatom. In another embodiment, Ar′ is an aromatic or nonaromatic cycloalkyl including 2 heteroatoms. In another embodiment, Ar′ is an aromatic or nonaromatic cycloalkyl including 3 heteroatoms.

In one embodiment, Ar″ is an aromatic cycloalkyl, for example phenyl. In another embodiment, Ar″ is an nonaromatic cycloalkyl, for example cyclopentyl or cyclohexyl. In another embodiment, Ar″ is an aromatic cycloalkyl including 1-3 heteroatoms, for example pyrrole, furan, thiophene, pyridine, pyrimidine, pyrazine, or pyridazine. In another embodiment, Ar″ is a nonaromatic cycloalkyl including 1-3 heteroatoms, for example pyrrolidine, pyrroline, dihydrofuran, tetrahydrofuran, dihydrothiophene, tetrahydrothiophene, piperidine, tetrahydropyran, pyran, thiane, thiiine, piperazine, oxazine, dithiane, or dioxane. In another embodiment, Ar″ is an aromatic or nonaromatic cycloalkyl including 1 heteroatom. In another embodiment, Ar″ is an aromatic or nonaromatic cycloalkyl including 2 heteroatoms. In another embodiment, Ar″ is an aromatic or nonaromatic cycloalkyl including 3 heteroatoms.

In one embodiment, Z′ is NRC(O)NR₂, for example NHC(O)NH₂ or NHC(O)N(CH₃)₂.

In another embodiment, Z and Ar″ are taken together and selected from the group consisting of:

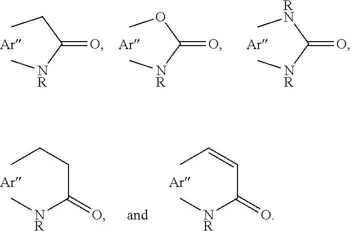

In a particular subembodiment, Ar″—Z′ is

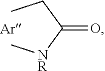

In another subembodiment, Ar″—Z′ is

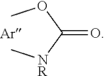

In another subembodiment, Ar″—Z′ is

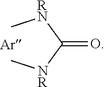

In another subembodiment, Ar″—Z′ is

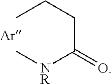

In another subembodiment, Ar″—Z′ is

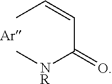

In another subembodiment, Ar″—Z′ is

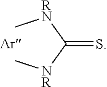

In a particular subembodiment of any of the above embodiments, R is H. In a particular subembodiment of any of the above embodiments, Ar″ is phenyl.

In one embodiment, each L′ is independently halo, C₁-C₆ alkyl, or C₁-C₆ haloalkyl. In a particular subembodiment Ar′ has at least one L′. In a particular subembodiment Ar′ is phenyl and is substituted with one or more L′ groups wherein one L′ is in the para position. In a particular embodiment, at least one L′ is halo, for example fluoro, chloro, bromo, or iodo. In a particular subembodiment, are least two L′ are halo and may be the same or different. In another embodiment, at least one L′ is C₁-C₆ alkyl, for example methyl, ethyl, propyl, isopropyl, butyl, isobutyl, pentyl, or hexyl. In another embodiment, at least one L′ is C₁-C₆ haloalkyl, for example, trifluoromethyl.

In one embodiment, Ar′ is unsubstituted. In another embodiment, k′ is 1. In a subembodiment, when k′ is 1 and Ar′ is phenyl, L′ is in the para position. In another embodiment, k′ is 2. In a subembodiment, when k′ is 2 and Ar′ is phenyl, one L′ is in the para position and one L′ is in a meta position. In another embodiment, k′ is 3. In another embodiment, k′ is 4. In another embodiment, k′ is 5.

In one embodiment, the compound is selected from the group consisting of:

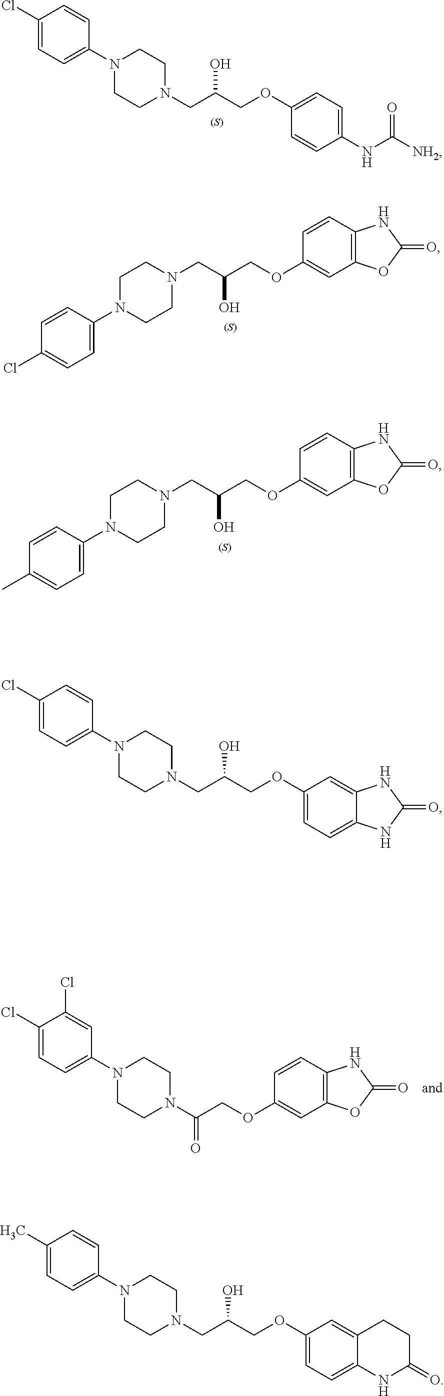

### Formula A

In one embodiment, methods of treatment or prophylaxis of neuropsychiatric disorders, in particular depression and anxiety are provided comprising administering a compound of Formula A or a pharmaceutically acceptable salt, ester, prodrug or derivative thereof to a host in need thereof:

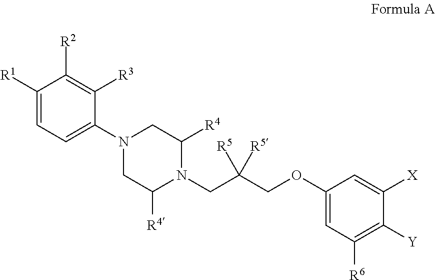

wherein R¹ is H, F, Cl, Br, CF₃, C₁₋₆ alkyl, C(O)CH₃, C(O)CO—(C₁₋₆ alkyl), CH₂OH, CN,
NH₂, N(C₁₋₆ alkyl)₂, OH, O—(C₁₋₆ alkyl), OCF₃, S—(C₁₋₆ alkyl), SO₂—(C₁₋₆ alkyl);
R² is H, F, Cl, methyl, CF₃;

### R³ is H, F, Cl, CH₃, CF₃, CN;

each of R⁴ and R^{4′} are independently selected from H or methyl;
each of R⁵ and R^{5′} can be H or OH, or R⁵ and R^{5′} can be taken together to form ═CH₂ or ═O;

### R⁶ is H or F;

### X is H or F;

### Y is OH, NHSO₂R⁷, or NHC(O)NHR⁸;

R⁷ is C₁₋₆ alkyl, C₆₋₁₂ aryl, or C₇₋₁₃ aralkyl;
R⁸ is H, C₁₋₆ alkyl, C₆₋₁₂ aryl, or C₇₋₁₃ aralkyl;
or X and Y are taken together to form a heterocycle wherein the moiety

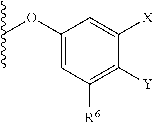

of a compound of Formula A is selected from the group consisting of:

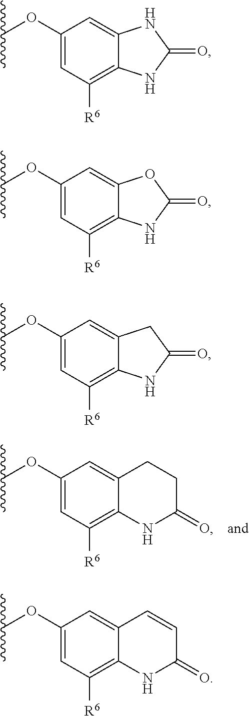

In one embodiment, C₁₋₆ alkyl includes, for example, methyl, ethyl, n-propyl, isopropyl, n-butyl, isobutyl, cyclopropyl. C₁₋₆ alkyl may also include tert-butyl, pentyl, cyclopentyl, hexyl, or cyclohexyl.

In one embodiment, R¹ is H. In one embodiment, R¹ is F. In one embodiment, R¹ is Cl. In another embodiment, R¹ is C₁₋₆ alkyl, for example methyl or isopropyl. In one embodiment, R¹ is OH. In one embodiment, R¹ is CF₃.

In one embodiment, R² is H. In one embodiment, R² is F. In one embodiment, R² is Cl. In another embodiment, R² is C₁₋₆ alkyl, for example methyl. In one embodiment, R² is CF₃.

In one embodiment, R³ is H. In one embodiment, R³ is F. In one embodiment, R³ is Cl. In another embodiment, R³ is C₁₋₆ alkyl, for example methyl. In one embodiment, R³ is CF₃. In another embodiment, R³ is CN.

In one embodiment, R⁴ is H. In one embodiment, R⁴ is methyl. In one embodiment, R^{4′} is H. In one embodiment, R^{4′} is methyl. In a particular embodiment, R⁴ and R^{4′} are both H. In another embodiment, one of R⁴ and R^{4′} is methyl.

In another embodiment, R⁶ is H. In another embodiment, R⁶ is F.

In another embodiment, X is H. In another embodiment, X is F.

In one embodiment, Y is OH. In one embodiment, Y is not OH. In one embodiment, Y is NHSO₂R⁷. In another embodiment, Y is not NHSO₂R⁷. In one embodiment, Y is NHC(O)NHR⁸.

In one embodiment, R⁷ is C₁₋₆ alkyl, for example methyl.

In one embodiment, R⁸ is H or C₁₋₆ alkyl, for example methyl, ethyl or propyl.

In a particular subembodiment, X and Y are taken together to form a heterocycle wherein the moiety

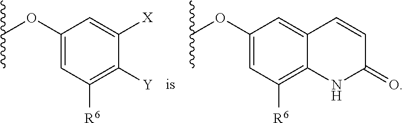

In a particular embodiment, the moiety

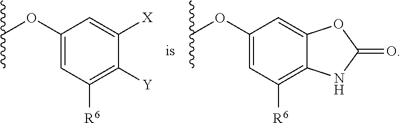

In another embodiment, the moiety

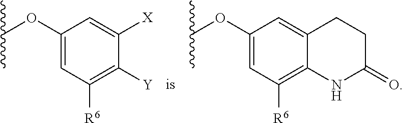

In one embodiment, the moiety

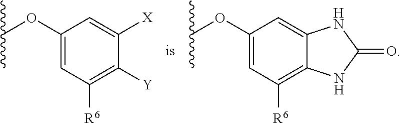

In one embodiment, the moiety

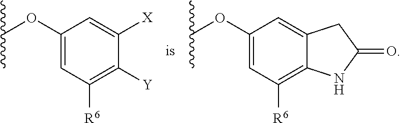

In one embodiment, the moiety

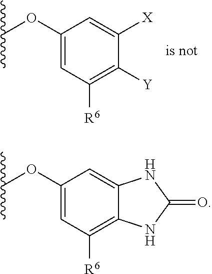

In one embodiment, the moiety

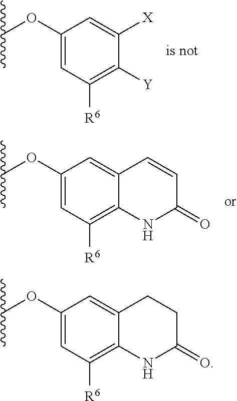

In one embodiment, the compound of Formula A, is selected from the compounds in Table 26, for example, the compound is selected from the group consisting of: NP10039, NP10165, NP10075, NP10153, NP10150, NP10146, NP10056, NP10122, NP10231, NP10002, NP10030, NP10070, NP10119, and NP10045.

### Formula B

In one embodiment, methods of treatment or prophylaxis of neuropsychiatric disorders, in particular depression and anxiety are provided comprising administering a compound of Formula B or a pharmaceutically acceptable salt, ester, prodrug or derivative thereof to a host in need thereof:

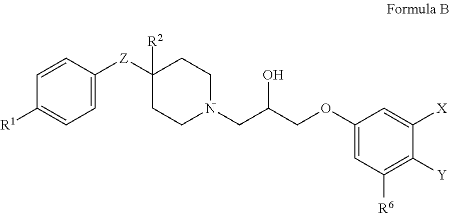

wherein R¹ is H, F, Cl, Br, CF₃, or C₁₋₆ alkyl;
Z is O, S, NH, CH₂ or a bond;

### R² is H or OH;

### R⁶ is H or F;

### X is H or F;

### Y is OH, NHSO₂R⁷ or NHC(O)NHR⁸;

R⁷ is C₁₋₆ alkyl, C₆₋₁₂ aryl, or C₇₋₁₃ aralkyl;
R⁸ is H, C₁₋₆ alkyl, C₆₋₁₂ aryl, or C₇₋₁₃ aralkyl;
or X and Y are taken together to form a heterocycle wherein the moiety

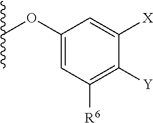

of a compound of Formula B is selected from the group consisting of:

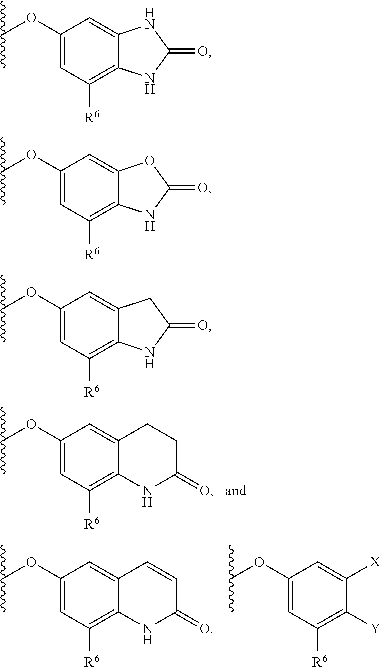

of a compound of Formula B is selected from the group consisting of:

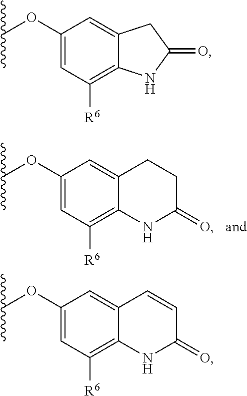

In one embodiment, R¹ is H. In one embodiment, R¹ is not H. In one embodiment, R¹ is Cl. In another embodiment, In R¹ is H or Cl. In one embodiment, R¹ is F, Cl or Br. In one embodiment, R¹ is CF₃. In one embodiment, R¹ is C₁₋₆ alkyl.

In one embodiment, Z is O. In another embodiment, Z is S. In another embodiment, Z is NH. In another embodiment, Z is CH₂. In another embodiment, Z is a bond. In one embodiment, Z is not a bond. In another embodiment, Z is not CH₂.

In one embodiment, R² is OH. In another embodiment, R² is H.

In another embodiment, R⁶ is H. In another embodiment, R⁶ is F.

In one embodiment, X is H. In a particular embodiment X is F.

In one embodiment, Y is OH. In one embodiment, Y is not OH. In one embodiment, Y is NHSO₂R⁷. In another embodiment, Y is not NHSO₂R⁷. In one embodiment, Y is NHC(O)NHR⁸.

In one embodiment, R⁷ is C₁₋₆ alkyl, for example methyl.

In one embodiment, R⁸ is H or C₁₋₆ alkyl, for example methyl, ethyl or propyl.

In one embodiment, X and Y are taken together to form a heterocycle wherein the moiety

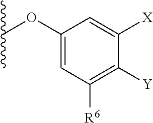

of a compound of Formula B is selected from the group consisting of:

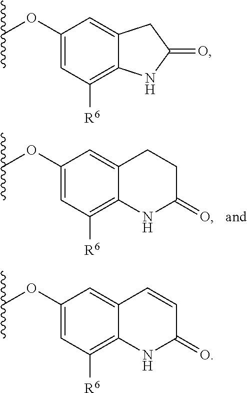

In a particular embodiment, the moiety

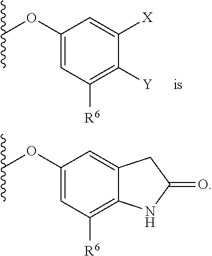

In another embodiment, the moiety

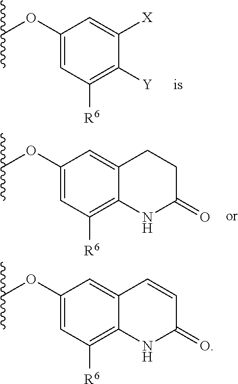

In one embodiment, the moiety

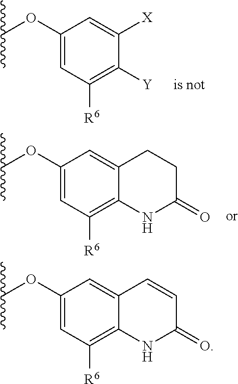

In one embodiment, the compound is selected from the compounds in Table 26, for example compounds NP10250 and NP10185.

### Formula C

In one embodiment, methods of treatment or prophylaxis of neuropsychiatric disorders, in particular depression and anxiety are provided comprising administering a compound of Formula C or a pharmaceutically acceptable salt, ester, prodrug or derivative thereof to a host in need thereof:

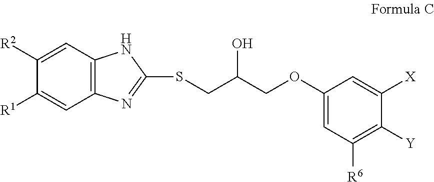

wherein each R¹ and R² is independently selected from H, F, Cl, Br, CF₃, or C₁₋₆ alkyl;

### R⁶ is H or F;

### X is H or F;

### Y is OH, NHSO₂R⁷ or NHC(O)NHR⁸;

R⁷ is C₁₋₆ alkyl, C₆₋₁₂ aryl, or C₂₋₁₃ aralkyl;
R⁸ is H, C₁₋₆ alkyl, C₆₋₁₂ aryl, or C₂₋₁₃ aralkyl;
or X and Y are taken together to form a heterocycle wherein the moiety

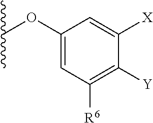

of a compound of Formula C is selected from the group consisting of:

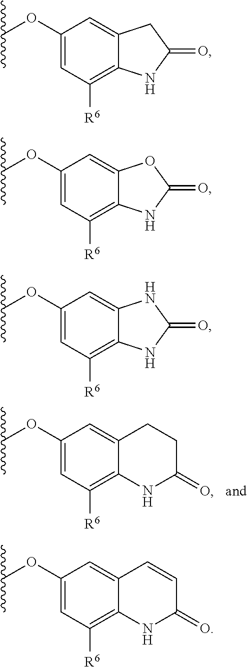

In one embodiment, R¹ is Cl. In one embodiment, R¹ is F. In one embodiment, R¹ is Br. In one embodiment, R¹ is H. In one embodiment, R¹ is not H. In one embodiment, R¹ is C₁₋₆ alkyl, for example methyl.

In one embodiment, R² is Cl. In one embodiment, R² is F. In one embodiment, R² is Br. In one embodiment, R² is H. In one embodiment, R² is not H.

In one embodiment, R⁶ is H. In another embodiment, R⁶ is F.

In one embodiment, X is H. In a particular embodiment X is F.

In one embodiment, Y is OH. In one embodiment, Y is not OH. In one embodiment, Y is NHSO₂R⁷. In another embodiment, Y is not NHSO₂R⁷. In one embodiment, Y is NHC(O)NHR⁸.

In one embodiment, R⁷ is C₁₋₆ alkyl, for example methyl.

In one embodiment, R⁸ is H or C₁₋₆ alkyl, for example methyl, ethyl or propyl.

In a particular subembodiment, X and Y are taken together to form a heterocycle wherein the moiety

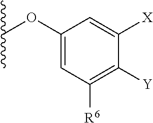

of a compound of Formula C is selected from the group consisting of:

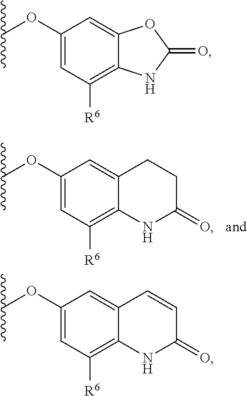

In a particular embodiment, the moiety

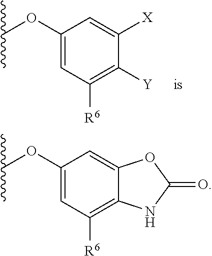

In another embodiment, the moiety

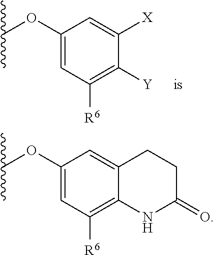

In one embodiment, the moiety

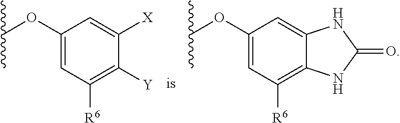

In one embodiment, the moiety

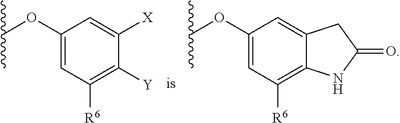

In one embodiment, the compound is

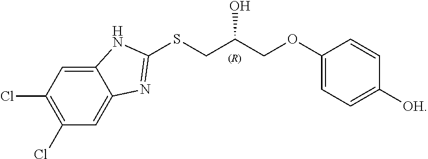

### Formula D-1

In one embodiment, methods of treatment or prophylaxis of neuropsychiatric disorders, in particular depression and anxiety are provided comprising administering a compound of Formula D-1 or a pharmaceutically acceptable salt, ester, prodrug or derivative thereof to a host in need thereof:

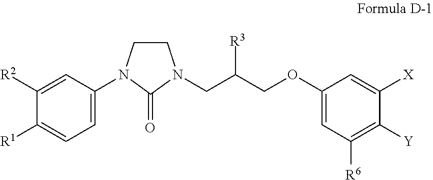

wherein each R¹ and R² is independently selected from H, F, Cl, Br, CF₃, or C₁₋₆ alkyl;

### R³ is H or OH;

### R⁶ is H or F;

### X is H or F;

### Y is OH, NH₂, N(R⁸)₂, NHSO₂R⁷ or NHC(O)NHR⁸;

R⁷ is C₁₋₆ alkyl, C₆₋₁₂ aryl, or C₇₋₁₃ aralkyl;
each R⁸ is independently selected from H, C₁₋₆ alkyl, C₆₋₁₂ aryl, or C₇₋₁₃ aralkyl;
or X and Y are taken together to form a heterocycle wherein the moiety

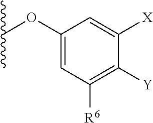

of a compound of Formula D-1 is selected from the group consisting of:

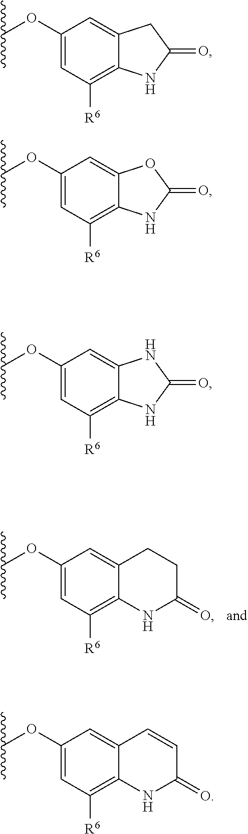

In one embodiment, R¹ is Cl. In one embodiment, R¹ is F. In one embodiment, R¹ is Br. In one embodiment, R¹ is H. In one embodiment, R¹ is not H. In one embodiment, R¹ is C₁₋₆ alkyl, for example methyl.

In one embodiment, R² is Cl. In one embodiment, R² is F. In one embodiment, R² is Br. In one embodiment, R² is H. In one embodiment, R² is not H.

In one embodiment, one of R¹ and R² is Cl. In another embodiment, both of R¹ and R² are Cl.

In one embodiment, R³ is H. In another embodiment, R³ is OH.

In one embodiment, R⁶ is H. In another embodiment, R⁶ is F.

In one embodiment, X is H. In a particular embodiment, X is F.

In one embodiment, Y is OH. In one embodiment, Y is not OH. In one embodiment, Y is NH₂. In one embodiment, Y is N(R⁸)₂. In one embodiment, Y is NHSO₂R⁷. In another embodiment, Y is not NHSO₂R⁷. In one embodiment, Y is NHC(O)NHR⁸.

In one embodiment, R⁷ is C₁₋₆ alkyl, for example methyl.

In one embodiment, R⁸ is H or C₁₋₆ alkyl, for example methyl, ethyl or propyl.

In a particular subembodiment, X and Y are taken together to form a heterocycle wherein the moiety

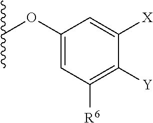

of a compound of Formula C is selected from the group consisting of:

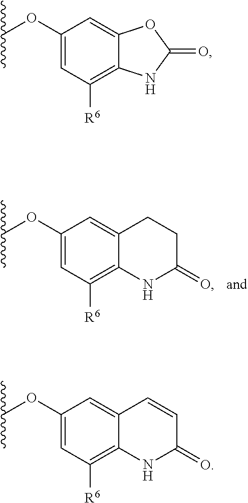

In a particular embodiment, the moiety

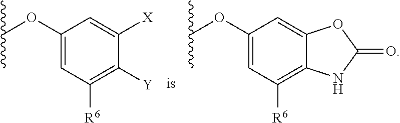

In another embodiment, the moiety

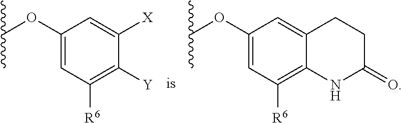

In one embodiment, the moiety

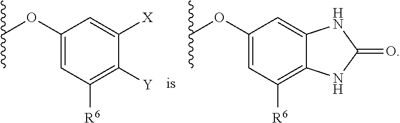

In one embodiment, the moiety

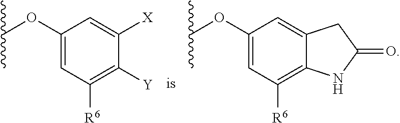

### Formula D-2

In one embodiment, methods of treatment or prophylaxis of neuropsychiatric disorders, in particular depression and anxiety are provided comprising administering a compound of Formula D-2 or a pharmaceutically acceptable salt, ester, prodrug or derivative thereof to a host in need thereof:

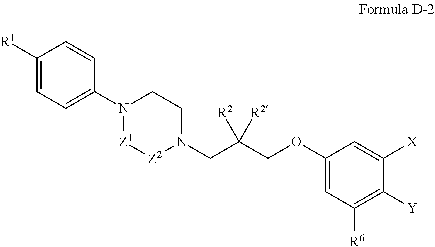

wherein R¹ is H, F, Cl, Br, CF₃, or C₁₋₆ alkyl;
Z¹ and Z² are each independently selected from the group consisting of —CH₂— or —C(═O)—;
each of R² and R^{2′} can be H or OH., or R² and R^{2′} can be taken together to form ═CH₂;

### R⁶ is H or F;

### X is H or F;

### Y is OH, NH₂, N(R⁸)₂, NHSO₂R⁷ or NHC(O)NHR⁸;

R⁷ is C₁₋₆ alkyl, C₆₋₁₂ aryl, or C₇₋₁₃ aralkyl;
each R⁸ is independently selected from H, C₁-C₆ alkyl, C₆₋₁₂ aryl, or C₇₋₁₃ aralkyl;
or X and Y are taken together to form a heterocycle wherein the moiety

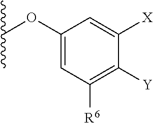

of a compound of Formula D-2 is selected from the group consisting of:

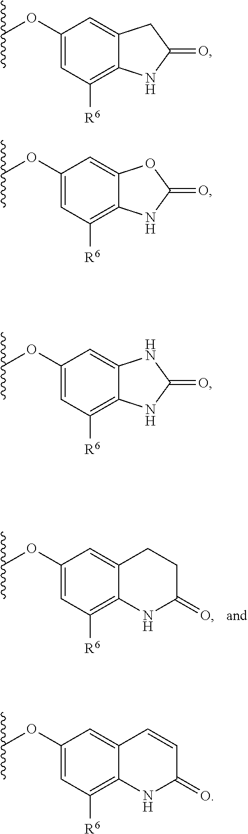

In one embodiment, R¹ is Cl. In one embodiment, R¹ is F. In one embodiment, R¹ is Br. In one embodiment, R¹ is H. In one embodiment, R¹ is not H. In one embodiment, R¹ is C₁₋₆ alkyl, for example methyl.

In one embodiment, R² is H. In one embodiment, R² is OH. In one embodiment, R^{2′} is H. In one embodiment, R^{2′} is OH. In one embodiment, one of R² and R^{2′} is OH. In another embodiment, both of R² and R^{2′} are H. In another embodiment, R² and R^{2′} are taken together to form ═CH₂.

In one embodiment, R⁶ is H. In another embodiment, R⁶ is F.

In one embodiment, X is H. In a particular embodiment, X is F.

In one embodiment, Y is OH. In one embodiment, Y is not OH. In one embodiment, Y is NH₂. In one embodiment, Y is N(R⁸)₂. In one embodiment, Y is NHSO₂R⁷. In another embodiment, Y is not NHSO₂R⁷. In one embodiment, Y is NHC(O)NHR⁸.

In one embodiment, R⁷ is C₁₋₆ alkyl, for example methyl.

In one embodiment, R⁸ is H or C₁₋₆ alkyl, for example methyl, ethyl or propyl.

In a particular subembodiment, X and Y are taken together to form a heterocycle wherein the moiety

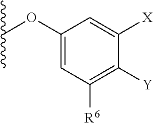

of a compound of Formula C is selected from the group consisting of:

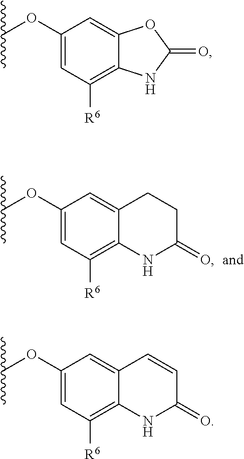

In a particular embodiment, the moiety

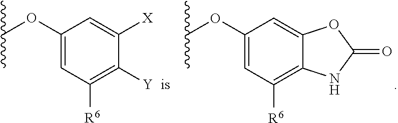

In another embodiment, the moiety

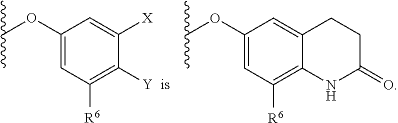

In one embodiment, the moiety

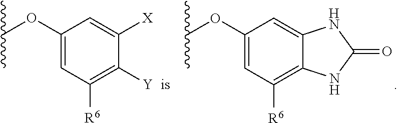

In one embodiment, the moiety

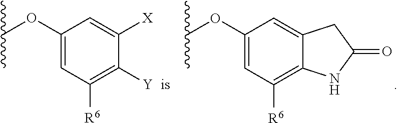

In one embodiment, the compound is selected from the compounds in Table 26, for example compounds NP10076 or NP10226.

### Formula F

In one embodiment, methods of treatment or prophylaxis of neuropsychiatric disorders, in particular depression and anxiety are provided comprising administering a compound of Formula F or a pharmaceutically acceptable salt, ester, prodrug or derivative thereof to a host in need thereof:

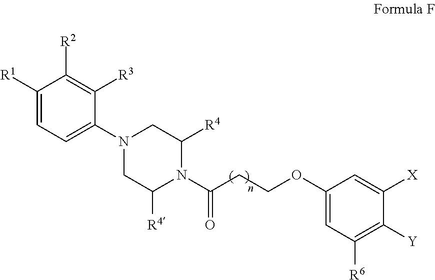

wherein R¹ is H, F, Cl, Br, CF₃, C₁₋₆ alkyl, C(O)CH₃, C(O)CO—(C₁₋₆ alkyl), CH₂OH, CN,
NH₂, N(C₁₋₆ alkyl)₂, OH, O—(C₁₋₆ alkyl), OCF₃, S—(C₁₋₆ alkyl), SO₂—(C₁₋₆ alkyl);
R² is H, F, Cl, methyl, CF₃;

### R³ is H, F, Cl, CH₃, CF₃, CN;

R⁴ is H or methyl;
n is 0, 1 or 2;

### R⁶ is H or F;

### X is H or F;

### Y is OH, NHSO₂R⁷, NHC(S)NHR⁸ or NHC(O)NHR⁸;

wherein R⁷ or R⁸ are each independently C₁₋₆ alkyl, C₆₋₁₂ aryl, C₇₋₁₃ aralkyl;
or X and Y are taken together to form a heterocycle wherein the moiety

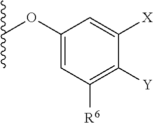

of a compound of Formula F is selected from the group consisting of:

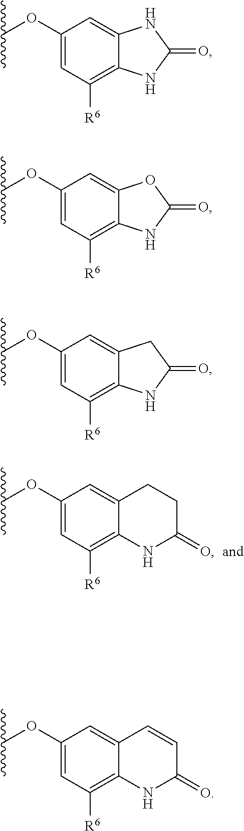

In one embodiment, C₁₋₆ alkyl includes, for example, methyl, ethyl, n-propyl, isopropyl, n-butyl, isobutyl, cyclopropyl. C₁₋₆ alkyl may also include tert-butyl, pentyl, cyclopentyl, hexyl, or cyclohexyl.

In one embodiment, R¹ is F. In one embodiment, R¹ is Cl. In one embodiment, R¹ is Br. In a particular embodiment, R¹ is CF₃. In a particular embodiment, R¹ is C₁₋₆ alkyl, for example methyl. In one embodiment, R¹ is not H. In one embodiment, R¹ is F, Cl or methyl.

In another embodiment, R² is H. In one embodiment, R² is F. In one embodiment, R² is Cl.

In another embodiment, R³ is H.

In one embodiment, n is 0. In one embodiment, n is 1. In one embodiment, n is 2.

In one embodiment, R⁴ is H. In one embodiment, R⁴ is methyl. In one embodiment, R^{4′} is H. In one embodiment, R^{4′} is methyl. In a particular embodiment, R⁴ and R^{4′} are both H. In another embodiment, one of R⁴ and R^{4′} is methyl.

In another embodiment, R⁶ is H. In another embodiment, R⁶ is F.

In another embodiment, X is H. In another embodiment, X is F.

In one embodiment, Y is OH. In one embodiment, Y is not OH. In one embodiment, Y is NHSO₂R⁷. In another embodiment, Y is not NHSO₂R⁷. In one embodiment, Y is NHC(O)NHR⁸. In one embodiment, Y is NHC(S)NHR⁸.

In a particular subembodiment, X and Y are taken together to form a heterocycle wherein the moiety

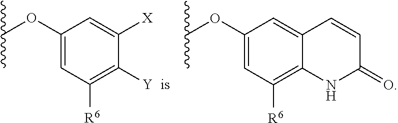

In a particular embodiment, the moiety

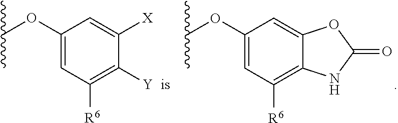

In another embodiment, the moiety

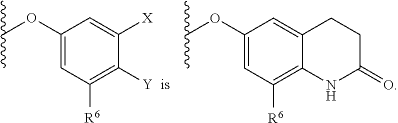

In one embodiment, the moiety

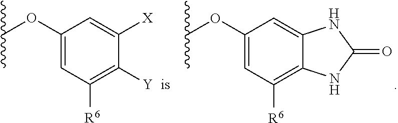

In one embodiment, the moiety

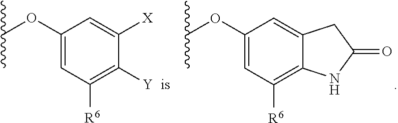

In one embodiment, the compound is

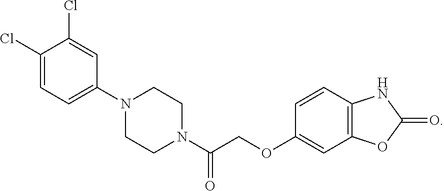

### Other Compound Embodiments

In one embodiment, methods of treatment or prophylaxis of neuropsychiatric disorders, in particular depression and anxiety are provided comprising administering a compound of described in WO 02/072542 to Emory University, the entire disclosure of which is hereby incorporated by reference, or a pharmaceutically acceptable salt, ester, prodrug or derivative thereof to a host in need thereof.

In one embodiment, methods of treatment or prophylaxis of neuropsychiatric disorders, in particular depression and anxiety are provided comprising administering a compound selected from the group consisting of

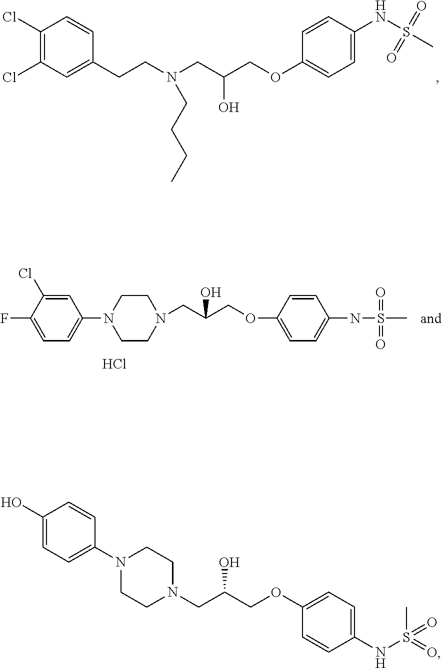

or a pharmaceutically acceptable salt, ester, prodrug or derivative thereof to a host in need thereof.

In another embodiment, methods of treatment or prophylaxis of neuropsychiatric disorders, in particular depression and anxiety are provided comprising administering a compound of described in WO 09/006,437 to Emory University and NeurOp, Inc., or a pharmaceutically acceptable salt, ester, prodrug or derivative thereof to a host in need thereof.

In one embodiment, methods of treatment or prophylaxis of neuropsychiatric disorders, in particular depression and anxiety are provided comprising administering a compound selected from the group consisting of

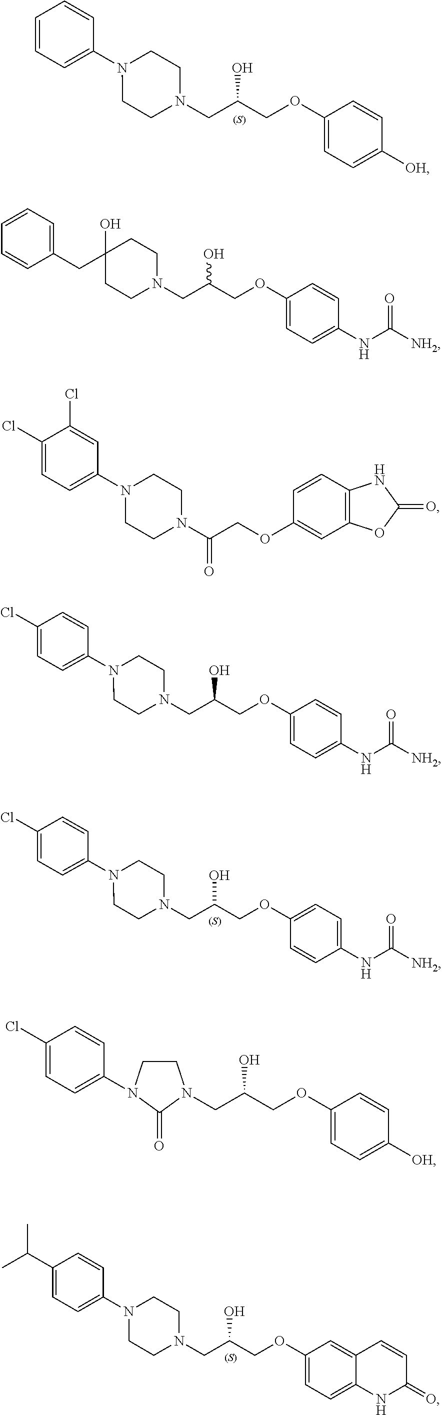

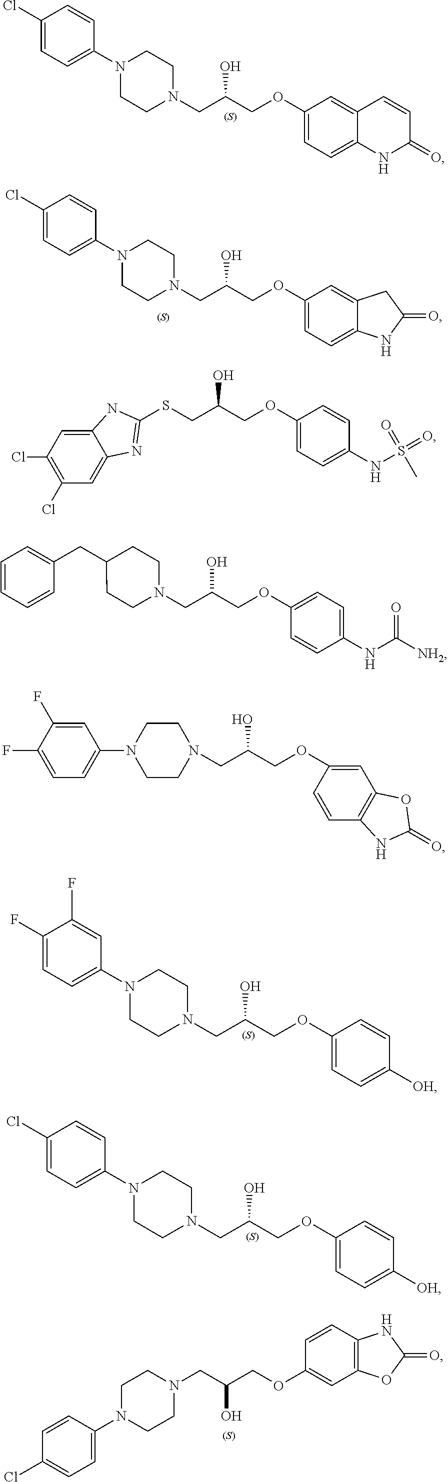

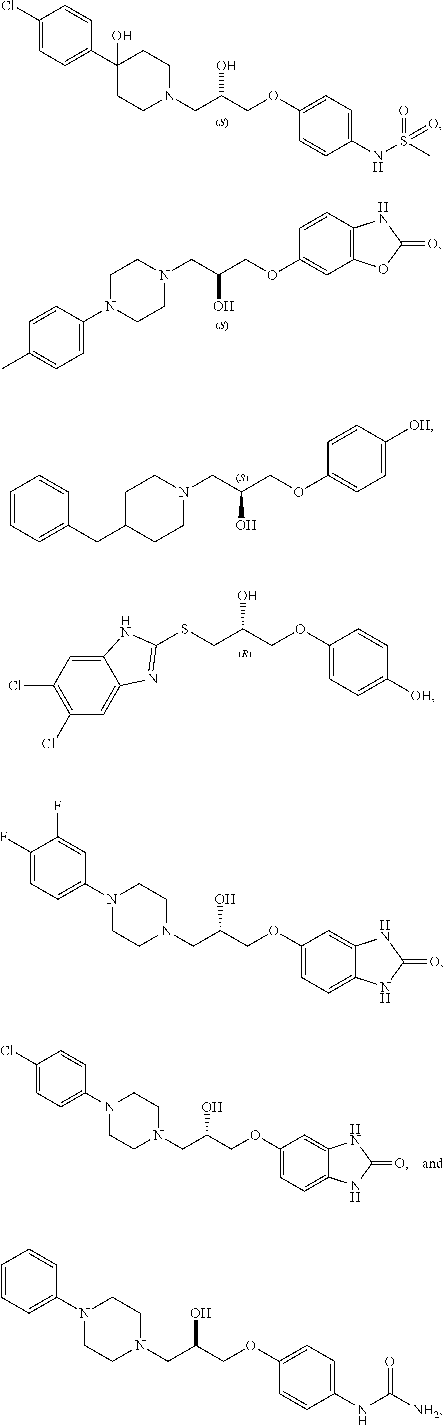

or a pharmaceutically acceptable salt, ester, prodrug or derivative thereof to a host in need thereof.

### Enantiomers

In certain embodiments, the compounds are provided as enantiomers. In one embodiment, the compound is provided as an enantiomer or mixture of enantiomers. In a particular embodiment, the compound is present as a racemic mixture. The enantiomer can be named by the configuration at the chiral center, such as R or S. In certain embodiments, the compound is present as a racemic mixture of R- and S- enantiomers. In certain embodiments, the compound is present as a mixture of two enantiomers. In one embodiment, the mixture has an enantiomeric excess in R. In one embodiment, the mixture has an enantiomeric excess in S. In certain other embodiments, the compound is in an enantiomeric excess of the R- or S- enantiomer. The enantiomeric excess can be 51% or more, such as 51% or more, 55% or more, 60% or more, 65% or more, 70% or more, 75% or more, 80% or more, 85% or more, 90% or more, 95% or more, 98% or more, or 99% or more in the single enantiomer. The enantiomeric excess can be 51% or more, such as 51% or more, 55% or more, 60% or more, 65% or more, 70% or more, 75% or more, 80% or more, 85% or more, 90% or more, 95% or more, 98% or more, or 99% or more in the R enantiomer. The enantiomeric excess can be 51% or more, such as 51% or more, 55% or more, 60% or more, 65% or more, 70% or more, 75% or more, 80% or more, 85% or more, 90% or more, 95% or more, 98% or more, or 99% or more in the S enantiomer.

In other embodiments, the compound is substantially in the form of a single enantiomer. In some embodiments, the compound is present substantially in the form of the R enantiomer. In some embodiments, the compound is present substantially in the form of the S enantiomer. The phrase “substantially in the form of a single enantiomer” is intended to mean at least 70% or more in the form of a single enantiomer, for example 70% or more, 75% or more, 80% or more, 85% or more, 90% or more, 95% or more, 98% or more, or 99% or more in either the R or S enantiomer.

The enantiomer can be named by the direction in which it rotates the plane of polarized light. If it rotates the light clockwise as seen by the viewer towards whom the light is traveling, the isomer can be labeled (+) and if it rotates the light counterclockwise, the isomer can be labeled (−). In certain embodiments, the compound is present as a racemic mixture of (+) and (−) isomers. In certain embodiments, the compound is present as a mixture of two isomers. In one embodiment, the mixture has an excess in (+). In one embodiment, the mixture has an excess in (−). In certain other embodiments, the compound is in an excess of the (+) or (−) isomer. The isomeric excess can be 51% or more, such as 51% or more, 55% or more, 60% or more, 65% or more, 70% or more, 75% or more, 80% or more, 85% or more, 90% or more, 95% or more, 98% or more, or 99% or more in the (+) isomer. The enantiomeric excess can be 51% or more, such as 51% or more, 55% or more, 60% or more, 65% or more, 70% or more, 75% or more, 80% or more, 85% or more, 90% or more, 95% or more, 98% or more, or 99% or more in the (−) isomer.

In other embodiments, the compound is substantially in the form of a single optical isomer. In some embodiments, the compound is present substantially in the form of the (+) isomer. In other embodiments, the compound is present substantially in the form of the (−) isomer. The phrase “substantially in the form of a single optical isomer” is intended to mean at least 70% or more in the form of a single isomer, for example 70% or more, 75% or more, 80% or more, 85% or more, 90% or more, 95% or more, 98% or more, or 99% or more of either the (+) or (−) isomer.

### Methods of Use

In certain embodiments, the compounds are used for the treatment or prevention of neuropsychiatric disorders. The compounds of the invention can generally be administered to a host at risk of, or suffering from, a neuropsychiatric disorder related to NMDA receptor activation. Representative neuropsychiatric disorders include, without limitation, depression, anxiety, schizophrenia, bipolar disorder, obsessive-compulsive disorder, alcohol and substance abuse, and attention-deficit disorders such as ADH or ADHD. In particular embodiments, the disorders are neuropsychiatric mood disorders, non-limiting examples of which include depression, including major depression, bipolar disorders including cyclothymia (a mild form of bipolar disorder), affective disorders such as SAD (seasonal affective disorder) and mania (euphoric, hyperactive, over inflated ego, unrealistic optimism). In certain embodiments, a method of treatment a neuropsychiatric disorder is provided including administering a compound of the invention, alone or in combination to a host diagnosed with the disorder. Uses of the compounds in the treatment or manufacture of a medicament for such disorders are also provided.

In certain embodiments, the compounds are used for the treatment of depression in a host diagnosed with the disorder. Depression, formally called major depression, major depressive disorder or clinical depression, is a medical illness that involves the mind and body. Most health professionals today consider depression a chronic illness that requires long-term treatment, much like diabetes or high blood pressure. Although some people experience only one episode of depression, most have repeated episodes of depression symptoms throughout their life. Depression is also a common feature of mental illness, whatever its nature and origin. In some instances, the host or patient has a history of a major psychiatric disorder, such as schizophrenia. In other instances, the host does not have a history of a major psychiatric disorder but has been diagnosed with suffering from at least one depressive episode. In other instances, the host has been diagnosed with bipolar disorder. The host may also have been diagnosed as suffering from panic attacks or anxiety.

In one embodiment, the compounds of the present invention are used to diminish the severity of a depressive episode.

In some instances, the host is not suffering from a chronic disorder but is at risk of a depressive episode, anxiety or a panic attack due to environmental circumstances. The compounds may be given prophylactically to prevent onset of such an episode. For instance, in certain instances the compounds can be provided to a host before a plane trip, a public speech, or other potential stressful even that could lead to an episode. In some embodiments, therefore, a method of prevention of a neuropsychiatric episode is provided, including administering a compound of the invention to a host at risk of suffering from such an episode.

In one embodiment, the compounds of the present invention are used to prevent a future depressive episode.

In certain embodiments, the compounds are administered to a host suffering from or at risk of suffering from age-related depression. The compounds can be administered prophylactically to a host over the age of 60, or over the age of 70, or over the age of 80 to prevent or reduce the severity of depressive episodes.

Depression is associated with physical illness as well. Chronic medical conditions associated with depression include heart disease, cancer, vitamin deficiencies, diabetes, hepatitis, and malaria. Depression also is a common effect of neurological disorders, including Parkinson's and Alzheimer's diseases, multiple sclerosis, strokes, and brain tumors. Even moderate depressive symptoms are associated with a higher than average rate of arteriosclerosis, heart attacks, and high blood pressure. Depression can mimic medical illness and any illness feels worse to someone suffering from depression. In certain other embodiments, the compounds are useful in the treatment or prophylaxis of a neuropsychiatric disorder associated with a medical condition, including but not limited to heart disease, cancer, vitamin deficiencies, diabetes, hepatitis, and malaria by admistering the compound to a host suffering from the medical condition. In other instances, the compounds are useful in treatment or prophylaxis of a neuropsychiatric disorder associated with a neurological disorder or physiological insult by administering the compound to a host suffering from a neurological disorder or physiological insult. In non-limiting embodiments, these can include Parkinson's and Alzheimer's diseases, multiple sclerosis, strokes, and brain tumors. In some instances, the compounds are useful for treatment or prophylaxis of disorders such as depression or bipolar disorder associated with an injury or with aging. The compounds may also be useful in treatment or prophylaxis of schizophrenia.

In certain other embodiments, the compounds are used for treatment of a bipolar disorder in a host diagnosed with the disorder. The compounds can also be used to diminish the severity of manic episodes or prevent future episodes.

In certain embodiments, methods of treating seasonal disorders is provided including administering the compound to a host at risk of suffering from a SAD. In particular, the compounds can be provided on a seasonal basis. In some embodiments, the host has been diagnosed or is at risk of SAD.

In certain embodiments, the host is suffering from an attention deficit disorders such as ADH or ADHD.

Certain NMDA receptor antagonists described herein have enhanced activity in tissue having lower-than-normal pH. The tissue can be brain tissue. In certain embodiments, the reduced pH is associated with neuropsychiatric conditions. In some embodiments, the conditions can be associated with a physiological insult. In other embodiments, the conditions are mood disorders.

The compounds provided herein block the NR2B-containing NMDA receptors, have varying activity against receptors containing NR2A or NR2D, and may be selective for other members of the NMDA receptor family (NR2C, NR3A and NR3B). In one embodiment, the compounds are selective NMDA receptor blockers. General blocking of NMDA receptors throughout the brain causes adverse effects such as ataxia, memory deficits, hallucinations and other neurological problems. In one embodiment, the compounds are NMDA receptors antagonists selective for NR2B, NR2A, NR2C, NR2D, NR3A, and/or NR3B that do not interact with other receptors or ion channels at therapeutic concentrations. In one embodiment, the compound is a selective NR1/NR2A NMDA receptor and/or a NR1/NR2B NMDA receptor antagonist. In one particular embodiment, the compounds can bind to the NR2B subunit of the NMDA receptor. In another particular embodiment, the compounds are selective for the NR2B subunit of the NMDA receptor. In one embodiment, the compound is not an NMDA receptor glutamate site antagonist. In another embodiment, the compound is not an NMDA receptor glycine site antagonist.

In one embodiment, the compound does not exhibit substantial toxic side effects, such as, for example, motor impairment or cognitive impairment. In a particular embodiment, the compound has a therapeutic index equal to or greater than at least 2. In another embodiment, the compound is at least 10 times more selective for binding to an NMDA receptor than any other glutamate receptor.

Further, compounds selected according to the methods or processes described herein can be used prophylactically to prevent or protect against such diseases or neurological conditions, such as those described herein. In one embodiment, patients with a predisposition for a neuropsychiatric disorder, in particular a mood disorder, such as a genetic predisposition, can be treated prophylactically with the methods and compounds described herein.

### Pharmaceutical Compositions

Mammals, and specifically humans, suffering from or at risk of neuropsychiatric disorders can be treated by either targeted or systemic administration, via oral, inhalation, topical, trans- or sub-mucosal, subcutaneous, parenteral, intramuscular, intravenous or transdermal administration of a composition comprising an effective amount of the compounds described herein or a pharmaceutically acceptable salt, ester or prodrug thereof, optionally in a pharmaceutically acceptable carrier.

The compounds or composition is typically administered by oral administration. Alternatively, compounds can be administered by inhalation. In another embodiment, the compound is administered transdermally (for example via a slow release patch), or topically. In yet another embodiment, the compound is administered subcutaneously, intravenously, intraperitoneally, intramuscularly, parenterally, or submucosally. In any of these embodiments, the compound is administered in an effective dosage range to treat the target condition.

In one embodiment, compounds of the present invention are administered orally. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition.

When the compound is administered orally in the form of a dosage unit such as a tablets, pills, capsules, troches and the like, these can contain any of the following ingredients, or compounds of a similar nature: a binder (such as microcrystalline cellulose, gum tragacanth or gelatin); an excipient (such as starch or lactose), a disintegrating agent (such as alginic acid, Primogel, or corn starch); a lubricant (such as magnesium stearate or Sterotes); a glidant (such as colloidal silicon dioxide); a sweetening agent (such as sucrose or saccharin); and/or a flavoring agent (such as peppermint, methyl salicylate, or orange flavoring). When the dosage unit form is a capsule, it can contain, in addition to material of the above type, a liquid carrier (such as a fatty oil). In addition, dosage unit forms can contain various other materials which modify the physical form of the dosage unit, for example, coatings of sugar, shellac, or other enteric agents.

The compound or its salts can also be administered orally as a component of an elixir, suspension, syrup, wafer, chewing gum or the like. A syrup may contain, in addition to the active compounds, a sweetening agent (such as sucrose, saccharine, etc.) and preservatives, dyes and colorings and flavors.

The compounds of the invention may be also administered in specific, measured amounts in the form of an aqueous suspension by use of a pump spray bottle. The aqueous suspension compositions of the present invention may be prepared by admixing the compounds with water and other pharmaceutically acceptable excipients. The aqueous suspension compositions according to the present invention may contain, inter alia, water, auxiliaries and/or one or more of the excipients, such as: suspending agents, e.g., microcrystalline cellulose, sodium carboxymethylcellulose, hydroxpropyl-methyl cellulose; humectants, e.g. glycerin and propylene glycol; acids, bases or buffer substances for adjusting the pH, e.g., citric acid, sodium citrate, phosphoric acid, sodium phosphate as well as mixtures of citrate and phosphate buffers; surfactants, e.g. Polysorbate 80; and antimicrobial preservatives, e.g., benzalkonium chloride, phenylethyl alcohol and potassium sorbate.

In a separate embodiment, the compounds of the invention are in the form of an inhaled dosage. In this embodiment, the compounds may be in the form of an aerosol suspension, a dry powder or liquid particle form. The compounds may be prepared for delivery as a nasal spray or in an inhaler, such as a metered dose inhaler. Pressurized metered-dose inhalers (“MDI”) generally deliver aerosolized particles suspended in chlorofluorocarbon propellants such as CFC-11, CFC-12, or the non-chlorofluorocarbons or alternate propellants such as the fluorocarbons, HFC-134A or HFC-227 with or without surfactants and suitable bridging agents. Dry-powder inhalers can also be used, either breath activated or delivered by air or gas pressure such as the dry-powder inhaler disclosed in the Schering Corporation International Patent Application No. PCT/US92/05225, published 7 Jan. 1993 as well as the Turbuhaler™ (available from Astra Pharmaceutical Products, Inc.) or the Rotahaler™ (available from Allen & Hanburys) which may be used to deliver the aerosolized particles as a finely milled powder in large aggregates either alone or in combination with some pharmaceutically acceptable carrier e.g. lactose; and nebulizers.

Solutions or suspensions used for parenteral, intradermal, subcutaneous, or topical application can include at least some of the following components: a sterile diluent (such as water for injection, saline solution, fixed oils, polyethylene glycols, glycerine, propylene glycol or other synthetic solvents); antibacterial agents (such as benzyl alcohol or methyl parabens); antioxidants (such as ascorbic acid or sodium bisulfate); chelating agents (such as ethylenediaminetetraacetic acid); buffers (such as acetates, citrates or phosphates); and/or agents for the adjustment of tonicity (such as sodium chloride or dextrose). The pH of the solution or suspension can be adjusted with acids or bases, such as hydrochloric acid or sodium hydroxide.

A parenteral preparation can be enclosed in ampoules, disposable syringes or multiple dose vials made of glass or plastic.

Suitable vehicles or carriers for topical application can be prepared by conventional techniques, such as lotions, suspensions, ointments, creams, gels, tinctures, sprays, powders, pastes, slow-release transdermal patches, suppositories for application to rectal, vaginal, nasal or oral mucosa. In addition to the other materials listed above for systemic administration, thickening agents, emollients, and stabilizers can be used to prepare topical compositions. Examples of thickening agents include petrolatum, beeswax, xanthan gum, or polyethylene, humectants such as sorbitol, emollients such as mineral oil, lanolin and its derivatives, or squalene.

If administered intravenously, carriers can be physiological saline, bacteriostatic water, Cremophor EL™ (BASF, Parsippany, N.J.) or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) are also preferred as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the compound is then introduced into the container. The container is then swirled by hand to free lipid material from the sides of the container and to disperse lipid aggregates, thereby forming the liposomal suspension.

### Dosing

The compound is administered for a sufficient time period to alleviate the undesired symptoms and the clinical signs associated with the condition being treated. In one embodiment, the compounds are administered less than three times daily. In one embodiment, the compounds are administered in one or two doses daily. In one embodiment, the compounds are administered once daily. In some embodiments, the compounds are administered in a single oral dosage once a day.

The active compound is included in the pharmaceutically acceptable carrier or diluent in an amount sufficient to deliver to a patient a therapeutic amount of compound in vivo in the absence of serious toxic effects. An effective dose can be readily determined by the use of conventional techniques and by observing results obtained under analogous circumstances. In determining the effective dose, a number of factors are considered including, but not limited to: the species of patient; its size, age, and general health; the specific disease involved; the degree of involvement or the severity of the disease; the response of the individual patient; the particular compound administered; the mode of administration; the bioavailability characteristics of the preparation administered; the dose regimen selected; and the use of concomitant medication.

Typical systemic dosages for the herein described conditions are those ranging from 0.01 mg/kg to 1500 mg/kg of body weight per day as a single daily dose or divided daily doses. Preferred dosages for the described conditions range from 0.5-1500 mg per day. A more particularly preferred dosage for the desired conditions ranges from 5-750 mg per day. Typical dosages can also range from 0.01 to 1500, 0.02 to 1000, 0.2 to 500, 0.02 to 200, 0.05 to 100, 0.05 to 50, 0.075 to 50, 0.1 to 50, 0.5 to 50, 1 to 50, 2 to 50, 5 to 50, 10 to 50, 25 to 50, 25 to 75, 25 to 100, 100 to 150, or 150 or more mg/kg/day, as a single daily dose or divided daily doses. In one embodiment, the daily dose is between 10 and 500 mg/day. In another embodiment, the dose is between about 10 and 400 mg/day, or between about 10 and 300 mg/day, or between about 20 and 300 mg/day, or between about 30 and 300 mg/day, or between about 40 and 300 mg/day, or between about 50 and 300 mg/day, or between about 60 and 300 mg/day, or between about 70 and 300 mg/day, or between about 80 and 300 mg/day, or between about 90 and 300 mg/day, or between about 100 and 300 mg/day, or about 200 mg/day. In one embodiment, the compounds are given in doses of between about 1 to about 5, about 5 to about 10, about 10 to about 25 or about 25 to about 50 mg/kg. Typical dosages for topical application are those ranging from 0.001 to 100% by weight of the active compound.

The concentration of active compound in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the claimed composition. The active ingredient may be administered at once, or may be divided into a number of smaller doses to be administered at varying intervals of time.

### Combination Treatment

The compound can also be mixed with other active materials which do not impair the desired action, or with materials that supplement the desired action. The active compounds can be administered in conjunction, i.e. combination or alternation, with other medications used in the treatment or prevention neuropsychiatric disorders, such as those in which NMDA receptor activation is involved. In certain embodiments, the combination can be synergistic.

In certain embodiments, the compound is administered in combination or alternation with a compound useful for treatment of neuropsychiatric disorders, such as a selective serotonin reuptake inhibitor (SSRI), a serotonin and norepinephrine reuptake inhibitor (SNRT), norepinephrine and dopamine reuptake inhibitor (NDRI), combined reuptake inhibitor and receptor blocker, tetracyclic antidepressant, tricyclic antidepressants (TCAs) (although TCAs tend to have numerous and severe side effects), or a monoamine oxidase inhibitor (MAOI).

Electroconvulsive therapy (ECT) can also be used to treat depression in conjunction with administration of a compound of the invention. Nontraditional treatment options include vagus nerve stimulation, transcranial magnetic stimulation and deep brain stimulation.

SSRIs include fluoxetine (Prozac, Sarafem), paroxetine (Paxil), sertraline (Zoloft), citalopram (Celexa) and escitalopram (Lexapro). SSRIs that have been approved by the Food and Drug Administration specifically to treat depression are: Citalopram (Celexa), Escitalopram (Lexapro), Fluoxetine (Prozac, Prozac Weekly), Paroxetine (Paxil, Paxil CR) and Sertraline (Zoloft). SNRIs that have been approved by the Food and Drug Administration specifically to treat depression are: Duloxetine (Cymbalta) and Venlafaxine (Effexor, Effexor XR). The only NDRI that has been approved by the Food and Drug Administration specifically to treat depression is Bupropion (Wellbutrin, Wellbutrin SR, Wellbutrin XL). The only tetracyclic antidepressant that has been approved by the Food and Drug Administration specifically to treat depression is Mirtazapine (Remeron, Remeron SolTab). Other compounds approved for treatment of neuropsychiatric disorders include Anafranil (clomipramine HCl); Aventyl (nortriptyline HCl); Desyrel (trazodone HCl); Elavil (amitriptyline HCl); Limbitrol (chlordiazepoxide/amitriptyline); Ludiomil (Maprotiline HCl); Luvox (fluvoxamine maleate); Marplan (isocarboxazid); Nardil (phenelzine sulfate); Norpramin (desipramine HCl); Pamelor (nortriptyline HCl); Parnate (tranylcypromine sulfate); Pexeva (paroxetine mesylate); Prozac (fluoxetine HCl); Sarafem (fluoxetine HCl); Serzone (nefazodone HCl); Sinequan (doxepin HCl); Surmontil (trimipramine); Symbyax (olanzapine/fluoxetine); Tofranil (imipramine HCl); Tofranil-PM (impiramine pamoate); Triavil (Perphenaine/Amitriptyline); Vivactil (protriptyline HCl); Wellbutrin (bupropion HCl); and Zyban (bupropion HCl). Combined inhibitors and blockers that have been approved by the Food and Drug Administration specifically to treat depression are: Trazodone, Nefazodone and Maprotiline.

Tricyclic antidepressants (TCAs) inhibit the reabsorption (reuptake) of serotonin and norepinephrine. They were among the earliest of antidepressants, hitting the market in the 1960s, and they remained the first line of treatment for depression through the 1980s, before newer antidepressants arrived. TCAs that have been approved by the Food and Drug Administration specifically to treat depression are: Amitriptyline, Amoxapine, Desipramine (Norpramin), Doxepin (Sinequan), Imipramine (Tofranil), Nortriptyline (Pamelor), Protriptyline (Vivactil) and Trimipramine (Surmontil)

MAOIs that have been specifically approved by the Food and Drug Administration to treat depression are: Phenelzine (Nardil), Tranylcypromine (Parnate), Isocarboxazid (Marplan) and Selegiline (Emsam). Emsam is the first skin (transdermal) patch for depression.

Any of the compounds of the invention can be administered in combination with another active agent. In certain embodiments, the second active is one that is effective in treatment of a neuropsychiatric disorder. However, in certain other embodiments, the second active is one that is effective against an underlying disorder that is associated with a neuropsychiatric symptom. Examples of such disorders are heart disease, Alzheimer's disease and Parkinson's diseases. In certain embodiments, the compounds can be administered in combination in a single dosage form or injection, or administered concurrently. In other embodiments, the compounds are administered in alternation.

### Side Effects

In an additional aspect of the methods and processes described herein, the compound does not exhibit substantial toxic an/or psychotic side effects. Toxic side effects include, but are not limited to: agitation, hallucination, confusion, stupor, paranoia, delirium, psychotomimetic-like symptoms, rotarod impairment, amphetamine-like stereotyped behaviors, stereotypy, psychosis memory impairment, motor impairment, anxiolytic-like effects, increased blood pressure, decreased blood pressure, increased pulse, decreased pulse, hematological abnormalities, electrocardiogram (ECG) abnormalities, cardiac toxicity, heart palpitations, motor stimulation, psychomotor performance, mood changes, short-term memory deficits, long-term memory deficits, arousal, sedation, extrapyramidal side-effects, ventricular tachycardia. Lengthening of cardiac repolarisation, ataxia, cognitive deficits and/or schizophrenia-like symptoms.

Further, in another embodiment, the compounds selected or identified according to the processes and methods described herein do not have substantial side effects associated with other classes of NMDA receptor antagonists. In one embodiments, such compounds do not substantially exhibit the side effects associated with NMDA antagonists of the glutamate site, such as selfotel, D-CPPene (SDZ EAA 494) and AR-R15896AR (ARL 15896AR), including, agitation, hallucination, confusion and stupor (Davis et al. (2000) Stroke 31(2):347-354; Diener et al. (2002), J Neurol 249(5):561-568); paranoia and delirium (Grotta et al. (1995), J Intern Med 237:89-94); psychotomimetic-like symptoms (Loscher et al. (1998), Neurosci Lett 240(1):33-36); poor therapeutic ratio (Dawson et al. (2001), Brain Res 892(2):344-350); amphetamine-like stereotyped behaviors (Potschka et al. (1999), Eur J Pharmacol 374(2):175-187). In another embodiment, such compounds do not exhibit the side effects associated with NMDA antagonists of the glycine site, such as HA-966, L-701,324, d-cycloserine, CGP-40116, and ACEA 1021, including significant memory impairment and motor impairment (Wlaz, P (1998), Brain Res Bull 46(6):535-540). In a still further embodiment, such compounds do not exhibit the side effects of NMDA high affinity receptor channel blockers, such as MK-801 and ketamine, including, psychosis-like effects (Hoffman, D C (1992), J Neural Transm Gen Sect 89:1-10); cognitive deficits (decrements in free recall, recognition memory, and attention; Malhotra et al (1996), Neuropsychopharmacology 14:301-307); schizophrenia-like symptoms (Krystal et al (1994), Arch Gen Psychiatry 51:199-214; Lahti et al. (2001), Neuropsychopharmacology 25:455-467), and hyperactivity and increased stereotypy (Ford et al (1989) Physiology and behavior 46: 755-758.

In a further additional or alternative embodiment, the compound has a therapeutic index equal to or greater than at least 2:1, at least 3:1, at least 4:1, at least 5:1, at least 6:1, at least 7:1, at least 8:1, at least 9:1, at least 10:1, at least 15:1, at least 20:1, at least 25:1, at least 30:1, at least 40:1, at least 50:1, at least 75:1, at least 100:1 or at least 1000:1. The therapeutic index can be defined as the ratio of the dose required to produce toxic or lethal effects to dose required to produce therapeutic responses. It can be the ratio between the median toxic dose (the dosage at which 50% of the group exhibits the adverse effect of the drug) and the median effective dose (the dosage at which 50% of the population respond to the drug in a specific manner). The higher the therapeutic index, the more safe the drug is considered to be. It simply indicates that it would take a higher dose to invoke a toxic response that it does to cause a beneficial effect.

The side effect profile of compounds can be determined by any method known to those skilled in the art. In one embodiment, motor impairment can be measured by, for example, measuring locomotor activity and/or rotorod performance. Rotorod experiments involve measuring the duration that an animal can remain on an accelerating rod. In another embodiment, memory impairment can be assessed, for example, by using a passive avoidance paradigm; Sternberg memory scanning and paired words for short-term memory, or delayed free recall of pictures for long-term memory. In a further embodiment, anxiolytic-like effects can be measured, for example, in the elevated plus maze task. In other embodiments, cardiac function can be monitored, blood pressure and/or body temperature measured and/or electrocardiograms conducted to test for side effects. In other embodiments, psychomotor functions and arousal can be measured, for example by analyzing critical flicker fusion threshold, choice reaction time, and/or body sway. In other embodiments, mood can be assessed using, for example, self-ratings. In further embodiments, schizophrenic symptoms can be evaluated, for example, using the PANSS, BPRS, and CGI, side-effects were assessed by the HAS and the S/A scale.

### EXAMPLES

The following examples are provided to illustrate the present invention and are not intended to limit the scope thereof. Those skilled in the art will readily understand that kown variations of the conditions and processes of the following preparative procedures can be used to manufacture the desired compounds. The materials required for the embodiments and the examples are known in the literature, readily commercially available, or can be made by known methods from the known starting materials by those skilled in the art.

### Synthesis of Compounds

The compounds for use in the methods described herein can be prepared by any methods known in the art, such as in accordance with the methods and general synthetic strategies provided in WO 02/072542 or WO 09/006,437, or by the following synthetic methods, or variations of those procedures readily understand to those skilled in the art.

### Examples 1 and 2

### N-(4-{3-[4-(3,4-Difluoro-phenyl)-piperazin-1-yl]-2-(S)-hydroxy-propoxy}-phenyl)-methanesulfonamide (Compound 1) and N-(4-{3-[2-(3,4-Dichloro-phenylamino)-ethylamino]-2-(S)-hydroxy-propoxy}phenyl)-methanesulfonamide (Compound 2).

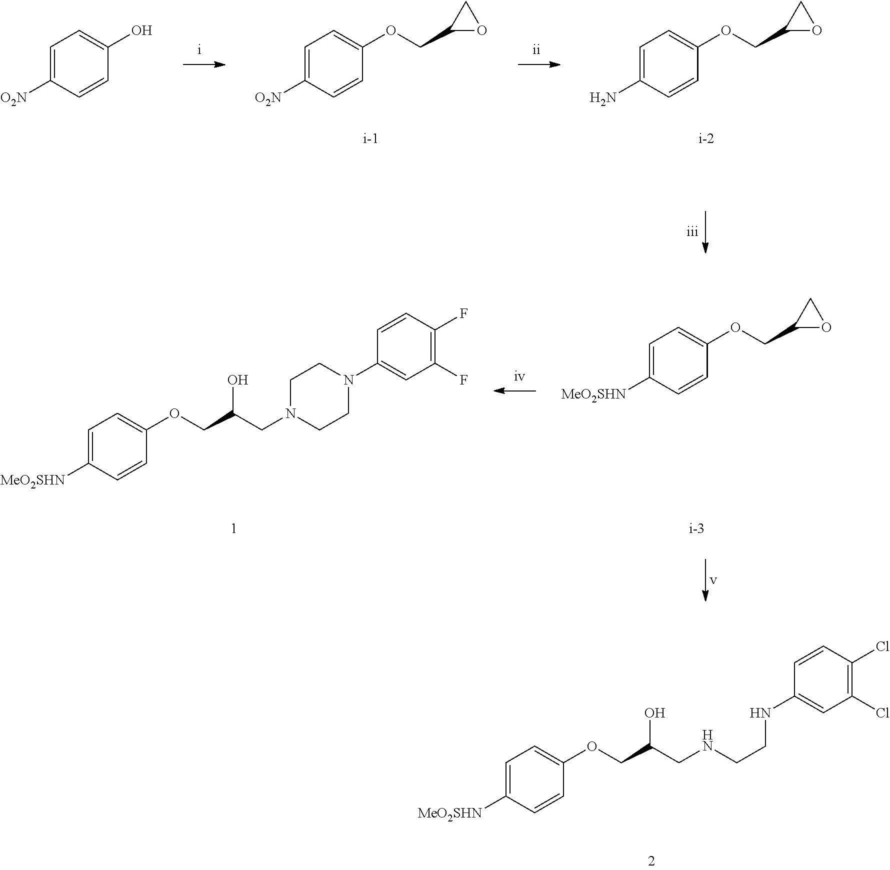

Step (i). 3-(4-Nitro-phenoxy)-2-(S)-propyleneoxide (i-1). 4-Nitrophenol (6.6 mmol) was dissolved in 5 ml anhydrous DMF. Cesium fluoride (19.9 mmol) was added to the reaction. The reaction mixture was stirred for 1 hour at room temperature and (S)-Glycidyl nosylate (6.6 mmol) was added to the reaction mixture. The reaction stirred for 24 hours at room temperature. Water (150 mL) was added and the solution was extracted with ethyl acetate. The organic phase was dried over MgSO₄ and evaporated. The residue was purified with column chromatograph using ethylacetate: hexane (50:50) solvent system to give the desired product i-1 This step can be substituted with (R)-Glycidyl nosylate to get the R isomer.
Step (ii). 3-(4-Amino-phenoxy)-2-(S)-propyleneoxide (i-2). (S)-Glycidyl-4-nitrophenyl ether (2.6 mmol, i-1) and 5% Pd/C(en)[{Sajiki et all, Chemistry—a europian journal 6(12):2200-2204 (2000).] (10% of the weight of starting material) in 5 ml anhydrous THF was hydrogenated at ambient pressure and temperature for 3 hours. The reaction mixture was filtered by using membrane filter (13, 0.22 μm) and the filtrate was concentrated in vacuum. The compound was afforded as a crude mixture of amino reduction compound i-2.
Step (iii). 3-(4-methansulfonylamido-phenoxy)-2-(S)-propyleneoxide (i-3). (S)-Glycidyl-4-aminophenyl ether (2.4 mmol, i-2) dissolved in 20 ml anhydrous DCM and N,N-diisopropyl-N-ethylamine (2.6 mmol) was added at 0° C. After stirring 15 minutes methanesulfonyl chloride (2.6 mmol) was added drop wise to the reaction mixture at 0° C. After stirring over night, the reaction extracted with water and washed with brine. Organic phase dried over magnesium sulfate and evaporated. The residue was purified with flash chromatography using Ethyl acetate: DCM (30:70) solvent system to give the desired product i-3.
Step (iv). N-(4-{3-[4-(3,4-Difluoro-phenyl)-piperazin-1-yl]-2-(S)-hydroxy-propoxy}-phenyl)-methanesulfonamide (Compound 1). Compound i-3 (2.00 mmol) and N-(3,4-Difluorophenyl)piperazine (2.00 mmol) were heated under reflux conditions in 20 ml ethanol for 8 hours. Then solvent was evaporated and residue was purified with flash chromatography using dichloromethane:methanol (90:10) solvent system to get compound 1. Compound 1 was dissolved in ethanol and bubbled HCl gas to get the HCl salt of the compound 1.
Step (v). N-(4-{3-[2-(3,4-Dichloro-phenylamino)-ethylamino]-2-(S)-hydroxy-propoxy}-phenyl)-methanesulfonamide (Compound 2). The epoxide (i-3, 1.58 mmol) was dissolved in EtOH (20 ml), and then the 3,4-dicholoro-ethylene diamine (1.58 mmol) (preparation: Isabel Perillo, M. Cristina Caterina, Julieta Lopez, Alejandra Salerno. Synthesis 2004, 6, 851-856) was added and the solution refluxed for 16 hours. The solvent was evaporated and the product purified with column chromatography using 10% MeOH/DCM+1% NH₄OH to give compound 2.
The following compounds were synthesized according to the procedures provided in examples 1 and 2.

| COMPOUND | NAME AND PHYSICAL DATA |
|---|---|
| 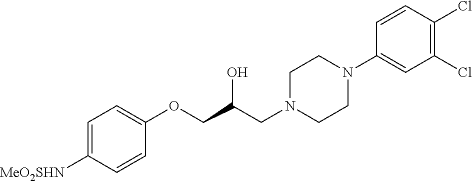 | N-(4-{3-[4-(3,4-Dichloro-phenyl)-piperazin-1-yl]-2- (S)-hydroxy-propoxy}-phenyl)-methanesulfonamide MS: 475.14486 |
| 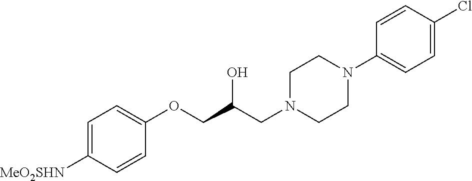 | N-(4-{3-[4-(4-Chloro-phenyl)-piperazin-1-yl]-2- (S)-hydroxy-propoxy}-phenyl)-methanesulfonamide MS: 440.14017 |
| . 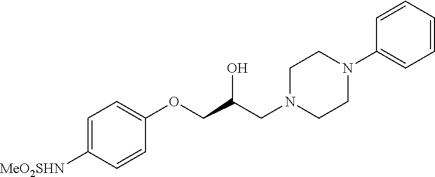 | N-(4-{3-[4-phenyl-piperazin-1-yl]-2-(S)-hydroxy- propoxy}-phenyl)-methanesulfonamide MS:406.20183 |
| 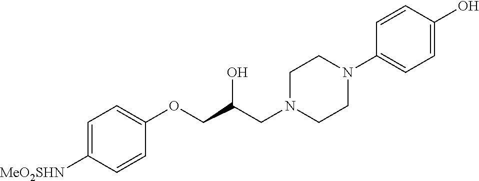 | N-(4-{3-[4-(4-Hydroxy-phenyl)-piperazin-1-yl]-2- (S)-hydroxy-propoxy}-phenyl)-methanesulfonamide |
| 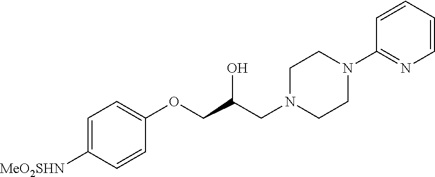 | N-(4-{3-[4-(2-Pyridyl)-piperazin-1-yl]-2-(S)- hydroxy-propoxy}-phenyl)-methanesulfonamide |
| 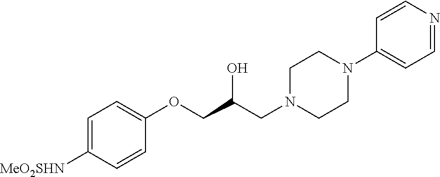 | N-(4-{3-[4-(4-Pyridyl)-piperazin-1-yl]-2-(S)- hydroxy-propoxy}-phenyl)-methanesulfonamide |
| 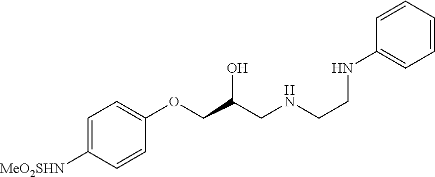 | N-{4-[2-(S)-Hydroxy-3-(2-phenylamino- ethylamino)-propoxy]-phenyl}-methanesulfonamide |
| 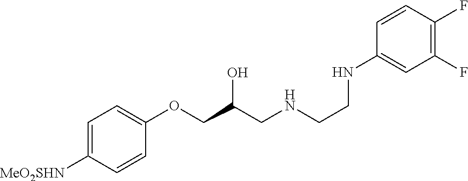 | N-{4-[2-(S)-Hydroxy-3-(2-(3,4-difluoro- phenyl)amino-ethylamino)-propoxy]-phenyl}- methanesulfonamide MS: 416.15842 |
| 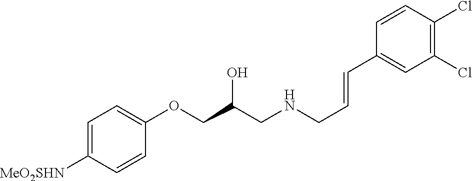 | N-(4-{3-[3-(3,4-Dichloro-phenyl)-allylamino]-2- (S)-hydroxy-propoxy}-phenyl)-methanesulfonamide MS: 446.09479 |
| 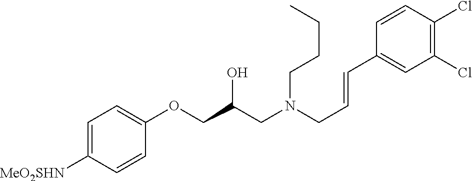 | N-[4-(3-{Butyl-[3-(3,4-dichloro-phenyl)-allyl]- amino}-2-(S)-hydroxy-propoxy)-phenyl]- methanesulfonamide MS: 501.13871 |
| 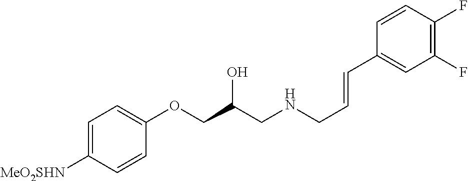 | N-(4-{3-[3-(3,4-Difluoro-phenyl)-allylamino]-2-(S)- hydroxy-propoxy}-phenyl)-methanesulfonamide MS: 413.58425 |

### Example 3

### 6-{3-[4-(4-Chloro-phenyl)-piperazin-1-yl]-2-(S)-hydroxy-propoxy}-3H-benzooxazol-2-one (Compound 3)

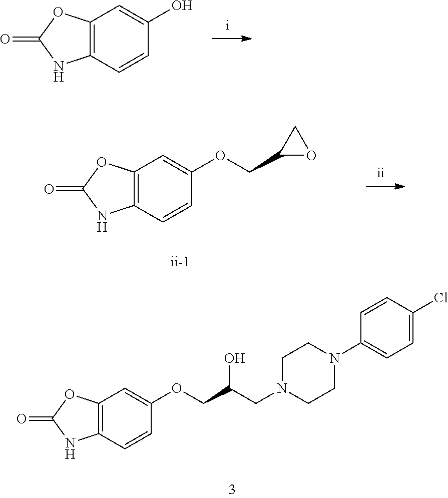

Step (i). 6-(2-(S)-Oxiranylmethoxy)-3H-benzooxazol-2-one (ii-1). 5-hydroxy-benzoxazole (310 mg) and cesium carbonate (780 mg) were combined in 6 mL of N,N-dimethylformamide. The reaction was stirred for room temperature for 1 hour. (S)-glycidal nosylate (520 mg) was added, and the reaction stirred at room temperature overnight. The reaction was quenched with NH₄Cl(aq) solution and extracted with ethyl acetate. The organic layer was washed with NH₄Cl(aq) and NaCl(aq) solutions, separated, and dried over Na₂SO₄(s). Filtration and solvent removal was followed by absorption onto silica gel. Elution with an ethyl actate/methanol mixture (4:1) followed by solvent removal gave 445 mg of a yellow, oily solid.
Step (ii). 6-{3-[4-(4-Chloro-phenyl)-piperazin-1-yl]-2-(S)-hydroxy-propoxy}-3H-benzooxazol-2-one (Compound 3). To a solution of 300 mg of epoxide (ii-1) in 10 mL of absolute ethanol was added 300 mg of 4-(4-chlorophenyl)-piperazine. The solution was heated to 70° C. for 8 hours. The reaction was cooled and the solvent removed under vacuum. The residue was purified by column chromatography on silica gel using ethyl acetate as solvent. Obtained 240 mg of a light brown solid (45% yield). 1HNMR (d6-DMSO, 400 MHz): δ 2.37 (dq, 2H, J=6 Hz, J=13 Hz), 2.51 (m, 4H), 3.02 (m, 4H), 3.68 (q, 1H, J=8 Hz 3.84 (dd, 1H, J=4 Hz, J=14 Hz), 4.02 (bs, 1H), 5.07 (d, 1H, J=5 Hz), 6.61 (dd, 1H, J=2 Hz, J=9 Hz 6.73 (d, 1H, J=2 Hz 6.91 (d, 2H, J=9 Hz 7.05 (d, 1H, J=8 Hz 7.21 (d, 2H, J=9 Hz 9.43 (s, 1H); MS (m/z): 404 (M+H), 406 (M+2+H); HRMS Calcd. for C20H23ClN304: 404.13771. Found: 404.13673.
The following compounds were synthesized according to the procedure in Example 3.

| COMPOUND | NAME AND PHYSICAL DATA |
|---|---|
| 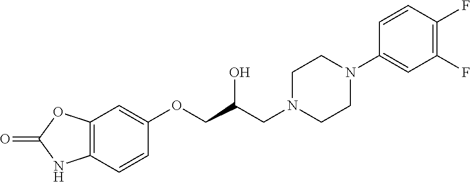 | 6-{3-[4-(3,4-Difluoro-phenyl)-piperazin-1-yl]-2-(S)- hydroxy-propoxy}-3H-benzooxazol-2-one MS: 406.15664 |
| 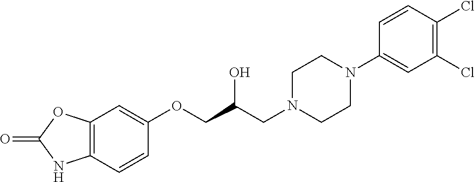 | 6-{3-[4-(3,4-Dichloro-phenyl)-piperazin-1-yl]-2- (S)-hydroxy-propoxy}-3H-benzooxazol-2-one |
| 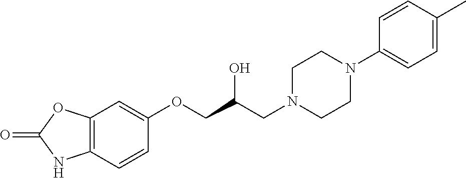 | 6-{3-[4-(4-Methyl-phenyl)-piperazin-1-yl]-2-(S)- hydroxy-propoxy}-3H-benzooxazol-2-one MS: 384.19077 |
| 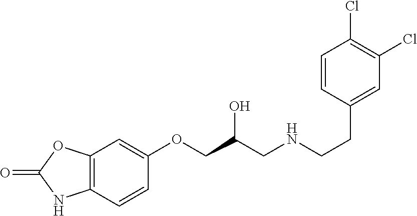 | 6-{3-[2-(3,4-Dichloro-phenyl)-ethylamino]-2-(S)- hydroxy-propoxy}-3H-benzooxazol-2-one |
| 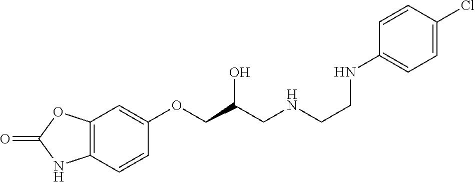 | 6-{3-[2-(4-Chloro-phenylamino)-ethylamino]-2-(S)- hydroxy-propoxy}-3H-benzooxazol-2-one MS: 378.12089 |

### Example 4

### 4-{3-[4-(3,4-Dichloro-phenyl)-piperazin-1-yl]-2-(S)-hydroxy-propoxy}-phenol (Compound 4)

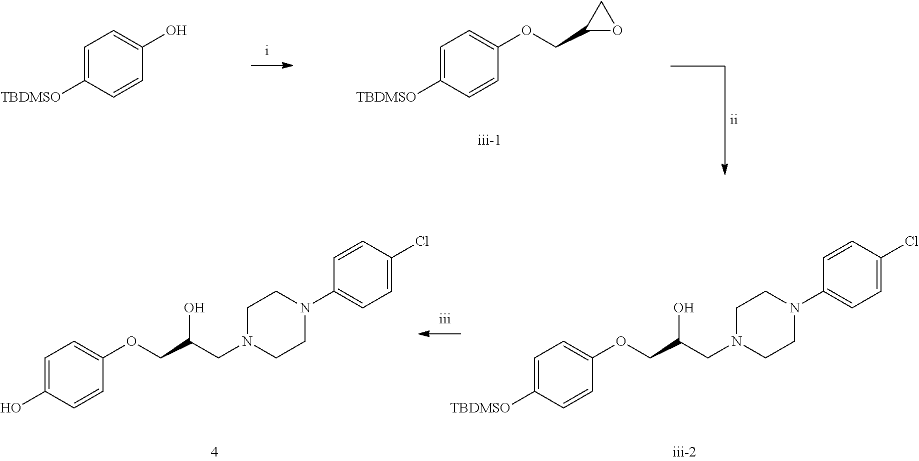

Step (i). 3-(4-tert-Butyldimethylsilyloxy-phenoxy)-2-(S)-propyleneoxide (iii-1). 4-(tert-Butyldimethylsiloxy)phenol (1.45 g, 6.25 mmol) in 5 ml anhydrous THF was added dropwise to the suspension of NaH (0.158 g, 6.25 mmol) in 5 ml THF. After stirring at room temperature for 2 hours glycidyl nosylate (1.30 g, 5 mmol) and then 15-crown-5 (25 mol %) were added to the reaction mixture. After stirring 24 hours reaction was poured to ice-water and extracted with ethyl acetate. Organic phase was washed with water and brine, then dried over sodium sulfate and evaporated. Product was purified by column chromatography using EtOAc:Hexane (1:9) (yield: 1.06 g 76%). ¹H-NMR (400 MHz, CDCl₃) δ 0.17 (6H, s), 0.98 (9H, s), 2.75 (1H, dd, J=2.4, 4.4 Hz), 2.89 (1H, q, J=4.4 Hz), 3.33-3.36 (1H, m), 3.90 (1H, dd, J=5.6, 10.8 Hz), 4.16 (1H, dd, J=3.6, 11.2 Hz), 6.69-6.81 (4H, m).
Step (ii). 4-{3-[4-(3,4-Dichloro-phenyl)-piperazin-1-yl]-2-(S)-hydroxy-propoxy}-phenoxy-tert-butyldimethyl silane (iii-2). Compound iii-1 (0.280 g, 1 mmol) and 1-(4-chlorophenyl)piperazine (0.200 g, 1 mmol) were dissolved in 5 ml EtOH and refluxed for 90 minutes. Solvent was evaporated and the material was used in the next step without purification.
Step (iii). 4-{3-[4-(3,4-Dichloro-phenyl)-piperazin-1-yl]-2-(S)-hydroxy-propoxy}-phenol (Compound 4). Compound iii-2 was dissolved in 5 ml THF and 2 ml TBAF in 1.0M THF solution was added, and stirred for 2 hours. Quenched with ammonium chloride solution, extracted with EtOAc. Organic phase was dried over sodium sulfate and evaporated. Product was purified using column chromatography using EtOAc:MeOH (95:5). 1H-NMR (400 MHz, DMSO-d6) δ 2.36-2.61 (6H, m), 3.11 (4H, t, J=4.8 Hz), 3.76 (1H, dd, J=4.0, 6.0 Hz), 386(1H, dd, J=4.4, 10.0 Hz), 3.91-3.95 (1H, m), 4.85 (1H, d, J=4.8 Hz), 6.66 (1H, dd, J=2.4, 6.8 Hz), 6.75 (1H, dd, J=2.4, 6.8 Hz), 6.92 (1H, dd, J=2.4, 6.8 Hz), 7.21 (1H, dd, J=2.4, 6.8 Hz), 8.90 (1H, s). HRMS: 362.1397 calculated. 362.14696 found.
The following compounds were synthesized according to Example 4.

| COMPOUND | NAME AND PHYSICAL DATA |
|---|---|
| 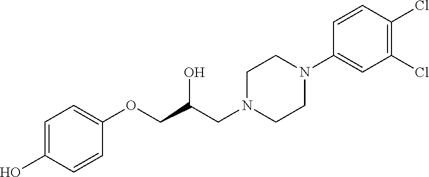 | 4-{3-[4-(3,4-Dichloro-phenyl)-piperazin-1-yl]-2- (S)-hydroxy-propoxy}-phenol MS: 397.10811 |
| 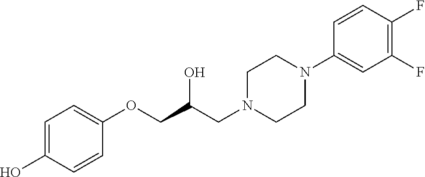 | 4-{3-[4-(3,4-Difluoro-phenyl)-piperazin-1-yl]-2-(S)- hydroxy-propoxy}-phenol MS:365.16672 |
| 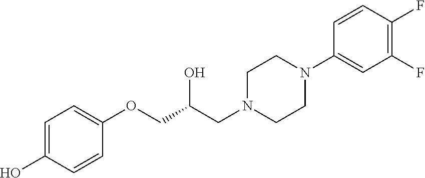 | 4-{3-[4-(3,4-Difluoro-phenyl)-piperazin-1-yl]-2- (R)-hydroxy-propoxy}-phenol MS: 365.16657 |
| 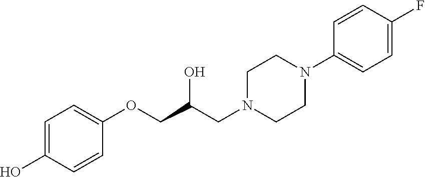 | 4-{3-[4-(4-Fluoro-phenyl)-piperazin-1-yl]-2-(S)- hydroxy-propoxy}-phenol MS: 347.17602 |
| 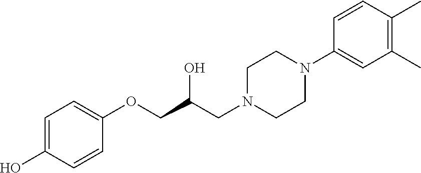 | 4-{3-[4-(3,4-Dimethyl-phenyl)-piperazin-1-yl]-2- (S)-hydroxy-propoxy}-phenol MS: 357.21716 |
| 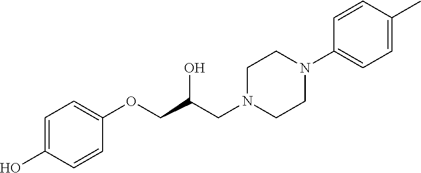 | 4-{3-[4-(4-Methyl-phenyl)-piperazin-1-yl]-2-(S)- hydroxy-propoxy}-phenol MS: 343.20093 |
| 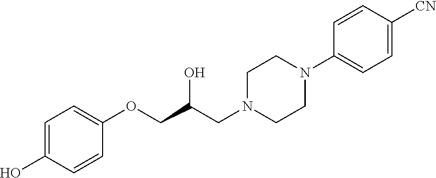 | 4-{3-[4-(4-Cyano-phenyl)-piperazin-1-yl]-2-(S)- hydroxy-propoxy}-phenol MS: 354.18073 |
| 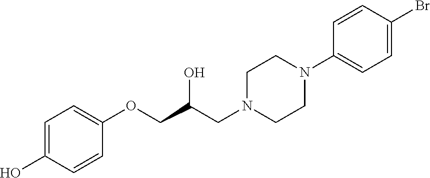 | 4-{3-[4-(4-Bromo-phenyl)-piperazin-1-yl]-2-(S)- hydroxy-propoxy}-phenol MS: 407.09663 |
| 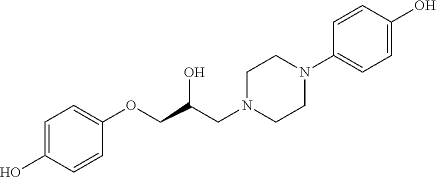 | 4-{3-[4-(4-Hydroxy-phenyl)-piperazin-1-yl]-2-(S)- hydroxy-propoxy}-phenol MS: 345.18061 |
| 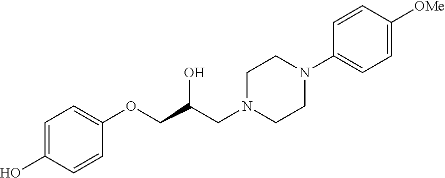 | 4-{3-[4-(4-Methoxy-phenyl)-piperazin-1-yl]-2-(S)- hydroxy-propoxy}-phenol MS: 359.19608 |
| 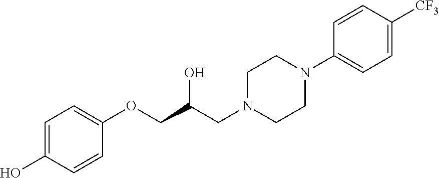 | 4-{3-[4-(4-Trifluoromethyl-phenyl)-piperazin-1-yl] - 2-(S)-hydroxy-propoxy}-phenol MS: 397.17327 |
| 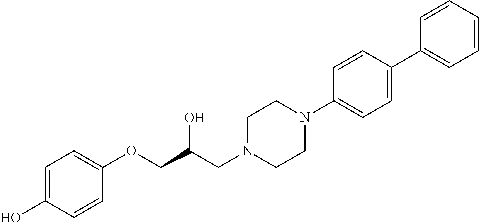 | 4-{3-[4-(4-Biphenyl)-piperazin-1-yl]-2-(S)- hydroxy-propoxy}-phenol MS: 405.21643 |
| 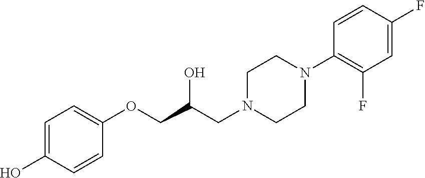 | 4-{3-[4-(2,4-Difluoro-phenyl)-piperazin-1-yl]-2-(S)- hydroxy-propoxy}-phenol MS: 365.16651 |
| 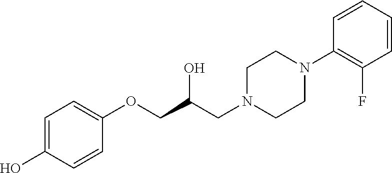 | 4-{3-[4-(2-Fluoro-phenyl)-piperazin-1-yl]-2-(S)- hydroxy-propoxy}-phenol MS: 347.17595 |
| 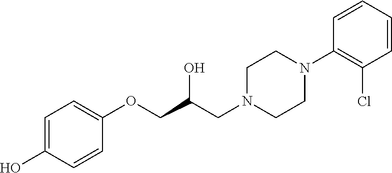 | 4-{3-[4-(2-Chloro-phenyl)-piperazin-1-yl]-2-(S)- hydroxy-propoxy}-phenol MS: 363.14695 |
| 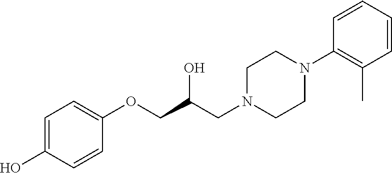 | 4-{3-[4-(2-Chloro-phenyl)-piperazin-1-yl]-2-(S)- hydroxy-propoxy}-phenol MS: 343.20108 |
| 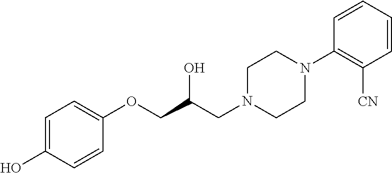 | 4-{3-[4-(2-Cyano-phenyl)-piperazin-1-yl]-2-(S)- hydroxy-propoxy}-phenol MS: 354.18070 |
| 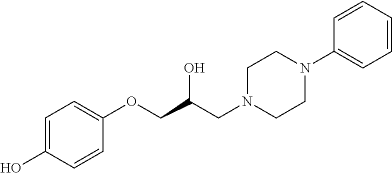 | 4-{3-[4-Phenyl-piperazin-1-yl]-2-(S)-hydroxy- propoxy}-phenol MS: 329.18547 |
| 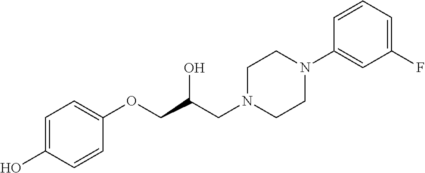 | 4-{3-[4-(3-Fluoro-phenyl)-piperazin-1-yl]-2-(S)- hydroxy-propoxy}-phenol MS: 347.17608 |
| 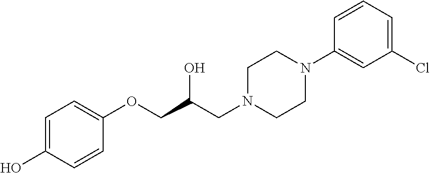 | 4-{3-[4-(3-Chloro-phenyl)-piperazin-1-yl]-2-(S)- hydroxy-propoxy}-phenol MS: 363.14741 |
| 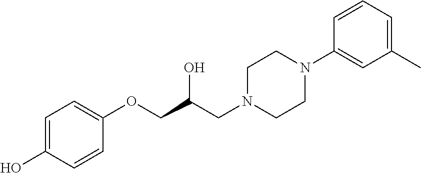 | 4-{3-[4-(3-Methyl-phenyl)-piperazin-1-yl]-2-(S)- hydroxy-propoxy}-phenol MS: 343.20109 |
| 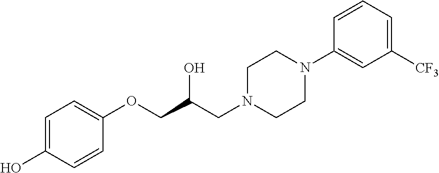 | 4-{3-[4-(3-Trifluoromethyl-phenyl)-piperazin-1-yl]- 2-(S)-hydroxy-propoxy}-phenol MS: 397.17269 |
| 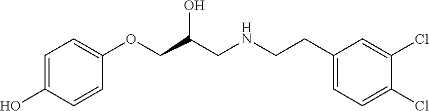 | 4-{3-[2-(3,4-Dichloro-phenyl)-ethylamino]-2-(S)- hydroxy-propoxy}-phenol MS: 357.10159 |
| 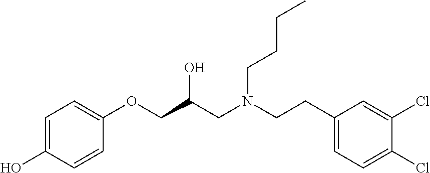 | 4-(3-{Butyl-[2-(3,4-dichloro-phenyl)-ethyl]- amino}-2-(S)-hydroxy-propoxy)-phenol MS: 413.12769 |
| 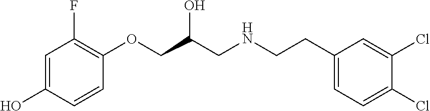 | 4-{3-[2-(3,4-Dichloro-phenyl)-ethylamino]-2-(S)- hydroxy-propoxy}-3-fluoro-phenol MS: 375.03418 |
| 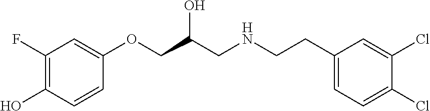 | 4-{3-[2-(3,4-Dichloro-phenyl)-ethylamino]-2-(S)- hydroxy-propoxy}-2-fluoro-phenol MS: 375.03409 |
| 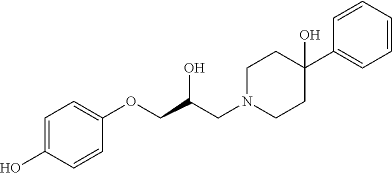 | 1-[2-(S)-Hydroxy-3-(4-hydroxy-phenoxy)-propyl]- 4-phenyl-piperidin-4-ol MS: 344.18509 |

### Example 5

### (4-{3-[4-(3,4-Difluoro-phenyl)-piperazin-1-yl]propoxy}-phenyl)-urea (Compound 5)

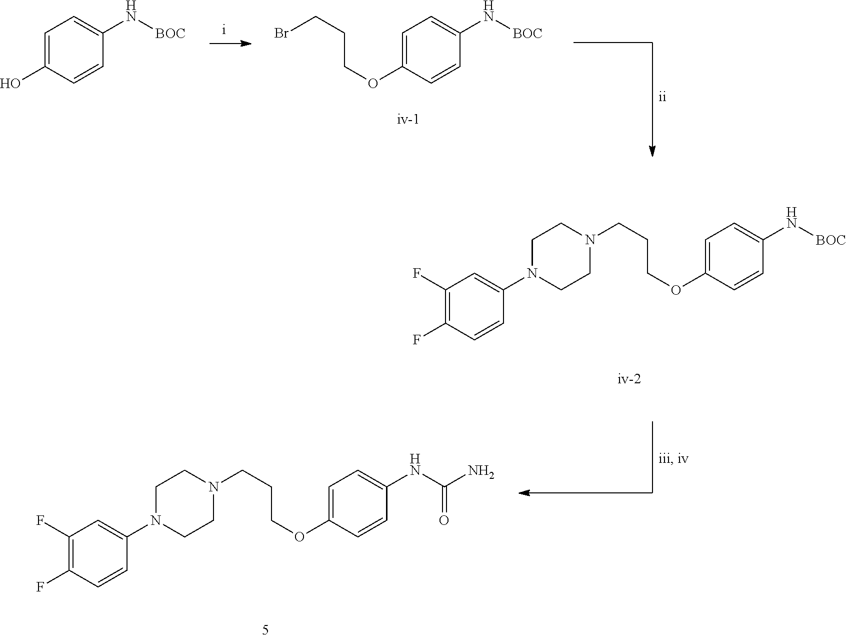

Step (i). [4-(3-Bromo-propoxy)-phenyl]-carbamic acid tert-butyl ester (iv-1). To a solution of 2.1 g of 4-t-butylcarbonylamino-phenol in 20 mL of acetonitrile was added 3.25 g of cesium carbonate. The reaction was stirred for one hour, and then 1.5 mL of 1,3-dibromopropane was added and the reaction stirred for 20 hours. The reaction was then quenched with NH₄Cl(aq.) solution. The mixture was extracted with ethyl acetate and washed with NH₄Cl(aq.) and NaCl(aq.) solutions. The organic layer was separated and dried over Na₂SO₄(s). Filtration and solvent removal gave a light brown oily solid. Hexanes were added and the resulting solids filtered and washed with Hexanes three times. Drying gave 2.4 g of an off-white solid.
Step (ii). (4-{3-[4-(3,4-Difluoro-phenyl)-piperazin-1-yl]-propoxy}-phenyl)-carbamic acid tert-butyl ester (iv-2). To 305 mg of 4-(3,4-Difluoro-phenyl)-piperazine and 335 mg of compound 1v-1 was added 5 mL of acetonitrile. The reaction was heated to 65° C. overnight. The reaction was cooled, and then extracted with ethyl acetate. The organic layers were washed with NaHCO₃(aq.) twice, and the organic layers separated and dried over Na₂SO₄(s). Filtration and solvent removal gave an light brown solid. Dilution with hexanes, filtration, and washing with hexanes gave 458 mg of a white solid (iv-2). MS (m/z): 430 (M+H); HRMS: Obsd for C₂₄H₃₃FN₃O₃: 430.24951.
Step (iii). Compound 1v-2 (430 mg) was dissolved in 6 mL of dichloromethane. Next, 4 mL of trifluoroacetic acid was added and the reaction was stirred for 6 hours. Then NaHCO₃(s) was added until the bubbling stopped. Then water was added to the reaction mixture and the reaction was extracted with dichloromethane and washed with NaHCO₃(aq.) twice. The organics were dried over Na₂SO₄(s), and then the solution was filtered and the solvent removed under vacuum. The residue was used in the next step without any purification.
Step (iv). (4-{3-[4-(3,4-Difluoro-phenyl)-piperazin-1-yl]-propoxy}-phenyl)-urea (Compound 5). The aniline from the previous step was dissolved in 10 mL of N,N-dimethyl formamide. Next, 1 mL of trimethylsilyl isocyanate was added, and the reaction was stirred at room temperature overnight. The reaction was then quenched with NaHCO₃(aq.) solution. The reaction was extracted with ethyl acetate and washed with NaHCO₃(aq.) solution twice. The organic layer was separated and dried over Na₂SO₄(s). Filtration and solvent removal gave a brown solid. Filtration over a plug of silica gel with ethyl acetate/methanol (4:1) was followed by solvent removal. Trituration of the resulting solids with ethyl ether and filtration gave 98 mg of an off-white solid. MS (m/z): 391 (M+H); HRMS: calcd. for C₂₀H₂₅F₂N₄O₂: 391.19456. Found: 391.19184.
The following compounds were synthesized according to the methods and variations of described for Example 5.

| COMPOUND | NAME AND PHYSICAL DATA |
|---|---|
| 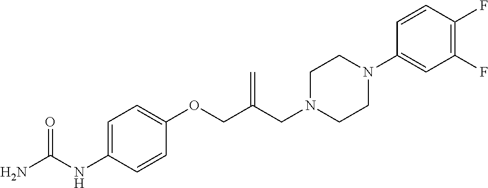 | (4-{2-[4-(3,4-Difluoro-phenyl)-piperazin-1- ylmethyl]-allyloxy}-phenyl)-urea MS: 403.19326 |
| 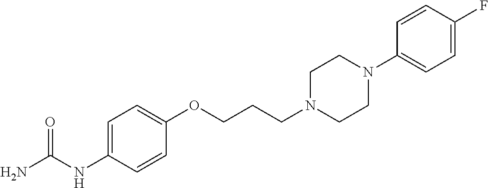 | (4-{3-[4-(4-Fluoro-phenyl)-piperazin-1-yl]- propoxy}-phenyl)-urea MS: 373.20319 |
| 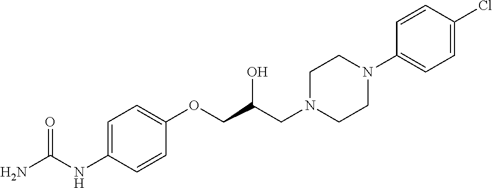 | (4-{3-[4-(4-Chloro-phenyl)-piperazin-1-yl]-2- hydroxy-propoxy}-phenyl)-urea |
| 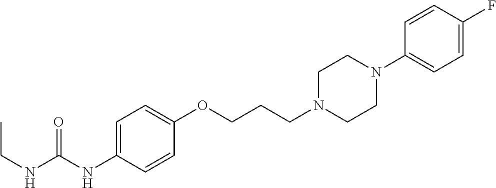 | 1-Ethyl-3-(4-{3-[4-(4-fluoro-phenyl)- piperazin-1-yl]-propoxy}-phenyl)-urea |
| 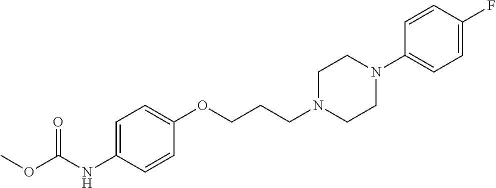 | (4-{3-[4-(4-Fluoro-phenyl)-piperazin-1-yl]- propoxy}-phenyl)-carbamic acid methyl ester MS: 388.20251 |

### Examples 6, 7, and 8

### N-[2-(3,4-Dichloro-phenylamino)-ethyl]-3-(4-methanesulfonylamino-phenyl)-propionamide (Compound 6), N-(4-{3-[2-(3,4-Dichloro-phenylamino)-ethylamino]-propyl}-phenyl)-methanesulfonamide (Compound 7), and N-(4-(3-(3-(3,4-dichlorophenyl)-2-oxoimidazolidin-1 I)propyl)phenyl)methanesulfonamide (Compound 8).

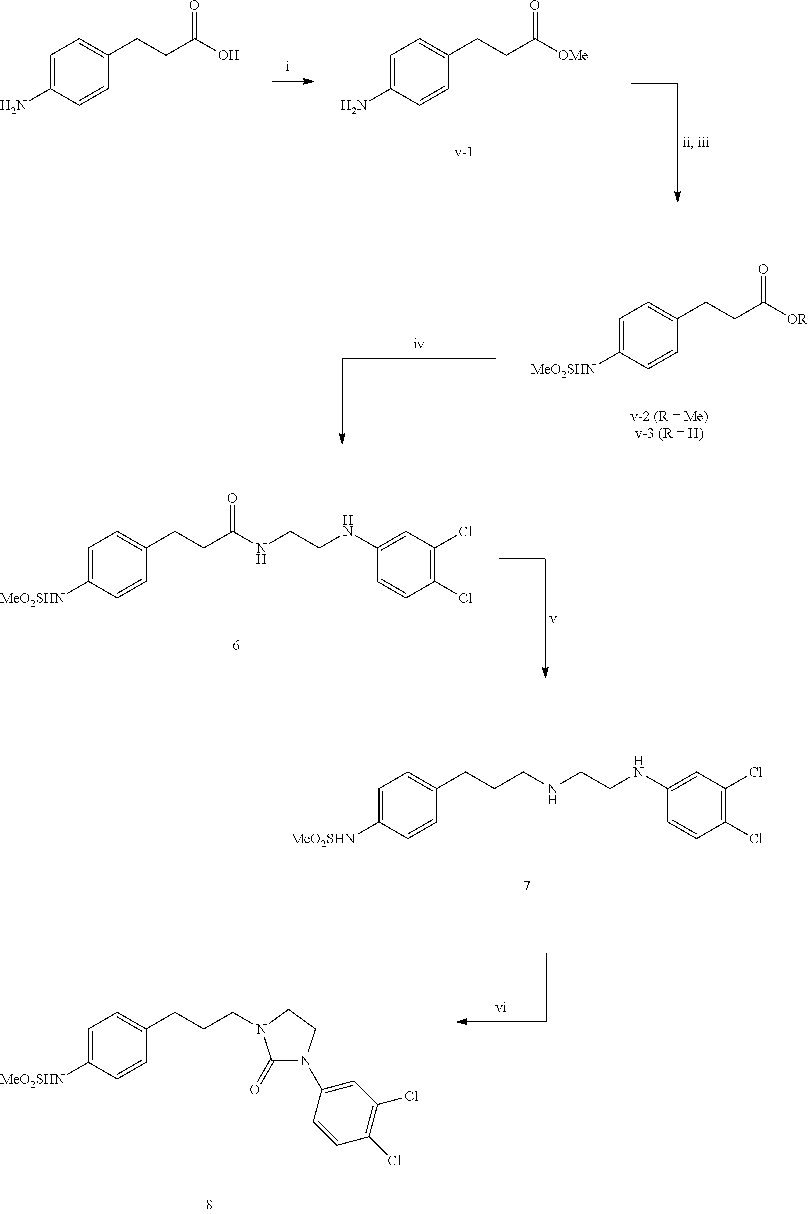

Step (i) Methyl 3-(4-aminophenyl)propanoate (v-1). Thionyl chloride (14.6 ml, 200 mmol, 3.3 equiv) was added dropwise to a solution of dry methanol (60 ml, 1453 mmol, 24 equiv) at −10° C. After stirring for 10 minutes, 3-(4-aminophenyl)propanoic acid (10.0 g, 61 mmol) was added to give a yellow suspension. The solution stirred for 1 hour and was slowly warmed to room temperature. The resulting solution was concentrated to give a yellow solid. The solid was suspended in ethyl acetate, and NaHCO₃ (aq.) was added until the salt dissolved fully. Solid sodium bicarbonate was added to give pH 8. The layers were separated and the organics were washed with brine (aq.). The resulting solution was dried over MgSO₄, filtered, and concentrated to give a yellow solid (10.6 g, 98%). ¹H NMR (300 MHz, CDCl₃) 7.00 (d, J=8.3 Hz, 2H), 6.63 (d, J=8.3 Hz, 2H), 3.67 (s, 3H), 3.59 (bs, NH₂, 2H), 2.85 (t, J=7.6 Hz, 2H), 2.58 (t, J=8.3 Hz, 2H). ¹³C NMR (300 MHz, CDCl₃) 173.8, 144.9, 130.7, 129.3, 115.5, 51.8, 36.4, 30.4. M.S. (ESI) m/z=180.102 (M+H).
Step (ii). Methyl 3-(4-(methylsulfonamido)phenyl)propanoate (v-2). The ester (7.38 g, 41.2 mmol) was dissolved in pyridine (17.0 ml, excess). After cooling to 0° C., methanesulfonyl chloride (4.55 ml, 57.7 mmol, 1.4 equiv) was added dropwise. The reaction was warmed to room temperature and stirred overnight. The reaction was quenched with water and diluted with DCM. The layers were separated and the organics were washed with brine. The resulting solution was concentrated to give a red solid. The crude material was purified using silica gel chromatography (1 EtOAc/1 Hexanes) to give a white solid (87%). ¹H NMR: (CDCl₃, 400 MHz) 7.20 (d, J=8.6 Hz, 2H), 7.15 (d, J=8.6 Hz, 2H), 6.45 (bs, NH, 1H), 3.68 (s, 3H), 3.00 (s, 3H), 2.94 (t, J=7.6 Hz, 2H), 2.63 (t, J=7.5 Hz, 2H). ¹³C NMR (CDCl₃, 400 MHz): 173.4, 137.6, 135.2, 129.4, 121.4, 51.7, 38.5, 35.5, 30.1. M.S. (ESI) m/z=257.56 (M+H)
Step (iii). 3-(4-(methylsulfonamido)phenyl)propanoic acid (v-3). The sulfonamide ester (1.16 g, 4.5 mmol) was dissolved in methanol (50 ml). To this solution, 1.0 N NaOH (17.0 ml, 17.0 mmol, 3.8 equiv) was added. The mixture was stirred at room temperature overnight. TLC indicated the reaction was finished. The pH of the solution was adjusted to 3 with a solution of aqueous HCl. The volume of methanol was reduced by rotary evaporation (40 mbar), upon which the product crashed out of solution. The yellow crystals were filtered off and dried (0.900 g, 82%). ¹H NMR (400 MHz, CD₃OD) 7.21 (d, J=8.6 Hz, 2H), 7.17 (d, J=8.6 Hz, 2H), 2.91 (s, 3H), 2.89 (t, J=7.6 Hz, 2H), 2.59 (t, J=7.6 Hz, 2H). ¹³C NMR (400 MHz, CD₃OD) 176.7, 139.0, 137.7, 130.5, 122.3, 39.1, 36.8, 31.4. M.S. (ESI) m/z=242.05 (M−H).
Step (iv). N-(2-(3,4-dichlorophenylamino)ethyl)-3-(4-(methylsulfonamido)phenyl) propanamide Compound 6). The carboxylic acid (0.700 g, 2.88 mmol) was dissolved in DMF (30.0 ml) and cooled to 0° C. To this solution, DMAP (0.352 g, 2.28 mmol, 1.1 equiv), and EDCI (0.552 g, 2.88 mmol, 1.0 equiv) were added to give a clear suspension. After stirring for 30 minutes, the amine (0.590 g, 2.88 mmol, 1.0 equiv) in THF (5.0 ml) was added dropwise to give a brown solution. The mixture was warmed to room temperature and stirred overnight. The reaction was monitored by TLC. To quench the reaction, 20 mL of 1.0 N HCl was added and the solution was extracted with 3×30 mL of EtOAc. The organic layer was dried with MgSO₄, filtered, and concentrated to give red oil. The crude material was purified by taking the residue up in DCM and stirring. Immediately a white powder precipitated out (0.920 g, 74%). ¹H NMR (400 MHz, CD₃OD) 7.14-7.10 (mult, 5H), 6.72 (d, J=2.9 Hz, 1H), 6.51 (dd, J₁=8.9 Hz, J₂=2.6 Hz, 1H), 3.27 (t, 2H), 3.10 (t, J=6.4 Hz, 2H), 2.88 (s, 3H), 2.87 (t, J=6.5 Hz, 2H), 2.46, (t, J=6.4 Hz, 2H). ¹³C NMR (300 MHz, CDCl₃) 175.8, 150.0, 138.7, 138.3, 131.7, 130.5, 122.3, 114.4, 113.6, 44.0, 39.8, 39.2, 39.0, 32.3. M.S. Calc'd 429.0681. Found (HRMS) 431.08143 (M+H). E.A. Calc'd: C, 50.24; H, 4.92; N, 9.76. Found: C, 49.94; H, 4.91; N, 9.74.
Step (v). N-(4-(3-(2-(3,4-dichlorophenylamino)ethylamino)propyl)phenyl)methanesulfonamide (Compound 7). The sulfonamide amide ((0.500 g, 1.2 mmol) was dissolved in THF (30.0 ml). After cooling to 0° C., a solution of Lithium Aluminum hydride (2.0 M solution in THF, 2.3 ml, 4.6 mmol, 4.0 equiv) was added dropwise. After stirring for 10 minutes at 0° C., the ice bath was removed and the reaction mixed was warmed to room temperature and stirred overnight. The mixture was diluted with DCM and water to give an emulsion. Rochelle's salt (sat'd solution) was added and the mixture stirred for 20 minutes before filtering over a pad of celite. The resulting liquid was separated, and the organics were washed with brine, dried over MgSO₄ and concentrated to give a white foam (0.358 g, 74%). The free base was converted to the HCl salt by bubbling HCl (g) through a solution of substrate dissolved in ethanol. The white powder precipitated out and was filtered off ¹H NMR (300 MHz, CDCl₃) 7.20-7.11 (mult, 5H), 6.69 (d, J=2.8 Hz, 1H), 6.46 (dd, J₁=8.8 Hz, J₂=2.8 Hz), 4.36 (bs, 1H, NH), 3.156 (mult, 2H), 3.00 (s, 3H), 2.88 (t, J=6.2 Hz, 2H), 2.66 (t, J=7.1 Hz, 4H), 1.86-1.79 (mult, 3H). ¹³C NMR (300 MHz, CDCl₃) 148.1, 139.4, 134.8, 132.8, 130.7, 129.7, 121.6, 119.7, 113.9, 112.9, 49.0, 48.2, 43.2, 39.3, 32.9, 31.5. M.S. Calc'd 416.088. Found (HRMS): 416.069.
Step (vi). N-(4-(3-(3-(3,4-dichlorophenyl)-2-oxoimidazolidin-1 1)propyl)phenyl)methanesulfonamide (Compound 8). The starting material diamine (0.113 g, 0.27 mmol) was dissolved in THF (10.0 ml). To this solution 1,1-carbonyldiimidazole (0.048 g, 0.30 mmol, 1.1 equiv) was added. The mixture stirred at room temperature overnight. After completion, the solution was evaporated to dryness and the residue was taken up in ethyl acetate, washed with brine (1×) and dried over Na₂SO₄, filtered, and concentrated to give a clear oil. The crude material was purified using silica gel chromatography (100% EtOAc) to give a white foam (0.070 g, 58%). ¹H (400 MHz, CDCl₃) 7.72 (s, 1H), 7.16 (d, J=8.6 Hz, 2H), 7.07 (d, J=8.6 Hz, 2H), 7.02 (s, 1H), 6.64 (d, J=2.9 Hz, 1H), 6.41 (dd, J₁=8.5 Hz, J₂=2.9 Hz), 3.64 (t, J=6.0 Hz, 2H), 3.40-3.36 (mult, 4H), 2.97 (s, 3H), 2.57 (t, J=7.3 Hz, 2H), 1.26 (t, J=7.3 Hz, 2H). ¹³C (75 MHz, CDCl₃) 152.6, 147.1, 137.3, 136.8, 135.6, 130.9, 129.5, 121.6, 118.0, 113.6, 112.5. M.S. (ESI) Calc'd: 441.0681. Found: 442.07527 (M+H).
Compounds in the following table were synthesized according to variations in methods described for Examples 6, 7, and 8.

| COMPOUND | NAME |
|---|---|
| 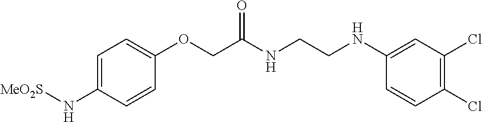 | N-[2-(3,4-Dichloro-phenylamino)-ethyl]-2-(4- methanesulfonylamino-phenoxy)-acetamide |
| 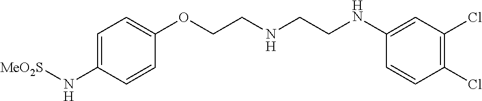 | N-(4-{2-[2-(3,4-Dichloro-phenylamino)- ethylamino]-ethoxy}-phenyl)-methanesulfonamide |
| 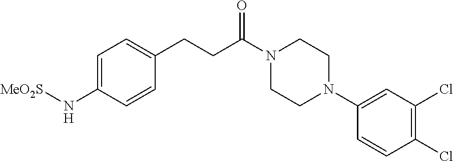 | N-(4-{3-[4-(3,4-Dichloro-phenyl)-piperazin-1-yl]-3- oxo-propyl}-phenyl)-methanesulfonamide |
| 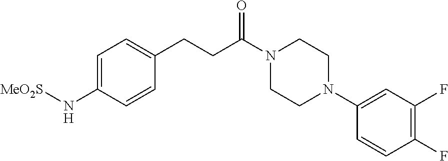 | N-(4-{3-[4-(3,4-DiFluoro-phenyl)-piperazin-1-yl]-3- oxo-propyl}-phenyl)-methanesulfonamide |
| 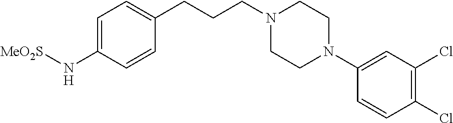 | N-(4-{3-[4-(3,4-Dichloro-phenyl)-piperazin-1-yl]- propyl}-phenyl)-methanesulfonamide |
| 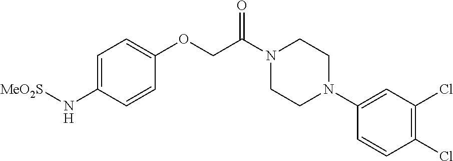 | N-(4-{2-[4-(3,4-Dichloro-phenyl)-piperazin-1-yl]-2- oxo-ethoxy}-phenyl)-methanesulfonamide |
| 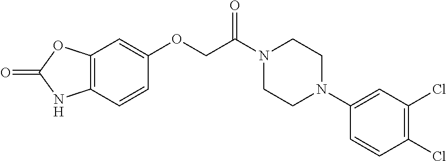 | 6-{2-[4-(3,4-Dichloro-phenyl)-piperazin-1-yl]-2-oxo- ethoxy}-3H-benzooxazol-2-one |
| 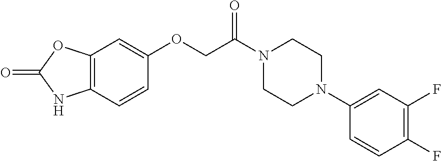 | 6-{2-[4-(3,4-DiFluoro-phenyl)-piperazin-1-yl]-2-oxo- ethoxy}-3H-benzooxazol-2-one |
| 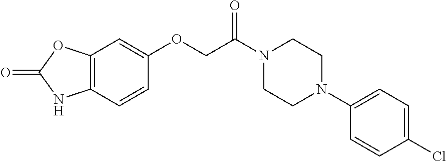 | 6-{2-[4-(4-Chloro-phenyl)-piperazin-1-yl]-2-oxo- ethoxy}-3H-benzooxazol-2-one |
| 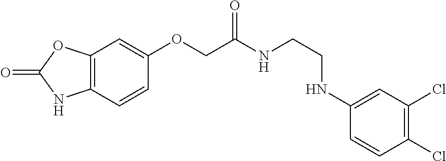 | N-[2-(3,4-Dichloro-phenylamino)-ethyl]-2-(2-oxo-2,3- dihydro-benzooxazol-6-yloxy)-acetamide |
| 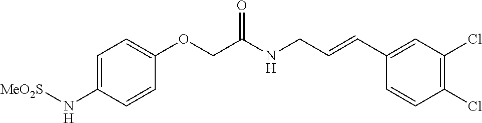 | N-[3-(3,4-Dichloro-phenyl)-allyl]-2-(4- methanesulfonylamino-phenoxy)-acetamide |
| 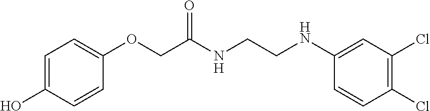 | N-[2-(3,4-Dichloro-phenylamino)-ethyl]-2-(4- hydroxy-phenoxy)-acetamide |
| 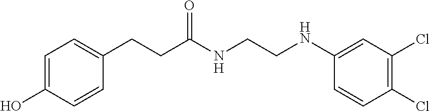 | N-[2-(3,4-Dichloro-phenylamino)-ethyl]-3-(4- hydroxy-phenyl)-propionamide |
| 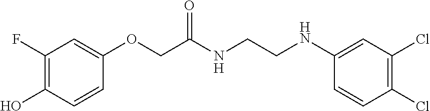 | N-[2-(3,4-Dichloro-phenylamino)-ethyl]-2-(3-fluoro- 4-hydroxy-phenoxy)-acetamide |

### Biological Data

### Example 9

### Expression of Glutamate Receptors in Xenopus laevis Oocytes

cRNA was synthesized from linearized template cDNA for rat glutamate receptor subunits according to manufacturer specifications (Ambion). Quality of synthesized cRNA was assessed by gel electrophoresis, and quantity was estimated by spectroscopy and gel electrophoresis. Stage V and VI oocytes were surgically removed from the ovaries of large, well-fed and healthy Xenopus laevis anesthetized with 3-amino-benzoic acid ethyl ester (3 gm/l) as previously described. Clusters of isolated oocytes were incubated with 292 U/ml Worthington (Freehold, N.J.) type IV collagenase or 1.3 mg/ml collagenase (Life Technologies, Gaithersburg, Md.; 17018-029) for 2 hr in Ca²⁺-free solution comprised of (in mM) 115 NaCl, 2.5 KCl, and 10 HEPES, pH 7.5, with slow agitation to remove the follicular cell layer. Oocytes were then washed extensively in the same solution supplemented with 1.8 mM CaCl₂ and maintained in Barth's solution comprised of (in mM): 88 NaCl, 1 KCl, 2.4 NaHCO₃, 10 HEPES, 0.82 MgSO₄, 0.33 Ca(NO₃)₂, and 0.91 CaCl₂ and supplemented with 100 μg/ml gentamycin, 10 μg/ml streptomycin, and 10 μg/ml penicillin. Oocytes were manually defolliculated and injected within 24 hrs of isolation with 3-5 ng of NR1 subunit cRNA and 7-10 ng of NR2 cRNA subunit in a 50 nl volume, or 5-10 ng of AMPA or kainate receptor cRNAs in a 50 nl volume, and incubated in Barth's solution at 18° C. for 1-7 d. Glass injection pipettes had tip sizes ranging from 10-20 microns, and were backfilled with mineral oil.

### Example 10

### Two Electrode Voltage Clamp Recording from Xenopus laevis Oocytes

Two electrode voltage-clamp recordings were made 2-7 days post-injection as previously described. Oocytes were placed in a dual-track plexiglass recording chamber with a single perfusion line that splits in a Y-configuration to perfuse two oocytes. Dual recordings were made at room temperature (23° C.) using two Warner OC725B two-electrode voltage clamp amplifiers, arranged as recommended by the manufacturer. Glass microelectrodes (1-10 Megaohms) were filled with 300 mM KCl (voltage electrode) or 3 M KCl (current electrode). The bath clamps communicated across silver chloride wires placed into each side of the recording chamber, both of which were assumed to be at a reference potential of 0 mV. Oocytes were perfused with a solution comprised of (in mM) 90 NaCl, 1 KCl, 10 HEPES, and 0.5 BaCl₂; pH was adjusted by addition of 1-3 M NaOH of HCl. Oocytes were recorded under voltage clamp at −40 mV. Final concentrations for control application of glutamate (50 μM) plus glycine (30 μM) were achieved by adding appropriate volumes from 100 and 30 mM stock solutions, respectively. In addition, 10 μM final EDTA was obtained by adding a 1:1000 dilution of 10 mM EDTA, in order to chelate contaminant divalent ions such as Zn²⁺. Concentration-response curves for experimental compounds were obtained by applying in successive fashion maximal glutamate/glycine, followed by glutamate/glycine plus variable concentrations of experimental compounds. Dose response curves consisting of 4 to 8 concentrations were obtained in this manner. The baseline leak current at −40 mV was measured before and after recording, and the full recording linearly corrected for any change in leak current. Oocytes with glutamate-evoked responses smaller than 50 nA were not included in the analysis. The level of inhibition by applied experimental compounds was expressed as a percent of the initial glutamate response, and averaged together across oocytes from a single frog. Each experiment consisted of recordings from 3 to 10 oocytes obtained from a single frog. Results from 3-6 experiments were pooled, and the average percent responses at antagonist concentrations were fitted by the equation,

Percent Response=(100−minimum)/(1+([conc]/IC₅₀)^{nH})+minimum

where minimum is the residual percent response in saturating concentration of the experimental compounds, IC₅₀ is the concentration of antagonist that causes half of the achievable inhibition, and nH is a slope factor describing steepness of the inhibition curve. Minimum was constrained to be greater than or equal to 0.

Assay results for test compounds are reported in Tables 17-21.

TABLE 17
pH Dependence of NMDA Antagonism

| COMPOUND | IC₅₀ at pH 6.9 (nM) | IC₅₀ at pH 7.6 (nM) |
|---|---|---|
| 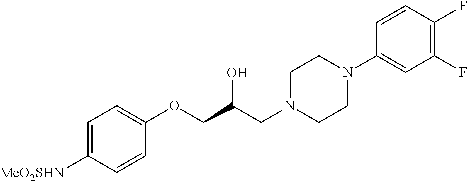 | 31 | 557 |
| 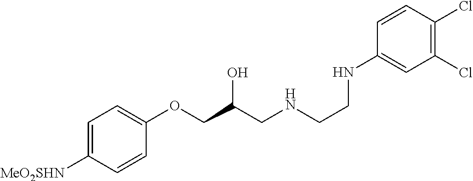 | 12 | 62 |
| 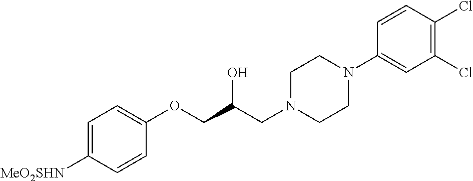 | 296 | 1,020 |
| 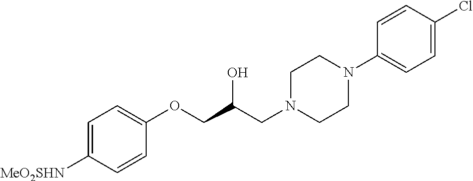 | 32 | 270 |
| 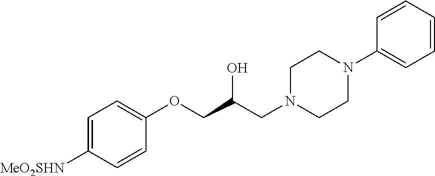 | 213 | 1,020 |
| 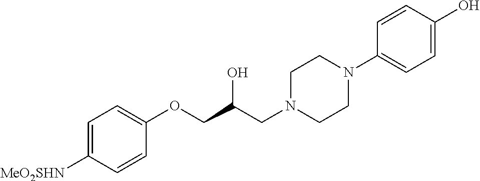 | 2,400 | 28,000 |
| 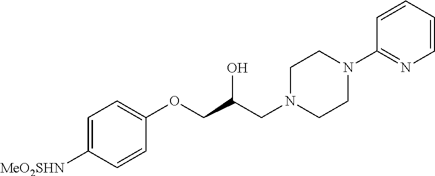 | 1,810 | 12,700 |
| 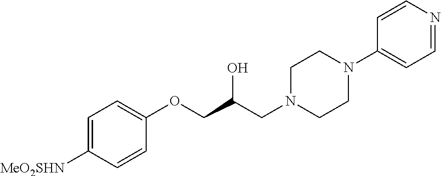 | >30,000 | >30,000 |
| 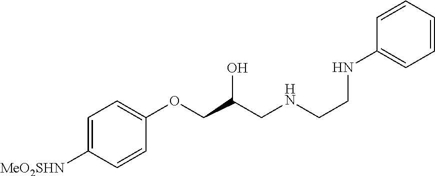 | 55 | 133 |
| 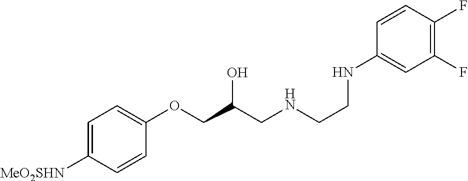 | 108 | 199 |
| 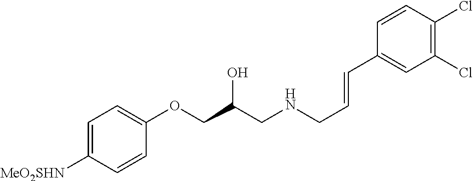 | 7 | 38 |
| 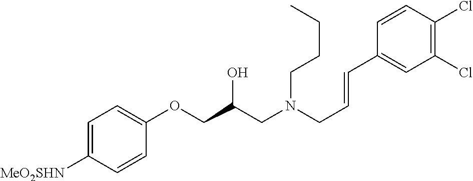 | 163 | 2,580 |
| 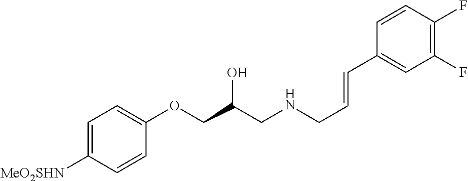 | 38 | 71 |

TABLE 18
pH Dependence of NMDA Antagonism

| COMPOUND | IC₅₀ at pH 6.9 (nM) | IC₅₀ at pH 7.6 (nM) |
|---|---|---|
| 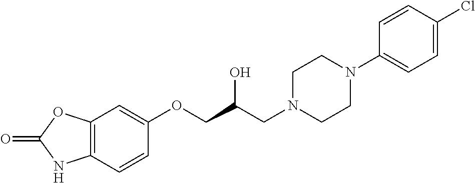 | 79 | 726 |
| 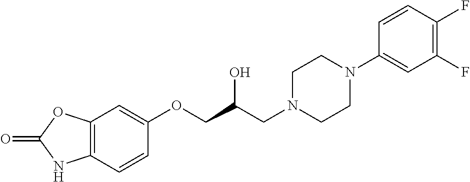 | 145 | 1,700 |
| 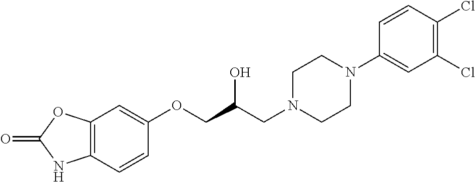 | 185 | 1,500 |
| 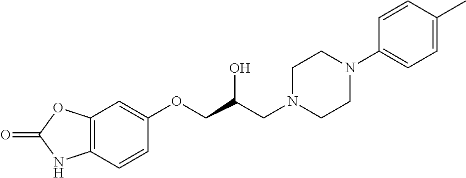 | 72 | 644 |
| 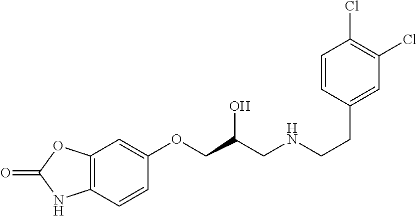 | 185 | 446 |
| 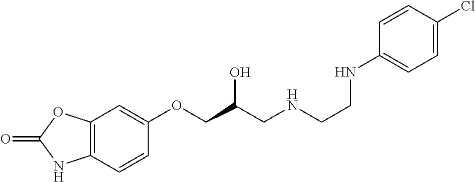 | 25 | 97 |

TABLE 19
pH Dependence of NMDA Antagonism

| COMPOUND | IC₅₀ at pH 6.9 (nM) | IC₅₀ at pH 7.6 (nM) |
|---|---|---|
| 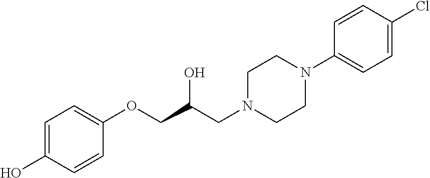 | 80 | 814 |
| 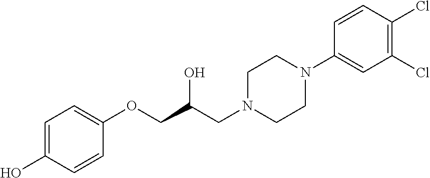 | 131 | 611 |
| 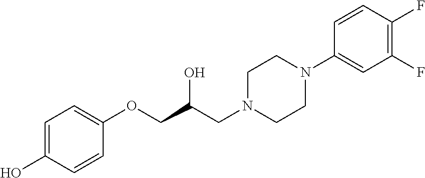 | 87 | 980 |
| 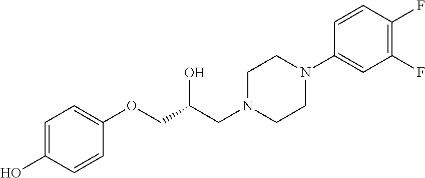 | 50 | 272 |
| 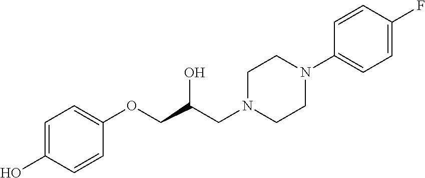 | 87 | 529 |
| 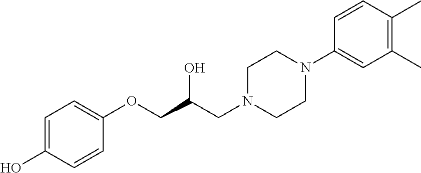 | 1,500 | 5,790 |
| 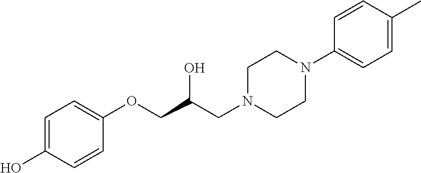 | 70 | 260 |
| 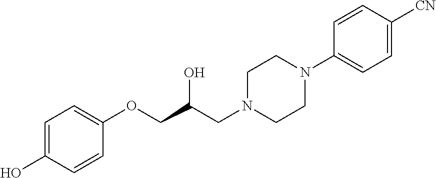 | 460 | 4,370 |
| 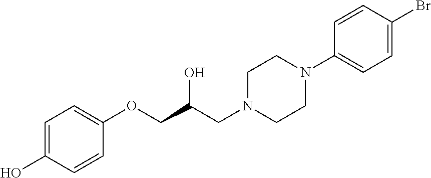 | 73 | 204 |
| 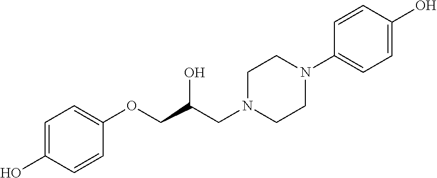 | 576 | 4,700 |
| 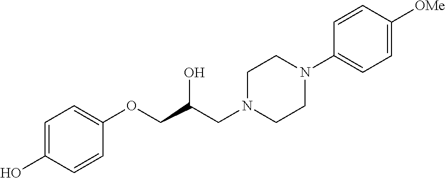 | 562 | 9,740 |
| 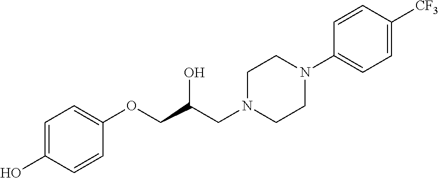 | 113 | 448 |
| 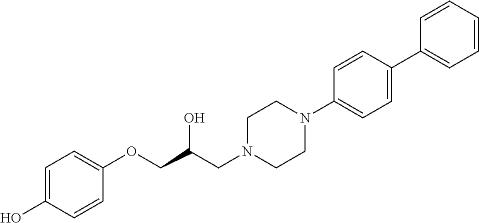 | 6,950 | 191,300 |
| 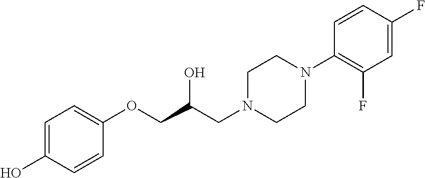 | 529 | 3,560 |
| 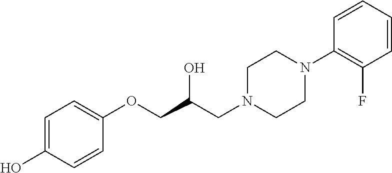 | 541 | 1,730 |
| 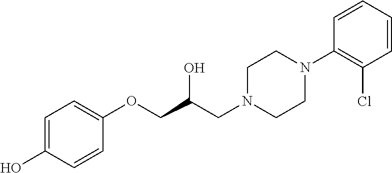 | 978 | 4,390 |
| 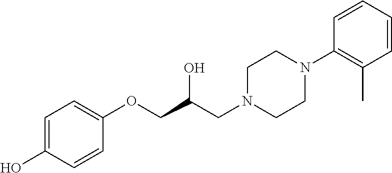 | 430 | 3,930 |
| 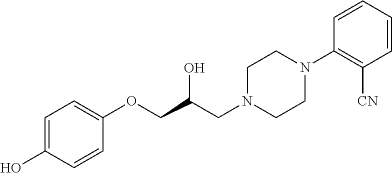 | 3,210 | 8,330 |
| 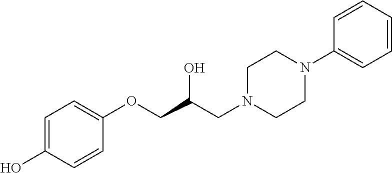 | 217 | 476 |
| 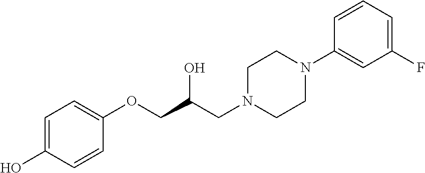 | 211 | 598 |
| 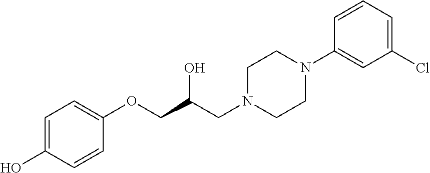 | 257 | 4,800 |
| 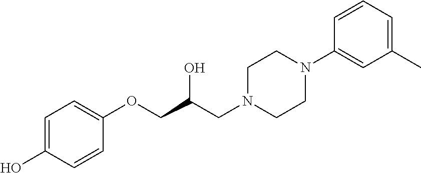 | 341 | 1,740 |
| 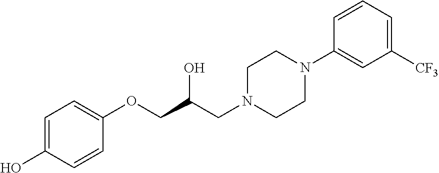 | 386 | 3,790 |
| 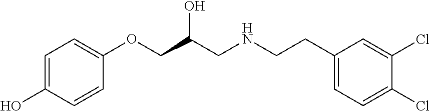 | 197 | 501 |
| 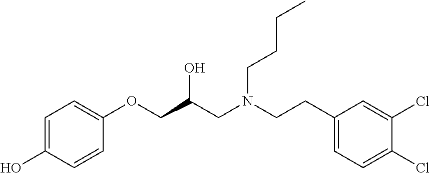 | 585 | 6,500 |
| 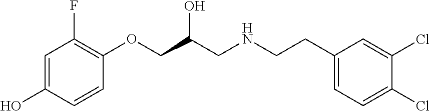 | 116 | 330 |
| 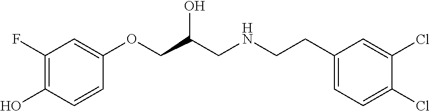 | 114 | 549 |
| 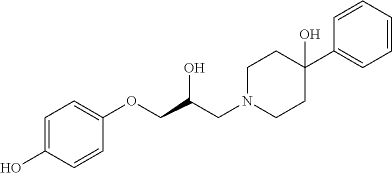 | 191 | 298 |

TABLE 20
pH Dependence of NMDA Antagonism

| COMPOUND | IC₅₀ at pH 6.9 (nM) | IC₅₀ at pH 7.6 (nM) |
|---|---|---|
| 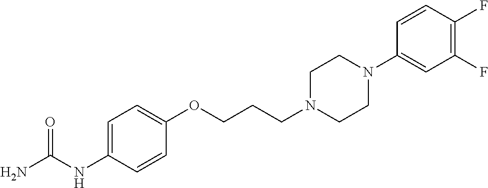 | 82 | 696 |
| 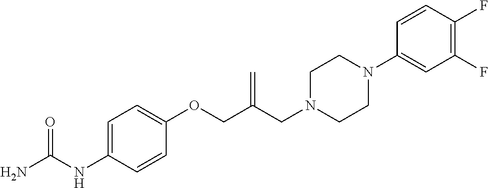 | 557 | 10,400 |
| 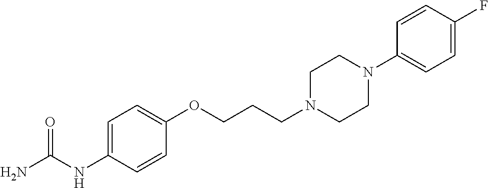 | 117 | 625 |
| 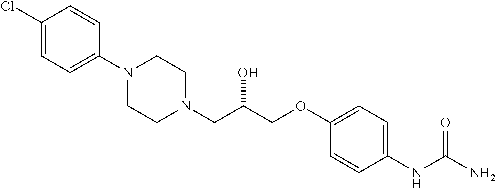 | 46 | 452 |
| 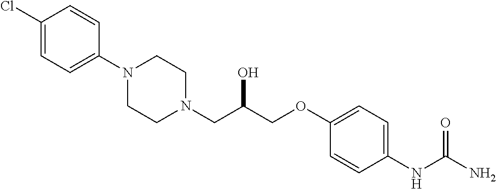 | 19 | 60 |
| 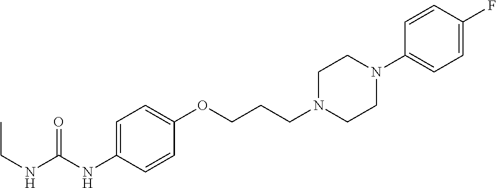 | 3,030 | 15,700 |
| 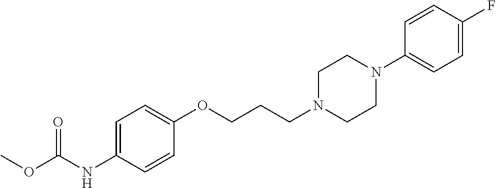 | 1,540 | 16,200 |

TABLE 21
pH Dependence of NMDA Antagonism

| COMPOUND | IC₅₀ at pH 6.9 | IC₅₀ at pH 7.6 |
|---|---|---|
| 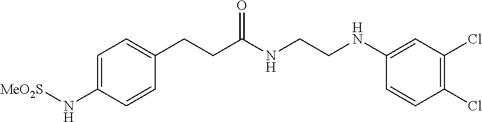 | 30 | 55 |
| 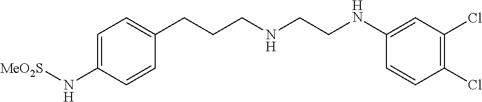 | 2 | 21 |
| 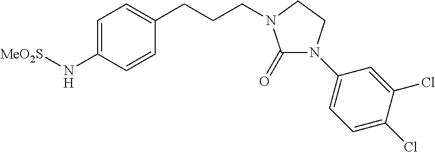 | 160 | 809 |
| 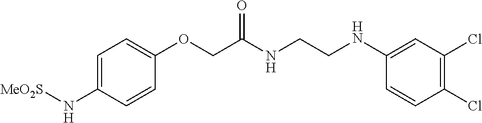 | 13 | 58 |
| 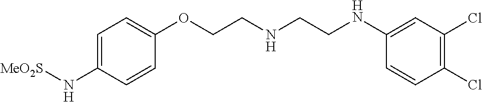 | 14 | 36 |
| 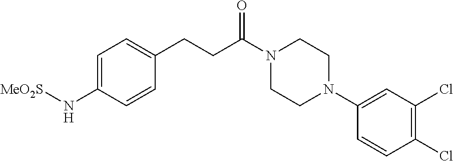 | 670 | 5,330 |
| 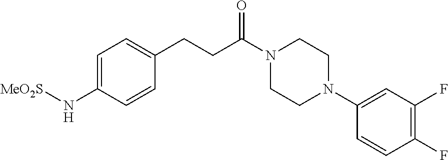 | 2,200 | 8,400 |
| 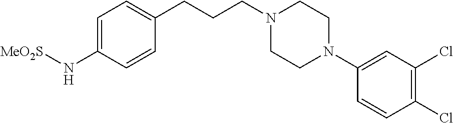 | 230 | 2,730 |
| 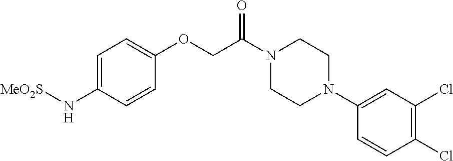 | 40 | 129 |
| 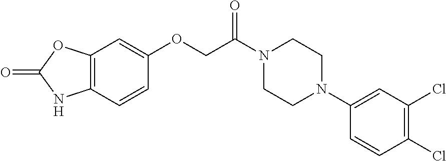 | 41 | 45 |
| 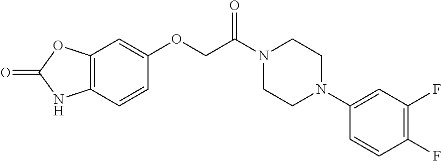 | 307 | 467 |
| 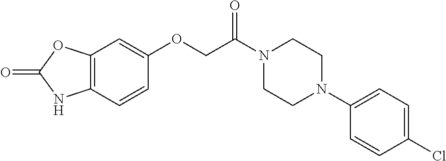 | 189 | 243 |
| 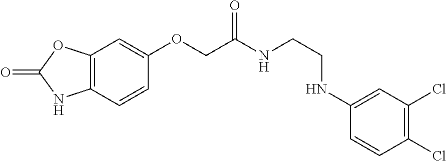 | 80 | 150 |
| 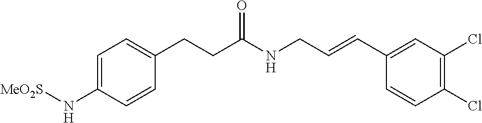 | 10 | 70 |
| 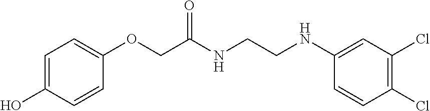 | 280 | 515 |
| 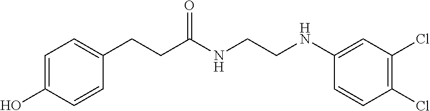 | 210 | 339 |

### Example 11

### In Vitro Binding Studies for Secondary Effects

Compounds were evaluated for binding to the human ether-a-go-go potassium channel (hERG) expressed in HEK293 cells by displacement of ³[H]-astemizole according to the methods by Finlayson et al. (K. Finlayson., L. Turnbull, C. T. January, J. Sharkey, J. S. Kelly; ³[H]Dofetilide binding to HERG transfected membranes: a potential high throughput preclinical screen. Eur. J. Pharmacol. 2001, 430, 147-148). Compounds were incubated at 1 or 10 μM final concentration, in duplicate, and the amount of displaced ³[H]-astemizole determined by liquid scintillation spectroscopy. In some cases, a seven concentration (each concentration in duplicate) displacement curve was generated to determine an IC₅₀.

Binding to the rat alpha-1 adrenergic receptor in rat brain membranes was determined by displacement of ³[H]-prazosin (P. Greengrass and R. Bremner; Binding characteristics of 3H-prazosin to rat brain a-adrenergic receptors. Eur. J. Pharmacol. 1979, 55: 323-326). Compounds were incubated at 0.3 or 3 μM final concentration, in duplicate, and the amount of displaced ³[H]-prazosin determined by liquid scintillation spectroscopy.

Binding IC₅₀ values were determined from displacement curves (four-six concentrations, each concentration in duplicate) fit by a non-linear, least squares, regression analysis using MathIQ (ID Business Solutions Ltd., UK). The binding Ki's were determined from the IC₅₀ according to the method of Cheng and Prusoff (Y. Cheng and W. H. Prusoff; Relationship between the inhibition constant (K1) and the concentration of inhibitor which causes 50 percent inhibition (IC₅₀) of an enzymatic reaction. Biochem. Pharmacol. 1973, 22: 3099-3108).

TABLE 22
NMDA antagonism at pH 6.9 and 7.6 and hERG and α1 adrenergic receptor binding

| | IC₅₀ pH | IC₅₀ pH | hERG | α1 |
|---|---|---|---|---|
| COMPOUND | 6.9 (nM) | 7.6 (nM) | Ki (nM) | IC₅₀ (nM) |
| 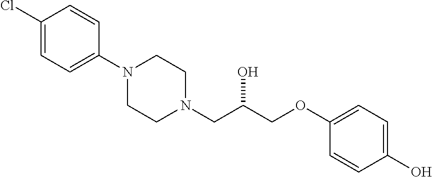 | 74 | 555 | 39,000 | 720 |
| 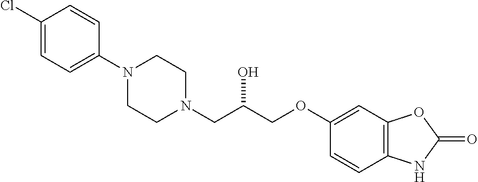 | 51 | 447 | 1,600 | 620 |
| 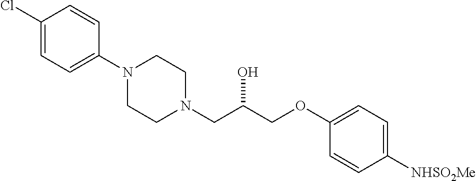 | 32 | 270 | 553 | 350 |
| 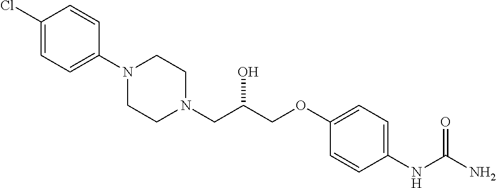 | 46 | 452 | 13,000 | 340 |
| 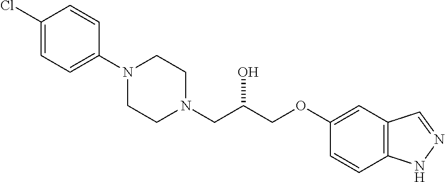 | 410 | 3,830 | ~1,000 | ~300 |
| 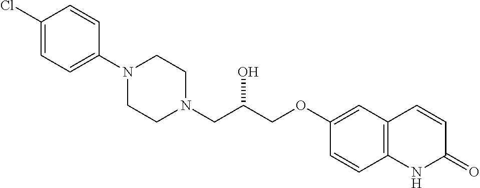 | 69 | 550 | ~7,500 | ~350 |
| 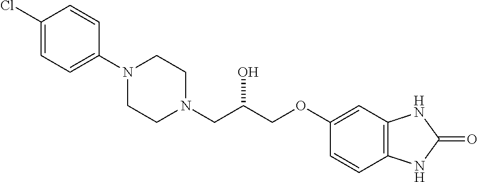 | 14 | 103 | ~7,500 | ~350 |
| 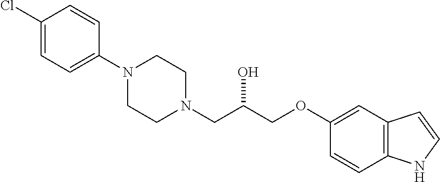 | >10,000 | >10,000 | — | — |
| 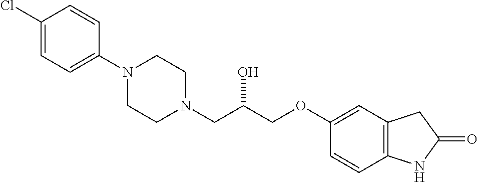 | 370 | 29 | ~10,000 | ~100 |
| 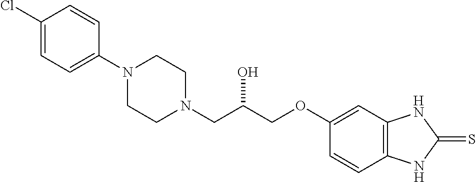 | 16 | 76 | ~7,500 | ~150 |
| 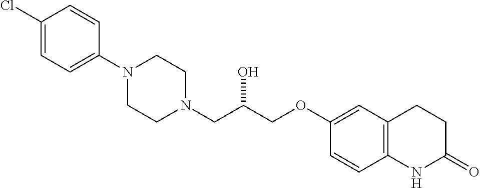 | 12 | 66 | ~5,000 | ~100 |

### Example 12

### Metabolic Stability

Compounds were incubated with pooled human (from at least 10 donors) or rat liver microsomes, 1.0 mg/ml microsomal protein, and 1 mM NADPH, in buffer at 37° C. in a shaking water bath according to the method of Clarke and Jeffrey (S. E. Clarke and P. Jeffrey; Utility of metabolic stability screening: comparison of in vitro and in vivo clearance. Xenobiotica 2001. 31: 591-598). At 60 min the samples were extracted and analyzed for the presence of the parent compound by LC-MS/MS. The parent material remaining in the sample at 60 min was compared to that at 0 min and expressed as a percentage. A control compound, testosterone, was run in parallel.

### Example 13

### Plasma Half-Life and Brain Exposure

Rats (n=3 per dose) were administered compounds at a doses of 1-4 mg/kg in a single bolus i.v. infusion (2 ml/kg body weight) via the tail vein formulated in 2% dimethyl acetamide/98% 2-hydroxy-propyl cyclodextrin (5%). Animals were fasted overnight prior to dose administration and food returned to the animals two hours after dosing. Following IV dosing, blood samples (ca 200 μL) were collected into separate tubes containing anticoagulant (K-EDTA) via the orbital plexus at various times post administration. Plasma samples were prepared immediately after collection by centrifugation for ten minutes using a tabletop centrifuge, and stored at −80° C. Brain tissue was weighed, homogenized on ice in 50 mM phosphate buffer (2 ml per brain) and the homogenate stored at −80° C. Plasma and brain homogenate samples were extracted by the addition of 5 volumes of cold acetonitrile, mixed well by vortexing and centrifuged at 4000 rpm for 15 minutes. The supernatant fractions were analyzed by LC-MS/MS operating in multiple reaction monitoring mode (MRM). The amount of parent compound in each sample was calculated by comparing the response of the analyte in the sample to that of a standard curve.

Penetration Classification (Table 24) using the in vitro cell permeability assay to predict bran penetration potential: Transwell® wells containing MDR1-MDCK cell monolayers that express the multidrug transporter P-gp were used for measuring the percent recovery of compound after dosing both sides of a cell monolayer with the test article. Monolayers were grown for 7-11 days at which time 5 μM of the test article was made by dilution from DMSO stocks into a Hank's balanced salt solution (pH 7.4), final DMSO not greater than 1%, and added to: a) the apical side for A-B permeability (apical to basal) assessment, or separately b) the basal side for the B-A permeability (basal to apical) assessment, all at pH 7.4. After a 2 hr incubation (37° C.) both the apical and the basal compartments were sampled and the amount of test article present determined by generic LC-MS/MS methods against a ≧4 point calibration curve. Experiments were done in duplicate. Apparent permeability (Pₐₚₚ units are reported×10−6 cm/s) are determined for the A−B and the B−A directions as well as the Efflux ratio (PₐₚₚB−A/Pₐₚₚ A−B). The blood-brain barrier penetration potential is classified as follows: “High” when Pₐₚₚ A−B≧3.0×10−6 cm/s, and efflux<3.0; “Moderate” when Pₐₚₚ A−B≧3.0×10−6 cm/s, and 10>efflux≧3.0; and “Low” when either Pₐₚₚ A−B≧3.0×10−6 cm/s, and efflux≧10, or when Pₐₚₚ A−B<3.0×10−6 cm/s.

TABLE 23
Plasma Stability Results

| | | i.v. | AUC (0-last h) | | |
|---|---|---|---|---|---|
| | PK-i.v. | Cmax | i.v. XMPK | | |
| Compound | t1/2 (hr) | ng/ml | (hr * ng/mL) | Dosing | Formulation |
| 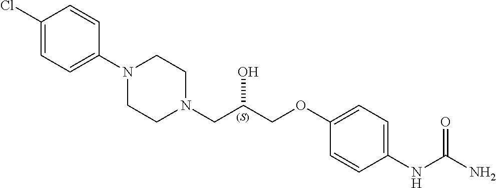 | 2.7 | 252 | 1031* | (a) 1 MPK, 0-4 hr i.v.; | 2% DMA/98% 2- HPBCD (5% in Water) |
| 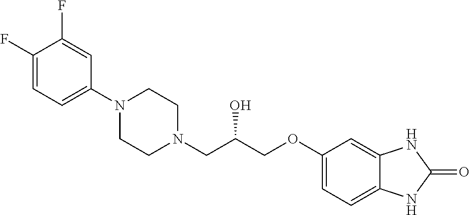 | 2.4 | 241 | 452 | (b) 1 MPK, 0-6 hr i.v.; | 5% DMA/95% 2- HPBCD (5% in Water) |
| 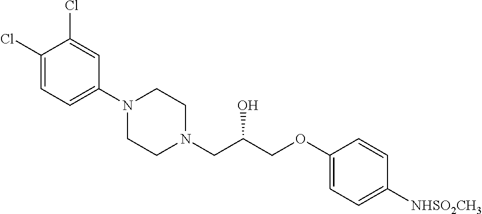 | 2.6 | 1069 | 2351 | (b) 3MPK, 0-4 hr i.v.; | 2% DMA/98% 2- HPBCD (5% in Water) |
| 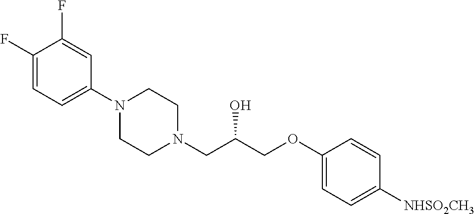 | >2 i.p.** | 13403 (30 m), 13057 (2 hr) | | 60 MPK, i.p. | 5% DMA/95% 2- HPBCD (5% in Water) |
| 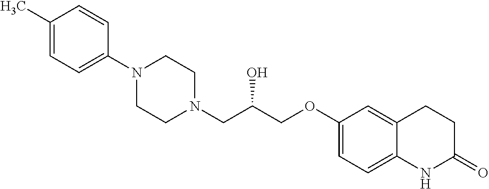 | 0.8 | 464 | 347 | (b) 1 MPK, 0-6 hr i.v.;. | 5% DMA/95% 2- HPBCD (5% in Water) |
| 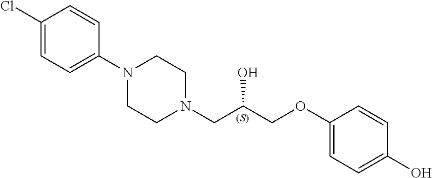 | 0.6 | 193 | 187 | (a) 1 MPK, 0-4 hr i.v.; | 2% DMA/98% 2- HPBCD (5% in Water) |
| 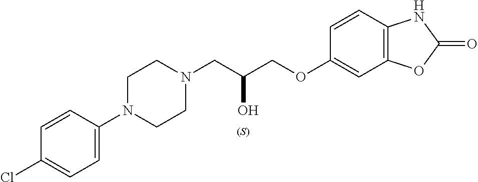 | 0.6 | 288 | 217 | (a) 1 MPK, 0-4 hr i.v. | 2% DMA/98% 2- HPBCD (5% in Water) |
| 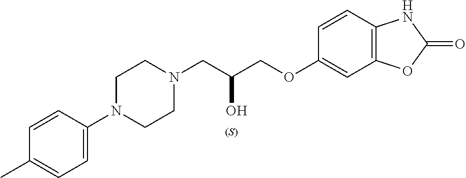 | 0.6 | 568 | 364 | (a) 1 MPK, 0-4 hr i.v.; | 2% DMA/98% 2- HPBCD (5% in Water) |
| 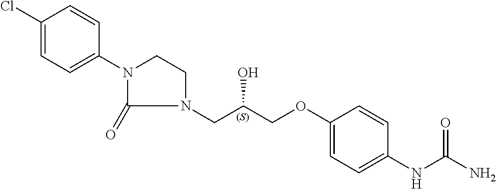 | 1.14 | 715 | 787.4 | (b) 1MPK, 0-6 hr i.v.; 5MPK, 0-6 hr | 10% DMA/10% EtOH/30% 2- HPBCD/50% water |
| 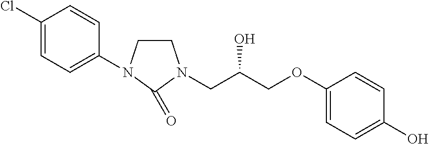 | ≦0.8 hr | 377 (30 m) | | 3 MPK i.v. | 2% DMA/98% 2- HPBCD (5% in Water) |
| 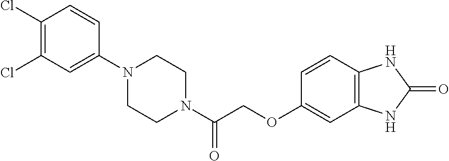 | 1.84 | 575.7 | 1096 | (b) 1MPK, 0-6 hr i.v.; 5MPK, 0-6 hr | 10% DMA/10% EtOH/30% 2- HPBCD/50% water |
| 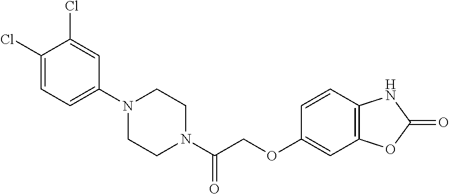 | ~0.83 i.p | 960 (15 m) | | 10 MPK i.p. | 50% DMSO/50% saline |
| 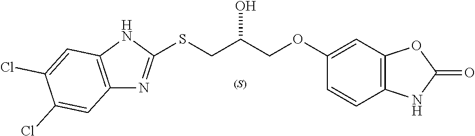 | ≦0.8 hr | 398 (30 m) | | 3 MPK i.v. | 2% DMSO/98% 2- HPBCD (5% in Water) |
| 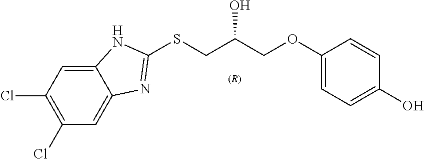 | ~0.5 hr | 413 (30 m) | | 3 MPK i.v. | 2% DMA/98% 2- HPBCD (5% in Water) |

TABLE 24
Brain Penetration

| | RATIO: | | | |
|---|---|---|---|---|
| | Brain:plasma | | | BBB Pene |
| Compound | (Direct) | Dosing | Formulation | Classification |
| 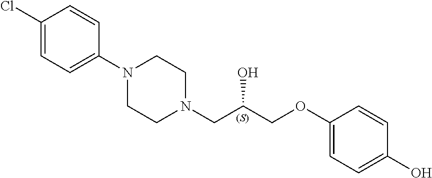 | 10.2 (avg); 11 (30 m), 9.5 (1 hr), | 3 MPK i.v. | 2% DMA/98% 2-HPBCD (5% in Water) | High |
| 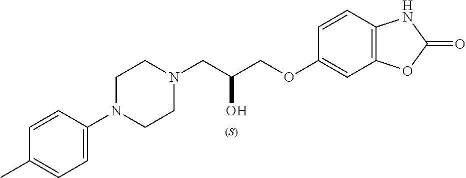 | 2.3 (avg); 2.3 (30 m), 2.9 (1 hr), 1.7 (2 hr) | 3 MPK i.v. | 2% DMA/98% 2-HPBCD (5% in Water) | Moderate |
| 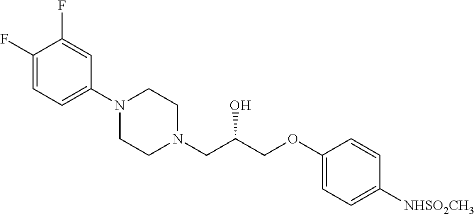 | 0.77 (avg); 0.7 (30 m), 0.7 (2 hr), 0.9 (4 hr) | 60 MPK, i.p. | 5% DMA/95% 2-HPBCD (5% in Water) | Moderate |
| 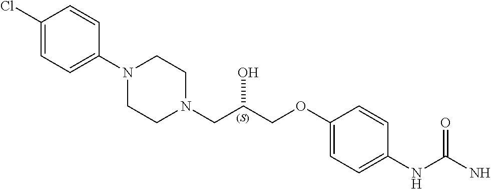 | 0.42 (avg); 0.24 (30 m), 0.53 (1 hr), 0.49 (6 hr) | 3 MPK i.v. @ 6 hr, 1MPK @ 0.5 hr and 2 hr | 2% DMA/98% 2-HPBCD (5% in Water) | Low |
| 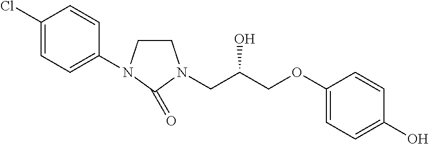 | 0.59 (avg); 0.68 (30 m), 0.49 (1 hr), | 3 MPK i.v. | 2% DMA/98% 2-HPBCD (5% in Water) | High |
| 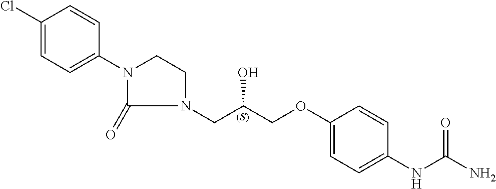 | Below Detection limit (~0) | 1 MPK i.v. | 10% DMA/10% EtOH/30% 2- HPBCD/50% water | Low |
| 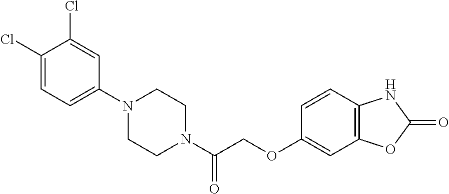 | 0.33 (avg); 0.25 (15 m), 0.49 (30 m), 0.30 (1 hr), 0.36 (2 hr) | 10 MPK i.p. | 50% DMSO/50% saline | Moderate |
| 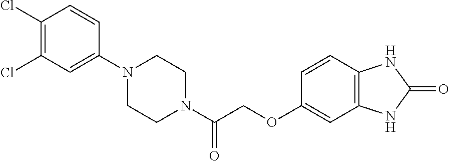 | 0.07 (avg.); 0.04 (15 m), 0.06 (30 m), 0.07 (1 hr), 0.10 (3 hr) | 1 MPG i.v. | 10% DMA/10% EtOH/30% 2- HPBCD/50% water | Moderate |
| 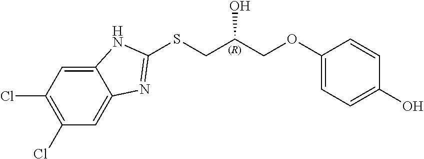 | 0.25 (avg); 0.15 (30 m), 0.35 (1 hr), BLQ (2 hr) | 3 MPK i.v. | 2% DMA/98% 2-HPBCD (5% in Water) | High |
| 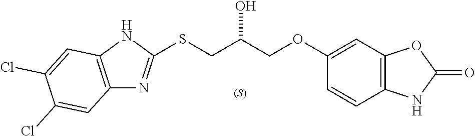 | BLQ (avg); BLQ (30 m), BLQ (1 hr), | 3 MPK i.v. | 2% DMA/98% 2-HPBCD (5% in Water) | Moderate |

TABLE 25
Oral Absorption

| | | | | | AUC | | |
|---|---|---|---|---|---|---|---|
| | % | | p.o. | p.o. | (0-last h) | | |
| | Absorbed | p.o. | Tmax | t1/2 | p.o. | | |
| Compound | (% F) | Cmax | hr | hr | (hr*ng/mL) | Dosing | Formulation |
| 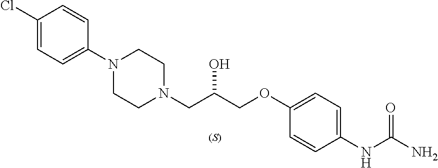 | 145.7** | 1556.6 | 2.67 | 6.46 | 8451 | 10 MPK, 0-8 hr p.o. | 2% DMA/98% 2-HPBCD (5% in Water) |
| 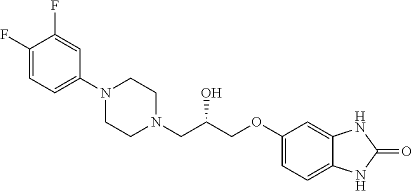 | 82.2 | 558 | 0.5 | 3.04 | 1856 | 5 MPK, 0-6 hr p.o. | 5% DMA/95% 2-HPBCD (5% in Water) |
| 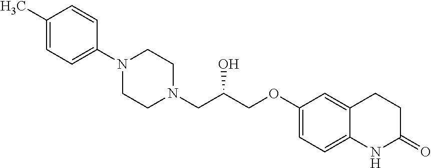 | 31.6 | 407 | 0.33 | 0.85 | 549 | 5 MPK, 0-6 hr p.o. | 5% DMA/95% 2-HPBCD (5% in Water) |
| 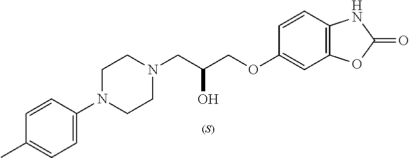 | 7.9** | 57.8 | 6.7 | nc | 284 | 10 MPK, 0-8 hr p.o. | 2% DMA/98% 2-HPBCD (5% in Water) |
| 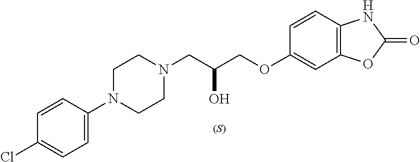 | 6.2** | 30.6 | 8 | nc | 132 | 10 MPK, 0-8 hr p.o. | 2% DMA/98% 2-HPBCD (5% in Water) |
| 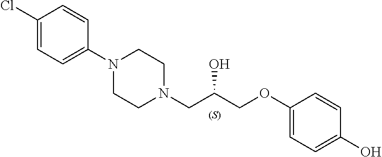 | 3.3** | 10.2 | 5.3 | nc | 56.4 | 10 MPK, 0-8 hr p.o. | 2% DMA/98% 2-HPBCD (5% in Water) |

TABLE 26
Structures of compounds referenced in Examples 14-22.

| Label | Compound |
|---|---|
| 93-31 | 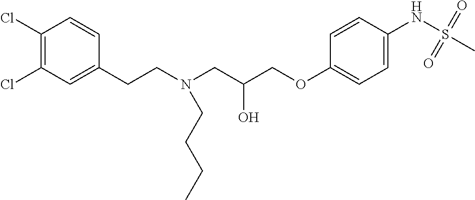 |
| 93-97 | 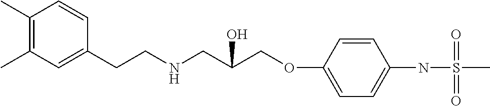 |
| 93-108 | 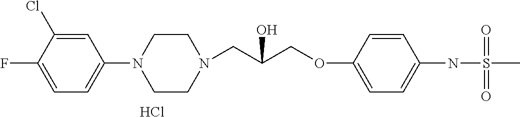 |
| NP10001 | 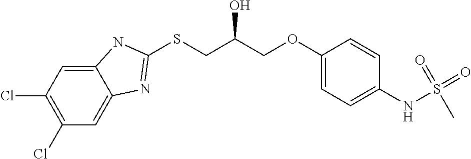 |
| NP10002 | 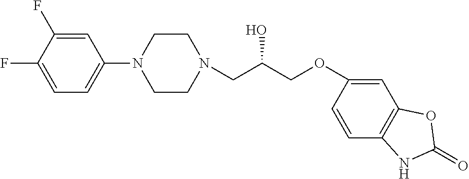 |
| NP10030 | 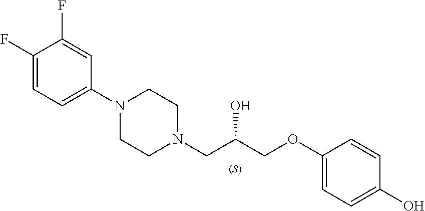 |
| NP10031 | 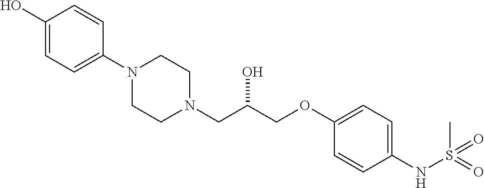 |
| NP10039 | 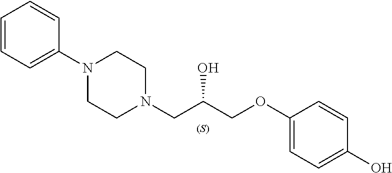 |
| NP10045 | 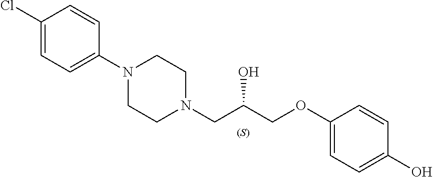 |
| NP10056 | 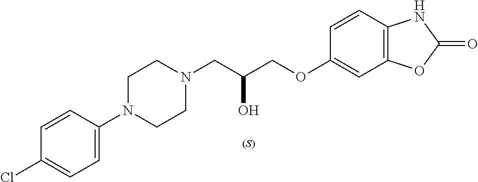 |
| NP10066 | 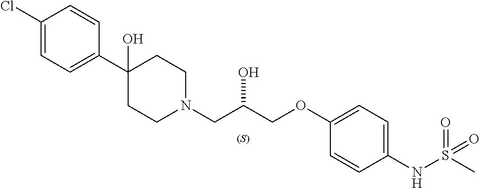 |
| NP10068 | 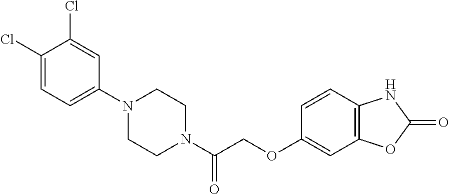 |
| NP10070 | 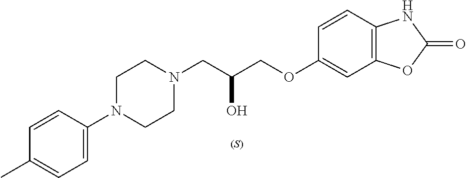 |
| NP10075 | 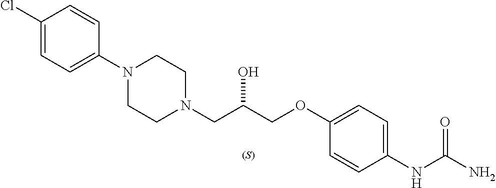 |
| NP10076 | 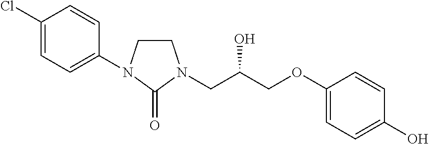 |
| NP10097 | 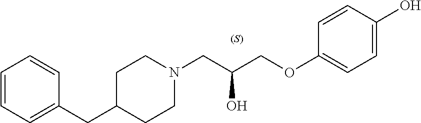 |
| NP10099 | 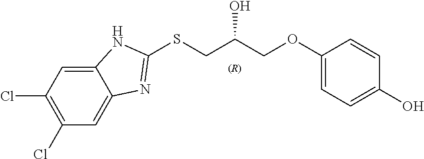 |
| NP10119 | 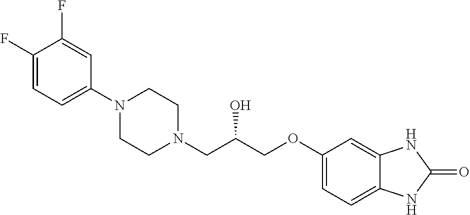 |
| NP10122 | 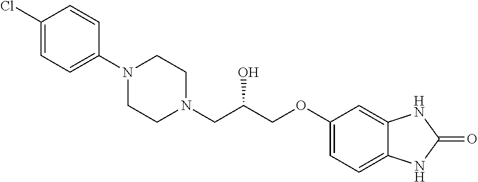 |
| NP10146 | 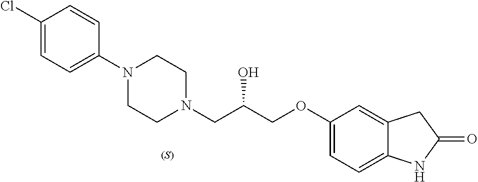 |
| NP10150 | 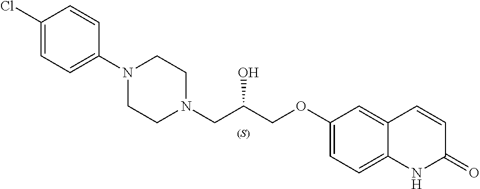 |
| NP10153 | 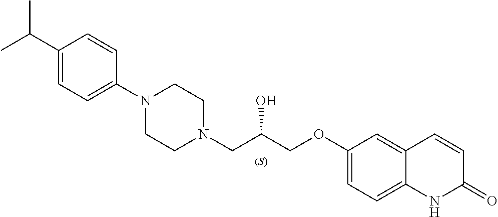 |
| NP10165 | 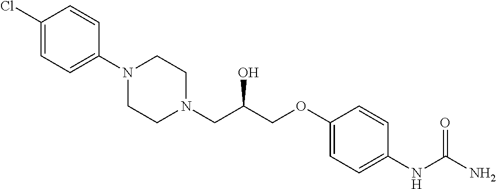 |
| NP10185 | 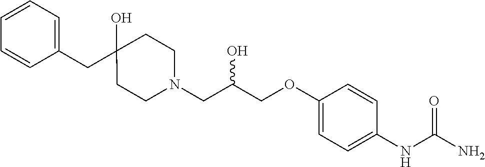 |
| NP10226 | 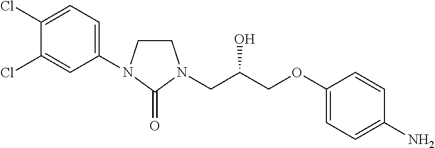 |
| NP10231 | 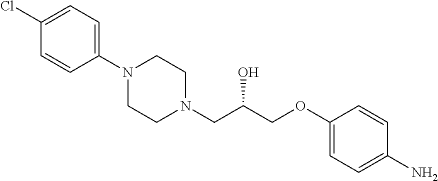 |
| NP10239 | 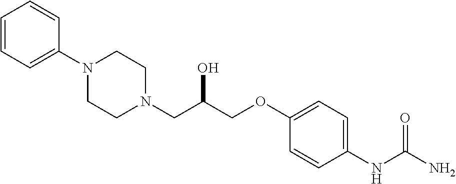 |
| NP10250 | 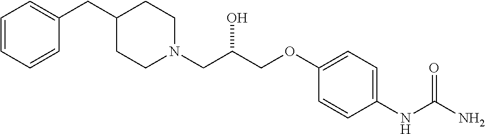 |
| NP10272 | 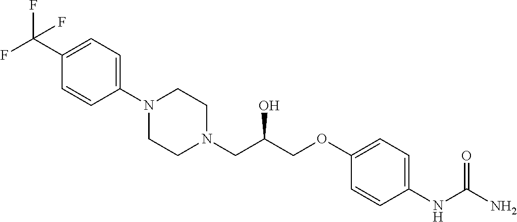 |

### Example 14

### Forced Swim Model

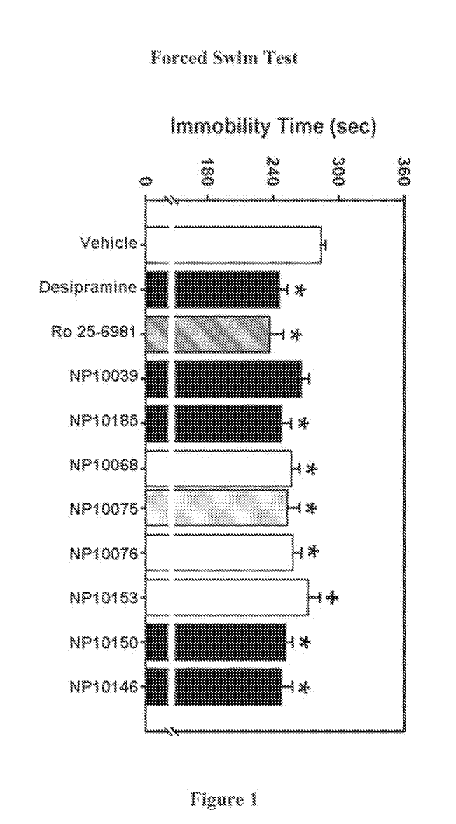

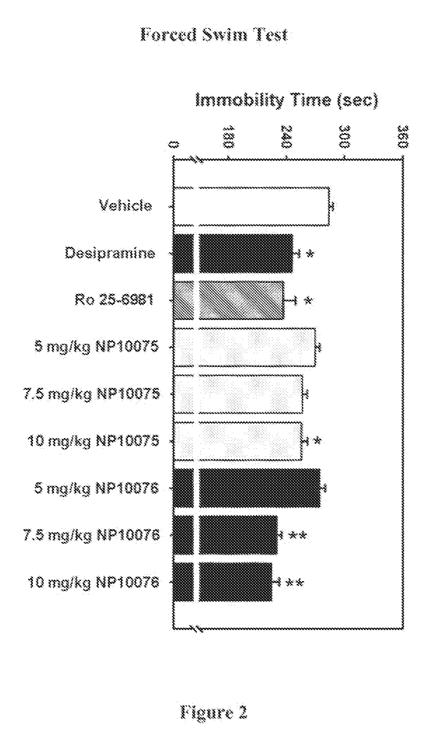

CD1 mice were adminstered a compounds shown in Table 11, desipramine, Ro 25-6981 or a control vehicle and subjected to a forced swim test. All compounds were administered as intraperitoneal injections. Animals were placed into a beaker (15 cm diameter) of water held at 25° C. with a depth of 15 cm 30 min after compound administration. Behavior was videotaped for 6 minutes from the side of the beaker and scored subsequently for struggling behavior. Results were analyzed by one-way ANOVA and post-hoc Bonferroni tests. Immobility time date from the forced swim tests is shown in FIGS. 1 and 2. Total immobility time refers to the time that the animal spends floating or engaged in minimal activity to keep afloat for at least 3 seconds. Subtle movements of feet, tail or head required to maintain the eyes, ears, and nose above the surface of the water were excluded as immobility. The video-tapes were scored by investigators unfamiliar with the treatments of the mice.

For the data in FIG. 1, test compounds were tested at a dose of 10 mg/ kg. Desipramine was tested at a dose of 20 mg/kg. Ro 25-6981 was tested at a dose of 5 mg/kg. Number of CD1 mice tested per groups was 8-10. ANOVA: F(11,98)=3.638, p<0.01. *=p<0.05 compared to vehicle. +=p<0.05 compared to desipramine.

For the data in FIG. 2, compounds NP10075 and NP10076 were tested at doses of 5 mg/kg, 7.5 mg/kg and 10 mg/kg; desipramine was tested at a dose of 20 mg/kg; and Ro 25-6981 was tested at a dose of 5 mg/kg. Number of CD1 mice tested per group was 8-10. *=p<0.05, **=p<0.01 ANOVA, post-hoc Bonferroni from control.

### Example 15

### Open Field Activity Test

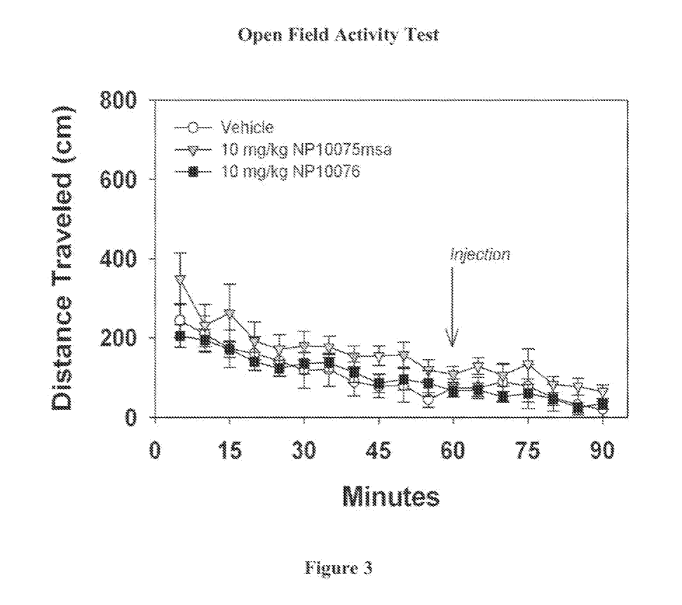

Spontaneous activity was evaluated in an automated Omnitech Digiscan apparatus (AccuScan Instruments, Columbus, Ohio). Animals were given vehicle, imipramine, or a dose of a test compound. All compounds were administered as intraperitoneal injections. Activity was summated at 5 minute intervals over the 90 min period of testing. At 60 minutes, the mice were injected with 10 mg/kg NP10075, 10 mg/kg NP10076 or a vehicle. Locomotion was measured in terms of the total distance traveled (horizontal activity). Results were analyzed by one-way ANOVA and post-hoc Bonferroni tests. Neither NP10075 or NP10076 altered mouse open field activity at a dosage of 10 mg/kg. Data for these tests is shown in FIG. 3.

### Example 16

### Plasma and Brain Exposure Assessment

Mice were administered a dose of test compound (10 mg/kg by i.p.) and blood and brain tissue samples collected at the indicated times post drug administration (n=3-5). Blood samples were collected in K-EDTA tubes and centrifuged for ten minutes immediately after collection, and the plasma was stored at −80° C. until analysis. Brains were immediately removed from the skull and the meninges and cerebellum removed, rinsed with ice-cold PBS, weighed and then homogenized at 4° C. in 2-3 volumes of 50 mM potassium phosphate buffer (pH 7.4) and stored at −80° C. until analysis. Plasma and brain homogenates were extracted by the addition of 5 volumes of cold acetonitrile, mixed well by vortexing and centrifuged at 4000 rpm for 15 minutes. The supernatant fractions were analyzed by LC-MS/MS operating in multiple reaction monitoring mode (MRM) and analyzed for the parent compound to determine the plasma or brain concentration. Internal standards were added to calibrate each sample. An eight point standard curve was prepared similarly in naïve plasma and brain for each compound of interest. Plasma and brain exposure assessment data is provided in Table 27. Based on occupancy studies of other NR2B antagonists in rodents (CP-101,606, Ro25-6981, and Merck20j), plus plasma levels achieved in Preskorn et al., brain exposures for the test compounds were consistent with “anticipated levels” required for efficacy. Results are shown in Table 27.

TABLE 27
Plasma and Brain Drug Concentration

| Depres- | | Rat | | Free | Free | |
|---|---|---|---|---|---|---|
| sion | | IC₅₀ | Plasma* | Plasma** | Plasma | Brain*** |
| Result | Label | (μM) | (ng/ml) | (ng/ml) | (μM) | (μM) |
| active | NP10075 | 0.108 | 3137 | 220 | 0.542 | 0.130 |
| active | NP10076 | 0.072 | 377 | 34 | 0.094 | 0.064 |
| active | NP10068 | 0.060 | 679 | 61 | 0.145 | 0.048 |

*= Plasma sample analysis by LC-MS/MS, in mice dosed 10 mg/kg, i.p., 30 minute time point.
**= Based on % free fraction measured in protein binding study, by LC-MS/MS or NR2B receptor bioassay.
***= Calculated from the brain:plasma ratio for the compounds determined by LC-MS/MS.

### Example 17

### Rotorod Test (In Vivo Safety)

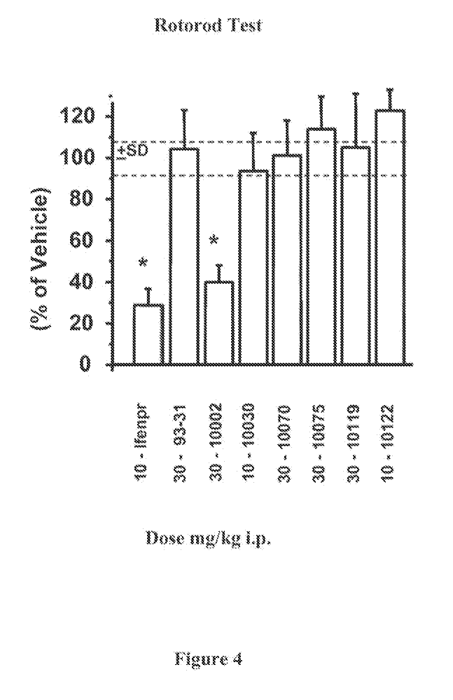

The rotorod test is a modification of the procedure described by Rozas and Labandeira-Garcia (1997). The test is initiated by placing mice on a rotating rod (5 rpm) that is 3.8 cm diameter by 8 cm wide and suspended 30 cm from the floor of a chamber. After 10 sec the rotation is accelerated from 5 to 35 rpm over a 5 minute period. The time the mouse falls from the rod (the latency time) is recorded automatically with a light-activated sensor in the bottom of the chamber. Animals were trained four times each day for two days, with a within-day inter-trial interval of 20-25 min and a between-day interval of 24 hrs. On day 3, mice were randomly assigned to groups and injected in a blinded fashion with either vehicle, positive control (0.3 mg/kg (+)MK−801 or 10 mg/kg ifenprodil), or doses of NP compound. All drugs were administered i.p. Results were analyzed by ANOVA and Dunnett's tests. Data is shown in FIG. 4.

### Example 18

### Cell Toxicity in Cortical Neuron Culture

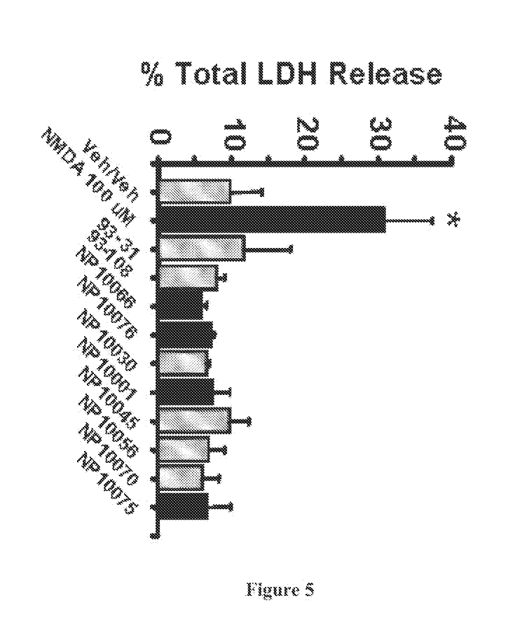

Primary cultures of rat cerebral cortex were prepared from Sprague-Dawley rat embryos (E16-E19). Cells were plated into 24 well plates at a density of 3×105 per well, in Neurobasal medium supplemented with L-glutamine (2 mM), penicillin (5 U/ml), streptomycin (10 μg/ml) and B-27. After 14-22 days in culture, cells were treated with test compounds (in triplicate wells) at 10 μM, final, and incubated for 24 hrs. Cell death was assessed by measuring the amount of lactate dehydrogenase (LDH) released into the culture medium (Tox-7 kit; Sigma Chemical Co, St. Louis, Mo.). Released LDH was expressed as the fraction of total LDH present in each well. Maximal cell death was determined by treating separate wells (in triplicate) with saturating concentrations of NMDA (100 μM) and glycine (10 μM) for 24 hrs. Results shown are the Mean±SEM from a minimum of three separate cultures cell toxicity was assessed by % total LDH release after 24 hr incubation of 10 μM compound in cell culture. For each compound, three cultures were treated with 10 μM compound. Data is shown in FIG. 5.

### Example 19

### Ames Test for Genotoxicity

The Ames test determines the ability of a compound to reverse an introduced mutation in two strains of Salmonella typhimurium (selected from TA98, TA100, TA15345, TA1537, and TA102). (See for example Maron, D. M. and Ames, B. N., Mutat. Res., 1983, 113, 173-215.) Compounds were tested at eight dose levels 1.5, 5, 15, 50, 150, 500, 1500, and 5000 μg/plate in both the presence and absence of S-9 microsomal fraction in two bacterial strains (TA98, TA102). After incubation at 37° the number of revertant colonies was compared with the number of spontaneous revertants on negative (vehicle) plates. Positive control plates containing a known mutagen active in each of the strains in the presence of S-9 extract (2-aminoanthracene at 1-5 ug/plate) were also run. Data is shown in Table 28.

TABLE 28
Genotocity Test Results

| | Conc #, range | +/−S9 | | Valid | |
|---|---|---|---|---|---|
| Label | (μg/plate) | Activation | Strains | Assay | Result |
| 93-31 | 8; 1.5-5000 | Both | TA98, | Yes | No positive |
| | | | TA102 | | response |
| NP10056 | 8; 1.5-5000 | Both | TA98, | Yes | No positive |
| | | | TA102 | | response |
| NP10075 | 8; 1.5-5000 | Both | TA98, | Yes | No positive |
| | | | TA102 | | response |
| NP10122 | 8; 1.5-5000 | Both | TA98, | Yes | No positive |
| | | | TA102 | | response |
| NP10226 | 8; 1.5-5000 | Both | TA98, | Yes | No positive |
| | | | TA102 | | response |
| NP10231 | 8; 1.5-5000 | Both | TA98, | Yes | No positive |
| | | | TA102 | | response |

### Example 20

### hERG Binding

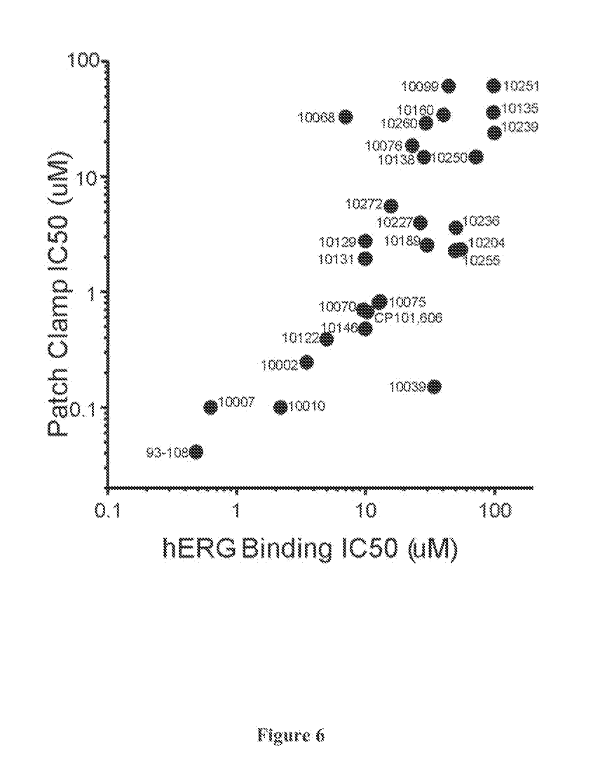

Compounds were evaluated for binding to the human ether-a-go-go potassium channel (hERG) expressed in HEK293 cells by displacement of 3 [H]-astemizole. Binding studies were performed either at a single concentration of 10 uM (in duplicate) or binding IC₅₀ values determined from displacement curves (four-six concentrations, each point in duplicate) fit by a non-linear, least squares, regression analysis using MathIQ (ID Business Solutions Ltd., UK). Data is shown in FIG. 6. Compounds were evaluated for binding to the human ether-a-go-go potassium channel (hERG) expressed in HEK293 cells by displacement of 3[H]-astemizole. Binding studies were performed either at a single concentration of 10 uM (in duplicate) or binding IC₅₀ values determined from displacement curves (four-six concentrations, each point in duplicate) fit by a non-linear, least squares, regression analysis using MathIQ (ID Business Solutions Ltd., UK). Functional hERG channel block was determined using patch clamp methods with stable hERG channel transfectants in HEK293 cells. All experiments were performed at ambient temperature. Each cell acted as its own control. Three to five concentrations of the test article was applied at 5 minute intervals via micropipette tips to cells expressing hERG (n≧3 cells/concentration). Duration of exposure to each test article concentration was 5 minutes. After vehicle application, the positive control was applied in the same manner to verify sensitivity to hERG blockade. Intracellular solution for whole cell recordings consisted of (composition in mM): potassium aspartate, 130; MgCl2, 5; EGTA, 5; ATP, 4; HEPES, 10; pH adjusted to 7.2 with KOH. After establishment of a whole-cell configuration, membrane currents were recorded using QPatch HT® system. Before digitization, the current records were low-pass filtered at one-fifth of the sampling frequency. Onset and block of hERG current was measured using a stimulus voltage pattern consisting of a 200 ms prepulse to −40 mV (leakage subtraction), a 2-second activating pulse to +40 mV, followed by a 2-second test pulse to −40 mV. The pulse pattern was repeated continuously at 10 s intervals, from a holding potential of −80 mV. Peak tail currents were measured during the −40 mV test pulse. Leakage current was calculated from the current amplitude evoked by the prepulse and subtracted from the total membrane current recorded. Data acquisition and analysis was performed using the suite of Assay Software programs (Sophion Bioscience A/S, Denmark). Steady state was defined by the limiting constant rate of change with time (linear time dependence). The steady state before and after test article application was used to calculate the percentage of current inhibited at each concentration. Concentration-response data were fit to the following equation:

% Block={1−1/[1+([Test]/IC50)N]}*100

where [Test] is the concentration of test article, IC50 is the concentration of the test article producing half-maximal inhibition, N is the Hill coefficient, and % Block is the percentage of hERG potassium current inhibited at each concentration of the test article. Data were fit by a nonlinear least squares fits with the Solver add-in for Excel 2000 (Microsoft, Redmond, Wash.). Data is shown in FIG. 6.

### Example 21

### Langendorff Heart Preparation and Measurement of QT Effects

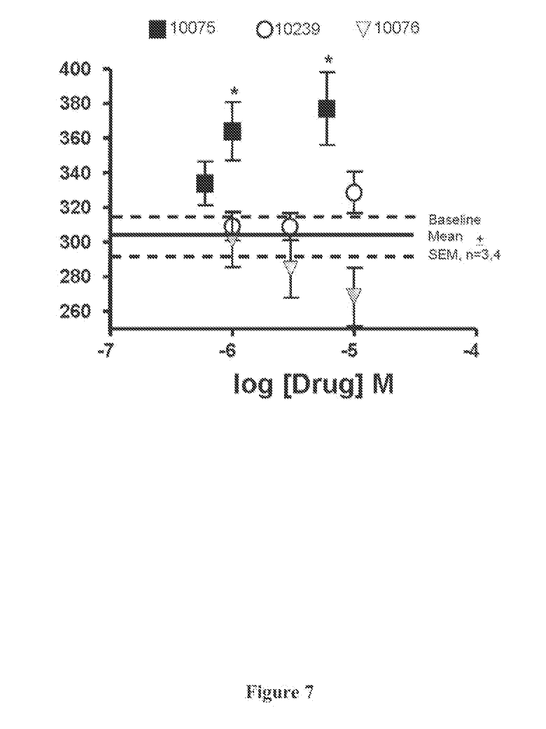

The effects of test compounds on the QT-interval of the electrocardiogram were evaluated in vitro using an isolated retrograde perfused rabbit (New Zealand white female) heart preparation (Langendorff) with an ablated AV node and stimulated at a basic cycle length of 1 s. Test article concentrations were prepared by diluting stock solutions in DMSO into Kreb-Henseleit (KH) solution (composition in mM): NaCl, 129; KCl, 3.7; CaCl₂, 1.3; MgSO4, 0.64; Na-Pyruvate, 2.0; NaHCO3, 17.8; Glucose, 5. The solution was aerated with a mixture of 95% O₂ and 5% CO2 (pH 7.3-7.45). All test solutions contained 0.3% DMSO, final. Briefly, rabbits were heparinized and anesthetized with sodium pentothal and hearts rapidly removed via a midsternal thoracotomy and placed in chilled oxygenated (95% O2+5% CO2) KH solution. The heart was mounted in a Langendorff heart perfusion apparatus and perfused at a constant flow with KH solution (37° C.) in a retrograde fashion through the aorta. The A-V node was ablated to slow the intrinsic heart rate to a ventricular escape less than 60 beats/min. Following immersion of the heart into the bath the volume-conducted ECG was recorded via bath-mounted electrodes. Three Ag/AgCl pellet electrodes were positioned in the bath chamber to form an equilateral triangle centered on the heart. Each heart was paced by repetitive electrical stimuli (0.1-5 ms, approximately 1.5×threshold) by a pulse generator. The ECG signals were conditioned by an AC-coupled preamplifier (Grass Model P511) with low-pass filtering to achieve a bandwidth of 10-300 Hz. A stabilization period was at least 30 minutes long before obtaining baseline control responses. Test article concentrations were applied sequentially, in ascending order for exposure periods of at least 15 minutes/concentration to allow equilibration with the tissue. The average responses from at least three hearts were analyzed for each test condition. The QT interval was calculated and the Mean±SEM values from the last four beats in the equilibration period were measured. Test results are shown in FIG. 7.

### Example 22

### PCP Discrimination Test

Development of N-methyl-D-aspartate (NMDA) antagonists for a variety of disorders has been hindered by their production of phencyclidine (PCP)-like psychological effects and abuse potential. Drug discrimination studies allow direct comparisons to be made among the discriminative stimulus effects of drugs (Balster, 1990; Holtzman, 1990) and are considered to be predictive of subjective effects in humans. Sprague-Dawley rats were trained to discriminate 2 mg/kg (i.p.) PCP or saline when administered intraperitoneally 15 min before the session under a double alternation schedule. Rats were placed in the operant chambers and the session initiated, as signaled by illumination of the chamber houselight. Completion of a FR32 on the correct lever resulted in delivery of a 45-mg food pellet (PJ Noyes Company, Inc., Lancaster, N.H., USA). Incorrect responding reset the FR for correct-lever responding. Training was continued until the animals respond reliably and complete the first FR with more than 80% of total responses on the correct lever during a minimum of four consecutive sessions. Subsequent to acquisition of the PCP-saline discrimination, test sessions commenced on when animals met the following criteria on the most recent PCP and saline training sessions: (i) first FR completed on the correct lever, and (ii) greater than 85% correct-lever responding over the entire session. The animals were tested with different doses of test drug (as shown), generally given in an ascending order across test days. Various doses of PCP and test compounds were administered intraperitoneally 15 min before session initiation. To demonstrate the degree of stimulus control, tests with 2 mg/kg PCP and saline were carried out before and after each dose-response curve. In addition vehicle was also tested. Between test sessions, animals continued to train with PCP and saline injections. Illumination of lights, recording of responses and pellet delivery was controlled by a microcomputer using MEDPC software (Med Associates). For data analysis the mean (±SE) percentage PCP-lever responding and response rate (resp/s) effects was evaluated for all test sessions. Full substitution for PCP required greater than 80% PCP-lever responding, partial substitution as producing between 20 and 80% PCP-lever responding, and less than 20% PCP-lever responding will be indicative of a lack of PCP-like discriminative stimulus effects. Additionally, the mean response rate for all animals during each test session was determined to reveal any nonspecific effects on behavior.

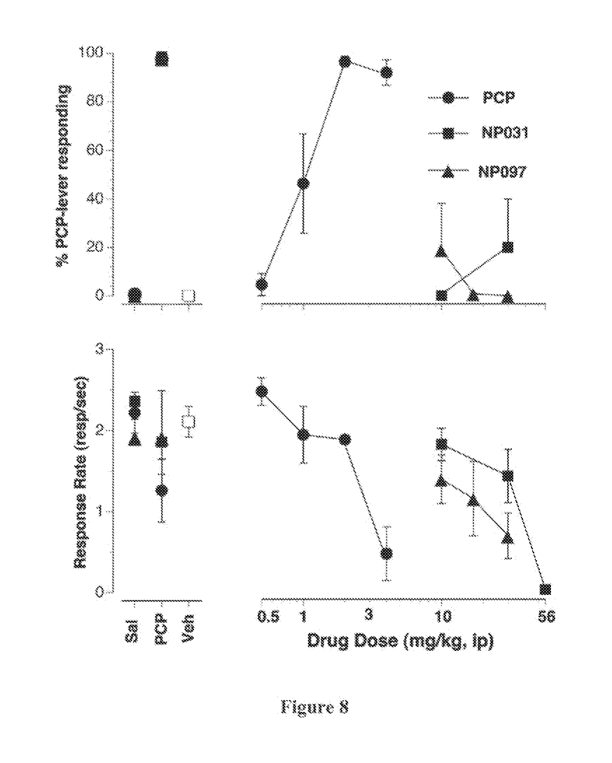

Data for 93-31 (NP031) and 93-97 (NP097) compared to PCP are shown in FIG. 8.

We claim:

1. A method of treatment or prophylaxis of a neuropsychiatric disorders comprising administering a compound of Formula I or II, or a pharmaceutically acceptable salt, ester, prodrug or derivative thereof to a host in need thereof: 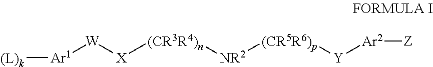
wherein:
each L is independently C₁-C₆ alkyl, C₁-C₆ alkoxy, C(═O)—(C₁-C₆)-alkyl, C₁-C₆ haloalkyl, alkaryl, hydroxy, —O-alkyl, —O-aryl, —SH, —S-alkyl, —S-aryl, fluoro, chloro, bromo, iodo, nitro, or cyano; or two L groups may be taken together with Ar¹ to form:
a dioxolane ring or a cyclobutane ring;
k=0, 1, 2, 3, 4 or 5;
each Ar¹ and Ar² is independently aryl or heteroaryl;
W is a bond, C₁-C₄ alkyl, or C₂-C₄ alkenyl;
X is a bond, NR¹ or O wherein each R¹ and R² is independently H, C₁-C₆ alkyl, C₂-C₆ alkenyl or C₆-C₁₂ aralkyl; or R¹ and R² can be taken together to form a 5-8 membered ring;
each R³ and R⁴ is independently H, C₁-C₆ alkyl, C₁-C₆ alkoxy, C(═O)—(C₁-C₆)-alkyl, C₁-C₆ haloalkyl, hydroxy, fluoro, chloro, bromo, iodo, nitro, or cyano; or CR³R⁴ is C═O;
n and p are independently 1, 2, 3 or 4;
each R⁵ and R⁶ is independently H, C₁-C₆ alkyl, C₁-C₆ alkoxy, C(═O)—(C₁-C₆)-alkyl, C₁-C₆ haloalkyl, hydroxy, fluoro, chloro, bromo, iodo, nitro, or cyano; or CR⁵R⁶ is
C═O or C═CH₂; or wherein —NR²—(CR⁵R⁶)ₚ— can be 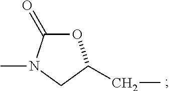
Y is a bond, O, S, SO, SO₂, CH₂, NH, N(C₁-C₆ alkyl), or NHC(═O);
Z is OH, NR⁶R⁷, NR⁸SO₂(C₁-C₆ alkyl), NR⁸C(O)NR⁶R⁷, NR⁸C(S)NR⁶R⁷, NR⁸C(O)O(C₁-C₆ alkyl), NR⁸-dihydrothiazole, or NR⁸-dihydroimidazole; wherein
each R⁶, R⁷ and R⁸ is independently H, C₁-C₆ alkyl or C₆-C₁₂ aralkyl; or 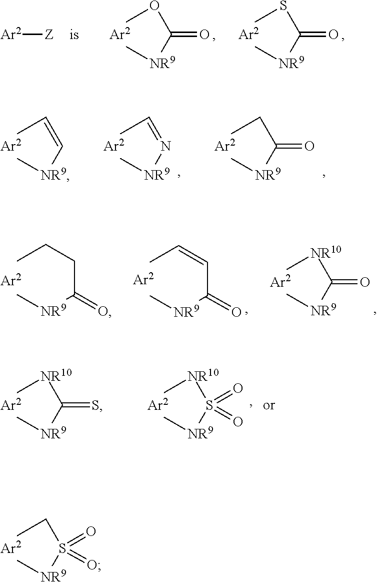
wherein R⁹ and R¹⁰ are each independently H, C₁-C₆ alkyl, aralkyl; or 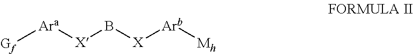
wherein:
each G is independently F, Cl, Br, I, C₁-C₄ alkyl, C₁-C₄ alkoxy, C₆-C₁₂ aralkyl, —O-aryl, —S-aryl, —NH-aryl;
f=0, 1, 2, 3, 4 or 5;
Ar^{a} and Ar^{b} are each independently aryl or heteroaryl;
B is selected from the group consisting of: 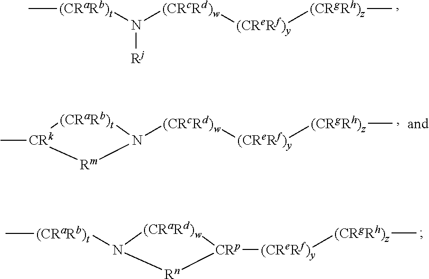
wherein R^{a}, R^{b}, R^{c}, R^{d}, R^{e}, R^{f}, R^{g}, R^{h}, R^{k} and R^{p} are each independently selected from H, C₁-C₆ alkyl, C₁-C₆ alkoxy, OH or halo;
R^{j} is H, C₁-C₆ alkyl, OH or P(O)(OC₁-C₄ alkyl)₂;
R^{m} is C₁-C₄ alkyl or C₂-C₄ alkenyl;
Rⁿ is C₁-C₄ alkyl, C₂-C₄ alkenyl, C₆-C₁₂ aralkyl, —CH₂O—, —CH(C₁-C₆ alkyl)O—, —CH(C₂-C₁₂ aralkyl)O—;
t, w, y and z each=0, 1, 2, or 3;
X and X′ are independently selected from a bond, O, S, SO, SO₂, CH₂, NH, N(C₁-C₆ alkyl), and NHC(═O);
M is OH, F, Cl, Br, I, NH₂, NR^{q}R^{r}, NO₂, O(C₁-C₆ alkyl), OCF₃, CN, C(O)OH, C(O)O(C₁-C₆ alkyl), C₆-C₁₂ aralkyl, NR^{s}C(O)CR^{t}₃, NR⁸SO₂(C₁-C₆ alkyl), or NR^{u}C(O)NR^{v}₂, wherein each R^{q}, R^{r}, R^{s}, R^{u} and R^{v} is each independently H or C₁-C₆ alkyl; and each R^{t} is independently H, C₁-C₆ alkyl or halo; or two M groups may be taken together with Ar^{b} to form: 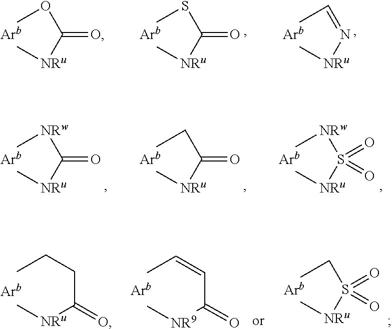
and wherein R^{a} and R^{w} are independently H, C₁-C₆ alkyl or C₆-C₁₂ aralkyl; and
h=1, 2, 3, 4 or 5.

2. The method of claim 1 wherein the compound is of Formula II and each G is independently F, Cl, Br, I and f=0, 1 or 2;
B is 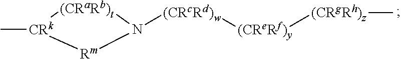
wherein R^{a-e, g, h, k, p} are each from H and R^{f} is selected from H, OH or halo;
R^{m} is C₁-C₄ alkyl or C₂-C₄ alkenyl;
t, w, y and z each=0, 1, 2, or 3;
X and X′ are independently selected from a bond, O, S, CH₂, and NH;
M is OH, F, Cl, Br, I, NH₂, NR^{q}R^{r}, NO₂, O(C₁-C₆ alkyl), OCF₃, CN, C(O)OH, C(O)O(C₁-C₆ alkyl), C₆-C₁₂ aralkyl, NR^{s}C(O)CR^{t}₃, NR⁸SO₂(C₁-C₆ alkyl), or NR^{u}C(O)NR^{v}₂; wherein each R^{q}, R^{r}, R^{s}, R^{u} and R^{v} is each independently H or C₁-C₆ alkyl; and each R^{t} is independently H, C₁-C₆ alkyl or halo; or two M groups may be taken together with Ar^{b} to form:

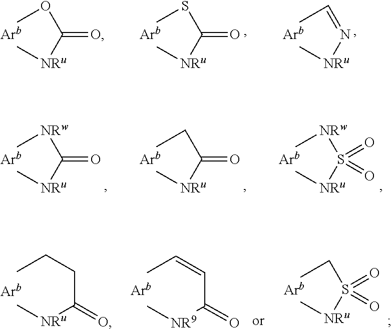

3. The method of claim 1, wherein the compound is a compound of Formula A: 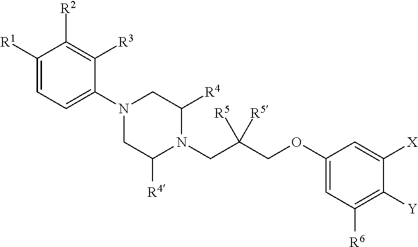
wherein:
R¹ is H, F, Cl, Br, CF₃, C₁₋₆ alkyl, C(O)CH₃, C(O)CO—(C₁₋₆ alkyl), CH₂OH, CN, NH₂, N(C₁-C₆ alkyl)₂, OH, O—(C₁₋₆ alkyl), OCF₃, S—(C₁₋₆ alkyl), SO₂—(C₁₋₆ alkyl);
R² is H, F, Cl, methyl, CF₃;
R³ is H, F, Cl, CH₃, CF₃, CN;
each of R⁴ and R^{4′} are independently selected from H or methyl;
each of R⁵ and R^{5′} can be H or OH, or R⁵ and R^{5′} can be taken together to form ═CH₂;
R⁶ is H or F;
X is H or F;
Y is OH, NHSO₂R⁷, or NHC(O)NHR⁸;
R⁷ is C₁₋₆ alkyl, C₆₋₁₂ aryl, or C₇₋₁₃ aralkyl;
R⁸ is H, C₁₋₆ alkyl, C₆₋₁₂ aryl, or C₇₋₁₃ aralkyl;
or X and Y are taken together to form a heterocycle wherein the moiety 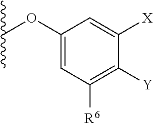
is selected from the group consisting of:

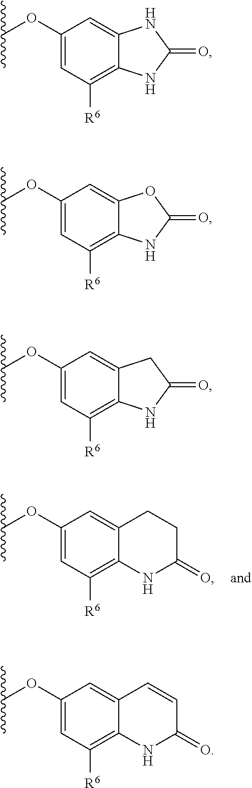

4. The method of claim 1, wherein the compound is a compound of Formula B: 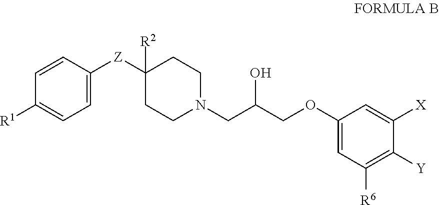
wherein:
R¹ is H, F, Cl, Br, CF₃, or C₁₋₆ alkyl;
Z is O, S, NH, CH₂ or a bond;
R² is H or OH;
R⁶ is H or F;
X is H or F;
Y is OH, NHSO₂R⁷ or NHC(O)NHR⁸;
R⁷ is C₁₋₆ alkyl, C₆₋₁₂ aryl, or C₇-C₁₃ aralkyl;
R⁸ is H, C₁₋₆ alkyl, C₆₋₁₂ aryl, or C₇₋₁₃ aralkyl;
or X and Y are taken together to form a heterocycle wherein the moeity 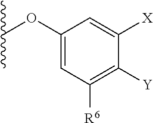
is selected from the group consisting of:

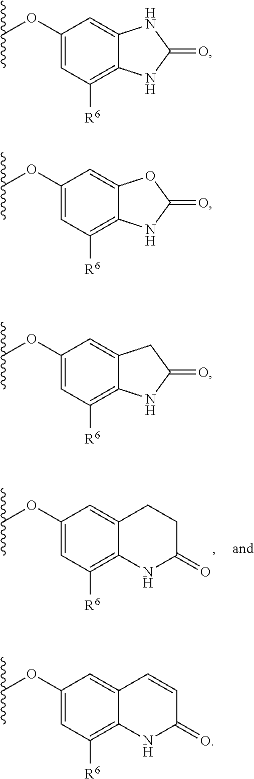

5. The method of claim 1 wherein the disorder is depression.

6. The method of claim 4 wherein the host has been diagnosed with a major depression.

7. The method of claim 1 wherein the compound is administered to a host at risk of suffering from a depressive episode.

8. The method of claim 1 wherein the compound is administered in combination with a pharmaceutically acceptable carrier.

9. The method of claim 1 wherein the compound is administered in combination or alternation with a second active agent.
